(19) United States
(12) Patent Application Publication
(10) Pub. No.: US 2023/0108783 A1
(43) Pub. Date: Apr. 6, 2023

(54) COMPOUNDS AND METHODS FOR THE TREATMENT OF DUCHENNE MUSCULAR DYSTROPHY

(71) Applicant: DTX PHARMA, INC., San Diego (US)

(72) Inventors: Arthur T. Suckow, San Diego, CA (US); Charles Allerson, San Diego, CA (US); Fabio C. Tucci, San Diego, CA (US)

(21) Appl. No.: 17/778,380

(22) PCT Filed: Nov. 25, 2020
(86) PCT No.: PCT/US2020/062333

§ 371 Date: May 19, 2022

Related U.S. Application Data
(60) Provisional application No. 62/941,549, filed on Nov. 27, 2019.

(51) Int. Cl. C07C 229/00; C07H 21/02; C07H 21/04; A61P 21/00

(52) U.S. Cl. CPC C07C 229/00; C07H 21/02; C07H 21/04; A61P 21/00

(57) ABSTRACT

Disclosed herein are compounds including a single-stranded oligonucleotide (A) having a nucleobase sequence complementary to a portion of the dystrophin pre-mRNA, their preparation, and uses thereof for the treatment of Duchenne muscular dystrophy.

### CROSS-REFERENCES TO RELATED APPLICATIONS

This application claims the benefit of U.S. Provisional Patent Application No. 62/941,549, filed Nov. 27, 2019, which is incorporated herein in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file DTX-004-01WO_ST25.TXT, created on Nov. 20, 2020, 46,377 bytes in size, machine format IBM-PC, MS Windows operating system, is hereby incorporated by reference.

### BACKGROUND

### Field

The present disclosure relates to the field of biologically active compounds including a single-stranded oligonucleotide. More specifically, the present disclosure relates to compounds including a single-stranded oligonucleotide, which are targeted to the dystrophin gene, and their use for the treatment of Duchenne muscular dystrophy.

### Background

Duchenne muscular dystrophy (DMD) is an X-linked, fatal muscle disease affecting roughly one in 5,000 newborn boys (Fairclough et al., 2013, Nat Rev Genet., 14: 373-378) and is caused by mutations in the dystrophin gene (Hoffman et al., 1987, Cell, 51: 919-928). Loss of functional dystrophin protein results primarily from frameshift mutations in the gene. Dystrophin is a crucial component of the dystrophin-associated glycoprotein complex (DGC), which connects the sarcolemma and extracellular matrix to the actin cytoskeleton, thereby maintaining muscle cell membrane integrity during contractions. Without functional dystrophin, DMD patients display skeletal muscle weakness that begins in early childhood. As patients age, they develop progressive loss of muscle mass, spinal curvature (kyphosis), paralysis and typically don't survive beyond age 30, succumbing to cardiac and respiratory complications of severe muscle weakness (Matsumura et al., 2011, Rinsho Shinkeigaku, 51: 743-750). Despite knowing the genetic cause for several decades, there is currently no cure for DMD and approved therapies are of limited efficacy (Landfeldt et al., 2014, Neurology, 83: 529-536).

A therapeutic strategy that is of considerable interest employs a type of splice-switching oligonucleotides, an exon-skipping oligonucleotide, to restore at least a partial level of dystrophin protein. Exon-skipping antisense oligonucleotides are designed to hybridize to and mask the splicing signals of a selected exon, preventing its inclusion in the final mRNA (i.e., induce ‘skipping’ of the exon) and leading to production of an in-frame, albeit shorter but functional protein. The targeted exon is chosen based on the nature of the mutation and resultant translation product, e.g. a mutation that results in a translation of a premature stop codon. In the case of dystrophin, an exon-skipping oligonucleotide targeted to a chosen splice signal of the dystrophin pre-mRNA results in the skipping of the exon and the translation of a shorter but at least partially functional dystrophin protein.

The splice alteration strategy appears to be applicable to a large proportion of DMD patients (˜83%) (Aartsma et al., 2009, Hum Mut., 2009, 30(3): 293-299), and there are several clinical development programs evaluating exon-skipping oligonucleotides specific to different mutations of the dystrophin gene. One such program by Sarepta Therapeutics resulted in the first exon-skipping oligonucleotide to be approved for the treatment of DMD, EXONDYS 51™, for DMD patients whose dystrophin gene contains a mutation amenable to exon 51 skipping. EXONDYS 51™ hybridizes to exon 51 of the dystrophin pre-mRNA, resulting in exclusion of exon 51 during mRNA processing and translation of an internally truncated dystrophin protein. While EXONDYS 51™ has been found to increase dystrophin protein levels in the muscle of DMD patients, a clinical benefit has yet to be sufficiently established. Other clinical programs based on exon-skipping oligonucleotides are ongoing. Despite the activity in this therapeutic area, no exon-skipping oligonucleotide has to date shown evidence of clinical benefit. Additionally, EXONDYS 51™ and other exon-skipping oligonucleotides are limited by cellular uptake and biodistribution challenges. Thus, there remains a need for exon-skipping oligonucleotides targeted to dystrophin that have a meaningful impact on disease progression in DMD patient.

### BRIEF SUMMARY

Provided herein are, inter alia, compounds, or compounds including a single-stranded oligonucleotide (A) covalently bonded to an uptake motif, wherein the single-stranded oligonucleotide targeted to the dystrophin pre-mRNA.

In embodiments, the uptake motif has the structure:

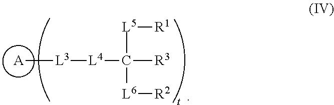

t is an integer from 1 to 5.

A is a single-stranded oligonucleotide having a nucleobase sequence complementary to the dystrophin pre-mRNA. L³ and L⁴ are independently a bond, —NH—, —O—, —S—, —C(O)—, —NHC(O)—, —NHC(O)NH—, —C(O)O—, —OC(O)—, —C(O)NH—, —OPO₂—O—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. L⁵ is -L^{5A}-L^{5B}-L^{5C}-L^{5D}-L^{5E}-. L⁶ is -L^{6A}-L^{6B}-L^{6C}-L^{6D}-L^{6E}-.

L^{5A}, L^{5B}, L^{5C}, L^{5D}, L^{5E}, L^{6A}, L^{6B}, L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

R¹ and R² are independently unsubstituted C₁-C₂₅ alkyl, wherein at least one of R¹ and R² is unsubstituted C₉-C₁₉ alkyl. R³ is hydrogen, —NH₂, —OH, —SH, —C(O)H, —C(O)NH₂, —NHC(O)H, —NHC(O)OH, —NHC(O)NH₂, —C(O) OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In an aspect, provided is a method including contacting a cell with the compound, or the compound including a single-stranded oligonucleotide (A), as described herein.

In an aspect, provided is a method of inducing exon skipping of the dystrophin pre-mRNA in a cell including contacting the cell with the compound, or the compound including a (A), as described herein.

In an aspect, provided is a method including administering to a subject the compound, or the compound including a single-stranded oligonucleotide (A), as described herein.

In an aspect, provided is a method including administering a subject the compound, or the compound including single-stranded oligonucleotide (A), as described herein. The method includes administering to a subject who has a mutation in the dystrophin gene amenable to exon skipping.

In an aspect, provided is a compound, or the compound including a single-stranded oligonucleotide (A) as described herein, for use in therapy.

In an aspect, provided is a method of introducing a single-stranded oligonucleotide into a cell within a subject. The method includes administering to said subject the compound including a single-stranded oligonucleotide (A) as described herein.

In an aspect, provided is a pharmaceutical composition including a pharmaceutically acceptable excipient and the compound including a single-stranded oligonucleotide (A) as described herein.

Other aspects are disclosed infra.

### DETAILED DESCRIPTION

### Definitions

Unless defined otherwise, all technical terms, scientific terms, abbreviations, chemical structures, and chemical formulae used herein have the same meaning as is commonly understood by one of ordinary skill in the art. The chemical structures and formulae set forth herein are constructed according to the standard rules of chemical valency known in the chemical arts. All patents, applications, published applications, and other publications referenced herein are incorporated by reference in their entirety unless stated otherwise. Unless otherwise indicated, conventional methods of mass spectroscopy, NMR, HPLC, protein chemistry, biochemistry, recombinant DNA techniques, and pharmacology are employed. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3 dioxanyl, 1,3 dioxolanyl, 1,3 dithiolanyl, 1,3 dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1 dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3 dihydrobenzofuran 2 yl, 2,3 dihydrobenzofuran 3 yl, indolin 1 yl, indolin 2 yl, indolin 3 yl, 2,3 dihydrobenzothien 2 yl, decahydroquinolinyl, decahydroisoquinolinyl, octahydro 1H indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, S, Si, or P), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol [symbol] denotes the point of attachment of a chemical moiety to the [symbol] remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

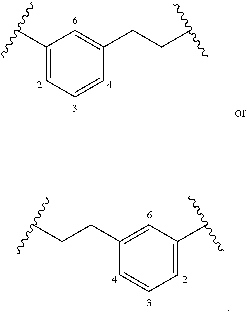 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH2, —NHC(O)NH2, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
   (i) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr2, —OCHI₂, —OCH₂F, —OCH₂Cl, —OCH₂Br, —OCH₂I, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl, heteroalkyl, cycloalkyl, heterocycloalkyl, aryl, heteroaryl, substituted with at least one substituent selected from:
      (a) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(b) alkyl, heteroalkyl, cycloalkyl, heterocycloalkyl, aryl, heteroaryl, substituted with at least one substituent selected from: oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₅ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In embodiments of the compounds herein, each substituted or unsubstituted alkyl may be a substituted (e.g., substituted with a substituent group, a size-limited substituent group, or lower substituent group) or unsubstituted C₁-C₂₀ alkyl, each substituted or unsubstituted heteroalkyl is a substituted (e.g., substituted with a substituent group, a size-limited substituent group, or lower substituent group) or unsubstituted 2 to 20 membered heteroalkyl, each substituted or unsubstituted cycloalkyl is a substituted (e.g., substituted with a substituent group, a size-limited substituent group, or lower substituent group) or unsubstituted C₃-C₈ cycloalkyl, each substituted or unsubstituted heterocycloalkyl is a substituted (e.g., substituted with a substituent group, a size-limited substituent group, or lower substituent group) or unsubstituted 3 to 8 membered heterocycloalkyl, each or unsubstituted aryl is a substituted (e.g., substituted with a substituent group, a size-limited substituent group, or lower substituent group) or unsubstituted C₆-C₁₀ aryl, and/or each substituted or unsubstituted heteroaryl is a substituted (e.g., substituted with a substituent group, a size-limited substituent group, or lower substituent group) or unsubstituted 5 to 10 membered heteroaryl. In embodiments herein, each substituted or unsubstituted alkylene is a substituted (e.g., substituted with a substituent group, a size-limited substituent group, or lower substituent group) or unsubstituted C₁-C₂₀ alkylene, each substituted or unsubstituted heteroalkylene is a substituted (e.g., substituted with a substituent group, a size-limited substituent group, or lower substituent group) or unsubstituted 2 to 20 membered heteroalkylene, each substituted or unsubstituted cycloalkylene is a substituted (e.g., substituted with a substituent group, a size-limited substituent group, or lower substituent group) or unsubstituted C₃-C₈ cycloalkylene, each substituted or unsubstituted heterocycloalkylene is a substituted (e.g., substituted with a substituent group, a size-limited substituent group, or lower substituent group) or unsubstituted 3 to 8 membered heterocycloalkylene, each substituted or unsubstituted arylene is a substituted (e.g., substituted with a substituent group, a size-limited substituent group, or lower substituent group) or unsubstituted C₆-C₁₀ arylene, and/or each substituted or unsubstituted heteroarylene is a substituted (e.g., substituted with a substituent group, a size-limited substituent group, or lower substituent group) or unsubstituted 5 to 10 membered heteroarylene.

In embodiments, each substituted or unsubstituted alkyl is a substituted (e.g., substituted with a substituent group, a size-limited substituent group, or lower substituent group) or unsubstituted C₁-C₈ alkyl, each substituted or unsubstituted heteroalkyl is a substituted (e.g., substituted with a substituent group, a size-limited substituent group, or lower substituent group) or unsubstituted 2 to 8 membered heteroalkyl, each substituted or unsubstituted cycloalkyl is a substituted (e.g., substituted with a substituent group, a size-limited substituent group, or lower substituent group) or unsubstituted C₃-C₇ cycloalkyl, each substituted or unsubstituted heterocycloalkyl is a substituted (e.g., substituted with a substituent group, a size-limited substituent group, or lower substituent group) or unsubstituted 3 to 7 membered heterocycloalkyl, each substituted or unsubstituted aryl is a substituted (e.g., substituted with a substituent group, a size-limited substituent group, or lower substituent group) or unsubstituted C₆-C₁₀ aryl, and/or each substituted or unsubstituted heteroaryl is a substituted (e.g., substituted with a substituent group, a size-limited substituent group, or lower substituent group) or unsubstituted 5 to 9 membered heteroaryl. In embodiments, each substituted or unsubstituted alkylene is a substituted (e.g., substituted with a substituent group, a size-limited substituent group, or lower substituent group) or unsubstituted C₁-C₈ alkylene, each substituted or unsubstituted heteroalkylene is a substituted (e.g., substituted with a substituent group, a size-limited substituent group, or lower substituent group) or unsubstituted 2 to 8 membered heteroalkylene, each substituted or unsubstituted cycloalkylene is a substituted (e.g., substituted with a substituent group, a size-limited substituent group, or lower substituent group) or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted (e.g., substituted with a substituent group, a size-limited substituent group, or lower substituent group) or unsubstituted C₆-C₁₀ arylene, and/or each substituted or unsubstituted heteroarylene is a substituted (e.g., substituted with a substituent group, a size-limited substituent group, or lower substituent group) or unsubstituted 5 to 9 membered heteroarylene. In embodiments, the compound is a chemical species set forth in the Examples section, figures, or tables below.

Certain compounds provided herein possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of provided herein do not include those that are known in art to be too unstable to synthesize and/or isolate. Compounds provided herein include those in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds provided herein may exist in tautomeric forms, all such tautomeric forms of the compounds being within the scope of the present disclosure.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Unless otherwise stated, structures depicted herein are also meant to include all stereochemical forms of the structure; i.e., the (R) and (S) configurations for each asymmetric center. Therefore, single stereochemical isomers as well as enantiomeric and diastereomeric mixtures of the present compounds, generally recognized as stable by those skilled in the art, are within the scope of the present disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, replacement of fluoride by ¹⁸F, or the replacement of a carbon by ¹³C- or ¹⁴C-enriched carbon are within the scope of the present disclosure.

The compounds provided herein may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds provided herein, whether radioactive or not, are included within the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman decimal symbol may be used to distinguish each appearance of that particular R group. For example, where multiple R¹³ substituents are present, each R¹³ substituent may be distinguished as R^{13.1}, R^{13.2}, R^{13.3}, R^{13.4}, etc., wherein each of R^{13.1}, R^{13.2}, R^{13.3}, R^{13.4}, etc. is defined within the scope of the definition of R¹³ and optionally differently. The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Description of compounds of provided herein is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

“Contacting” is used in accordance with its plain ordinary meaning and refers to the process of allowing at least two distinct species (e.g. chemical compounds, biomolecules or cells) to become sufficiently proximal to react, interact or physically touch. For example, contacting includes the process of allowing a compound to become sufficiently proximal to a cell to bind to a cell-surface receptor.

As used herein, “contacting a cell” refers to a condition in which a compound or other composition of matter is in direct contact with a cell, or is close enough to induce a desired biological effect in a cell.

As defined herein, the term “inhibition”, “inhibit”, “inhibiting” and the like mean negatively affecting (e.g. decreasing) activity or function relative to the activity or function in the absence of the inhibitor. In embodiments inhibition means negatively affecting (e.g. decreasing) the concentration or levels of a biomolecule, such as a protein or mRNA, relative to the concentration or level of the biomolecule in the absence of the inhibitor. For example, inhibition includes decreasing the level of mRNA expression in a cell. In embodiments, inhibition refers to a reduction in the activity of a particular biomolecule target, such as a protein target or an mRNA target. Thus, inhibition includes, at least in part, partially or totally blocking stimulation, decreasing, preventing, or delaying activation, or inactivating, desensitizing, or down-regulating signal transduction or enzymatic activity or the amount of a biomolecule. In embodiments, inhibition refers to a reduction of activity of a target biomolecule resulting from a direct interaction (e.g. an inhibitor binds to a target protein). In embodiments, inhibition refers to a reduction of activity of a target biomolecule from an indirect interaction (e.g. an inhibitor binds to a protein that activates a target protein, thereby preventing target protein activation).

The term “inhibitor” also refers to a compound, composition, or substance capable of detectably decreasing the expression or activity of a given gene or protein. For example, an inhibitor may decrease expression or activity 10%, 20%, 30%, 40%, 50%, 60%, 70%, 80%, 90% or more in comparison to a control in the absence of the inhibitor. Inhibitors include, for example, synthetic or biological molecules, such as oligonucleotides.

The terms “expression” and “gene expression” as used herein refer to the steps involved in the translation of a nucleic acid into a protein, including mRNA expression and protein expression. Expression can be detected using conventional techniques for detecting nucleic acids or proteins (e.g., PCR, ELISA, Southern blotting, Western blotting, flow cytometry, FISH, immunofluorescence, immunohistochemistry).

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist.

The term “in vivo” used herein means a process that takes place within a subject's body.

The term “subject” used herein means a human or non-human animal selected for treatment or therapy. In embodiments, a subject is a human.

The term “ex vivo” used herein means a process that takes place in vitro in isolated tissue or cells where the treated tissue or cells comprise primary cells. As is known in the art, any medium used in this process can be aqueous and non-toxic so as not to render the tissue or cells non-viable. In embodiments, the ex vivo process takes place in vitro using primary cells.

The term “administration” means providing a pharmaceutical agent or composition to a subject, and includes administration performed by a medical professional and self-administration.

The term “therapy” means the application of one or more specific procedures used for the amelioration of at least one indicator or a disease or condition. In embodiments, the specific procedure is the administration of one or more pharmaceutical agents.

The term “modulate” is used herein in its ordinary sense as understood by a person of ordinary skill in the art, and thus refers to the act of changing or varying one or more properties. For example, in the context of a modulator's effects on a target molecule, to modulate means to change by increasing or decreasing a property or function of the target molecule or the amount of the target molecule. A modulator of a disease decreases a symptom, cause, or characteristic of the targeted disease.

The term “nucleic acid” means compounds containing at least two nucleotide monomers covalently linked together. Nucleic acids include polynucleotides and oligonucleotides, including double-stranded oligonucleotides and single-stranded oligonucleotides, and modified versions thereof.

The term “polynucleotide” means a longer length nucleic acid, e.g., 200, 300, 500, 1000, 2000, 3000, 5000, 7000, or 10,000 nucleotides in length. Non-limiting examples of polynucleotides include a gene, a gene fragment, an exon, an intron, intergenic DNA (including, without limitation, heterochromatic DNA), messenger RNA (mRNA), a long non-coding RNA, transfer RNA, ribosomal RNA, a ribozyme, cDNA, a recombinant polynucleotide, a branched polynucleotide, a plasmid, a vector, isolated DNA of a sequence, and an isolated RNA of a sequence. Polynucleotides useful in the methods of the disclosure may include natural nucleic acid sequences and variants thereof, artificial nucleic acid sequences, or a combination of such sequences.

The term “oligonucleotide” means a shorter length nucleic acid, e.g. of less than 100 nucleotides in length. Oligonucleotides may be single-stranded or double-stranded. An oligonucleotide may comprise naturally occurring ribonucleotides, naturally occurring deoxyribonucleotides, and/or nucleotides having one or more modifications to a naturally occurring terminus, sugar, nucleobase, and/or internucleotide linkage. Non-limiting examples of oligonucleotides include double-stranded oligonucleotides, single-stranded oligonucleotides, antisense oligonucleotides, small interfering RNA (siRNA), microRNA mimics, short hairpin RNAs (shRNA), single-strand small interfering RNA (ssRNAi), RNaseH oligonucleotides, anti-microRNA oligonucleotides, steric blocking oligonucleotides, exon-skipping oligonucleotides, CRISPR guide RNAs, and aptamers.

The term “single-stranded oligonucleotide” means an oligonucleotide that is not hybridized to a complementary strand. Non-limiting examples of single-stranded oligonucleotides include single-strand small interfering RNA (ssRNAi), RNaseH oligonucleotides (oligonucleotides chemically modified to elicit RNaseH-mediated degradation of a target RNA), anti-microRNA oligonucleotides (oligonucleotides complementary to microRNAs), steric blocking oligonucleotides (oligonucleotides that interfere with target RNA activity without degrading the target RNA), exon-skipping oligonucleotides (oligonucleotides that hybridized to an exon annealing site and alter splicing), CRISPR guide RNAs, and aptamers.

The term “hybridize” means the annealing of one nucleic acid to another nucleic acid based on nucleobase sequence complementarity. In embodiments, an antisense strand is hybridized to a sense strand. In embodiments, an antisense strand hybridizes to a target mRNA sequence.

The term “complementary” means nucleobases having the capacity to pair non-covalently via hydrogen bonding.

The term “fully complementary” means each nucleobase of a first nucleic acid is complementary to each nucleobase of a second nucleic acid. In embodiments, an antisense strand is fully complementary to its target mRNA. In embodiments, a sense strand and an antisense strand of a double-stranded oligonucleotide are fully complementary over their entire lengths. In embodiments, a sense strand and an antisense strand of double-stranded oligonucleotide are fully complementary over the entire length of the double-stranded region of the siRNA, and one or both termini of either strand comprises single-stranded nucleotides.

The term “nucleoside” means a monomer of a nucleobase and a pentofuranosyl sugar (e.g., either ribose or deoxyribose). Nucleosides may be modified at the nucleobase and/or and the sugar. In embodiments, a nucleoside is a deoxyribonucleoside. In embodiments, a nucleoside is a ribonucleoside.

The term “nucleotide” means a nucleoside covalently linked to a phosphate group at the 5′-carbon of the pentafuranosyl sugar. Nucleotides may be modified at one or more of the nucleobase, sugar, or phosphate group. A nucleotide may have a ligand attached, either directly or through a linker. In embodiments, a nucleotide is a deoxyribonucleotide. In embodiments, a nucleotide is a ribonucleotide.

The term “nucleobase” means the heterocyclic base moiety of a nucleoside or nucleotide. Non-limiting examples of nucleobases includes cytosine or a derivative thereof (e.g., cytosine analogue), guanine or a derivative thereof (e.g., guanine analogue), adenine or a derivative thereof (e.g., adenine analogue), thymine or a derivative thereof (e.g., thymine analogue), uracil or a derivative thereof (e.g., uracil analogue), hypoxanthine or a derivative thereof (e.g, hypoxanthine analogue), xanthine or a derivative thereof (e.g., xanthine analogue), 7-methylguanine or a derivative thereof (e.g., 7-methylguanine analogue), deaza-adenine or a derivative thereof (e.g., deaza-adenine analogue), deaza-guanine or a derivative thereof (e.g., deaza-guanine), deaza-hypoxanthine or a derivative thereof, 5,6-dihydrouracil or a derivative thereof (e.g., 5,6-dihydrouracil analogue), 5-methylcytosine or a derivative thereof (e.g., 5-methylcytosine analogue), or 5-hydroxymethylcytosine or a derivative thereof (e.g., 5-hydroxymethylcytosine analogue) moieties. In embodiments, the nucleobase is adenine, guanine, hypoxanthine, xanthine, theobromine, caffeine, uric acid, or isoguanine, which may be optionally substituted or modified. In embodiments, the nucleobase is

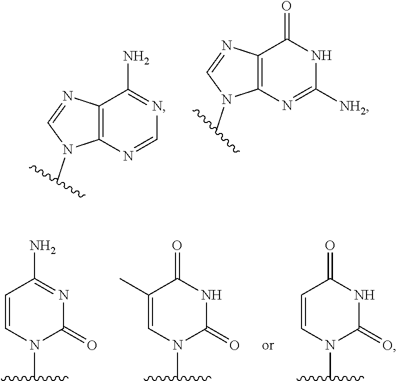

which may be optionally substituted or modified.

The term “modified nucleotide” means a nucleotide having one or more modifications relative to a naturally occurring nucleotide. A modification may be present in an internucleoside linkage, a nucleobase, and/or a sugar moiety of the nucleotide. A modified nucleotide may be selected over an unmodified form because of desirable properties such as, for example, enhanced cellular uptake, enhanced affinity for other oligonucleotides or nucleic acid targets, increased stability in the presence of nucleases, and/or reduced immune stimulation. A modified nucleotide may have a modified sugar moiety and an unmodified phosphate group. A modified nucleotide may have an unmodified sugar moiety and a modified phosphate group. A modified nucleotide may have a modified sugar moiety and an unmodified nucleobase. A modified nucleotide may have a modified sugar moiety and a modified phosphate group. Nucleic acids, polynucleotides and oligonucleotides may comprise one or more modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that participate in nucleobase-pairing (i.e., about 60% complementarity, preferably 65%, 70%, 75%, 80%, 85%, 90%, 91%, 92%, 93%, 94%, 95%, 96%, 97%, 98%, 99%, or higher complementarity over a specified region).

“Hybridize” shall mean the annealing of one single-stranded nucleic acid (such as a primer) to another nucleic acid based on the well-understood principle of sequence complementarity. In an embodiment the other nucleic acid is a single-stranded nucleic acid. The propensity for hybridization between nucleic acids depends on the temperature and ionic strength of their miliu, the length of the nucleic acids and the degree of complementarity. The effect of these parameters on hybridization is described in, for example, Sambrook J, Fritsch EF, Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith.

The terms “identical” or percent “identity,” in the context of two or more nucleic acids or polypeptide sequences, refer to two or more sequences or subsequences that are the same or have a specified percentage of amino acid residues or nucleotides that are the same (i.e., at least 60% identity, or at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or within a range defined by any of two of the preceding values, identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). This definition also refers to, or may be applied to, the complement of a test sequence. The definition also includes sequences that have deletions and/or additions, as well as those that have substitutions. As described below, the preferred algorithms can account for gaps, insertions and the like. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The term “dystrophin pre-mRNA” means the dystrophin transcript containing exons 1 through 79, and the flanking introns. The human dystrophin pre-mRNA is encoded by the gene defined by the NCBI Reference Sequence NG_012232.1, created on Jul. 17, 2019.

The term “amenable to exon skipping” means one or more mutations in the dystrophin gene which causes the reading frame to be out-of-frame, thereby disrupting translation of the pre-mRNA leading to an inability to produce functional or semi-functional dystrophin. Inducing exon skipping of the dystrophin pre-mRNA containing such mutations restores the reading frame, allowing for the production of functional or semi-functional dystrophin. Determining whether a subject has a mutation amenable to exon skipping may be made through a standard diagnostic workup for Duchenne muscular dystrophy (see, for example Bello et al., 2016, Neurology, 87:401-409).

The term “annealing site” means the region of dystrophin pre-mRNA to which the nucleobase sequence of an oligonucleotide is complementary. An annealing site is described by the notation S #A/D (+/−x: +/−y), where “S” represents the species, “#” represents the exon number, “A” indicates splice acceptor site, “D” indicates splice donor site, “x” and “y” indicate the annealing coordinates, a “+” indicates an exonic nucleotide position, and a “−” indicates an intronic nucleotide position. As an example of an annealing site including a splice acceptor site, A(−3+22) indicates the last three nucleotides of the intron preceding the named exon and the first 22 bases of the named exon. As an example of an annealing site including a splice donor site, D(+5-20) indicated the last five nucleotides of the named exon and the first 20 nucleotides of the intron following the named exon. For example, the annealing site H51A(+66+95) indicates the site between the 66^{th} and 95^{th} nucleotide from the start of exon 51 of human dystrophin pre-mRNA. Information regarding the location of introns and exons within the dystrophin gene may be found in the Ensembl database, under gene record ENSG00000198947.

The term “intron” means the nucleotide sequence flanking the coding sequences of a gene. Introns have two distinct nucleotides at either end. At the 5′ end the DNA nucleotides are “GT” (GU in the pre-mRNA), and at the 3′ end they are “AG”. These nucleotides are part of the splicing sites. Each intron contains three sites that are necessary for splicing to occur: a splice donor site, a splice acceptor site, and a splicing branch point.

The term “exon” means the coding sequence of a gene.

The term “splice donor site” means the splicing site at the 5′ end of an intron, with the sequence “NGT in DNA or “NGU” in a pre-mRNA (where “N” is A, C, G, T, or U), with splicing occurring before the “G.”

The term “splice acceptor site” means the splicing site at the 3′ end of an intron, with the sequence “NAGNN” (where “N” is A, C, G, T, or U), with splicing occurring after the “G.” The “AG” sequence indicates the end of the intron.

The term “exonic splicing enhancer” or “ESE” means a non-splice site to which SR proteins bind to promote exonic splice site recognition.

The term “splicing branch point” means the nucleotide of an intron that participates in splicing by promoting the formation of a branched RNA lariat.

### Compounds

In an aspect, the compound has a formula (IV)

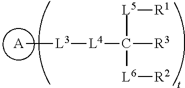

wherein t is an integer from 1 to 5.

In embodiments, t is 1. In embodiments, t is 2. In embodiments, t is 3. In embodiments, t is 4. In embodiments, t is 5.

A is a single-stranded oligonucleotide having a nucleobase sequence complementary to the dystrophin pre-mRNA.

L³ and L⁴ are independently a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. Each R²³, R²⁴ and R²⁵ is independently hydrogen or unsubstituted C₁-C₁₀ alkyl.

L⁵ is -L^{5A}-L^{5B}-L^{5C}-L^{5D}-L^{5E}- and L⁶ is -L^{6A}-L^{6B}-L^{6C}-L^{6D}-L^{6E}-. L^{5A}, L^{5B}, L^{5C}, L^{5D}, L^{5E}, L^{6A}, L^{6B}, L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

R¹ and R² are independently unsubstituted C₁-C₂₅ alkyl, wherein at least one of R¹ and R² is unsubstituted C₉-C₁₉ alkyl. In embodiments, R¹ and R² are independently unsubstituted C₁-C₂₀ alkyl, wherein at least one of R¹ and R² is unsubstituted C₉-C₁₉ alkyl.

R³ is

hydrogen, —NH₂, —OH, —SH, —C(O)H, —C(O)NH₂, —NHC(O)H, —NHC(O)OH, —NHC(O)NH₂, —C(O) OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, one L³ is attached to a 3′ carbon of the single-stranded oligonucleotide at the 3′ end.

In embodiments, one L³ is attached to a 3′ nitrogen of the single-stranded oligonucleotide (e.g., the 3′ nitrogen of a morpholino moiety) at the 3′ end of the single-stranded oligonucleotide.

In embodiments, one L³ is attached to a 5′ carbon of the single-stranded oligonucleotide at the 5′ end.

In embodiments, one L³ is attached to a 6′ carbon of the single-stranded oligonucleotide (e.g., the 6′ carbon of a morpholino moiety) at the 5′ end.

In embodiments, one L³ is attached to a 2′ carbon of the single-stranded oligonucleotide. In embodiments, one L³ is attached to a 2′ carbon of the single-stranded oligonucleotide at the 5′ end. In embodiments, one L³ is attached to a 2′ carbon of the single-stranded oligonucleotide at the 3′ end.

In embodiments, one L³ is attached to a nucleobase of the single-stranded oligonucleotide. In embodiments, one L³ is attached to a nucleobase of the single-stranded oligonucleotide at the of 3′ end. In embodiments, one L³ is attached to a nucleobase of the single-stranded oligonucleotide at the 5′ end.

In embodiments, L³ is a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L³ is a bond. In embodiments, L³ is —N(R²³)—. In embodiments, L³ is —O— or —S—. In embodiments, L³ is —C(O)—. In embodiments, L³ is —N(R²³)C(O)— or —C(O)N(R²⁴)—. In embodiments, L³ is —N(R²³)C(O)N(R²⁴)—. In embodiments, L³ is —C(O)O— or —OC(O)—. In embodiments, L³ is —N(R²³)C(O)O— or —OC(O)N(R²⁴)—. In embodiments, L³ is —OPO₂—O—, —O—P(O)(S)—O—, —O—P(O)(R²⁵)—O—, —O—P(O)(NR²³R²⁴)—N—, or O—P(O)(NR²³R²⁴)—O—. In embodiments, L³ is —P(O)(NR²³R²⁴)—N—, —P(S)(NR²³R²⁴)—N—, —P(O)(NR²³R²⁴)—O— or —P(S)(NR²³R²⁴)—O—. In embodiments, L³ is —S—S—.

In embodiments, L³ is independently substituted or unsubstituted alkylene (e.g., C₁-C₂₃, C₁-C₁₂, C₁-C₅, C₁-C₆, C₁-C₄, or C₁-C₂). In embodiments, L³ is independently substituted alkylene (e.g., C₁-C₂₃, C₁-C₁₂, C₁-C₅, C₁-C₆, C₁-C₄, or C₁-C₂). In embodiments, L³ is independently unsubstituted alkylene (e.g., C₁-C₂₃, C₁-C₁₂, C₁-C₈, C₁-C₆, C₁-C₄, or C₁-C₂). In embodiments, L³ is independently substituted or unsubstituted C₁-C₂₃ alkylene. In embodiments, L³ is independently substituted C₁-C₂₃ alkylene. In embodiments, L³ is independently unsubstituted C₁-C₂₃ alkylene. In embodiments, L³ is independently substituted or unsubstituted C₁-C₁₂ alkylene. In embodiments, L³ is independently substituted C₁-C₁₂ alkylene. In embodiments, L³ is independently unsubstituted C₁-C₁₂ alkylene. In embodiments, L³ is independently substituted or unsubstituted C₁-C₅ alkylene. In embodiments, L³ is independently substituted C₁-C₅ alkylene. In embodiments, L³ is independently unsubstituted C₁-C₈ alkylene. In embodiments, L³ is independently substituted or unsubstituted C₁-C₆ alkylene. In embodiments, L³ is independently substituted C₁-C₆ alkylene. In embodiments, L³ is independently unsubstituted C₁-C₆ alkylene. In embodiments, L³ is independently substituted or unsubstituted C₁-C₄ alkylene. In embodiments, L³ is independently substituted C₁-C₄ alkylene. In embodiments, L³ is independently unsubstituted C₁-C₄ alkylene. In embodiments, L³ is independently substituted or unsubstituted ethylene. In embodiments, L³ is independently substituted ethylene. In embodiments, L³ is independently unsubstituted ethylene. In embodiments, L³ is independently substituted or unsubstituted methylene. In embodiments, L³ is independently substituted methylene. In embodiments, L³ is independently unsubstituted methylene.

In embodiments, L³ is independently substituted or unsubstituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L³ is independently substituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L³ is independently unsubstituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L³ is independently substituted or unsubstituted 2 to 23 membered heteroalkylene. In embodiments, L³ is independently substituted 2 to 23 membered heteroalkylene. In embodiments, L³ is independently unsubstituted 2 to 23 membered heteroalkylene. In embodiments, L³ is independently substituted or unsubstituted 2 to 8 membered heteroalkylene. In embodiments, L³ is independently substituted 2 to 8 membered heteroalkylene. In embodiments, L³ is independently unsubstituted 2 to 8 membered heteroalkylene. In embodiments, L³ is independently substituted or unsubstituted 2 to 6 membered heteroalkylene. In embodiments, L³ is independently substituted 2 to 6 membered heteroalkylene. In embodiments, L³ is independently unsubstituted 2 to 6 membered heteroalkylene. In embodiments, L³ is independently substituted or unsubstituted 4 to 6 membered heteroalkylene. In embodiments, L³ is independently substituted 4 to 6 membered heteroalkylene. In embodiments, L³ is independently unsubstituted 4 to 6 membered heteroalkylene. In embodiments, L³ is independently substituted or unsubstituted 2 to 3 membered heteroalkylene. In embodiments, L³ is independently substituted 2 to 3 membered heteroalkylene. In embodiments, L³ is independently unsubstituted 2 to 3 membered heteroalkylene. In embodiments, L³ is independently substituted or unsubstituted 4 to 5 membered heteroalkylene. In embodiments, L³ is independently substituted 4 to 5 membered heteroalkylene. In embodiments, L³ is independently unsubstituted 4 to 5 membered heteroalkylene.

In embodiments, L⁴ is a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L⁴ is a bond. In embodiments, L⁴ is —N(R²³)—. In embodiments, L⁴ is —O— or —S—. In embodiments, L⁴ is —C(O)—. In embodiments, L⁴ is —N(R²³)C(O)— or —C(O)N(R²⁴)—. In embodiments, L⁴ is —N(R²³)C(O)N(R²⁴)—. In embodiments, L⁴ is —C(O)O— or —OC(O)—. In embodiments, L⁴ is —N(R²³)C(O)O— or —OC(O)N(R²⁴)—. In embodiments, L⁴ is —OPO₂—O—, —O—P(O)(S)—O—, —O—P(O)(R²⁵)—O—, —O—P(O)(NR²³R²⁴)—N—, or O—P(O)(NR²³R²⁴)—O—. In embodiments, L⁴ is —P(O)(NR²³R²⁴)—N—, —P(S)(NR²³R²⁴)—N—, —P(O)(NR²³R²⁴)—O— or —P(S)(NR²³R²⁴)—O—. In embodiments, L⁴ is —S—S—.

In embodiments, L⁴ is independently substituted or unsubstituted alkylene (e.g., C₁-C₂₃, C₁-C₁₂, C₁-C₈, C₁-C₆, C₁-C₄, or C₁-C₂). In embodiments, L⁴ is independently substituted alkylene (e.g., C₁-C₂₃, C₁-C₁₂, C₁-C₈, C₁-C₆, C₁-C₄, or C₁-C₂). In embodiments, L⁴ is independently unsubstituted alkylene (e.g., C₁-C₂₃, C₁-C₁₂, C₁-C₈, C₁-C₆, C₁-C₄, or C₁-C₂). In embodiments, L⁴ is independently substituted or unsubstituted C₁-C₂₃ alkylene. In embodiments, L⁴ is independently substituted C₁-C₂₃ alkylene. In embodiments, L⁴ is independently unsubstituted C₁-C₂₃ alkylene. In embodiments, L⁴ is independently substituted or unsubstituted C₁-C₁₂ alkylene. In embodiments, L⁴ is independently substituted C₁-C₁₂ alkylene. In embodiments, L⁴ is independently unsubstituted C₁-C₁₂ alkylene. In embodiments, L⁴ is independently substituted or unsubstituted C₁-C₈ alkylene. In embodiments, L⁴ is independently substituted C₁-C₈ alkylene. In embodiments, L⁴ is independently unsubstituted C₁-C₈ alkylene. In embodiments, L⁴ is independently substituted or unsubstituted C₁-C₆ alkylene. In embodiments, L⁴ is independently substituted C₁-C₆ alkylene. In embodiments, L⁴ is independently unsubstituted C₁-C₆ alkylene. In embodiments, L⁴ is independently substituted or unsubstituted C₁-C₄ alkylene. In embodiments, L⁴ is independently substituted C₁-C₄ alkylene. In embodiments, L⁴ is independently unsubstituted C₁-C₄ alkylene. In embodiments, L⁴ is independently substituted or unsubstituted ethylene. In embodiments, L⁴ is independently substituted ethylene. In embodiments, L⁴ is independently unsubstituted ethylene. In embodiments, L⁴ is independently substituted or unsubstituted methylene. In embodiments, L⁴ is independently substituted methylene. In embodiments, L⁴ is independently unsubstituted methylene.

In embodiments, L⁴ is independently substituted or unsubstituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L⁴ is independently substituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L⁴ is independently unsubstituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L⁴ is independently substituted or unsubstituted 2 to 23 membered heteroalkylene. In embodiments, L⁴ is independently substituted 2 to 23 membered heteroalkylene. In embodiments, L⁴ is independently unsubstituted 2 to 23 membered heteroalkylene. In embodiments, L⁴ is independently substituted or unsubstituted 2 to 8 membered heteroalkylene. In embodiments, L⁴ is independently substituted 2 to 8 membered heteroalkylene. In embodiments, L⁴ is independently unsubstituted 2 to 8 membered heteroalkylene. In embodiments, L⁴ is independently substituted or unsubstituted 2 to 6 membered heteroalkylene. In embodiments, L⁴ is independently substituted 2 to 6 membered heteroalkylene. In embodiments, L⁴ is independently unsubstituted 2 to 6 membered heteroalkylene. In embodiments, L⁴ is independently substituted or unsubstituted 4 to 6 membered heteroalkylene. In embodiments, L⁴ is independently substituted 4 to 6 membered heteroalkylene. In embodiments, L⁴ is independently unsubstituted 4 to 6 membered heteroalkylene. In embodiments, L⁴ is independently substituted or unsubstituted 2 to 3 membered heteroalkylene. In embodiments, L⁴ is independently substituted 2 to 3 membered heteroalkylene. In embodiments, L⁴ is independently unsubstituted 2 to 3 membered heteroalkylene. In embodiments, L⁴ is independently substituted or unsubstituted 4 to 5 membered heteroalkylene. In embodiments, L⁴ is independently substituted 4 to 5 membered heteroalkylene. In embodiments, L⁴ is independently unsubstituted 4 to 5 membered heteroalkylene.

R²³ is independently hydrogen or unsubstituted alkyl (e.g., C₁-C₂₃, C₁-C₁₂, C₁-C₈, C₁-C₆, C₁-C₄, or C₁-C₂). In embodiments, R²³ is independently hydrogen. In embodiments, R²³ is independently unsubstituted C₁-C₂₃ alkyl. In embodiments, R²³ is independently hydrogen or unsubstituted C₁-C₁₂ alkyl. In embodiments, R²³ is independently hydrogen or unsubstituted C₁-C₁₀ alkyl. In embodiments, R²³ is independently hydrogen or unsubstituted C₁-C₈ alkyl. In embodiments, R²³ is independently hydrogen or unsubstituted C₁-C₆ alkyl. In embodiments, R²³ is independently hydrogen or unsubstituted C₁-C₄ alkyl. In embodiments, R²³ is independently hydrogen or unsubstituted C₁-C₂ alkyl.

R²⁴ is independently hydrogen or unsubstituted alkyl (e.g., C₁-C₂₄, C₁-C₁₂, C₁-C₈, C₁-C₆, C₁-C₄, or C₁-C₂). In embodiments, R²⁴ is independently hydrogen. In embodiments, R²⁴ is independently unsubstituted C₁-C₂₄ alkyl. In embodiments, R²⁴ is independently hydrogen or unsubstituted C₁-C₁₂ alkyl. In embodiments, R²⁴ is independently hydrogen or unsubstituted C₁-C₁₀ alkyl. In embodiments, R²⁴ is independently hydrogen or unsubstituted C₁-C₈ alkyl. In embodiments, R²⁴ is independently hydrogen or unsubstituted C₁-C₆ alkyl. In embodiments, R²⁴ is independently hydrogen or unsubstituted C₁-C₄ alkyl. In embodiments, R²⁴ is independently hydrogen or unsubstituted C₁-C₂ alkyl.

R²⁵ is independently hydrogen or unsubstituted alkyl (e.g., C₁-C₂₅, C₁-C₁₂, C₁-C₈, C₁-C₆, C₁-C₄, or C₁-C₂). In embodiments, R²⁵ is independently hydrogen. In embodiments, R²⁵ is independently unsubstituted C₁-C₂₅ alkyl. In embodiments, R²⁵ is independently hydrogen or unsubstituted C₁-C₁₂ alkyl. In embodiments, R²⁵ is independently hydrogen or unsubstituted C₁-C₁₀ alkyl. In embodiments, R²⁵ is independently hydrogen or unsubstituted C₁-C₈ alkyl. In embodiments, R²⁵ is independently hydrogen or unsubstituted C₁-C₆ alkyl. In embodiments, R²⁵ is independently hydrogen or unsubstituted C₁-C₄ alkyl. In embodiments, R²⁵ is independently hydrogen or unsubstituted C₁-C₂ alkyl.

In embodiments, L³ and L⁴ are independently a bond, —NH—, —O—, —C(O)—, —C(O)O—, —OC(O)—, —OPO₂—O—, —O—P(O)(S)—O—, —O—P(O)(CH₃)—O—, —O—P(S)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, — P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene or substituted or unsubstituted heteroalkylene. In embodiments, L³ is independently a bond, —NH—, —O—, —C(O)—, —C(O)O—, —OC(O)—, —OPO₂—O—, —O—P(O)(S)—O—, —O—P(O)(CH₃)—O—, —O—P(S)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, — P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene or substituted or unsubstituted heteroalkylene. In embodiments, L⁴ is independently a bond, —NH—, —O—, —C(O)—, —C(O)O—, —OC(O)—, —OPO₂—O—, —O—P(O)(S)—O—, —O—P(O)(CH₃)—O—, —O—P(S)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, — P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene or substituted or unsubstituted heteroalkylene.

In embodiments, L³ is independently

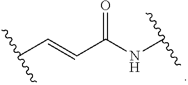 [SMILES: */C=C/C(=O)N*]

In embodiments, L³ is independently —OPO₂—O—. In embodiments, L³ is independently —O—P(O)(S)—O—. In embodiments, L³ is independently —O—. In embodiments, L³ is independently —S—.

In embodiments, L³ is attached to the 3′nitrogen of a morpholino moiety. In embodiments, L³ is independently —C(O)—. In embodiments, L³ is attached to the 6′ carbon of a morpholino moiety. In embodiments, L³ is independently —O—P(O)(N(CH₃)₂)—N—. In embodiments, L³ is independently —O—P(O)(N(CH₃)₂)—O—. In embodiments, L³ is independently —P(O)(N(CH₃)₂)—N—. In embodiments, L³ is independently —P(O)(N(CH₃)₂)—O—.

In embodiments, L⁴ is independently substituted or unsubstituted alkylene or substituted or unsubstituted heteroalkylene. In embodiments, L⁴ is independently -L⁷-NH—C(O)— or -L⁷-C(O)—NH—. In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, L⁷ is independently substituted alkylene (e.g., C₁-C₂₀, C₁-C₁₂, C₁-C₈, C₁-C₆, C₁-C₄, or C₁-C₂). In embodiments, L⁷ is independently unsubstituted alkylene (e.g., C₁-C₂₀, C₁-C₁₂, C₁-C₈, C₁-C₆, C₁-C₄, or C₁-C₂).

In embodiments, L⁴ is independently substituted or unsubstituted heteroalkylene (e.g., 2 to 20 membered, 2 to 12 membered, 2 to 10 membered, 2 to 8 membered, 2 to 6 membered, or 2 to 4 membered). In embodiments, L⁴ is independently substituted heteroalkylene (e.g., 2 to 20 membered, 2 to 12 membered, 2 to 10 membered, 2 to 8 membered, 2 to 6 membered, or 2 to 4 membered). In embodiments, L⁴ is independently oxo-substituted heteroalkylene (e.g., 2 to 20 membered, 2 to 12 membered, 2 to 10 membered, 2 to 8 membered, 2 to 6 membered, or 2 to 4 membered). In embodiments, L⁴ is independently unsubstituted heteroalkylene (e.g., 2 to 20 membered, 2 to 12 membered, 2 to 10 membered, 2 to 8 membered, 2 to 6 membered, or 2 to 4 membered).

In embodiments, L⁴ is independently -L⁷-NH—C(O)— or -L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, L⁴ is independently -L⁷-NH—C(O)—; and L⁷ is independently substituted or unsubstituted alkylene (e.g., C₁-C₂₀, C₁-C₁₂, C₁-C₈, C₁-C₆, C₁-C₄, or C₁-C₂). In embodiments, L⁴ is independently -L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂).

In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, L⁷ is independently substituted alkylene (e.g., C₁-C₂₀, C₁-C₁₂, C₁-C₈, C₁-C₆, C₁-C₄, or C₁-C₂). In embodiments, L⁷ is independently unsubstituted alkylene (e.g., C₁-C₂₀, C₁-C₁₂, C₁-C₈, C₁-C₆, C₁-C₄, or C₁-C₂). In embodiments, L⁷ is independently substituted or unsubstituted C₁-C₂₀ alkylene. In embodiments, L⁷ is independently substituted C₁-C₂₀ alkylene. In embodiments, L⁷ is independently hydroxy(OH)-substituted C₁-C₂₀ alkylene. In embodiments, L⁷ is independently hydroxymethyl-substituted C₁-C₂₀ alkylene. In embodiments, L⁷ is independently unsubstituted C₁-C₂₀ alkylene. In embodiments, L⁷ is independently substituted or unsubstituted C₁-C₁₂ alkylene. In embodiments, L⁷ is independently substituted C₁-C₁₂ alkylene. In embodiments, L⁷ is independently hydroxy(OH)-substituted C₁-C₁₂ alkylene. In embodiments, L⁷ is independently hydroxymethyl-substituted C₁-C₁₂ alkylene. In embodiments, L⁷ is independently unsubstituted C₁-C₁₂ alkylene. In embodiments, L⁷ is independently substituted or unsubstituted C₁-C₈ alkylene. In embodiments, L⁷ is independently substituted C₁-C₈ alkylene. In embodiments, L⁷ is independently hydroxy(OH)-substituted C₁-C₈ alkylene. In embodiments, L⁷ is independently hydroxymethyl-substituted C₁-C₈ alkylene. In embodiments, L⁷ is independently unsubstituted C₁-C₈ alkylene. In embodiments, L⁷ is independently substituted or unsubstituted C₁-C₆ alkylene. In embodiments, L⁷ is independently substituted C₁-C₆ alkylene. In embodiments, L⁷ is independently hydroxy(OH)-substituted C₁-C₆ alkylene. In embodiments, L⁷ is independently hydroxymethyl-substituted C₁-C₆ alkylene. In embodiments, L⁷ is independently unsubstituted C₁-C₆ alkylene. In embodiments, L⁷ is independently substituted or unsubstituted C₁-C₄ alkylene. In embodiments, L⁷ is independently substituted C₁-C₄ alkylene. In embodiments, L⁷ is independently hydroxy(OH)-substituted C₁-C₄ alkylene. In embodiments, L⁷ is independently hydroxymethyl-substituted C₁-C₄ alkylene. In embodiments, L⁷ is independently unsubstituted C₁-C₄ alkylene. In embodiments, L⁷ is independently substituted or unsubstituted C₁-C₂ alkylene. In embodiments, L⁷ is independently substituted C₁-C₂ alkylene. In embodiments, L⁷ is independently hydroxy(OH)-substituted C₁-C₂ alkylene. In embodiments, L⁷ is independently hydroxymethyl-substituted C₁-C₂ alkylene. In embodiments, L⁷ is independently unsubstituted C₁-C₂ alkylene.

In embodiments, L⁴ is independently -L⁷-NH—C(O)— or -L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, L⁴ is independently -L⁷-NH—C(O)— or -L⁷-C(O)—NH—; and L⁷ is independently substituted or unsubstituted C₁-C₈ alkylene. In embodiments, L⁴ is independently -L⁷-NH—C(O)— or -L⁷-C(O)—NH—; and L⁷ is independently substituted C₁-C₈ alkylene. In embodiments, L⁴ is independently -L⁷-NH—C(O)— or -L⁷-C(O)—NH—; and L⁷ is independently hydroxy(OH)-substituted C₁-C₈ alkylene. In embodiments, L⁴ is independently -L⁷-NH—C(O)— or -L⁷-C(O)—NH—; and L⁷ is independently hydroxymethyl-substituted C₁-C₈ alkylene. In embodiments, L⁴ is independently -L⁷-NH—C(O)— or -L⁷-C(O)—NH—; and L⁷ is independently unsubstituted C₁-C₈ alkylene.

In embodiments, L⁴ is independently -L⁷-NH—C(O)— or -L⁷-C(O)—NH—; and L⁷ is independently substituted or unsubstituted C₃-C₅ alkylene. In embodiments, L⁴ is independently -L⁷-NH—C(O)— or -L⁷-C(O)—NH—; and L⁷ is independently substituted C₃-C₅ alkylene. In embodiments, L⁴ is independently -L⁷-NH—C(O)— or -L⁷-C(O)—NH—; and L⁷ is independently hydroxy(OH)-substituted C₃-C₅ alkylene. In embodiments, L⁴ is independently -L⁷-NH—C(O)— or -L⁷-C(O)—NH—; and L⁷ is independently hydroxymethyl-substituted C₃-C₅ alkylene. In embodiments, L⁴ is independently -L⁷-NH—C(O)— or -L⁷-C(O)—NH—; and L⁷ is independently unsubstituted C₃-C₅ alkylene.

In embodiments, L⁴ is independently -L⁷-NH—C(O)— or -L⁷-C(O)—NH—; and L⁷ is independently substituted or unsubstituted C₅-C₈alkylene. In embodiments, L⁴ is independently -L⁷-NH—C(O)— or -L⁷-C(O)—NH—; and L⁷ is independently substituted C₅-C₈alkylene. In embodiments, L⁴ is independently -L⁷-NH—C(O)— or -L⁷-C(O)—NH—; and L⁷ is independently hydroxy(OH)-substituted C₅-C₈alkylene. In embodiments, L⁴ is independently -L⁷-NH—C(O)— or -L⁷-C(O)—NH—; and L⁷ is independently hydroxymethyl-substituted C₅-C₈alkylene. In embodiments, L⁴ is independently -L⁷-NH—C(O)— or -L⁷-C(O)—NH—; and L⁷ is independently unsubstituted C₅-C₈alkylene.

In embodiments, L⁴ is independently -L⁷-NH—C(O)— or -L⁷-C(O)—NH—; and L⁷ is independently substituted or unsubstituted octylene. In embodiments, L⁴ is independently -L⁷-NH—C(O)— or -L⁷-C(O)—NH—; and L⁷ is independently substituted octylene. In embodiments, L⁴ is independently -L⁷-NH—C(O)— or -L⁷-C(O)—NH—; and L⁷ is independently hydroxy(OH)-substituted octylene. In embodiments, L⁴ is independently -L⁷-NH—C(O)— or -L⁷-C(O)—NH—; and L⁷ is independently unsubstituted octylene. In embodiments, L⁴ is independently -L⁷-NH—C(O)— and L⁷ is independently hydroxy(OH)-substituted octylene. In embodiments, L⁴ is independently -L⁷-NH—C(O)— and L⁷ is independently hydroxymethyl-substituted octylene. In embodiments, L⁴ is independently -L⁷-NH—C(O)— and L⁷ is independently unsubstituted octylene.

In embodiments, L⁴ is independently -L⁷-NH—C(O)— or -L⁷-C(O)—NH—; and L⁷ is independently substituted or unsubstituted heptylene. In embodiments, L⁴ is independently -L⁷-NH—C(O)— or -L⁷-C(O)—NH—; and L⁷ is independently substituted heptylene. In embodiments, L⁴ is independently -L⁷-NH—C(O)— or -L⁷-C(O)—NH—; and L⁷ is independently hydroxy(OH)-substituted heptylene. In embodiments, L⁴ is independently -L⁷-NH—C(O)— or -L⁷-C(O)—NH—; and L⁷ is independently unsubstituted heptylene. In embodiments, L⁴ is independently -L⁷-NH—C(O)— and L⁷ is independently hydroxy(OH)-substituted heptylene. In embodiments, L⁴ is independently -L⁷-NH—C(O)— and L⁷ is independently hydroxymethyl-substituted heptylene. In embodiments, L⁴ is independently -L⁷-NH—C(O)— and L⁷ is independently unsubstituted heptylene.

In embodiments, L⁴ is independently -L⁷-NH—C(O)— or -L⁷-C(O)—NH—; and L⁷ is independently substituted or unsubstituted hexylene. In embodiments, L⁴ is independently -L⁷-NH—C(O)— or -L⁷-C(O)—NH—; and L⁷ is independently substituted hexylene. In embodiments, L⁴ is independently -L⁷-NH—C(O)— or -L⁷-C(O)—NH—; and L⁷ is independently hydroxy(OH)-substituted hexylene. In embodiments, L⁴ is independently -L⁷-NH—C(O)— or -L⁷-C(O)—NH—; and L⁷ is independently unsubstituted hexylene. In embodiments, L⁴ is independently -L⁷-NH—C(O)— and L⁷ is independently hydroxy(OH)-substituted hexylene. In embodiments, L⁴ is independently -L⁷-NH—C(O)— and L⁷ is independently hydroxymethyl-substituted hexylene. In embodiments, L⁴ is independently -L⁷-NH—C(O)— and L⁷ is independently unsubstituted hexylene.

In embodiments, L⁴ is independently -L⁷-NH—C(O)— or -L⁷-C(O)—NH—; and L⁷ is independently substituted or unsubstituted pentylene. In embodiments, L⁴ is independently -L⁷-NH—C(O)— or -L⁷-C(O)—NH—; and L⁷ is independently substituted pentylene. In embodiments, L⁴ is independently -L⁷-NH—C(O)— or -L⁷-C(O)—NH—; and L⁷ is independently hydroxy(OH)-substituted pentylene. In embodiments, L⁴ is independently -L⁷-NH—C(O)— or -L⁷-C(O)—NH—; and L⁷ is independently unsubstituted pentylene. In embodiments, L⁴ is independently -L⁷-NH—C(O)— and L⁷ is independently hydroxy(OH)-substituted pentylene. In embodiments, L⁴ is independently -L⁷-NH—C(O)— and L⁷ is independently hydroxymethyl-substituted pentylene. In embodiments, L⁴ is independently -L⁷-NH—C(O)— and L⁷ is independently unsubstituted pentylene.

In embodiments, L⁴ is independently

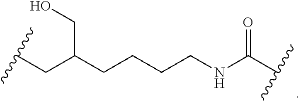 [SMILES: *CC(CO)CCCCNC(*)=O]

In embodiments, L⁴ is independently

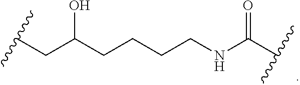 [SMILES: *CC(O)CCCCNC(*)=O]

In embodiments, L⁴ is independently

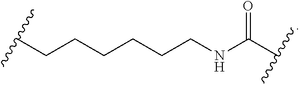 [SMILES: *CCCCCCNC(*)=O]

In embodiments, L⁴ is independently

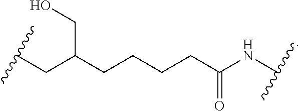 [SMILES: *CC(CO)CCCCC(=O)N*]

In embodiments, L⁴ is independently

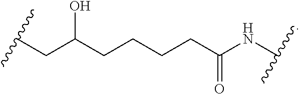 [SMILES: *CC(O)CCCCC(=O)N*]

In embodiments, L⁴ is independently

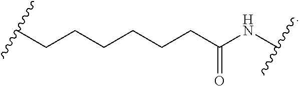 [SMILES: *CCCCCCC(=O)N*]

In embodiments, L⁴ is independently

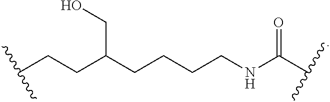 [SMILES: *CCC(CO)CCCCNC(*)=O]

In embodiments, L⁴ is independently

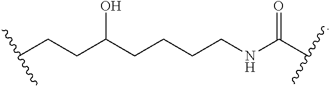 [SMILES: *CCC(O)CCCCNC(*)=O]

In embodiments, L⁴ is independently

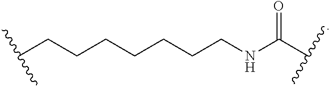 [SMILES: *CCCCCCCNC(*)=O]

In embodiments, L⁴ is independently

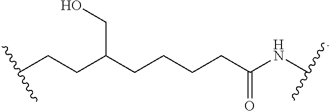 [SMILES: *CCC(CO)CCCCC(=O)N*]

In embodiments, L⁴ is independently

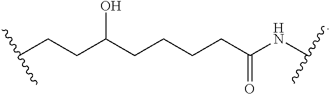 [SMILES: *CCC(O)CCCCC(=O)N*]

In embodiments, L⁴ is independently

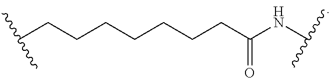 [SMILES: *CCCCCCCC(=O)N*]

In embodiments, -L³-L⁴- is independently -L⁷-NH—C(O)— or -L⁷-C(O)—NH—. In embodiments, L⁷ is independently substituted or unsubstituted heteroalkylene (e.g., 2 to 20 membered, 2 to 12 membered, 2 to 10 membered, 2 to 8 membered, 2 to 6 membered, or 2 to 4 membered). In embodiments, L⁷ is independently substituted heteroalkylene (e.g., 2 to 20 membered, 2 to 12 membered, 2 to 10 membered, 2 to 8 membered, 2 to 6 membered, or 2 to 4 membered). In embodiments, L⁷ is independently oxo-substituted heteroalkylene (e.g., 2 to 20 membered, 2 to 12 membered, 2 to 10 membered, 2 to 8 membered, 2 to 6 membered, or 2 to 4 membered). In embodiments, L⁷ is independently unsubstituted heteroalkylene (e.g., 2 to 20 membered, 2 to 12 membered, 2 to 10 membered, 2 to 8 membered, 2 to 6 membered, or 2 to 4 membered). In embodiments, L⁷ is independently substituted or unsubstituted heteroalkenylene (e.g., 2 to 20 membered, 2 to 12 membered, 2 to 10 membered, 2 to 8 membered, 2 to 6 membered, or 2 to 4 membered). In embodiments, L⁷ is independently substituted heteroalkenylene (e.g., 2 to 20 membered, 2 to 12 membered, 2 to 10 membered, 2 to 8 membered, 2 to 6 membered, or 2 to 4 membered). In embodiments, L⁷ is independently oxo-substituted heteroalkenylene (e.g., 2 to 20 membered, 2 to 12 membered, 2 to 10 membered, 2 to 8 membered, 2 to 6 membered, or 2 to 4 membered). In embodiments, L⁷ is independently unsubstituted heteroalkenylene (e.g., 2 to 20 membered, 2 to 12 membered, 2 to 10 membered, 2 to 8 membered, 2 to 6 membered, or 2 to 4 membered).

In embodiments, L⁷ is independently substituted or unsubstituted 2 to 20 membered heteroalkylene. In embodiments, L⁷ is independently substituted 2 to 20 membered heteroalkylene. In embodiments, L⁷ is independently oxo-substituted 2 to 20 membered heteroalkylene. In embodiments, L⁷ is independently unsubstituted 2 to 20 membered heteroalkylene. In embodiments, L⁷ is independently substituted or unsubstituted 2 to 12 membered heteroalkylene. In embodiments, L⁷ is independently substituted 2 to 12 membered heteroalkylene. In embodiments, L⁷ is independently oxo-substituted 2 to 12 membered heteroalkylene. In embodiments, L⁷ is independently unsubstituted 2 to 12 membered heteroalkylene. In embodiments, L⁷ is independently substituted or unsubstituted 2 to 10 membered heteroalkylene. In embodiments, L⁷ is independently substituted 2 to 10 membered heteroalkylene. In embodiments, L⁷ is independently oxo-substituted 2 to 10 membered heteroalkylene. In embodiments, L⁷ is independently unsubstituted 2 to 10 membered heteroalkylene. In embodiments, L⁷ is independently substituted or unsubstituted 2 to 8 membered heteroalkylene. In embodiments, L⁷ is independently substituted 2 to 8 membered heteroalkylene. In embodiments, L⁷ is independently oxo-substituted 2 to 8 membered heteroalkylene. In embodiments, L⁷ is independently unsubstituted 2 to 8 membered heteroalkylene. In embodiments, L⁷ is independently substituted or unsubstituted 2 to 6 membered heteroalkylene. In embodiments, L⁷ is independently substituted 2 to 6 membered heteroalkylene. In embodiments, L⁷ is independently oxo-substituted 2 to 6 membered heteroalkylene. In embodiments, L⁷ is independently unsubstituted 2 to 6 membered heteroalkylene. In embodiments, L⁷ is independently substituted or unsubstituted 2 to 4 membered heteroalkylene. In embodiments, L⁷ is independently substituted 2 to 4 membered heteroalkylene. In embodiments, L⁷ is independently oxo-substituted 2 to 4 membered heteroalkylene. In embodiments, L⁷ is independently unsubstituted 2 to 4 membered heteroalkylene.

In embodiments, L⁷ is independently substituted or unsubstituted 2 to 20 membered heteroalkenylene. In embodiments, L⁷ is independently substituted 2 to 20 membered heteroalkenylene. In embodiments, L⁷ is independently oxo-substituted 2 to 20 membered heteroalkenylene. In embodiments, L⁷ is independently unsubstituted 2 to 20 membered heteroalkenylene. In embodiments, L⁷ is independently substituted or unsubstituted 2 to 12 membered heteroalkenylene. In embodiments, L⁷ is independently substituted 2 to 12 membered heteroalkenylene. In embodiments, L⁷ is independently oxo-substituted 2 to 12 membered heteroalkenylene. In embodiments, L⁷ is independently unsubstituted 2 to 12 membered heteroalkenylene. In embodiments, L⁷ is independently substituted or unsubstituted 2 to 10 membered heteroalkenylene. In embodiments, L⁷ is independently substituted 2 to 10 membered heteroalkenylene. In embodiments, L⁷ is independently oxo-substituted 2 to 10 membered heteroalkenylene. In embodiments, L⁷ is independently unsubstituted 2 to 10 membered heteroalkenylene. In embodiments, L⁷ is independently substituted or unsubstituted 2 to 8 membered heteroalkenylene. In embodiments, L⁷ is independently substituted 2 to 8 membered heteroalkenylene. In embodiments, L⁷ is independently oxo-substituted 2 to 8 membered heteroalkenylene. In embodiments, L⁷ is independently unsubstituted 2 to 8 membered heteroalkenylene. In embodiments, L⁷ is independently substituted or unsubstituted 2 to 6 membered heteroalkenylene. In embodiments, L⁷ is independently substituted 2 to 6 membered heteroalkenylene. In embodiments, L⁷ is independently oxo-substituted 2 to 6 membered heteroalkenylene. In embodiments, L⁷ is independently unsubstituted 2 to 6 membered heteroalkenylene. In embodiments, L⁷ is independently substituted or unsubstituted 2 to 4 membered heteroalkenylene. In embodiments, L⁷ is independently substituted 2 to 4 membered heteroalkenylene. In embodiments, L⁷ is independently oxo-substituted 2 to 4 membered heteroalkenylene. In embodiments, L⁷ is independently unsubstituted 2 to 4 membered heteroalkenylene.

In embodiments, -L³-L⁴- is independently —O-L⁷-NH—C(O)— or —O-L⁷-C(O)—NH—. In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —O-L⁷-NH—C(O)— or —O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —O-L⁷-NH—C(O)—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂).

In embodiments, -L³-L⁴- is independently —O-L⁷-C(O)—NH—; and L⁷ is independently substituted or unsubstituted C₁-C₈ alkylene. In embodiments, -L³-L⁴- is independently —O-L⁷-C(O)—NH—; and L⁷ is independently substituted C₁-C₈ alkylene. In embodiments, -L³-L⁴- is independently —O-L⁷-C(O)—NH—; and L⁷ is independently hydroxy(OH)-substituted C₁-C₈ alkylene. In embodiments, -L³-L⁴- is independently —O-L⁷-C(O)— NH— and L⁷ is independently hydroxymethyl-substituted C₁-C₈ alkylene. In embodiments, -L³-L⁴- is independently —O-L⁷-C(O)—NH—; and L⁷ is independently unsubstituted C₁-C₈ alkylene.

In embodiments, -L³-L⁴- is independently —O-L⁷-C(O)—NH—; and L⁷ is independently substituted or unsubstituted C₃-C₅ alkylene. In embodiments, -L³-L⁴- is independently —O-L⁷-C(O)—NH—; and L⁷ is independently substituted C₃-C₅ alkylene. In embodiments, -L³-L⁴- is independently —O-L⁷-C(O)—NH—; and L⁷ is independently hydroxy(OH)-substituted C₃-C₅ alkylene. In embodiments, -L³-L⁴- is independently —O-L⁷-C(O)—NH— and L⁷ is independently hydroxymethyl-substituted C₃-C₅ alkylene. In embodiments, -L³-L⁴- is independently —O-L⁷-C(O)—NH—; and L⁷ is independently unsubstituted C₃-C₈ alkylene.

In embodiments, -L³-L⁴- is independently —O-L⁷-C(O)—NH—; and L⁷ is independently substituted or unsubstituted C₅-C₈alkylene. In embodiments, -L³-L⁴- is independently —O-L⁷-C(O)—NH—; and L⁷ is independently substituted C₅-C₈alkylene. In embodiments, -L³-L⁴- is independently —O-L⁷-C(O)—NH—; and L⁷ is independently hydroxy(OH)-substituted C₅-C₈ alkylene. In embodiments, -L³-L⁴- is independently —O-L⁷-C(O)—NH— and L⁷ is independently hydroxymethyl-substituted C₅-C₈alkylene. In embodiments, -L³-L⁴- is independently —O-L⁷-C(O)—NH—; and L⁷ is independently unsubstituted C₅-C₈ alkylene.

In embodiments, -L³-L⁴- is independently —O-L⁷-NH—C(O)—; and L⁷ is independently substituted or unsubstituted C₁-C₈ alkylene. In embodiments, -L³-L⁴- is independently —O-L⁷-NH—C(O)—; and L⁷ is independently substituted C₁-C₈ alkylene. In embodiments, -L³-L⁴- is independently —O-L⁷-NH—C(O)—; and L⁷ is independently hydroxy(OH)-substituted C₁-C₈ alkylene. In embodiments, -L³-L⁴- is independently —O-L⁷-NH—C(O)—; and L⁷ is independently hydroxymethyl-substituted C₁-C₈ alkylene. In embodiments, -L³-L⁴- is independently —O-L⁷-NH—C(O)—; and L⁷ is independently unsubstituted C₁-C₈ alkylene.

In embodiments, -L³-L⁴- is independently —O-L⁷-NH—C(O)—; and L⁷ is independently substituted or unsubstituted C₃-C₅ alkylene. In embodiments, -L³-L⁴- is independently —O-L⁷-NH—C(O)—; and L⁷ is independently substituted C₃-C₅ alkylene. In embodiments, -L³-L⁴- is independently —O-L⁷-NH—C(O)—; and L⁷ is independently hydroxy(OH)-substituted C₃-C₅ alkylene. In embodiments, -L³-L⁴- is independently —O-L⁷-NH—C(O)—; and L⁷ is independently hydroxymethyl-substituted C₃-C₈ alkylene. In embodiments, -L³-L⁴- is independently —O-L⁷-NH—C(O)—; and L⁷ is independently unsubstituted C₃-C₅ alkylene.

In embodiments, -L³-L⁴- is independently

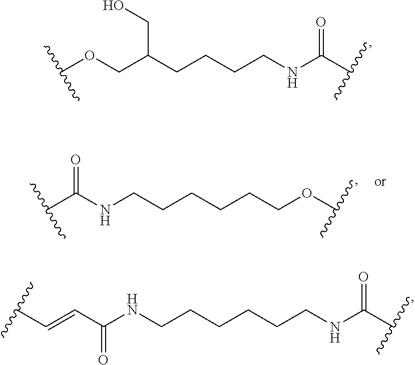 [SMILES: *OCC(CO)CCCCNC(*)=O ; *OCCCCCCNC(*)=O ; */C=C/C(=O)NCCCCCCNC(*)=O]

In embodiments, -L³-L⁴-is independently

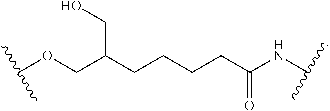 [SMILES: *NC(=O)CCCCC(CO)CO*]

In embodiments, -L³-L⁴- is independently

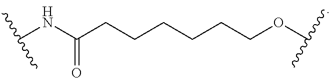 [SMILES: *NC(=O)CCCCCCO*]

In embodiments, -L³-L⁴- is independently

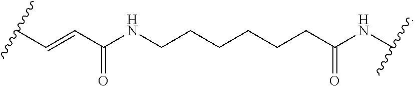 [SMILES: */C=C/C(=O)NCCCCCCC(=O)N*]

In embodiments, -L³-L⁴- is independently —OPO₂—O-L⁷-NH—C(O)—, —OP(O)(S)—O-L⁷-NH—C(O)—, —OPO₂—O-L⁷-C(O)—NH— or —OP(O)(S)—O-L⁷-C(O)—NH—. In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OPO₂—O-L⁷-NH—C(O)— or —OP(O)(S)—O-L⁷-NH—C(O)—; and L⁷ is independently substituted or unsubstituted alkylene. In embodiments, -L³-L⁴- is independently —OPO₂—O-L⁷-NH—C(O)—; and L⁷ is independently substituted or unsubstituted alkylene. In embodiments, -L³-L⁴- is independently —OP(O)(S)—O-L⁷-NH—C(O)—; and L⁷ is independently substituted or unsubstituted alkylene. In embodiments, -L³-L⁴- is independently —OPO₂—O-L⁷-C(O)—NH— or —OP(O)(S)—O-L⁷-C(O)—NH—; and L⁷ is independently substituted or unsubstituted alkylene. In embodiments, -L³-L⁴- is independently —OPO₂—O-L⁷-C(O)—NH—; and L⁷ is independently substituted or unsubstituted alkylene. In embodiments, -L³-L⁴- is independently —OP(O)(S)—O-L⁷-C(O)—NH—; and L⁷ is independently substituted or unsubstituted alkylene.

In embodiments, -L³-L⁴- is independently —OPO₂—O-L⁷-NH—C(O)— or —OPO₂—O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OPO₂—O-L⁷-NH—C(O)—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OPO₂—O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂).

In embodiments, -L³-L⁴- is independently —OP(O)(S)—O-L⁷-NH—C(O)— or —OP(O)(S)—O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OP(O)(S)—O-L⁷-NH—C(O)—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OP(O)(S)—O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂).

In embodiments, -L³-L⁴- is independently —OPO₂—O-L⁷-C(O)—NH—; and L⁷ is independently substituted or unsubstituted C₁-C₅ alkylene. In embodiments, -L³-L⁴- is independently —OPO₂—O-L⁷-C(O)—NH—; and L⁷ is independently substituted C₁-C₅ alkylene. In embodiments, -L³-L⁴- is independently —OPO₂—O-L⁷-C(O)—NH—; and L⁷ is independently hydroxy(OH)-substituted C₁-C₅ alkylene. In embodiments, -L³-L⁴- is independently —OPO₂—O-L⁷-C(O)—NH—; and L⁷ is independently hydroxymethyl-substituted C₁-C₈ alkylene. In embodiments, -L³-L⁴- is independently —OPO₂—O-L⁷-C(O)—NH—; and L⁷ is independently unsubstituted C₁-C₅ alkylene.

In embodiments, -L³-L⁴- is independently —OP(O)(S)—O-L⁷-C(O)—NH—; and L⁷ is independently substituted or unsubstituted C₁-C₅ alkylene. In embodiments, -L³-L⁴- is independently —OP(O)(S)—O-L⁷-C(O)—NH—; and L⁷ is independently substituted C₁-C₅ alkylene. In embodiments, -L³-L⁴- is independently —OP(O)(S)—O-L⁷-C(O)—NH—; and L⁷ is independently hydroxy(OH)-substituted C₁-C₅ alkylene. In embodiments, -L³-L⁴- is independently —OP(O)(S)—O-L⁷-C(O)—NH—; and L⁷ is independently hydroxymethyl-substituted C₁-C₈ alkylene. In embodiments, -L³-L⁴- is independently —OP(O)(S)—O-L⁷-C(O)—NH—; and L⁷ is independently unsubstituted C₁-C₈ alkylene.

In embodiments, -L³-L⁴- is independently —OPO₂—O-L⁷-C(O)—NH—; and L⁷ is independently substituted or unsubstituted C₃-C₈ alkylene. In embodiments, -L³-L⁴- is independently —OPO₂—O-L⁷-C(O)—NH—; and L⁷ is independently substituted C₃-C₈ alkylene. In embodiments, -L³-L⁴- is independently —OPO₂—O-L⁷-C(O)—NH—; and L⁷ is independently hydroxy(OH)-substituted C₃-C₈ alkylene. In embodiments, -L³-L⁴- is independently —OPO₂—O-L⁷-C(O)—NH—; and L⁷ is independently hydroxymethyl-substituted C₃-C₈ alkylene. In embodiments, -L³-L⁴- is independently —OPO₂—O-L⁷-C(O)—NH—; and L⁷ is independently unsubstituted C₃-C₈ alkylene.

In embodiments, -L³-L⁴- is independently —OP(O)(S)—O-L⁷-C(O)—NH—; and L⁷ is independently substituted or unsubstituted C₃-C₅ alkylene. In embodiments, -L³-L⁴- is independently —OP(O)(S)—O-L⁷-C(O)—NH—; and L⁷ is independently substituted C₃-C₅ alkylene. In embodiments, -L³-L⁴- is independently —OP(O)(S)—O-L⁷-C(O)—NH—; and L⁷ is independently hydroxy(OH)-substituted C₃-C₈ alkylene. In embodiments, -L³-L⁴- is independently —OP(O)(S)—O-L⁷-C(O)—NH—; and L⁷ is independently hydroxymethyl-substituted C₃-C₈ alkylene. In embodiments, -L³-L⁴- is independently —OP(O)(S)—O-L⁷-C(O)—NH—; and L⁷ is independently unsubstituted C₃-C₈ alkylene.

In embodiments, -L³-L⁴- is independently —OPO₂—O-L⁷-C(O)—NH—; and L⁷ is independently substituted or unsubstituted C₅-C₈alkylene. In embodiments, -L³-L⁴- is independently —OPO₂—O-L⁷-C(O)—NH—; and L⁷ is independently substituted C₅-C₈alkylene. In embodiments, -L³-L⁴- is independently —OPO₂—O-L⁷-C(O)—NH—; and L⁷ is independently hydroxy(OH)-substituted C₅-C₈alkylene. In embodiments, -L³-L⁴- is independently —OPO₂—O-L⁷-C(O)—NH—; and L⁷ is independently hydroxymethyl-substituted C₅-C₈ alkylene. In embodiments, -L³-L⁴- is independently —OPO₂—O-L⁷-C(O)—NH—; and L⁷ is independently unsubstituted C₅-C₈alkylene.

In embodiments, -L³-L⁴- is independently —OP(O)(S)—O-L⁷-C(O)—NH—; and L⁷ is independently substituted or unsubstituted C₅-C₈alkylene. In embodiments, -L³-L⁴- is independently —OP(O)(S)—O-L⁷-C(O)—NH—; and L⁷ is independently substituted C₅-C₈alkylene. In embodiments, -L³-L⁴- is independently —OP(O)(S)—O-L⁷-C(O)—NH—; and L⁷ is independently hydroxy(OH)-substituted C₅-C₈alkylene. In embodiments, -L³-L⁴- is independently —OP(O)(S)—O-L⁷-C(O)—NH—; and L⁷ is independently hydroxymethyl-substituted C₅-C₈alkylene. In embodiments, -L³-L⁴- is independently —OP(O)(S)—O-L⁷-C(O)—NH—; and L⁷ is independently unsubstituted C₅-C₈alkylene.

In embodiments, -L³-L⁴- is independently —OPO₂—O-L⁷-NH—C(O)—; and L⁷ is independently substituted or unsubstituted C₁-C₈ alkylene. In embodiments, -L³-L⁴- is independently —OPO₂—O-L⁷-NH—C(O)—; and L⁷ is independently substituted C₁-C₈ alkylene. In embodiments, -L³-L⁴- is independently —OPO₂—O-L⁷-NH—C(O)—; and L⁷ is independently hydroxy(OH)-substituted C₁-C₈ alkylene. In embodiments, -L³-L⁴- is independently —OPO₂—O-L⁷-NH—C(O)—; and L⁷ is independently hydroxymethyl-substituted C₁-C₈ alkylene. In embodiments, -L³-L⁴- is independently —OPO₂—O-L⁷-NH—C(O)—; and L⁷ is independently unsubstituted C₁-C₈ alkylene.

In embodiments, -L³-L⁴- is independently —OP(O)(S)—O-L⁷-NH—C(O)—; and L⁷ is independently substituted or unsubstituted C₁-C₈ alkylene. In embodiments, -L³-L⁴- is independently —OP(O)(S)—O-L⁷-NH—C(O)—; and L⁷ is independently substituted C₁-C₅ alkylene. In embodiments, -L³-L⁴- is independently —OP(O)(S)₂—O-L⁷-NH—C(O)—; and L⁷ is independently hydroxy(OH)-substituted C₁-C₅ alkylene. In embodiments, -L³-L⁴- is independently —OP(O)(S)—O-L⁷-NH—C(O)—; and L⁷ is independently hydroxymethyl-substituted C₁-C₈ alkylene. In embodiments, -L³-L⁴- is independently —OP(O)(S)—O-L⁷-NH—C(O)—; and L⁷ is independently unsubstituted C₁-C₈ alkylene.

In embodiments, -L³-L⁴- is independently —OPO₂—O-L⁷-NH—C(O)—; and L⁷ is independently substituted or unsubstituted C₃-C₈ alkylene. In embodiments, -L³-L⁴- is independently —OPO₂—O-L⁷-NH—C(O)—; and L⁷ is independently substituted C₃-C₈ alkylene. In embodiments, -L³-L⁴- is independently —OPO₂—O-L⁷-NH—C(O)—; and L⁷ is independently hydroxy(OH)-substituted C₃-C₈ alkylene. In embodiments, -L³-L⁴- is independently —OPO₂—O-L⁷-NH—C(O)—; and L⁷ is independently hydroxymethyl-substituted C₃-C₈ alkylene. In embodiments, -L³-L⁴- is independently —OPO₂—O-L⁷-NH—C(O)—; and L⁷ is independently unsubstituted C₃-C₅ alkylene.

In embodiments, -L³-L⁴- is independently —OP(O)(S)—O-L⁷-NH—C(O)—; and L⁷ is independently substituted or unsubstituted C₃-C₈ alkylene. In embodiments, -L³-L⁴- is independently —OP(O)(S)—O-L⁷-NH—C(O)—; and L⁷ is independently substituted C₃-C₅ alkylene. In embodiments, -L³-L⁴- is independently —OP(O)(S)—O-L⁷-NH—C(O)—; and L⁷ is independently hydroxy(OH)-substituted C₃-C₈ alkylene. In embodiments, -L³-L⁴- is independently —OP(O)(S)—O-L⁷-NH—C(O)—; and L⁷ is independently hydroxymethyl-substituted C₃-C₈ alkylene. In embodiments, -L³-L⁴- is independently —OP(O)(S)—O-L⁷-NH—C(O)—; and L⁷ is independently unsubstituted C₃-C₈ alkylene.

In embodiments, -L³-L⁴- is independently —OPO₂—O-L⁷-NH—C(O)—; and L⁷ is independently substituted or unsubstituted C₅-C₈alkylene. In embodiments, -L³-L⁴- is independently —OPO₂—O-L⁷-NH—C(O)—; and L⁷ is independently substituted C₅-C₈alkylene. In embodiments, -L³-L⁴- is independently —OPO₂—O-L⁷-NH—C(O)—; and L⁷ is independently hydroxy(OH)-substituted C₅-C₈alkylene. In embodiments, -L³-L⁴- is independently —OPO₂—O-L⁷-NH—C(O)—; and L⁷ is independently hydroxymethyl-substituted C₅-C₈ alkylene. In embodiments, -L³-L⁴- is independently —OPO₂—O-L⁷-NH—C(O)—; and L⁷ is independently unsubstituted C₅-C₈alkylene.

In embodiments, -L³-L⁴- is independently —OP(O)(S)—O-L⁷-NH—C(O)—; and L⁷ is independently substituted or unsubstituted C₅-C₈alkylene. In embodiments, -L³-L⁴- is independently —OP(O)(S)—O-L⁷-NH—C(O)—; and L⁷ is independently substituted C₅-C₈alkylene. In embodiments, -L³-L⁴- is independently —OP(O)(S)—O-L⁷-NH—C(O)—; and L⁷ is independently hydroxy(OH)-substituted C₅-C₈alkylene. In embodiments, -L³-L⁴- is independently —OP(O)(S)—O-L⁷-NH—C(O)—; and L⁷ is independently hydroxymethyl-substituted C₅-C₈alkylene. In embodiments, -L³-L⁴- is independently —OP(O)(S)—O-L⁷-NH—C(O)—; and L⁷ is independently unsubstituted C₅-C₈alkylene.

In embodiments, -L³-L⁴- is independently

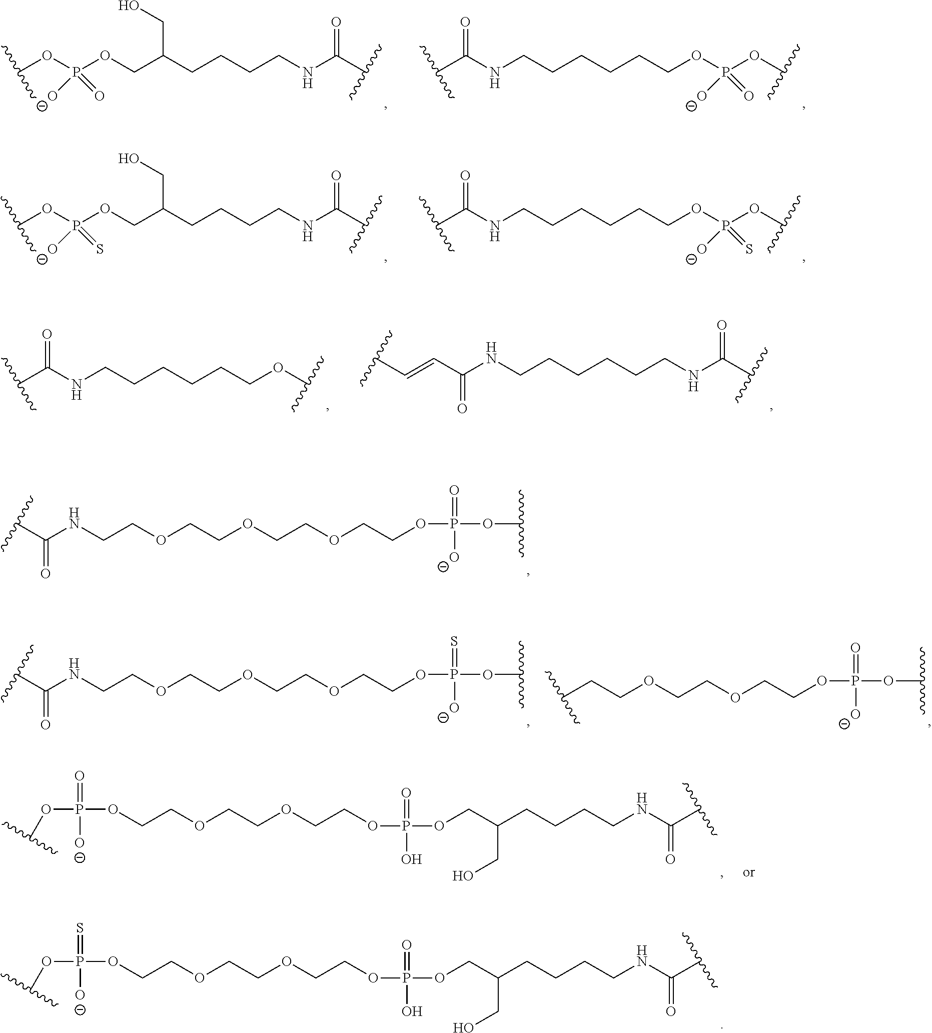

In embodiments, -L³-L⁴- is independently

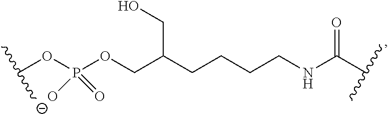 [SMILES: *OP(=O)([O-])OCC(CO)CCCCNC(*)=O]

In embodiments, -L³-L⁴- is independently

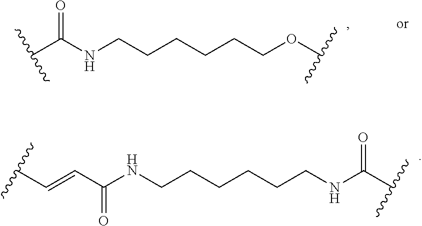 [SMILES: *OCCCCCCNC(*)=O ; */C=C/C(=O)NCCCCCCNC(*)=O]

In embodiments, -L³-L⁴- is independently

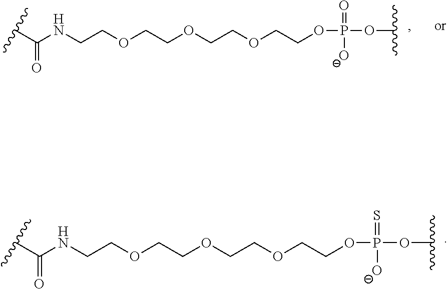 [SMILES: *OP(=O)([O-])OCCOCCOCCOCCNC(*)=O ; *OP([O-])(=S)OCCOCCOCCOCCNC(*)=O]

In embodiments, -L³-L⁴- is independently

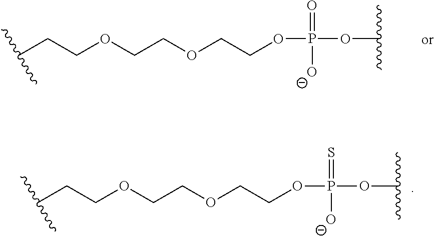 [SMILES: *CCOCCOCCOP(=O)([O-])O* ; *CCOCCOCCOP([O-])(=S)O*]

In embodiments, -L³-L⁴- is independently

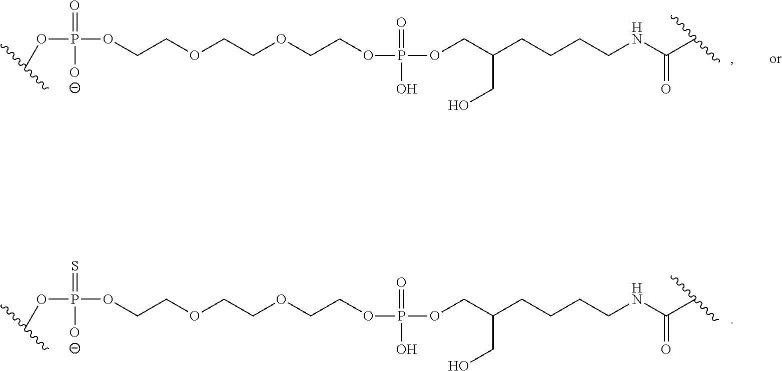 [SMILES: *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]

In embodiments, -L³-L⁴- is independently

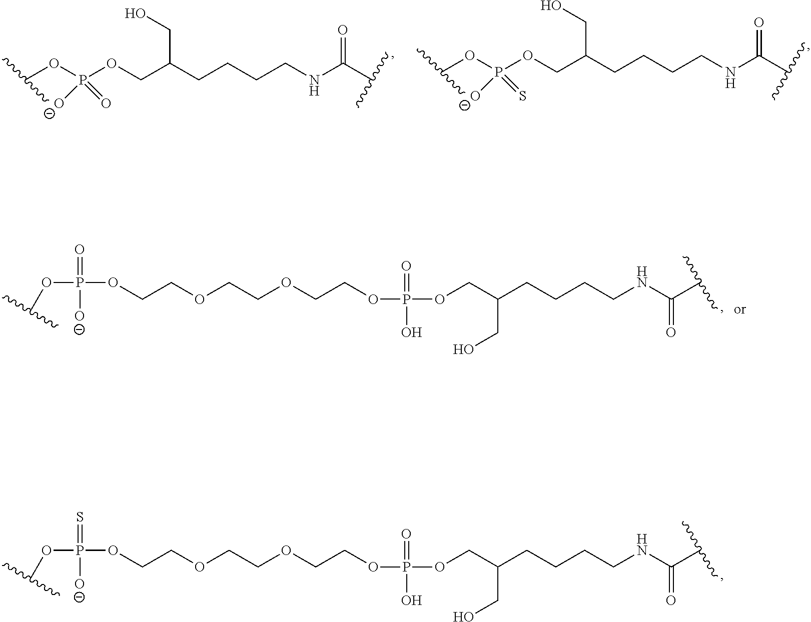 [SMILES: *OP(=O)([O-])OCC(CO)CCCCNC(*)=O ; *OP([O-])(=S)OCC(CO)CCCCNC(*)=O ; *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]

and is attached to a 3′ carbon of the oligonucleotide. In embodiments, -L³-L⁴- is independently

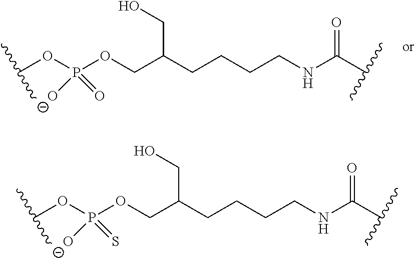 [SMILES: *OP(=O)([O-])OCC(CO)CCCCNC(*)=O ; *OP([O-])(=S)OCC(CO)CCCCNC(*)=O]

that is attached to a 3′ carbon of the oligonucleotide. In embodiments, -L³-L⁴- is independently

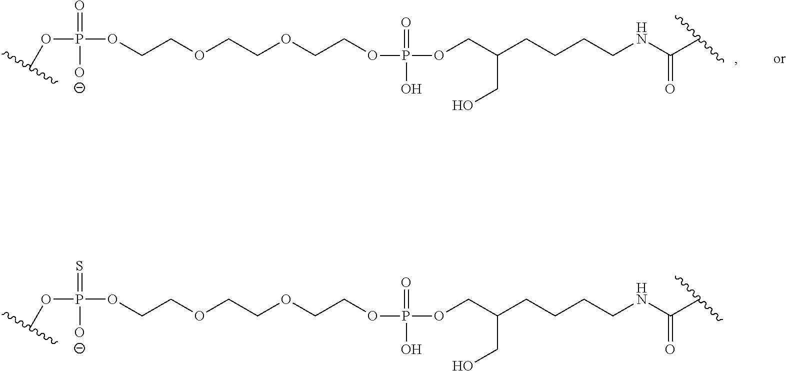 [SMILES: *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]

that is attached to a 3′ carbon of the oligonucleotide.

In embodiments, -L³-L⁴- is independently

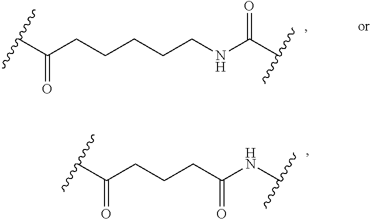 [SMILES: *C(=O)CCCCCNC(*)=O ; *NC(=O)CCCC(*)=O]

and is attached to a 3′ nitrogen of the oligonucleotide (e.g., the 3′ nitrogen of a morpholino moiety).
In embodiments, -L³-L⁴- is independently

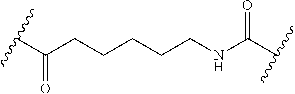 [SMILES: *C(=O)CCCCCNC(*)=O]

attached to a 3′ nitrogen of the oligonucleotide (e.g., the 3′ nitrogen of a morpholino moiety). In embodiments, -L³-L⁴- is independently

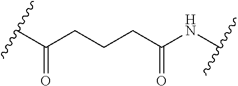 [SMILES: *NC(=O)CCCC(*)=O]

that is attached to a 3′ nitrogen of the oligonucleotide (e.g., the 3′ nitrogen of a morpholino moiety).

In embodiments, an -L³-L⁴- is independently

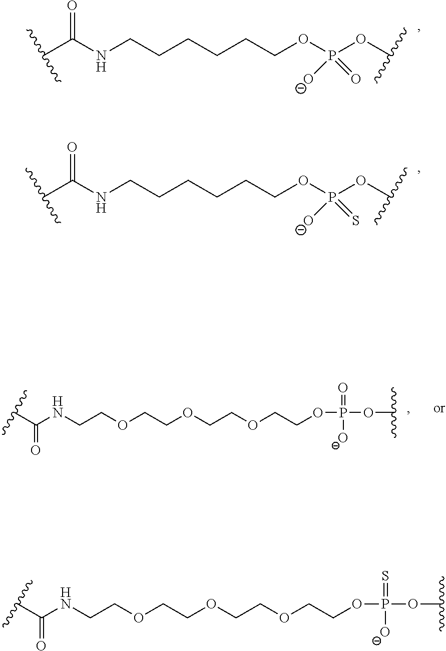 [SMILES: *OP(=O)([O-])OCCCCCCNC(*)=O ; *OP([O-])(=S)OCCCCCCNC(*)=O ; *OP(=O)([O-])OCCOCCOCCOCCNC(*)=O ; *OP([O-])(=S)OCCOCCOCCOCCNC(*)=O]

and is attached to a 5′ carbon of the oligonucleotide. In embodiments, an -L³-L⁴- is independently

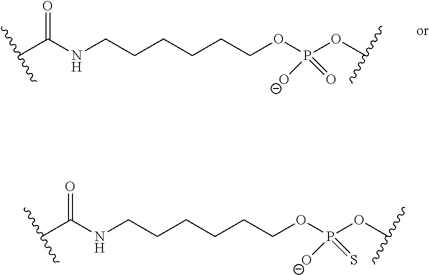 [SMILES: *OP(=O)([O-])OCCCCCCNC(*)=O ; *OP([O-])(=S)OCCCCCCNC(*)=O]

that is attached to a 5′ carbon of the oligonucleotide. In embodiments, an -L³-L⁴- is independently

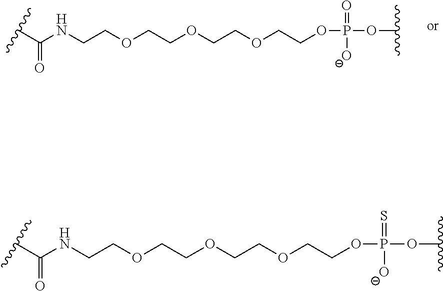 [SMILES: *OP(=O)([O-])OCCOCCOCCOCCNC(*)=O ; *OP([O-])(=S)OCCOCCOCCOCCNC(*)=O]

that is attached to a 5′ carbon of the oligonucleotide.

In embodiments, an -L³-L⁴- is independently

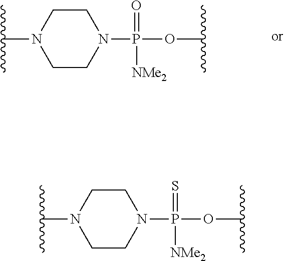 [SMILES: *OP(=O)(N(C)C)N1CCN(*)CC1 ; *OP(=S)(N(C)C)N1CCN(*)CC1]

that is attached to a 6′ carbon of the oligonucleotide (e.g., the 6′ carbon of a morpholino moiety). In embodiments, an -L³-L⁴- is independently

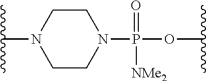 [SMILES: *OP(=O)(N(C)C)N1CCN(*)CC1]

that is attached to a 6′ carbon of the oligonucleotide (e.g., the 6′ carbon of a morpholino moiety). In embodiments, an -L³-L⁴- is independently

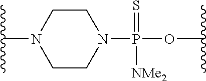 [SMILES: *OP(=S)(N(C)C)N1CCN(*)CC1]

that is attached to a 6′ carbon of the oligonucleotide (e.g., the 6′ carbon of a morpholino moiety).

In embodiments, an -L³-L⁴- is independently is attached to a nucleobase of the oligonucleotide. In embodiments, an -L³-L⁴- is independently

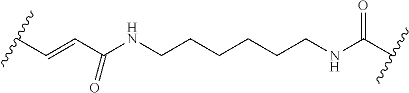 [SMILES: */C=C/C(=O)NCCCCCCNC(*)=O]

and is attached to a nucleobase of the oligonucleotide.

In embodiments, -L³-L⁴- is attached to a 3′ carbon of the single-stranded oligonucleotide at the 3′ end.

In embodiments, -L³-L⁴- is attached to a 3′ nitrogen of the single-stranded oligonucleotide at the 3′ end (e.g., the 3′ nitrogen of a morpholino moiety).

In embodiments, -L³-L⁴- is attached to a 5′ carbon of the single-stranded oligonucleotide at the 5′ end of the single-stranded oligonucleotide.

In embodiments, -L³-L⁴- is attached to a 6′ carbon of the single-stranded oligonucleotide at its 5′ end (e.g., the 6′ carbon of a morpholino moiety). In embodiments, -L³-L⁴- is independently

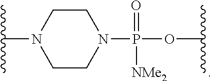 [SMILES: *OP(=O)(N(C)C)N1CCN(*)CC1]

In embodiments, -L³-L⁴- is attached to a 2′ carbon of the single-stranded oligonucleotide. In embodiments, -L³-L⁴- is attached to a 2′ carbon of the single-stranded oligonucleotide at the 3′ end. In embodiments, -L³-L⁴- is attached to a 2′ carbon of the single-stranded oligonucleotide at the 5′ end.

In embodiments, -L³-L⁴- is attached to a nucleobase of the single-stranded oligonucleotide. In embodiments, -L³-L⁴- is attached to a nucleobase of the single-stranded oligonucleotide at its of 3′ end. In embodiments, -L³-L⁴- is attached to a nucleobase of the single-stranded oligonucleotide at its 5′ end.

In embodiments, -L³-L⁴- is independently

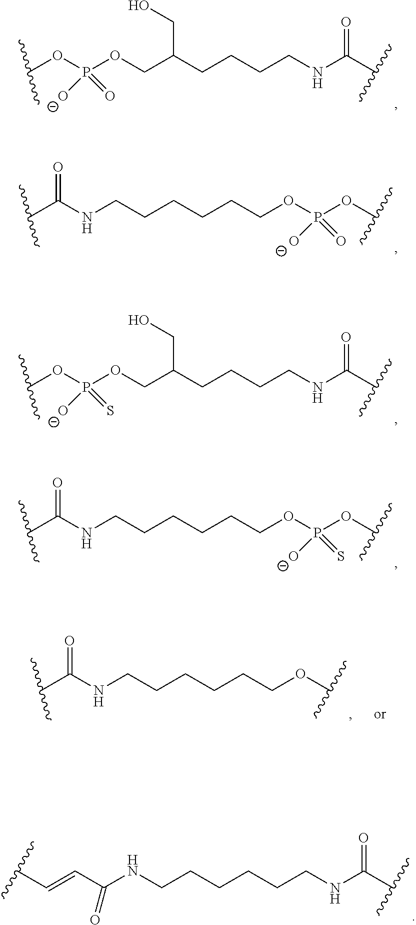 [SMILES: *OP(=O)([O-])OCC(CO)CCCCNC(*)=O ; *OP(=O)([O-])OCCCCCCNC(*)=O ; *OP([O-])(=S)OCC(CO)CCCCNC(*)=O ; *OP([O-])(=S)OCCCCCCNC(*)=O ; *OCCCCCCNC(*)=O ; */C=C/C(=O)NCCCCCCNC(*)=O]

In embodiments, -L³-L⁴- is independently

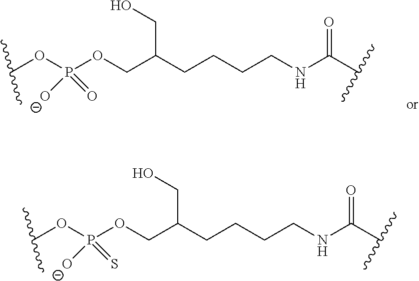 [SMILES: *OP(=O)([O-])OCC(CO)CCCCNC(*)=O ; *OP([O-])(=S)OCC(CO)CCCCNC(*)=O]

that is attached to a 3′ carbon of the oligonucleotide. In embodiments, -L³-L⁴- is independently

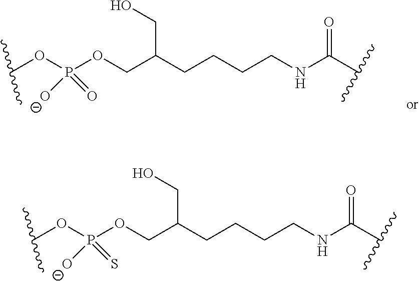 [SMILES: *OP(=O)([O-])OCC(CO)CCCCNC(*)=O ; *OP([O-])(=S)OCC(CO)CCCCNC(*)=O]

that is attached to a 5′ carbon of the oligonucleotide.

In embodiments, -L³-L⁴- is independently

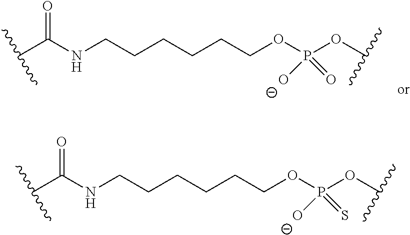 [SMILES: *OP(=O)([O-])OCCCCCCNC(*)=O ; *OP([O-])(=S)OCCCCCCNC(*)=O]

that is attached to a 3′ carbon of the oligonucleotide. In embodiments, -L³-L⁴- is independently

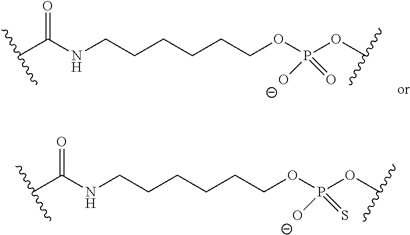 [SMILES: *OP(=O)([O-])OCCCCCCNC(*)=O ; *OP([O-])(=S)OCCCCCCNC(*)=O]

that is attached to a 5′ carbon of the oligonucleotide.

In embodiments, -L³-L⁴- is independently

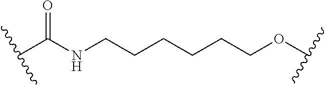 [SMILES: *OCCCCCCNC(*)=O]

H that is attached to a 3′ carbon of the oligonucleotide. In embodiments, -L³-L⁴- is independently

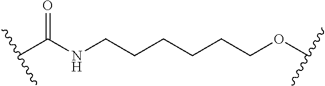 [SMILES: *OCCCCCCNC(*)=O]

and is attached to a 5′ carbon of the oligonucleotide. In embodiments, -L³-L⁴- is independently

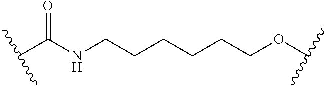 [SMILES: *OCCCCCCNC(*)=O]

that is attached to a 6′ carbon of the oligonucleotide (e.g., the 6′ carbon of a morpholino moiety). In embodiments, -L³-L⁴- is independently

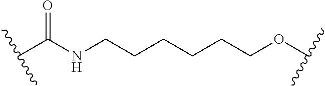 [SMILES: *OCCCCCCNC(*)=O]

and is attached to a 2′ carbon of the oligonucleotide.

In embodiments, -L³-L⁴- is independently

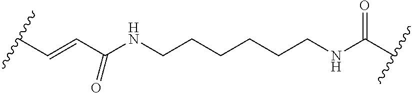 [SMILES: */C=C/C(=O)NCCCCCCNC(*)=O]

that is attached to a 2′ carbon of the oligonucleotide. In embodiments, -L³-L⁴- is independently

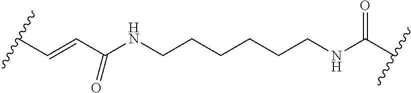 [SMILES: */C=C/C(=O)NCCCCCCNC(*)=O]

and is attached to a nucleobase of the oligonucleotide.

In embodiments, R³ is independently hydrogen, —NH₂, —OH, —SH, —C(O)H, —C(O)NH₂, —NHC(O)H, —NHC(O)OH, —NHC(O)NH₂, —C(O) OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³ is independently hydrogen. In embodiments, R³ is independently —NH₂. In embodiments, R³ is independently —OH. In embodiments, R³ is independently —SH. In embodiments, R³ is independently —C(O)H. In embodiments, R³ is independently —C(O)NH₂. In embodiments, R³ is independently —NHC(O)H. In embodiments, R³ is independently —NHC(O)OH. In embodiments, R³ is independently —NHC(O)NH₂. In embodiments, R³ is independently —C(O)OH. In embodiments, R³ is independently —OC(O)H. In embodiments, R³ is independently —N₃.

In embodiments, R³ is independently substituted or unsubstituted alkyl (e.g., C₁-C₂₀, C₁-C₁₂, C₁-C₈, C₁-C₆, C₁-C₄, or C₁-C₂). In embodiments, R³ is independently substituted or unsubstituted C₁-C₂₀ alkyl. In embodiments, R³ is independently substituted C₁-C₂₀ alkyl. In embodiments, R³ is independently unsubstituted C₁-C₂₀ alkyl. In embodiments, R³ is independently substituted or unsubstituted C₁-C₁₂ alkyl. In embodiments, R³ is independently substituted C₁-C₁₂ alkyl. In embodiments, R³ is independently unsubstituted C₁-C₁₂ alkyl. In embodiments, R³ is independently substituted or unsubstituted C₁-C₈ alkyl. In embodiments, R³ is independently substituted C₁-C₈ alkyl. In embodiments, R³ is independently unsubstituted C₁-C₈ alkyl. In embodiments, R³ is independently substituted or unsubstituted C₁-C₆ alkyl. In embodiments, R³ is independently substituted C₁-C₆ alkyl. In embodiments, R³ is independently unsubstituted C₁-C₆ alkyl. In embodiments, R³ is independently substituted or unsubstituted C₁-C₄ alkyl. In embodiments, R³ is independently substituted C₁-C₄ alkyl. In embodiments, R³ is independently unsubstituted C₁-C₄ alkyl. In embodiments, R³ is independently substituted or unsubstituted ethyl. In embodiments, R³ is independently substituted ethyl. In embodiments, R³ is independently unsubstituted ethyl. In embodiments, R³ is independently substituted or unsubstituted methyl. In embodiments, R³ is independently substituted methyl. In embodiments, R³ is independently unsubstituted methyl.

In embodiments, L⁶ is independently —NHC(O)—. In embodiments, L⁶ is independently —C(O)NH—. In embodiments, L⁶ is independently substituted or unsubstituted alkylene. In embodiments, L⁶ is independently substituted or unsubstituted heteroalkylene.

In embodiments, L⁶ is independently substituted or unsubstituted alkylene (e.g., C₁-C₂₀, C₁-C₁₂, C₁-C₈, C₁-C₆, C₁-C₄, or C₁-C₂). In embodiments, L⁶ is independently substituted alkylene (e.g., C₁-C₂₀, C₁-C₁₂, C₁-C₈, C₁-C₆, C₁-C₄, or C₁-C₂). In embodiments, L⁶ is independently unsubstituted alkylene (e.g., C₁-C₂₀, C₁-C₁₂, C₁-C₈, C₁-C₆, C₁-C₄, or C₁-C₂). In embodiments, L⁶ is independently substituted or unsubstituted C₁-C₂₀ alkylene. In embodiments, L⁶ is independently substituted C₁-C₂₀ alkylene. In embodiments, L⁶ is independently unsubstituted C₁-C₂₀ alkylene. In embodiments, L⁶ is independently substituted or unsubstituted C₁-C₁₂ alkylene. In embodiments, L⁶ is independently substituted C₁-C₁₂ alkylene. In embodiments, L⁶ is independently unsubstituted C₁-C₁₂ alkylene. In embodiments, L⁶ is independently substituted or unsubstituted C₁-C₈ alkylene. In embodiments, L⁶ is independently substituted C₁-C₈ alkylene. In embodiments, L⁶ is independently unsubstituted C₁-C₈ alkylene. In embodiments, L⁶ is independently substituted or unsubstituted C₁-C₆ alkylene. In embodiments, L⁶ is independently substituted C₁-C₆ alkylene. In embodiments, L⁶ is independently unsubstituted C₁-C₆ alkylene. In embodiments, L⁶ is independently substituted or unsubstituted C₁-C₄ alkylene. In embodiments, L⁶ is independently substituted C₁-C₄ alkylene. In embodiments, L⁶ is independently unsubstituted C₁-C₄ alkylene. In embodiments, L⁶ is independently substituted or unsubstituted ethylene. In embodiments, L⁶ is independently substituted ethylene. In embodiments, L⁶ is independently unsubstituted ethylene. In embodiments, L⁶ is independently substituted or unsubstituted methylene. In embodiments, L⁶ is independently substituted methylene. In embodiments, L⁶ is independently unsubstituted methylene.

In embodiments, L⁶ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently substituted or unsubstituted 2 to 20 membered heteroalkylene. In embodiments, L⁶ is independently substituted 2 to 20 membered heteroalkylene. In embodiments, L⁶ is independently unsubstituted 2 to 20 membered heteroalkylene. In embodiments, L⁶ is independently substituted or unsubstituted 2 to 8 membered heteroalkylene. In embodiments, L⁶ is independently substituted 2 to 8 membered heteroalkylene. In embodiments, L⁶ is independently unsubstituted 2 to 8 membered heteroalkylene. In embodiments, L⁶ is independently substituted or unsubstituted 2 to 6 membered heteroalkylene. In embodiments, L⁶ is independently substituted 2 to 6 membered heteroalkylene. In embodiments, L⁶ is independently unsubstituted 2 to 6 membered heteroalkylene. In embodiments, L⁶ is independently substituted or unsubstituted 4 to 6 membered heteroalkylene. In embodiments, L⁶ is independently substituted 4 to 6 membered heteroalkylene. In embodiments, L⁶ is independently unsubstituted 4 to 6 membered heteroalkylene. In embodiments, L⁶ is independently substituted or unsubstituted 2 to 3 membered heteroalkylene. In embodiments, L⁶ is independently substituted 2 to 3 membered heteroalkylene. In embodiments, L⁶ is independently unsubstituted 2 to 3 membered heteroalkylene. In embodiments, L⁶ is independently substituted or unsubstituted 4 to 5 membered heteroalkylene. In embodiments, L⁶ is independently substituted 4 to 5 membered heteroalkylene. In embodiments, L⁶ is independently unsubstituted 4 to 5 membered heteroalkylene.

In embodiments, L^{6A} is independently a bond or unsubstituted alkylene; L^{6B} is independently a bond, —NHC(O)—, or unsubstituted arylene; L^{6C} is independently a bond, unsubstituted alkylene, or unsubstituted arylene; L^{6D} is independently a bond or unsubstituted alkylene; and L^{6E} is independently a bond or —NHC(O)—. In embodiments, L^{6A} is independently a bond or unsubstituted alkylene. In embodiments, L^{6B} is independently a bond, —NHC(O)—, or unsubstituted arylene. In embodiments, L^{6C} is independently a bond, unsubstituted alkylene, or unsubstituted arylene. In embodiments, L^{6D} is independently a bond or unsubstituted alkylene. In embodiments, L^{6E} is independently a bond or —NHC(O)—.

In embodiments, L^{6A} is independently a bond or unsubstituted alkylene (e.g., C₁-C₂₀, C₁-C₁₂, C₁-C₈, C₁-C₆, C₁-C₄, or C₁-C₂). In embodiments, L^{6A} is independently unsubstituted C₁-C₂₀ alkylene. In embodiments, L^{6A} is independently unsubstituted C₁-C₁₂ alkylene. In embodiments, L^{6A} is independently unsubstituted C₁-C₈ alkylene. In embodiments, L^{6A} is independently unsubstituted C₁-C₆ alkylene. In embodiments, L^{6A} is independently unsubstituted C₁-C₄ alkylene. In embodiments, L^{6A} is independently unsubstituted ethylene. In embodiments, L^{6A} is independently unsubstituted methylene. In embodiments, L^{6A} is independently a bond.

In embodiments, L^{6B} is independently a bond. In embodiments, L^{6B} is independently —NHC(O)—. In embodiments, L^{6B} is independently unsubstituted arylene (e.g., C₆-C₁₂, C₆-C₁₀, or phenyl). In embodiments, L^{6B} is independently unsubstituted C₆-C₁₂ arylene. In embodiments, L^{6B} is independently unsubstituted C₆-C₁₀ arylene. In embodiments, L^{6B} is independently unsubstituted phenylene. In embodiments, L^{6B} is independently unsubstituted naphthylene. In embodiments, L^{6B} is independently unsubstituted biphenylene.

In embodiments, L^{6C} is independently a bond or unsubstituted alkylene (e.g., C₁-C₂₀, C₁-C₁₂, C₁-C₈, C₁-C₆, C₁-C₄, or C₁-C₂). In embodiments, L^{6C} is independently unsubstituted C₁-C₂₀ alkylene. In embodiments, L^{6C} is independently unsubstituted C₁-C₁₂ alkylene. In embodiments, L^{6C} is independently unsubstituted C₁-C₈ alkylene. L^{6C} is independently unsubstituted C₂-C₈ alkynylene. In embodiments, L^{6C} is independently unsubstituted C₁-C₆ alkylene. In embodiments, L^{6C} is independently unsubstituted C₁-C₄ alkylene. In embodiments, L^{6C} is independently unsubstituted ethylene. In embodiments, L^{6C} is independently unsubstituted methylene. In embodiments, L^{6C} is independently a bond or unsubstituted alkynylene (e.g., C₂-C₂₀, C₂-C₁₂, C₂-C₅, C₂-C₆, C₂-C₄, or C₂-C₂). In embodiments, L^{6C} is independently unsubstituted C₂-C₂₀ alkynylene. In embodiments, L^{6C} is independently unsubstituted C₂-C₁₂ alkynylene. In embodiments, L^{6C} is independently unsubstituted C₂-C₅ alkynylene. In embodiments, L^{6C} is independently unsubstituted C₂-C₆ alkynylene. In embodiments, L^{6C} is independently unsubstituted C₂-C₄ alkynylene. In embodiments, L^{6C} is independently unsubstituted ethynylene. In embodiments, L^{6C} is independently unsubstituted arylene (e.g., C₆-C₁₂, C₆-C₁₀, or phenyl). In embodiments, L^{6C} is independently unsubstituted C₆-C₁₂ arylene. In embodiments, L^{6C} is independently unsubstituted C₆-C₁₀ arylene. In embodiments, L^{6C} is independently unsubstituted phenylene. In embodiments, L^{6C} is independently unsubstituted naphthylene. In embodiments, L^{6C} is independently a bond.

In embodiments, L^{6D} is independently a bond or unsubstituted alkylene (e.g., C₁-C₂₀, C₁-C₁₂, C₁-C₈, C₁-C₆, C₁-C₄, or C₁-C₂). In embodiments, L^{6D} is independently unsubstituted C₁-C₂₀ alkylene. In embodiments, L^{6D} is independently unsubstituted C₁-C₁₂ alkylene. In embodiments, L^{6A} is independently unsubstituted C₁-C₈ alkylene. In embodiments, L^{6D} is independently unsubstituted C₁-C₆ alkylene. In embodiments, L^{6D} is independently unsubstituted C₁-C₄ alkylene. In embodiments, L^{6D} is independently unsubstituted ethylene. In embodiments, L^{6D} is independently unsubstituted methylene. In embodiments, L^{6D} is independently a bond.

In embodiments, L^{6E} is independently a bond. In embodiments, L^{6E} is independently —NHC(O)—.

In embodiments, L^{6A} is independently a bond or unsubstituted C₁-C₈ alkylene. In embodiments, L^{6B} is independently a bond, —NHC(O)—, or unsubstituted phenylene. In embodiments, L^{6C} is independently a bond, unsubstituted C₂-C₈ alkynylene, or unsubstituted phenylene. In embodiments, L^{6D} is independently a bond or unsubstituted C₁-C₈ alkylene. In embodiments, L^{6E} is independently a bond or —NHC(O)—.

In embodiments, L⁶ is independently a bond,

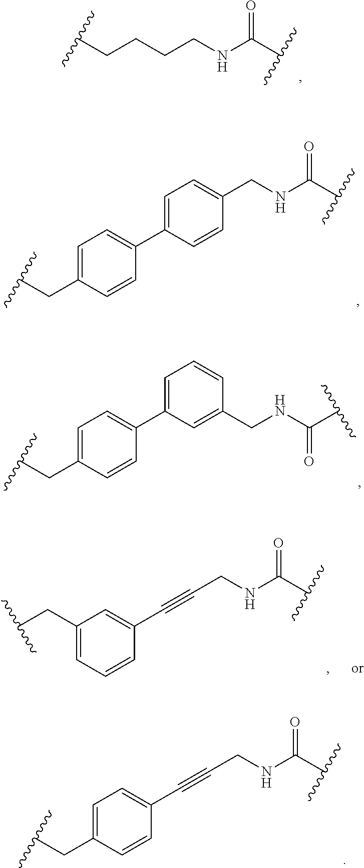 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

In embodiments, L⁶ is independently a bond. In embodiments, L⁶ is independently

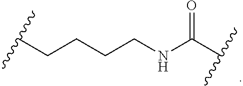 [SMILES: *CCCCNC(*)=O]

In embodiments, L⁶ is independently

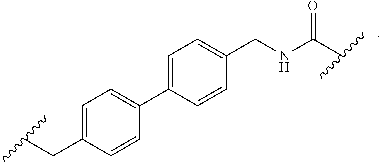 [SMILES: *Cc1ccc(-c2ccc(CNC(*)=O)cc2)cc1]

In embodiments, L⁶ is independently

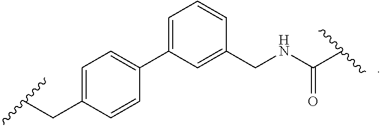 [SMILES: *Cc1ccc(-c2cccc(CNC(*)=O)c2)cc1]

In embodiments, L⁶ is independently

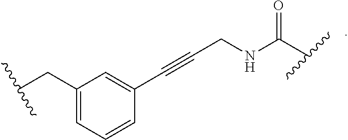 [SMILES: *Cc1cccc(C#CCNC(*)=O)c1]

In embodiments, L⁶ is independently

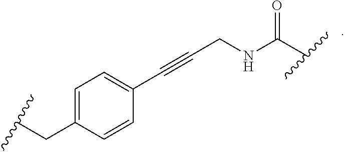 [SMILES: *Cc1ccc(C#CCNC(*)=O)cc1]

In embodiments, L⁵ is independently —NHC(O)—. In embodiments, L⁵ is independently —C(O)NH—. In embodiments, L⁵ is independently substituted or unsubstituted alkylene. In embodiments, L⁵ is independently substituted or unsubstituted heteroalkylene.

In embodiments, L⁵ is independently substituted or unsubstituted alkylene (e.g., C₁-C₂₀, C₁-C₁₂, C₁-C₈, C₁-C₆, C₁-C₄, or C₁-C₂). In embodiments, L⁵ is independently substituted alkylene (e.g., C₁-C₂₀, C₁-C₁₂, C₁-C₈, C₁-C₆, C₁-C₄, or C₁-C₂). In embodiments, L⁵ is independently unsubstituted alkylene (e.g., C₁-C₂₀, C₁-C₁₂, C₁-C₈, C₁-C₆, C₁-C₄, or C₁-C₂). In embodiments, L⁵ is independently substituted or unsubstituted C₁-C₂₀ alkylene. In embodiments, L⁵ is independently substituted C₁-C₂₀ alkylene. In embodiments, L⁵ is independently unsubstituted C₁-C₂₀ alkylene. In embodiments, L⁵ is independently substituted or unsubstituted C₁-C₁₂ alkylene. In embodiments, L⁵ is independently substituted C₁-C₁₂ alkylene. In embodiments, L⁵ is independently unsubstituted C₁-C₁₂ alkylene. In embodiments, L⁵ is independently substituted or unsubstituted C₁-C₈ alkylene. In embodiments, L⁵ is independently substituted C₁-C₈ alkylene. In embodiments, L⁵ is independently unsubstituted C₁-C₈ alkylene. In embodiments, L⁵ is independently substituted or unsubstituted C₁-C₆ alkylene. In embodiments, L⁵ is independently substituted C₁-C₆ alkylene. In embodiments, L⁵ is independently unsubstituted C₁-C₆ alkylene. In embodiments, L⁵ is independently substituted or unsubstituted C₁-C₄ alkylene. In embodiments, L⁵ is independently substituted C₁-C₄ alkylene. In embodiments, L⁵ is independently unsubstituted C₁-C₄ alkylene. In embodiments, L⁵ is independently substituted or unsubstituted ethylene. In embodiments, L⁵ is independently substituted ethylene. In embodiments, L⁵ is independently unsubstituted ethylene. In embodiments, L⁵ is independently substituted or unsubstituted methylene. In embodiments, L⁵ is independently substituted methylene. In embodiments, L⁵ is independently unsubstituted methylene.

In embodiments, L⁵ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁵ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁵ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁵ is independently substituted or unsubstituted 2 to 20 membered heteroalkylene. In embodiments, L⁵ is independently substituted 2 to 20 membered heteroalkylene. In embodiments, L⁵ is independently unsubstituted 2 to 20 membered heteroalkylene. In embodiments, L⁵ is independently substituted or unsubstituted 2 to 8 membered heteroalkylene. In embodiments, L⁵ is independently substituted 2 to 8 membered heteroalkylene. In embodiments, L⁵ is independently unsubstituted 2 to 8 membered heteroalkylene. In embodiments, L⁵ is independently substituted or unsubstituted 2 to 6 membered heteroalkylene. In embodiments, L⁵ is independently substituted 2 to 6 membered heteroalkylene. In embodiments, L⁵ is independently unsubstituted 2 to 6 membered heteroalkylene. In embodiments, L⁵ is independently substituted or unsubstituted 4 to 6 membered heteroalkylene. In embodiments, L⁵ is independently substituted 4 to 6 membered heteroalkylene. In embodiments, L⁵ is independently unsubstituted 4 to 6 membered heteroalkylene. In embodiments, L⁵ is independently substituted or unsubstituted 2 to 3 membered heteroalkylene. In embodiments, L⁵ is independently substituted 2 to 3 membered heteroalkylene. In embodiments, L⁵ is independently unsubstituted 2 to 3 membered heteroalkylene. In embodiments, L⁵ is independently substituted or unsubstituted 4 to 5 membered heteroalkylene. In embodiments, L⁵ is independently substituted 4 to 5 membered heteroalkylene. In embodiments, L⁵ is independently unsubstituted 4 to 5 membered heteroalkylene.

In embodiments, L^{5A} is independently a bond or unsubstituted alkylene; L^{5B} is independently a bond, —NHC(O)—, or unsubstituted arylene; L^{5C} is independently a bond, unsubstituted alkylene, or unsubstituted arylene; L^{5D} is independently a bond or unsubstituted alkylene; and L^{5E} is independently a bond or —NHC(O)—. In embodiments, L^{5A} is independently a bond or unsubstituted alkylene. In embodiments, L^{5B} is independently a bond, —NHC(O)—, or unsubstituted arylene. In embodiments, L^{5C} is independently a bond, unsubstituted alkylene, or unsubstituted arylene. In embodiments, L^{5D} is independently a bond or unsubstituted alkylene. In embodiments, L^{5E} is independently a bond or —NHC(O)—.

In embodiments, L^{5A} is independently a bond or unsubstituted alkylene (e.g., C₁-C₂₀, C₁-C₁₂, C₁-C₈, C₁-C₆, C₁-C₄, or C₁-C₂). In embodiments, L^{5A} is independently unsubstituted C₁-C₂₀ alkylene. In embodiments, L^{5A} is independently unsubstituted C₁-C₁₂ alkylene. In embodiments, L^{5A} is independently unsubstituted C₁-C₈ alkylene. In embodiments, L^{5A} is independently unsubstituted C₁-C₆ alkylene. In embodiments, L^{5A} is independently unsubstituted C₁-C₄ alkylene. In embodiments, L^{5A} is independently unsubstituted ethylene. In embodiments, L^{5A} is independently unsubstituted methylene. In embodiments, L^{5A} is independently a bond.

In embodiments, L^{5B} is independently a bond. In embodiments, L^{5B} is independently —NHC(O)—. In embodiments, L^{5B} is independently unsubstituted arylene (e.g., C₆-C₁₂, C₆-C₁₀, or phenyl). In embodiments, L^{5B} is independently unsubstituted C₆-C₁₂ arylene. In embodiments, L^{5B} is independently unsubstituted C₆-C₁₀ arylene. In embodiments, L^{5B} is independently unsubstituted phenylene. In embodiments, LB is independently unsubstituted naphthylene.

In embodiments, L^{5C} is independently a bond or unsubstituted alkylene (e.g., C₁-C₂₀, C₁-C₁₂, C₁-C₈, C₁-C₆, C₁-C₄, or C₁-C₂). In embodiments, L^{5C} is independently unsubstituted C₁-C₂₀ alkylene. In embodiments, L^{5C} is independently unsubstituted C₁-C₁₂ alkylene. In embodiments, L^{5C} is independently unsubstituted C₁-C₅ alkylene. L^{5C} is independently unsubstituted C₂-C₅ alkynylene. In embodiments, L^{5C} is independently unsubstituted C₁-C₆ alkylene. In embodiments, L^{5C} is independently unsubstituted C₁-C₄ alkylene. In embodiments, L^{5C} is independently unsubstituted ethylene. In embodiments, L^{5C} is independently unsubstituted methylene. In embodiments, L^{5C} is independently a bond or unsubstituted alkynylene (e.g., C₂-C₂₀, C₂-C₁₂, C₂-C₅, C₂-C₆, C₂-C₄, or C₂-C₂). In embodiments, L^{5C} is independently unsubstituted C₂-C₂₀ alkynylene. In embodiments, L^{5C} is independently unsubstituted C₂-C₁₂ alkynylene. In embodiments, L^{5C} is independently unsubstituted C₂-C₈ alkynylene. In embodiments, L^{5C} is independently unsubstituted C₂-C₆ alkynylene. In embodiments, L^{5C} is independently unsubstituted C₂-C₄ alkynylene. In embodiments, L^{5C} is independently unsubstituted ethynylene. In embodiments, L^{5C} is independently unsubstituted arylene (e.g., C₆-C₁₂, C₆-C₁₀, or phenyl). In embodiments, L^{5C} is independently unsubstituted C₆-C₁₂ arylene. In embodiments, L^{5C} is independently unsubstituted C₆-C₁₀ arylene. In embodiments, L^{5C} is independently unsubstituted phenylene. In embodiments, L^{5C} is independently unsubstituted naphthylene. In embodiments, L^{5C} is independently a bond.

In embodiments, L^{5D} is independently a bond or unsubstituted alkylene (e.g., C₁-C₂₀, C₁-C₁₂, C₁-C₈, C₁-C₆, C₁-C₄, or C₁-C₂). In embodiments, L^{5D} is independently unsubstituted C₁-C₂₀ alkylene. In embodiments, L^{5D} is independently unsubstituted C₁-C₁₂ alkylene. In embodiments, L^{5A} is independently unsubstituted C₁-C₈ alkylene. In embodiments, L^{5D} is independently unsubstituted C₁-C₆ alkylene. In embodiments, L^{5D} is independently unsubstituted C₁-C₄ alkylene. In embodiments, L^{5D} is independently unsubstituted ethylene. In embodiments, L^{5D} is independently unsubstituted methylene. In embodiments, L^{5D} is independently a bond.

In embodiments, L^{5E} is independently a bond. In embodiments, L^{5E} is independently —NHC(O)—.

In embodiments, L^{5A} is independently a bond or unsubstituted C₁-C₈ alkylene. In embodiments, L^{5B} is independently a bond, —NHC(O)—, or unsubstituted phenylene. In embodiments, L^{5C} is independently a bond, unsubstituted C₂-C₅ alkynylene, or unsubstituted phenylene. In embodiments, L^{ID} is independently a bond or unsubstituted C₁-C₈ alkylene. In embodiments, L^{5E} is independently a bond or —NHC(O)—.

In embodiments, L^{S} is independently a bond,

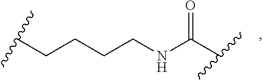 [SMILES: *CCCCNC(*)=O]

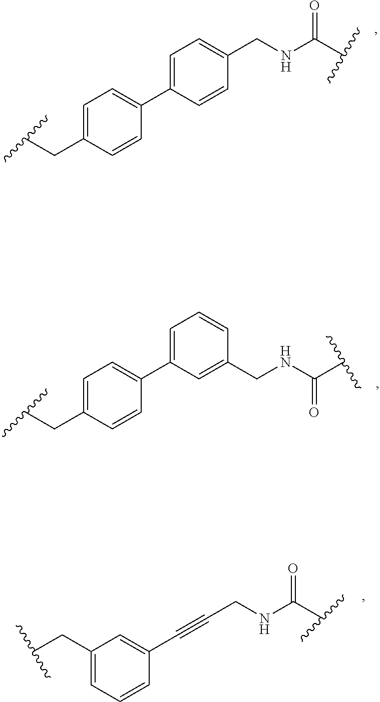 [SMILES: *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1]

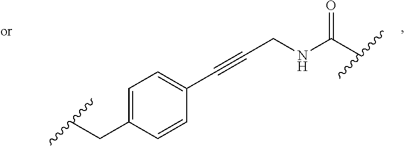 [SMILES: *Cc1ccc(C#CCNC(*)=O)cc1]

In embodiments, L^{S} is independently a bond. In embodiments, L⁵ is independently

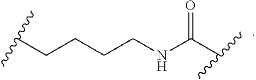 [SMILES: *CCCCNC(*)=O]

In embodiments, L⁵ is independently

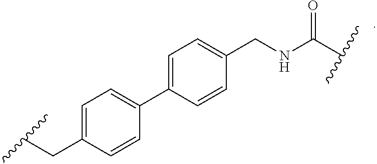 [SMILES: *Cc1ccc(-c2ccc(CNC(*)=O)cc2)cc1]

In embodiments, L⁵ is independently

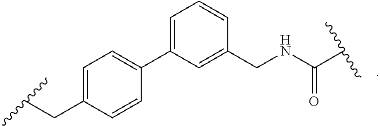 [SMILES: *Cc1ccc(-c2cccc(CNC(*)=O)c2)cc1]

In embodiments, L⁵ is independently

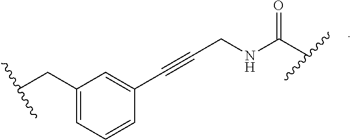 [SMILES: *Cc1cccc(C#CCNC(*)=O)c1]

In embodiments, L⁵ is independently

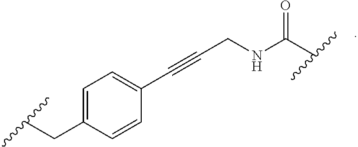 [SMILES: *Cc1ccc(C#CCNC(*)=O)cc1]

In embodiments, R¹ is unsubstituted alkyl (e.g., C₁-C₂₅, C₁-C₂₀, C₁-C₁₇, C₁-C₁₂, C₁-C₈, C₁-C₆, C₁-C₄, or C₁-C₂). In embodiments, R¹ is unsubstituted unbranched alkyl (e.g., C₁-C₂₅, C₁-C₂₀, C₁-C₁₇, C₁-C₁₂, C₁-C₈, C₁-C₆, C₁-C₄, or C₁-C₂). In embodiments, R¹ is unsubstituted unbranched saturated alkyl (e.g., C₁-C₂₅, C₁-C₂₀, C₁-C₁₇, C₁-C₁₂, C₁-C₈, C₁-C₆, C₁-C₄, or C₁-C₂). In embodiments, R¹ is unsubstituted unbranched unsaturated alkyl (e.g., C₁-C₂₅, C₁-C₂₀, C₁-C₁₇, C₁-C₁₂, C₁-C₈, C₁-C₆, C₁-C₄, or C₁-C₂).

In embodiments, R¹ is unsubstituted C₁-C₁₇ alkyl. In embodiments, R¹ is unsubstituted C₁₁-C₁₇ alkyl. In embodiments, R¹ is unsubstituted C₁₃-C₁₇ alkyl. In embodiments, R¹ is unsubstituted C₁₄-C₁₅ alkyl. In embodiments, R¹ is unsubstituted Cis alkyl. In embodiments, R¹ is unsubstituted C₁₄ alkyl.

In embodiments, R¹ is unsubstituted unbranched C₁-C₁₇ alkyl. In embodiments, R¹ is unsubstituted unbranched C₁-C₁₇ alkyl. In embodiments, R¹ is unsubstituted unbranched C₁₃-C₁₇ alkyl. In embodiments, R¹ is unsubstituted unbranched C₁₄-C₁₅ alkyl. In embodiments, R¹ is unsubstituted unbranched C₁₄ alkyl. In embodiments, R¹ is unsubstituted unbranched C₁₅ alkyl.

In embodiments, R¹ is unsubstituted unbranched saturated C₁-C₁₇ alkyl. In embodiments, R¹ is unsubstituted unbranched saturated C₁-C₁₇ alkyl. In embodiments, R¹ is unsubstituted unbranched saturated C₁₃-C₁₇ alkyl. In embodiments, R¹ is unsubstituted unbranched saturated C₁₄-C₁₅ alkyl. In embodiments, R¹ is unsubstituted unbranched saturated C₁₄ alkyl. In embodiments, R¹ is unsubstituted unbranched saturated C₁₅ alkyl.

In embodiments, R¹ is unsubstituted unbranched unsaturated C₁-C₁₇ alkyl. In embodiments, R¹ is unsubstituted unbranched unsaturated C₁-C₁₇ alkyl. In embodiments, R¹ is unsubstituted unbranched unsaturated C₁₃-C₁₇ alkyl. In embodiments, R¹ is unsubstituted unbranched unsaturated C₁₄-C₁₅ alkyl. In embodiments, R¹ is unsubstituted unbranched unsaturated C₁₄ alkyl. In embodiments, R¹ is unsubstituted unbranched unsaturated Cis alkyl.

In embodiments, R² is unsubstituted alkyl (e.g., C₁-C₂₅, C₁-C₂₀, C₁-C₁₇, C₁-C₁₂, C₁-C₈, C₁-C₆, C₁-C₄, or C₁-C₂). In embodiments, R² is unsubstituted unbranched alkyl (e.g., C₁-C₂₅, C₁-C₂₀, C₁-C₁₇, C₁-C₁₂, C₁-C₈, C₁-C₆, C₁-C₄, or C₁-C₂). In embodiments, R² is unsubstituted unbranched saturated alkyl (e.g., C₁-C₂₅, C₁-C₂₀, C₁-C₁₇, C₁-C₁₂, C₁-C₈, C₁-C₆, C₁-C₄, or C₁-C₂). In embodiments, R² is unsubstituted unbranched unsaturated alkyl (e.g., C₁-C₂₅, C₁-C₂₀, C₁-C₁₇, C₁-C₁₂, C₁-C₈, C₁-C₆, C₁-C₄, or C₁-C₂).

In embodiments, R² is unsubstituted C₁-C₁₇ alkyl. In embodiments, R² is unsubstituted C₁₁-C₁₇ alkyl. In embodiments, R² is unsubstituted C₁₃-C₁₇ alkyl. In embodiments, R² is unsubstituted C₁₄-C₁₅ alkyl. In embodiments, R² is unsubstituted C₁₄ alkyl. In embodiments, R² is unsubstituted Cis alkyl.

In embodiments, R² is unsubstituted unbranched C₁-C₁₇ alkyl. In embodiments, R² is unsubstituted unbranched C₁-C₁₇ alkyl. In embodiments, R² is unsubstituted unbranched C₁₃-C₁₇ alkyl. In embodiments, R² is unsubstituted unbranched C₁₄-Cis alkyl. In embodiments, R² is unsubstituted unbranched C₁₄ alkyl. In embodiments, R² is unsubstituted unbranched C₁₅ alkyl.

In embodiments, R² is unsubstituted unbranched saturated C₁-C₁₇ alkyl. In embodiments, R² is unsubstituted unbranched saturated C₁-C₁₇ alkyl. In embodiments, R² is unsubstituted unbranched saturated C₁₃-C₁₇ alkyl. In embodiments, R² is unsubstituted unbranched saturated C₁₄-C₁₅ alkyl. In embodiments, R² is unsubstituted unbranched saturated C₁₄ alkyl. In embodiments, R² is unsubstituted unbranched saturated C₁₅ alkyl.

In embodiments, R² is unsubstituted unbranched unsaturated C₁-C₁₇ alkyl. In embodiments, R² is unsubstituted unbranched unsaturated C₁-C₁₇ alkyl. In embodiments, R² is unsubstituted unbranched unsaturated C₁₃-C₁₇ alkyl. In embodiments, R² is unsubstituted unbranched unsaturated C₁₄-C₁₅ alkyl. In embodiments, R² is unsubstituted unbranched unsaturated C₁₄ alkyl. In embodiments, R² is unsubstituted unbranched unsaturated C₁₅ alkyl.

In embodiments, at least one of R¹ and R² is unsubstituted C₁-C₁₉ alkyl. In embodiments, at least one of R¹ and R² is unsubstituted C₉-C₁₉ alkyl. In embodiments, at least one of R¹ and R² is unsubstituted C₁₁-C₁₉ alkyl. In embodiments, at least one of R¹ and R² is unsubstituted C₁₃-C₁₉ alkyl.

In embodiments, R¹ is unsubstituted C₁-C₁₉ alkyl. In embodiments, R¹ is unsubstituted C₉-C₁₉ alkyl. In embodiments, R¹ is unsubstituted C₁₁-C₁₉ alkyl. In embodiments, R¹ is unsubstituted C₁₃-C₁₉ alkyl. In embodiments, R¹ is unsubstituted unbranched C₁-C₁₉ alkyl. In embodiments, R¹ is unsubstituted unbranched C₉-C₁₉ alkyl. In embodiments, R¹ is unsubstituted unbranched C₁₁-C₁₉ alkyl. In embodiments, R¹ is unsubstituted unbranched C₁₃-C₁₉ alkyl. In embodiments, R¹ is unsubstituted unbranched saturated C₁-C₁₉ alkyl. In embodiments, R¹ is unsubstituted unbranched saturated C₉-C₁₉ alkyl. In embodiments, R¹ is unsubstituted unbranched saturated C₁₁-C₁₉ alkyl. In embodiments, R¹ is unsubstituted unbranched saturated C₁₃-C₁₉ alkyl. In embodiments, R¹ is unsubstituted unbranched unsaturated C₁-C₁₉ alkyl. In embodiments, R¹ is unsubstituted unbranched unsaturated C₉-C₁₉ alkyl. In embodiments, R¹ is unsubstituted unbranched unsaturated C₁-C₁₉ alkyl. In embodiments, R¹ is unsubstituted unbranched unsaturated C₁₃-C₁₉ alkyl.

In embodiments, R² is unsubstituted C₁-C₁₉ alkyl. In embodiments, R² is unsubstituted C₉-C₁₉ alkyl. In embodiments, R² is unsubstituted C₁₁-C₁₉ alkyl. In embodiments, R² is unsubstituted C₁₃-C₁₉ alkyl. In embodiments, R² is unsubstituted unbranched C₁-C₁₉ alkyl. In embodiments, R² is unsubstituted unbranched C₉-C₁₉ alkyl. In embodiments, R² is unsubstituted unbranched C₁₁-C₁₉ alkyl. In embodiments, R² is unsubstituted unbranched C₁₃-C₁₉ alkyl. In embodiments, R² is unsubstituted unbranched saturated C₁-C₁₉ alkyl. In embodiments, R² is unsubstituted unbranched saturated C₉-C₁₉ alkyl. In embodiments, R² is unsubstituted unbranched saturated C₁₁-C₁₉ alkyl. In embodiments, R² is unsubstituted unbranched saturated C₁₃-C₁₉ alkyl. In embodiments, R² is unsubstituted unbranched unsaturated C₁-C₁₉ alkyl. In embodiments, R² is unsubstituted unbranched unsaturated C₉-C₁₉ alkyl. In embodiments, R² is unsubstituted unbranched unsaturated C₁₁-C₁₉ alkyl. In embodiments, R² is unsubstituted unbranched unsaturated C₁₃-C₁₉ alkyl.

L³ is independently a bond, a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L³ is independently a bond, a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L³ is independently a bond, a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, unsubstituted alkylene (e.g., C₁-C₂₀, C₁-C₁₂, C₁-C₅, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L³ is substituted, L³ is substituted with a substituent group. In embodiments, when L³ is substituted, L³ is substituted with a size-limited substituent group. In embodiments, when L³ is substituted, L³ is substituted with a lower substituent group.

L⁴ is independently a bond, a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁴ is a bond, a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L⁴ is a bond, a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L⁴ is substituted, L⁴ is substituted with a substituent group. In embodiments, when L⁴ is substituted, L⁴ is substituted with a size-limited substituent group. In embodiments, when L⁴ is substituted, L⁴ is substituted with a lower substituent group.

R²³ is independently hydrogen or unsubstituted alkyl (e.g., C₁-C₂₃, C₁-C₁₂, C₁-C₈, C₁-C₆, C₁-C₄, or C₁-C₂). In embodiments, R²³ is independently hydrogen. In embodiments, R²³ is independently unsubstituted C₁-C₂₃ alkyl. In embodiments, R²³ is independently hydrogen or unsubstituted C₁-C₁₂ alkyl. In embodiments, R²³ is independently hydrogen or unsubstituted C₁-C₁₀ alkyl. In embodiments, R²³ is independently hydrogen or unsubstituted C₁-C₈ alkyl. In embodiments, R²³ is independently hydrogen or unsubstituted C₁-C₆ alkyl. In embodiments, R²³ is independently hydrogen or unsubstituted C₁-C₄ alkyl. In embodiments, R²³ is independently hydrogen or unsubstituted C₁-C₂ alkyl.

R²⁴ is independently hydrogen or unsubstituted alkyl (e.g., C₁-C₂₃, C₁-C₁₂, C₁-C₈, C₁-C₆, C₁-C₄, or C₁-C₂). In embodiments, R²⁴ is independently hydrogen. In embodiments, R²⁴ is independently unsubstituted C₁-C₂₃ alkyl. In embodiments, R²⁴ is independently hydrogen or unsubstituted C₁-C₁₂ alkyl. In embodiments, R²⁴ is independently hydrogen or unsubstituted C₁-C₁₀ alkyl. In embodiments, R²⁴ is independently hydrogen or unsubstituted C₁-C₈ alkyl. In embodiments, R²⁴ is independently hydrogen or unsubstituted C₁-C₆ alkyl. In embodiments, R²⁴ is independently hydrogen or unsubstituted C₁-C₄ alkyl. In embodiments, R²⁴ is independently hydrogen or unsubstituted C₁-C₂ alkyl.

R²⁵ is independently hydrogen or unsubstituted alkyl (e.g., C₁-C₂₃, C₁-C₁₂, C₁-C₈, C₁-C₆, C₁-C₄, or C₁-C₂). In embodiments, R²⁵ is independently hydrogen. In embodiments, R²⁵ is independently unsubstituted C₁-C₂₃ alkyl. In embodiments, R²⁵ is independently hydrogen or unsubstituted C₁-C₁₂ alkyl. In embodiments, R²⁵ is independently hydrogen or unsubstituted C₁-C₁₀ alkyl. In embodiments, R²⁵ is independently hydrogen or unsubstituted C₁-C₈ alkyl. In embodiments, R²⁵ is independently hydrogen or unsubstituted C₁-C₆ alkyl. In embodiments, R²⁵ is independently hydrogen or unsubstituted C₁-C₄ alkyl. In embodiments, R²⁵ is independently hydrogen or unsubstituted C₁-C₂ alkyl.

L⁵ is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁵ is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L⁵ is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L⁵ is substituted, L⁵ is substituted with a substituent group. In embodiments, when L⁵ is substituted, L⁵ is substituted with a size-limited substituent group. In embodiments, when L⁵ is substituted, L⁵ is substituted with a lower substituent group.

L^{5A} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5A} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5A} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5A} is substituted, L^{5A} is substituted with a substituent group. In embodiments, when L^{5A} is substituted, L^{5A} is substituted with a size-limited substituent group. In embodiments, when L⁵A is substituted, L^{5A} is substituted with a lower substituent group.

L^{5B} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5B} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5B} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5B} is substituted, L^{5B} is substituted with a substituent group. In embodiments, when L⁵B is substituted, L^{5B} is substituted with a size-limited substituent group. In embodiments, when L^{5B} is substituted, L^{5B} is substituted with a lower substituent group.

L⁵c is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁵c is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5C} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5C} is substituted, L^{5C} is substituted with a substituent group. In embodiments, when L^{5C} is substituted, L^{5C} is substituted with a size-limited substituent group. In embodiments, when L^{5C} is substituted, L^{5C} is substituted with a lower substituent group.

L^{5D} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5D} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{D} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5D} is substituted, L^{5D} is substituted with a substituent group. In embodiments, when L⁵D is substituted, L^{5D} is substituted with a size-limited substituent group. In embodiments, when L^{5D} is substituted, L^{5D} is substituted with a lower substituent group.

L^{5E} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5E} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5E} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5E} is substituted, L^{5E} is substituted with a substituent group. In embodiments, when L^{5E} is substituted, L^{5E} is substituted with a size-limited substituent group. In embodiments, when L^{5E} is substituted, L^{5E} is substituted with a lower substituent group.

L⁶ is independently a

bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁶ is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L⁶ is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L⁶ is substituted, L⁶ is substituted with a substituent group. In embodiments, when L⁶ is substituted, L⁶ is substituted with a size-limited substituent group. In embodiments, when L⁶ is substituted, L⁶ is substituted with a lower substituent group.

L^{6A} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁶A is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₅, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6A} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₅, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6A} is substituted, L^{6A} is substituted with a substituent group. In embodiments, when L^{6A} is substituted, L^{6A} is substituted with a size-limited substituent group. In embodiments, when L^{6A} is substituted, L^{6A} is substituted with a lower substituent group.

L^{6B} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6B} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6B} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6B} is substituted, L^{6B} is substituted with a substituent group. In embodiments, when L^{6B} is substituted, L^{6B} is substituted with a size-limited substituent group. In embodiments, when L^{6B} is substituted, L^{6B} is substituted with a lower substituent group.

L^{6C} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6C} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6C} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6C} is substituted, L^{6C} is substituted with a substituent group. In embodiments, when L^{6C} is substituted, L^{6C} is substituted with a size-limited substituent group. In embodiments, when L^{6C} is substituted, L^{6C} is substituted with a lower substituent group.

L^{6D} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6D} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6D} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6D} is substituted, L^{6D} is substituted with a substituent group. In embodiments, when L^{6D} is substituted, L^{6D} is substituted with a size-limited substituent group. In embodiments, when L^{6D} is substituted, L^{6D} is substituted with a lower substituent group.

L^{6E} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6E} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6E} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₅, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6E} is substituted, L^{6E} is substituted with a substituent group. In embodiments, when L^{6E} is substituted, L^{6E} is substituted with a size-limited substituent group. In embodiments, when L^{6E} is substituted, L^{6E} is substituted with a lower substituent group.

In embodiments, L⁷ is independently substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂). In embodiments, L⁷ is independently substituted (e.g., substituted with a substituent group, a size-limited substituent group, or lower substituent group) alkylene (e.g., C₁-C₂₀, C₁-C₁₂, C₁-C₅, C₁-C₆, C₁-C₄, or C₁-C₂). In embodiments, L⁷ is independently unsubstituted alkylene (e.g., C₁-C₂₀, C₁-C₁₂, C₁-C₈, C₁-C₆, C₁-C₄, or C₁-C₂).

In embodiments, L⁷ is independently substituted (e.g., substituted with a substituent group, a size-limited substituent group, or lower substituent group) or unsubstituted heteroalkylene (e.g., 2 to 20 membered, 2 to 12 membered, 2 to 10 membered, 2 to 8 membered, 2 to 6 membered, or 2 to 4 membered). In embodiments, L⁷ is independently substituted (e.g., substituted with a substituent group, a size-limited substituent group, or lower substituent group) heteroalkylene (e.g., 2 to 20 membered, 2 to 12 membered, 2 to 10 membered, 2 to 8 membered, 2 to 6 membered, or 2 to 4 membered). In embodiments, L⁷ is independently unsubstituted heteroalkylene (e.g., 2 to 20 membered, 2 to 12 membered, 2 to 10 membered, 2 to 8 membered, 2 to 6 membered, or 2 to 4 membered). In embodiments, L⁷ is independently substituted (e.g., substituted with a substituent group, a size-limited substituent group, or lower substituent group) or unsubstituted heteroalkenylene (e.g., 2 to 20 membered, 2 to 12 membered, 2 to 10 membered, 2 to 8 membered, 2 to 6 membered, or 2 to 4 membered). In embodiments, L⁷ is independently substituted (e.g., substituted with a substituent group, a size-limited substituent group, or lower substituent group) heteroalkenylene (e.g., 2 to 20 membered, 2 to 12 membered, 2 to 10 membered, 2 to 8 membered, 2 to 6 membered, or 2 to 4 membered). In embodiments, L⁷ is independently unsubstituted heteroalkenylene (e.g., 2 to 20 membered, 2 to 12 membered, 2 to 10 membered, 2 to 8 membered, 2 to 6 membered, or 2 to 4 membered). In embodiments, when L⁷ is substituted, L⁷ is substituted with a substituent group. In embodiments, when L⁷ is substituted, L⁷ is substituted with a size-limited substituent group. In embodiments, when L⁷ is substituted, L⁷ is substituted with a lower substituent group.

In embodiments, R¹ is unsubstituted alkyl (e.g., C₁-C₂₅, C₁-C₂₀, C₁-C₁₂, C₁-C₈, C₁-C₆, C₁-C₄, or C₁-C₂). In embodiments, R¹ is unsubstituted C₁-C₂₅ alkyl. In embodiments, R¹ is unsubstituted C₁-C₂₀ alkyl. In embodiments, R¹ is unsubstituted C₁-C₁₂ alkyl. In embodiments, R¹ is unsubstituted C₁-C₈ alkyl. In embodiments, R¹ is unsubstituted C₁-C₆ alkyl. In embodiments, R¹ is unsubstituted C₁-C₄ alkyl. In embodiments, R¹ is unsubstituted C₁-C₂ alkyl.

In embodiments, R¹ is unsubstituted branched alkyl (e.g., C₁-C₂₅, C₁-C₂₀, C₁-C₁₂, C₁-C₈, C₁-C₆, C₁-C₄, or C₁-C₂). In embodiments, R¹ is unsubstituted branched C₁-C₂₅ alkyl. In embodiments, R¹ is unsubstituted branched C₁-C₂₀ alkyl. In embodiments, R¹ is unsubstituted branched C₁-C₁₂ alkyl. In embodiments, R¹ is unsubstituted branched C₁-C₈ alkyl. In embodiments, R¹ is unsubstituted branched C₁-C₆ alkyl. In embodiments, R¹ is unsubstituted branched C₁-C₄ alkyl. In embodiments, R¹ is unsubstituted branched C₁-C₂ alkyl.

In embodiments, R¹ is unsubstituted unbranched alkyl (e.g., C₁-C₂₅, C₁-C₂₀, C₁-C₁₂, C₁-C₈, C₁-C₆, C₁-C₄, or C₁-C₂). In embodiments, R¹ is unsubstituted unbranched C₁-C₂₅ alkyl. In embodiments, R¹ is unsubstituted unbranched C₁-C₂₀ alkyl. In embodiments, R¹ is unsubstituted unbranched C₁-C₁₂ alkyl. In embodiments, R¹ is unsubstituted unbranched C₁-C₈ alkyl. In embodiments, R¹ is unsubstituted unbranched C₁-C₆ alkyl. In embodiments, R¹ is unsubstituted unbranched C₁-C₄ alkyl. In embodiments, R¹ is unsubstituted unbranched C₁-C₂ alkyl.

In embodiments, R¹ is unsubstituted branched saturated alkyl (e.g., C₁-C₂₅, C₁-C₂₀, C₁-C₁₂, C₁-C₈, C₁-C₆, C₁-C₄, or C₁-C₂). In embodiments, R¹ is unsubstituted branched saturated C₁-C₂₅ alkyl. In embodiments, R¹ is unsubstituted branched saturated C₁-C₂₀ alkyl. In embodiments, R¹ is unsubstituted branched saturated C₁-C₁₂ alkyl. In embodiments, R¹ is unsubstituted branched saturated C₁-C₈ alkyl. In embodiments, R¹ is unsubstituted branched saturated C₁-C₆ alkyl. In embodiments, R¹ is unsubstituted branched saturated C₁-C₄ alkyl. In embodiments, R¹ is unsubstituted branched saturated C₁-C₂ alkyl.

In embodiments, R¹ is unsubstituted branched unsaturated alkyl (e.g., C₁-C₂₅, C₁-C₂₀, C₁-C₁₂, C₁-C₈, C₁-C₆, C₁-C₄, or C₁-C₂). In embodiments, R¹ is unsubstituted branched unsaturated C₁-C₂₅ alkyl. In embodiments, R¹ is unsubstituted branched unsaturated C₁-C₂₀ alkyl. In embodiments, R¹ is unsubstituted branched unsaturated C₁-C₁₂ alkyl. In embodiments, R¹ is unsubstituted branched unsaturated C₁-C₈ alkyl. In embodiments, R¹ is unsubstituted branched unsaturated C₁-C₆ alkyl. In embodiments, R¹ is unsubstituted branched unsaturated C₁-C₄ alkyl. In embodiments, R¹ is unsubstituted branched saturated C₁-C₂ alkyl.

In embodiments, R¹ is unsubstituted unbranched saturated alkyl (e.g., C₁-C₂₅, C₁-C₂₀, C₁-C₁₂, C₁-C₈, C₁-C₆, C₁-C₄, or C₁-C₂). In embodiments, R¹ is unsubstituted unbranched saturated C₁-C₂₅ alkyl. In embodiments, R¹ is unsubstituted unbranched saturated C₁-C₂₀ alkyl. In embodiments, R¹ is unsubstituted unbranched saturated C₁-C₁₂ alkyl. In embodiments, R¹ is unsubstituted unbranched saturated C₁-C₈ alkyl. In embodiments, R¹ is unsubstituted unbranched saturated C₁-C₆ alkyl. In embodiments, R¹ is unsubstituted unbranched saturated C₁-C₄ alkyl. In embodiments, R¹ is unsubstituted unbranched saturated C₁-C₂ alkyl.

In embodiments, R¹ is unsubstituted unbranched unsaturated alkyl (e.g., C₁-C₂₅, C₁-C₂₀, C₁-C₁₂, C₁-C₈, C₁-C₆, C₁-C₄, or C₁-C₂). In embodiments, R¹ is unsubstituted unbranched unsaturated C₁-C₂₅ alkyl. In embodiments, R¹ is unsubstituted unbranched unsaturated C₁-C₂₀ alkyl. In embodiments, R¹ is unsubstituted unbranched unsaturated C₁-C₁₂ alkyl. In embodiments, R¹ is unsubstituted unbranched unsaturated C₁-C₈ alkyl. In embodiments, R¹ is unsubstituted unbranched unsaturated C₁-C₆ alkyl. In embodiments, R¹ is unsubstituted unbranched unsaturated C₁-C₄ alkyl. In embodiments, R¹ is unsubstituted unbranched unsaturated C₁-C₂ alkyl.

In embodiments, R¹ is unsubstituted C₉-C₁₉ alkyl. In embodiments, R¹ is unsubstituted branched C₉-C₁₉ alkyl. In embodiments, R¹ is unsubstituted unbranched C₉-C₁₉ alkyl. In embodiments, R¹ is unsubstituted branched saturated C₉-C₁₉ alkyl. In embodiments, R¹ is unsubstituted branched unsaturated C₉-C₁₉ alkyl. In embodiments, R¹ is unsubstituted unbranched saturated C₉-C₁₉ alkyl. In embodiments, R¹ is unsubstituted unbranched unsaturated C₉-C₁₉ alkyl.

In embodiments, R² is unsubstituted alkyl (e.g., C₁-C₂₅, C₁-C₂₀, C₁-C₁₂, C₁-C₈, C₁-C₆, C₁-C₄, or C₁-C₂). In embodiments, R² is unsubstituted C₁-C₂₅ alkyl. In embodiments, R² is unsubstituted C₁-C₂₀ alkyl. In embodiments, R² is unsubstituted C₁-C₁₂ alkyl. In embodiments, R² is unsubstituted C₁-C₈ alkyl. In embodiments, R² is unsubstituted C₁-C₆ alkyl. In embodiments, R² is unsubstituted C₁-C₄ alkyl. In embodiments, R² is unsubstituted C₁-C₂ alkyl.

In embodiments, R² is unsubstituted branched alkyl (e.g., C₁-C₂₅, C₁-C₂₀, C₁-C₁₂, C₁-C₈, C₁-C₆, C₁-C₄, or C₁-C₂). In embodiments, R² is unsubstituted branched C₁-C₂₅ alkyl. In embodiments, R² is unsubstituted branched C₁-C₂₀ alkyl. In embodiments, R² is unsubstituted branched C₁-C₁₂ alkyl. In embodiments, R² is unsubstituted branched C₁-C₈ alkyl. In embodiments, R² is unsubstituted branched C₁-C₆ alkyl. In embodiments, R² is unsubstituted branched C₁-C₄ alkyl. In embodiments, R² is unsubstituted branched C₁-C₂ alkyl.

In embodiments, R² is unsubstituted unbranched alkyl (e.g., C₁-C₂₅, C₁-C₂₀, C₁-C₁₂, C₁-C₈, C₁-C₆, C₁-C₄, or C₁-C₂). In embodiments, R² is unsubstituted unbranched C₁-C₂₅ alkyl. In embodiments, R² is unsubstituted unbranched C₁-C₂₀ alkyl. In embodiments, R² is unsubstituted unbranched C₁-C₁₂ alkyl. In embodiments, R² is unsubstituted unbranched C₁-C₈ alkyl. In embodiments, R² is unsubstituted unbranched C₁-C₆ alkyl. In embodiments, R² is unsubstituted unbranched C₁-C₄ alkyl. In embodiments, R² is unsubstituted unbranched C₁-C₂ alkyl.

In embodiments, R² is unsubstituted branched saturated alkyl (e.g., C₁-C₂₅, C₁-C₂₀, C₁-C₁₂, C₁-C₈, C₁-C₆, C₁-C₄, or C₁-C₂). In embodiments, R² is unsubstituted branched saturated C₁-C₂₅ alkyl. In embodiments, R² is unsubstituted branched saturated C₁-C₂₀ alkyl. In embodiments, R² is unsubstituted branched saturated C₁-C₁₂ alkyl. In embodiments, R² is unsubstituted branched saturated C₁-C₈ alkyl. In embodiments, R² is unsubstituted branched saturated C₁-C₆ alkyl. In embodiments, R² is unsubstituted branched saturated C₁-C₄ alkyl. In embodiments, R² is unsubstituted branched saturated C₁-C₂ alkyl.

In embodiments, R² is unsubstituted branched unsaturated alkyl (e.g., C₁-C₂₅, C₁-C₂₀, C₁-C₁₂, C₁-C₈, C₁-C₆, C₁-C₄, or C₁-C₂). In embodiments, R² is unsubstituted branched unsaturated C₁-C₂₅ alkyl. In embodiments, R² is unsubstituted branched unsaturated C₁-C₂₀ alkyl. In embodiments, R² is unsubstituted branched unsaturated C₁-C₁₂ alkyl. In embodiments, R² is unsubstituted branched unsaturated C₁-C₈ alkyl. In embodiments, R² is unsubstituted branched unsaturated C₁-C₆ alkyl. In embodiments, R² is unsubstituted branched unsaturated C₁-C₄ alkyl. In embodiments, R² is unsubstituted branched saturated C₁-C₂ alkyl.

In embodiments, R² is unsubstituted unbranched saturated alkyl (e.g., C₁-C₂₅, C₁-C₂₀, C₁-C₁₂, C₁-C₈, C₁-C₆, C₁-C₄, or C₁-C₂). In embodiments, R² is unsubstituted unbranched saturated C₁-C₂₅ alkyl. In embodiments, R² is unsubstituted unbranched saturated C₁-C₂₀ alkyl. In embodiments, R² is unsubstituted unbranched saturated C₁-C₁₂ alkyl. In embodiments, R² is unsubstituted unbranched saturated C₁-C₈ alkyl. In embodiments, R² is unsubstituted unbranched saturated C₁-C₆ alkyl. In embodiments, R² is unsubstituted unbranched saturated C₁-C₄ alkyl. In embodiments, R² is unsubstituted unbranched saturated C₁-C₂ alkyl.

In embodiments, R² is unsubstituted unbranched unsaturated alkyl (e.g., C₁-C₂₅, C₁-C₂₀, C₁-C₁₂, C₁-C₈, C₁-C₆, C₁-C₄, or C₁-C₂). In embodiments, R² is unsubstituted unbranched unsaturated C₁-C₂₅ alkyl. In embodiments, R² is unsubstituted unbranched unsaturated C₁-C₂₀ alkyl. In embodiments, R² is unsubstituted unbranched unsaturated C₁-C₁₂ alkyl. In embodiments, R² is unsubstituted unbranched unsaturated C₁-C₈ alkyl. In embodiments, R² is unsubstituted unbranched unsaturated C₁-C₆ alkyl. In embodiments, R² is unsubstituted unbranched unsaturated C₁-C₄ alkyl. In embodiments, R² is unsubstituted unbranched unsaturated C₁-C₂ alkyl.

In embodiments, R² is unsubstituted C₉-C₁₉ alkyl. In embodiments, R² is unsubstituted branched C₉-C₁₉ alkyl. In embodiments, R² is unsubstituted unbranched C₉-C₁₉ alkyl. In embodiments, R² is unsubstituted branched saturated C₉-C₁₉ alkyl. In embodiments, R² is unsubstituted branched unsaturated C₉-C₁₉ alkyl. In embodiments, R² is unsubstituted unbranched saturated C₉-C₁₉ alkyl. In embodiments, R² is unsubstituted unbranched unsaturated C₉-C₁₉ alkyl.

In embodiments, R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted (e.g., substituted with a substituent group, a size-limited substituent group, or lower substituent group) or unsubstituted alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted aryl (e.g., C₆-C₁₂, C₆-C₁₀, or phenyl), or substituted (e.g., substituted with a substituent group, a size-limited substituent group, or lower substituent group) or unsubstituted heteroaryl (e.g., 5 to 12 membered, 5 to 10 membered, 5 to 9 membered, or 5 to 6 membered). In embodiments, R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted (e.g., substituted with a substituent group, a size-limited substituent group, or lower substituent group) alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) aryl (e.g., C₆-C₁₂, C₆-C₁₀, or phenyl), or substituted (e.g., substituted with a substituent group, a size-limited substituent group, or lower substituent group) heteroaryl (e.g., 5 to 12 membered, 5 to 10 membered, 5 to 9 membered, or 5 to 6 membered).

In embodiments, R³ is
hydrogen, —NH₂, —OH, —SH, —C(O)H, —C(O)NH₂, —NHC(O)H, —NHC(O)OH, —NHC(O)NH₂, —C(O) OH, —OC(O)H, —N₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, when R³ is substituted, R³ is substituted with a substituent group. In embodiments, when R³ is substituted, R³ is substituted with a size-limited substituent group. In embodiments, when R³ is substituted, R³ is substituted with a lower substituent group (e.g., oxo).

In embodiments, the compound including a single-stranded oligonucleotide includes a motif (e.g. formula (IV), or (IV-a)) described above including in any aspects, embodiments, claims, figures, tables (e.g., Tables 1-5 and A-O), examples, or schemes (e.g., Schemes I, II, III, and IV).

In embodiments, a uptake domain is represented by the structure of:

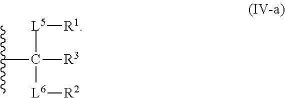

R¹, R², R³, L⁵, and L⁶ are as described above.

In embodiments, the compound including a single-stranded oligonucleotide includes one or more uptake domains having a structure shown in Table 1 below. In embodiments, the compound including a single-stranded oligonucleotide includes a DTx-01-01 domain in Table 1. In embodiments, the compound including a single-stranded oligonucleotide includes a DTx-01-03 domain 1 of Table 1. In embodiments, the compound including a single-stranded oligonucleotide includes a DTx-01-06 domain in Table 1. In embodiments, the compound including a single-stranded oligonucleotide includes a DTx-01-08 domain in Table 1. In embodiments, the compound including a single-stranded oligonucleotide includes a DTx-01-11 domain in Table 1. In embodiments, the compound including a single-stranded oligonucleotide includes a DTx-01-13 domain in Table 1. In embodiments, the compound including a single-stranded oligonucleotide includes a DTx-01-30 domain in Table 1. In embodiments, the compound including a single-stranded oligonucleotide includes a DTx-01-31 domain in Table 1. In embodiments, the compound including a single-stranded oligonucleotide includes a DTx-01-32 domain in Table 1. In embodiments, the compound including a single-stranded oligonucleotide includes a DTx-01-33 domain in Table 1. In embodiments, the compound including a single-stranded oligonucleotide includes a DTx-01-34 domain in Table 1. In embodiments, the compound including a single-stranded oligonucleotide includes a DTx-01-35 domain in Table 1. In embodiments, the compound including a single-stranded oligonucleotide includes a DTx-01-36 domain in Table 1. In embodiments, the compound including a single-stranded oligonucleotide includes a DTx-01-39 domain in Table 1. In embodiments, the compound including a single-stranded oligonucleotide includes a DTx-01-43 domain in Table 1. In embodiments, the compound including a single-stranded oligonucleotide includes a DTx-01-44 domain in Table 1. In embodiments, the compound including a single-stranded oligonucleotide includes a DTx-01-45 domain in Table 1. In embodiments, the compound including a single-stranded oligonucleotide includes a DTx-01-46 domain in Table 1. In embodiments, the compound including a single-stranded oligonucleotide includes a DTx-01-50 domain in Table 1. In embodiments, the compound including a single-stranded oligonucleotide includes a DTx-01-51 domain in Table 1. In embodiments, the compound including a single-stranded oligonucleotide includes a DTx-01-52 domain in Table 1. In embodiments, the compound including a single-stranded oligonucleotide includes a DTx-01-53 domain in Table 1. In embodiments, the compound including a single-stranded oligonucleotide includes a DTx-01-54 domain in Table 1. In embodiments, the compound including a single-stranded oligonucleotide includes a DTx-01-55 domain in Table 1. In embodiments, the compound including a single-stranded oligonucleotide includes a DTx-03-06 domain in Table 1. In embodiments, the compound including a single-stranded oligonucleotide includes a DTx-03-50 domain in Table 1. In embodiments, the compound including a single-stranded oligonucleotide includes a DTx-03-51 domain in Table 1. In embodiments, the compound including a single-stranded oligonucleotide includes a DTx-03-52 domain in Table 1. In embodiments, the compound including a single-stranded oligonucleotide includes a DTx-03-53 domain in Table 1. In embodiments, the compound including a single-stranded oligonucleotide includes a DTx-03-54 domain in Table 1. In embodiments, the compound including a single-stranded oligonucleotide includes a DTx-03-55 domain in Table 1. In embodiments, the compound including a single-stranded oligonucleotide includes a DTx-04-01 domain in Table 1. In embodiments, the compound including a single-stranded oligonucleotide includes a DTx-05-01 domain in Table 1. In embodiments, the compound including a single-stranded oligonucleotide includes a DTx-06-06 domain in Table 1. In embodiments, the compound including a single-stranded oligonucleotide includes a DTx-06-50 domain in Table 1. In embodiments, the compound including a single-stranded oligonucleotide includes a DTx-06-51 domain in Table 1. In embodiments, the compound including a single-stranded oligonucleotide includes a DTx-06-52 domain in Table 1. In embodiments, the compound including a single-stranded oligonucleotide includes a DTx-06-53 domain in Table 1. In embodiments, the compound including a single-stranded oligonucleotide includes a DTx-06-54 domain in Table 1. In embodiments, the compound including a single-stranded oligonucleotide includes a DTx-06-55 domain in Table 1. In embodiments, the compound including a single-stranded oligonucleotide includes a DTx-08-01 domain in Table 1. In embodiments, the compound including a single-stranded oligonucleotide includes a DTx-09-01 domain in Table 1. In embodiments, the compound including a single-stranded oligonucleotide includes a DTx-10-01 domain in Table 1. In embodiments, the compound including a single-stranded oligonucleotide includes a DTx-11-01 domain in Table 1. In embodiments, the compound including a single-stranded oligonucleotide includes a DTx-01-60 domain in Table 1. In embodiments, the compound including a single-stranded oligonucleotide includes a DTx-01-61 domain in Table 1. In embodiments, the compound including a single-stranded oligonucleotide includes a DTx-01-62 domain in Table 1. In embodiments, the compound including a single-stranded oligonucleotide includes a DTx-01-63 domain in Table 1. In embodiments, the compound including a single-stranded oligonucleotide includes a DTx-01-64 domain in Table 1. In embodiments, the compound including a single-stranded oligonucleotide includes a DTx-01-65 domain in Table 1. In embodiments, the compound including a single-stranded oligonucleotide includes a DTx-01-66 domain in Table 1. In embodiments, the compound including a single-stranded oligonucleotide includes a DTx-01-67 domain in Table 1. In embodiments, the compound including a single-stranded oligonucleotide includes a DTx-01-68 domain in Table 1. In embodiments, the compound including a single-stranded oligonucleotide includes a DTx-01-69 domain in Table 1. In embodiments, the compound including a single-stranded oligonucleotide includes a DTx-01-70 domain in Table 1. In embodiments, the compound including a single-stranded oligonucleotide includes a DTx-01-71 domain in Table 1. In embodiments, the compound including a single-stranded oligonucleotide includes a DTx-01-72 domain in Table 1. In embodiments, the compound including a single-stranded oligonucleotide includes a DTx-01-73 domain in Table 1. In embodiments, the compound including a single-stranded oligonucleotide includes a DTx-01-74 domain in Table 1. In embodiments, the compound including a single-stranded oligonucleotide includes a DTx-01-75 domain in Table 1. In embodiments, the compound including a single-stranded oligonucleotide includes a DTx-01-76 domain in Table 1. In embodiments, the compound including a single-stranded oligonucleotide includes a DTx-01-77 domain in Table 1. In embodiments, the compound including a single-stranded oligonucleotide includes a DTx-01-78 domain in Table 1. In embodiments, the compound including a single-stranded oligonucleotide includes a DTx-01-79 domain in Table 1. In embodiments, the compound including a single-stranded oligonucleotide includes a DTx-01-80 domain in Table 1. In embodiments, the compound including a single-stranded oligonucleotide includes a DTx-01-81 domain in Table 1. In embodiments, the compound including a single-stranded oligonucleotide includes a DTx-01-82 domain in Table 1. In embodiments, the compound including a single-stranded oligonucleotide includes a DTx-01-83 domain in Table 1. In embodiments, the compound including a single-stranded oligonucleotide includes a DTx-01-84 domain in Table 1. In embodiments, the compound including a single-stranded oligonucleotide includes a DTx-01-85 domain in Table 1. In embodiments, the compound including a single-stranded oligonucleotide includes a DTx-01-86 domain in Table 1. In embodiments, the compound including a single-stranded oligonucleotide includes a DTx-01-87 domain in Table 1. In embodiments, the compound including a single-stranded oligonucleotide includes a DTx-01-88 domain in Table 1. In embodiments, the compound including a single-stranded oligonucleotide includes a DTx-01-89 domain in Table 1. In embodiments, the compound including a single-stranded oligonucleotide includes a DTx-01-90 domain in Table 1. In embodiments, the compound including a single-stranded oligonucleotide includes a DTx-01-91 domain in Table 1. In embodiments, the compound including a single-stranded oligonucleotide includes a DTx-01-92 domain in Table 1. In embodiments, the compound including a single-stranded oligonucleotide includes a DTx-01-93 domain in Table 1. In embodiments, the compound including a single-stranded oligonucleotide includes a DTx-01-94 domain in Table 1. In embodiments, the compound including a single-stranded oligonucleotide includes a DTx-01-95 domain in Table 1. In embodiments, the compound including a single-stranded oligonucleotide includes a DTx-01-96 domain in Table 1. In embodiments, the compound including a single-stranded oligonucleotide includes a DTx-01-97 domain in Table 1. In embodiments, the compound including a single-stranded oligonucleotide includes a DTx-01-98 domain in Table 1. In embodiments, the compound including a single-stranded oligonucleotide includes a DTx-01-99 domain in Table 1. In embodiments, the compound including a single-stranded oligonucleotide includes a DTx-01-100 domain in Table 1. In embodiments, the compound including a single-stranded oligonucleotide includes a DTx-01-101 domain in Table 1.

TABLE 1
Uptake Domain

| Motif | |
|---|---|
| Name | Structure |
| DTx-01-01 | 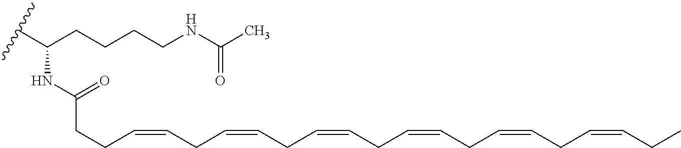 [SMILES: *[C@H](CCCCNC(C)=O)NC(=O)CC/C=C\C/C=C\C/C=C\C/C=C\C/C=C\C/C=C\CC] |
| DTx-01-03 | 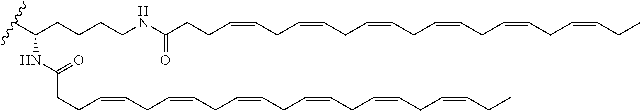 [SMILES: *[C@H](CCCCNC(=O)CC/C=C\C/C=C\C/C=C\C/C=C\C/C=C\C/C=C\CC)NC(=O)CC/C=C\C/C=C\C/C=C\C/C=C\C/C=C\C/C=C\CC] |
| DTx-01-06 | 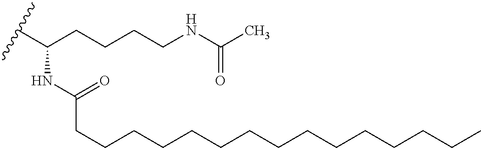 [SMILES: *[C@H](CCCCNC(C)=O)NC(=O)CCCCCCCCCCCCCCC] |
| DTx-01-08 | 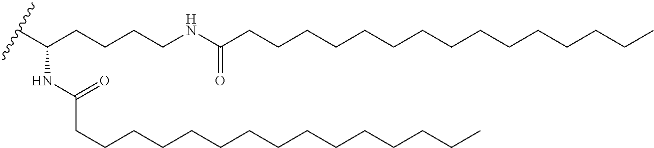 [SMILES: *[C@H](CCCCNC(=O)CCCCCCCCCCCCCCC)NC(=O)CCCCCCCCCCCCCCC] |
| DTx-01-11 | 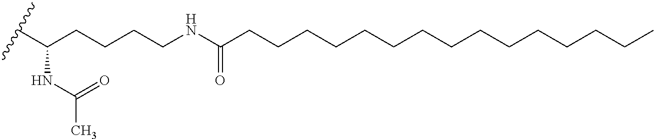 [SMILES: *[C@H](CCCCNC(=O)CCCCCCCCCCCCCCC)NC(C)=O] |
| DTx-01-13 | 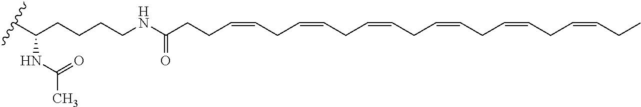 [SMILES: *[C@H](CCCCNC(=O)CC/C=C\C/C=C\C/C=C\C/C=C\C/C=C\C/C=C\CC)NC(C)=O] |
| DTx-01-30 | 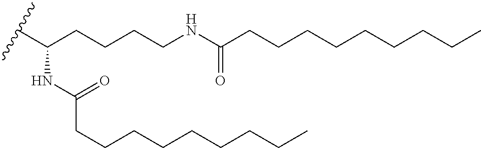 [SMILES: *[C@H](CCCCNC(=O)CCCCCCCCC)NC(=O)CCCCCCCCC] |
| DTx-01-31 | 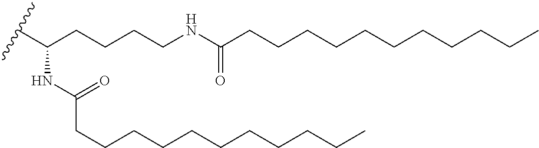 [SMILES: *[C@H](CCCCNC(=O)CCCCCCCCCCC)NC(=O)CCCCCCCCCCC] |
| DTx-01-32 | 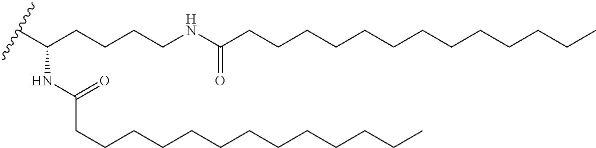 [SMILES: *[C@H](CCCCNC(=O)CCCCCCCCCCCCC)NC(=O)CCCCCCCCCCCCC] |
| DTx-01-33 | 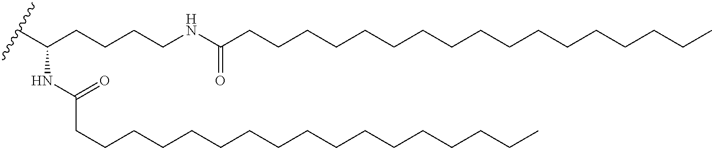 [SMILES: *[C@H](CCCCNC(=O)CCCCCCCCCCCCCCCCC)NC(=O)CCCCCCCCCCCCCCCCC] |
| DTx-01-34 | 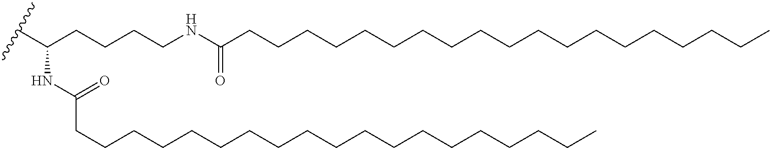 [SMILES: *[C@H](CCCCNC(=O)CCCCCCCCCCCCCCCCCCC)NC(=O)CCCCCCCCCCCCCCCCCCC] |
| DTx-01-35 | 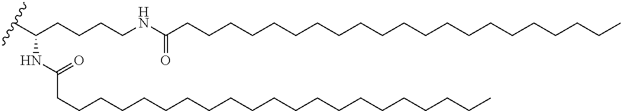 [SMILES: *[C@H](CCCCNC(=O)CCCCCCCCCCCCCCCCCCCCC)NC(=O)CCCCCCCCCCCCCCCCCCCCC] |
| DTx-01-36 | 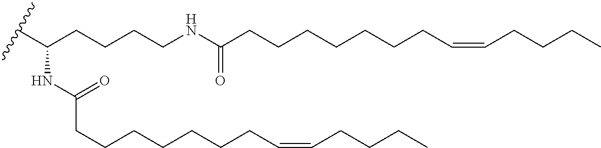 [SMILES: *[C@H](CCCCNC(=O)CCCCCCC/C=C\CCCC)NC(=O)CCCCCCC/C=C\CCCC] |
| DTx-01-39 | 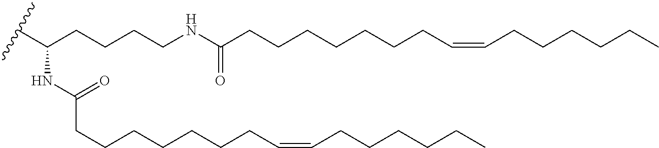 [SMILES: *[C@H](CCCCNC(=O)CCCCCCC/C=C\CCCCCC)NC(=O)CCCCCCC/C=C\CCCCCC] |
| DTx-01-43 | 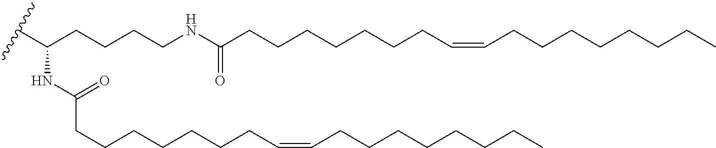 [SMILES: *[C@H](CCCCNC(=O)CCCCCCC/C=C\CCCCCCCC)NC(=O)CCCCCCC/C=C\CCCCCCCC] |
| DTx-01-44 | 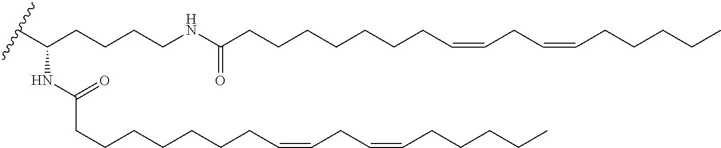 [SMILES: *[C@H](CCCCNC(=O)CCCCCCC/C=C\C/C=C\CCCCC)NC(=O)CCCCCCC/C=C\C/C=C\CCCCC] |
| DTx-01-45 | 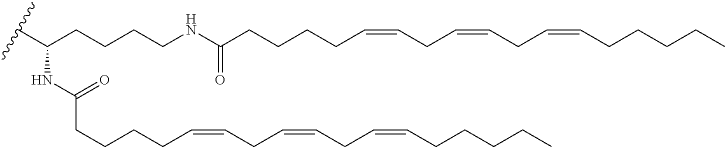 [SMILES: *[C@H](CCCCNC(=O)CCCC/C=C\C/C=C\C/C=C\CCCCC)NC(=O)CCCC/C=C\C/C=C\C/C=C\CCCCC] |
| DTx-01-46 | 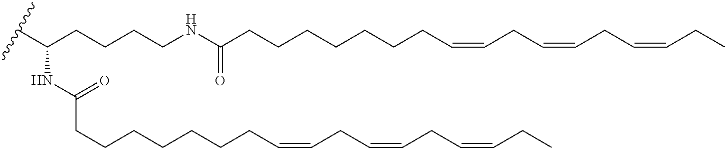 [SMILES: *[C@H](CCCCNC(=O)CCCCCCC/C=C\C/C=C\C/C=C\CC)NC(=O)CCCCCCC/C=C\C/C=C\C/C=C\CC] |
| DTx-01-50 | 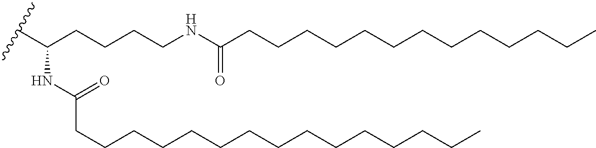 [SMILES: *[C@H](CCCCNC(=O)CCCCCCCCCCCCC)NC(=O)CCCCCCCCCCCCCCC] |
| DTx-01-51 | 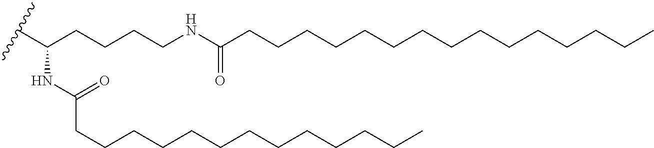 [SMILES: *[C@H](CCCCNC(=O)CCCCCCCCCCCCCCC)NC(=O)CCCCCCCCCCCCC] |
| DTx-01-52 | 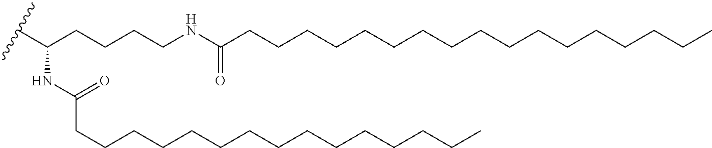 [SMILES: *[C@H](CCCCNC(=O)CCCCCCCCCCCCCCCCC)NC(=O)CCCCCCCCCCCCCCC] |
| DTx-01-53 | 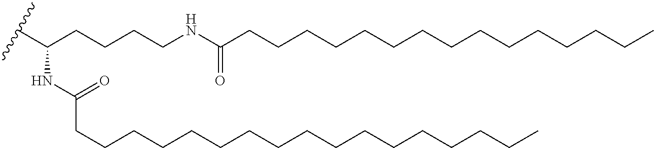 [SMILES: *[C@H](CCCCNC(=O)CCCCCCCCCCCCCCC)NC(=O)CCCCCCCCCCCCCCCCC] |
| DTx-01-54 | 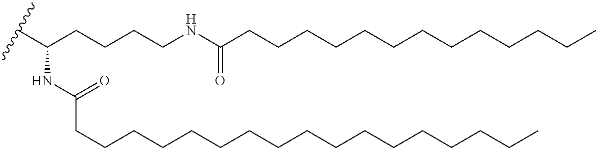 [SMILES: *[C@H](CCCCNC(=O)CCCCCCCCCCCCC)NC(=O)CCCCCCCCCCCCCCCCC] |
| DTx-01-55 | 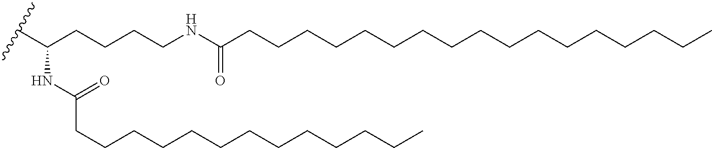 [SMILES: *[C@H](CCCCNC(=O)CCCCCCCCCCCCCCCCC)NC(=O)CCCCCCCCCCCCC] |
| DTx-03-06 | 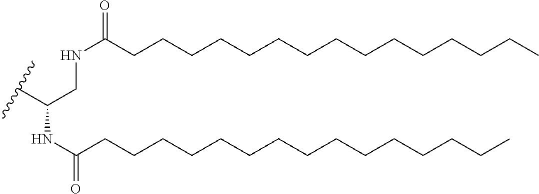 [SMILES: *[C@H](CNC(=O)CCCCCCCCCCCCCCC)NC(=O)CCCCCCCCCCCCCCC] |
| DTx-03-08 | 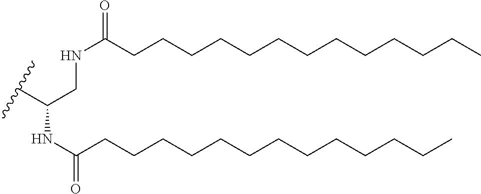 [SMILES: *[C@H](CNC(=O)CCCCCCCCCCCCC)NC(=O)CCCCCCCCCCCCC] |
| DTx-03-09 | 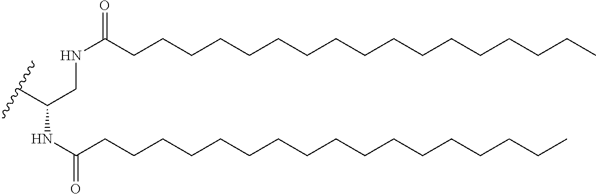 [SMILES: *[C@H](CNC(=O)CCCCCCCCCCCCCCCCC)NC(=O)CCCCCCCCCCCCCCCCC] |
| DTx-03-50 | 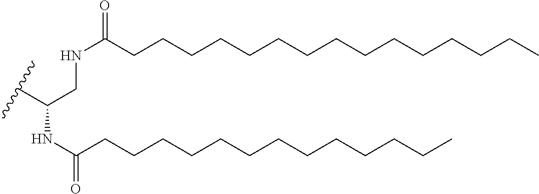 [SMILES: *[C@H](CNC(=O)CCCCCCCCCCCCCCC)NC(=O)CCCCCCCCCCCCC] |
| DTx-03-51 | 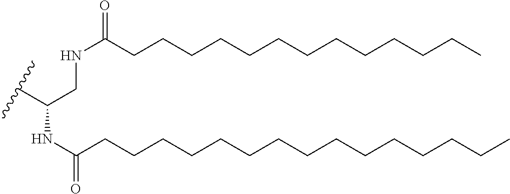 [SMILES: *[C@H](CNC(=O)CCCCCCCCCCCCC)NC(=O)CCCCCCCCCCCCCCC] |
| DTx-03-52 | 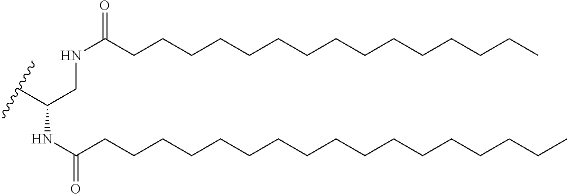 [SMILES: *[C@H](CNC(=O)CCCCCCCCCCCCCCC)NC(=O)CCCCCCCCCCCCCCCCC] |
| DTx-03-53 | 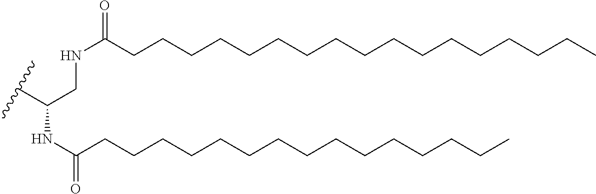 [SMILES: *[C@H](CNC(=O)CCCCCCCCCCCCCCCCC)NC(=O)CCCCCCCCCCCCCCC] |
| DTx-03-54 | 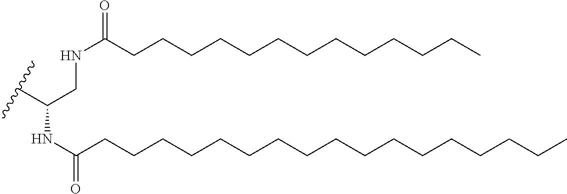 [SMILES: *[C@H](CNC(=O)CCCCCCCCCCCCC)NC(=O)CCCCCCCCCCCCCCCCC] |
| DTx-03-55 | 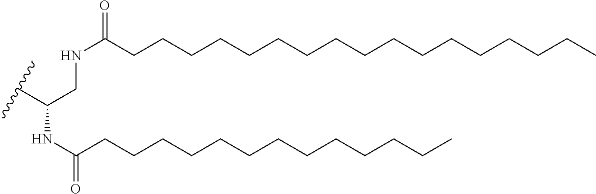 [SMILES: *[C@H](CNC(=O)CCCCCCCCCCCCCCCCC)NC(=O)CCCCCCCCCCCCC] |
| DTx-04-01 | 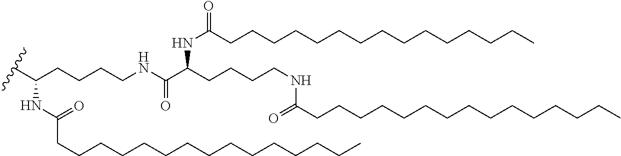 [SMILES: *[C@H](CCCCNC(=O)[C@H](CCCCNC(=O)CCCCCCCCCCCCCCC)NC(=O)CCCCCCCCCCCCCCC)NC(=O)CCCCCCCCCCCCCCC] |
| DTx-05-01 | 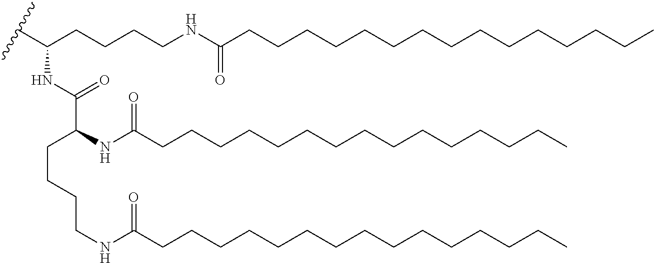 [SMILES: *[C@H](CCCCNC(=O)CCCCCCCCCCCCCCC)NC(=O)[C@H](CCCCNC(=O)CCCCCCCCCCCCCCC)NC(=O)CCCCCCCCCCCCCCC] |
| DTx-06-06 | 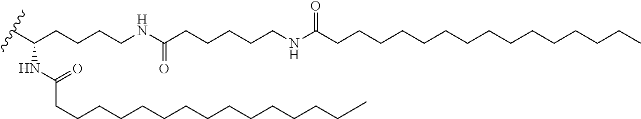 [SMILES: *[C@H](CCCCNC(=O)CCCCCNC(=O)CCCCCCCCCCCCCCC)NC(=O)CCCCCCCCCCCCCCC] |
| DTx-06-08 | 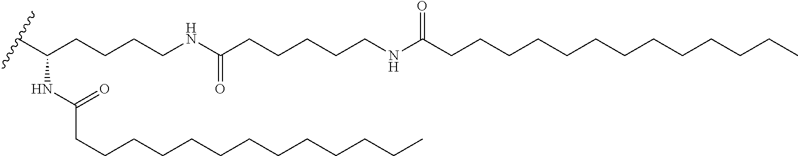 [SMILES: *[C@H](CCCCNC(=O)CCCCCNC(=O)CCCCCCCCCCCCC)NC(=O)CCCCCCCCCCCCC] |
| DTx-06-09 | 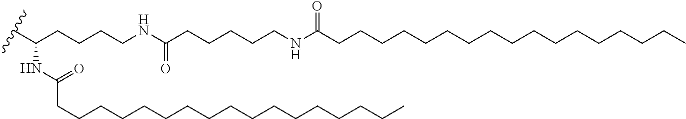 [SMILES: *[C@H](CCCCNC(=O)CCCCCNC(=O)CCCCCCCCCCCCCCCCC)NC(=O)CCCCCCCCCCCCCCCCC] |
| DTx-06-50 | 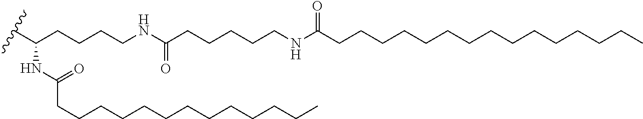 [SMILES: *[C@H](CCCCNC(=O)CCCCCNC(=O)CCCCCCCCCCCCCCC)NC(=O)CCCCCCCCCCCCC] |
| DTx-06-51 | 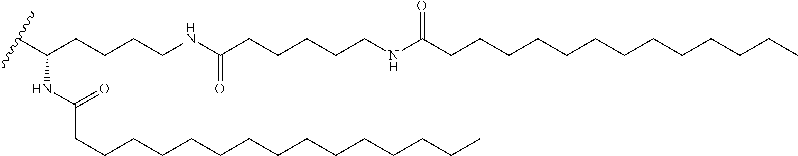 [SMILES: *[C@H](CCCCNC(=O)CCCCCNC(=O)CCCCCCCCCCCCC)NC(=O)CCCCCCCCCCCCCCC] |
| DTx-06-52 | 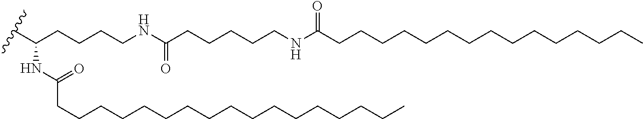 [SMILES: *[C@H](CCCCNC(=O)CCCCCNC(=O)CCCCCCCCCCCCCCC)NC(=O)CCCCCCCCCCCCCCCCC] |
| DTx-06-53 | 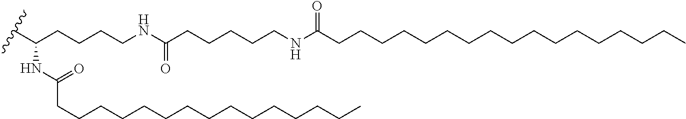 [SMILES: *[C@H](CCCCNC(=O)CCCCCNC(=O)CCCCCCCCCCCCCCCCC)NC(=O)CCCCCCCCCCCCCCC] |
| DTx-06-54 | 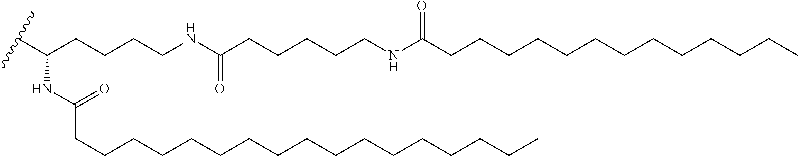 [SMILES: *[C@H](CCCCNC(=O)CCCCCNC(=O)CCCCCCCCCCCCC)NC(=O)CCCCCCCCCCCCCCCCC] |
| DTx-06-55 | 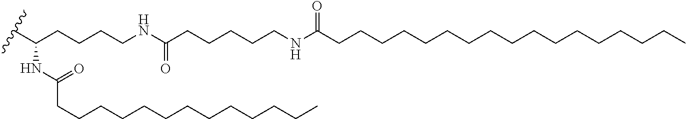 [SMILES: *[C@H](CCCCNC(=O)CCCCCNC(=O)CCCCCCCCCCCCCCCCC)NC(=O)CCCCCCCCCCCCC] |
| DTx-08-01 | 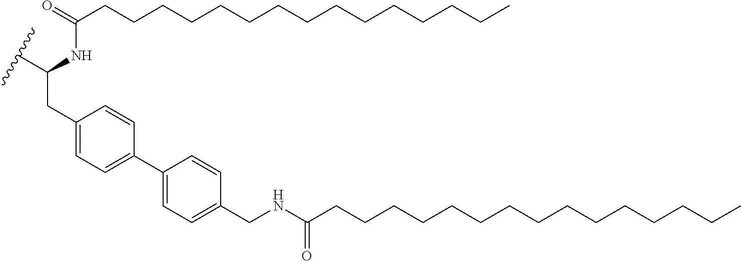 [SMILES: *[C@H](Cc1ccc(-c2ccc(CNC(=O)CCCCCCCCCCCCCCC)cc2)cc1)NC(=O)CCCCCCCCCCCCCCC] |
| DTx-09-01 | 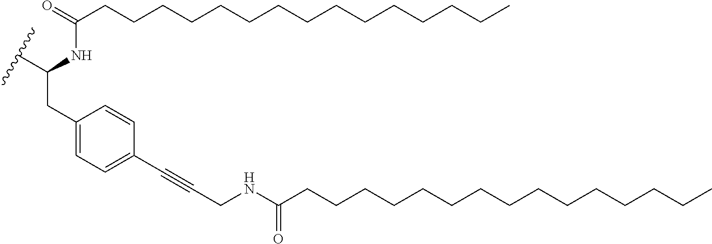 [SMILES: *[C@H](Cc1ccc(C#CCNC(=O)CCCCCCCCCCCCCCC)cc1)NC(=O)CCCCCCCCCCCCCCC] |
| DTx-10-01 | 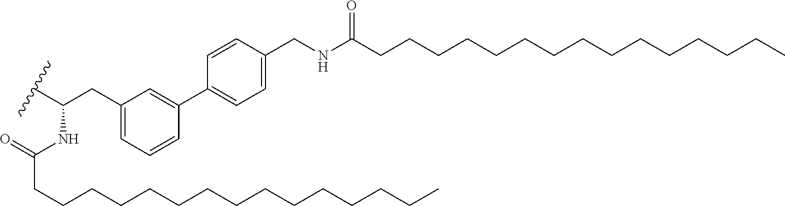 [SMILES: *[C@H](Cc1cccc(-c2ccc(CNC(=O)CCCCCCCCCCCCCCC)cc2)c1)NC(=O)CCCCCCCCCCCCCCC] |
| DTx-11-01 | 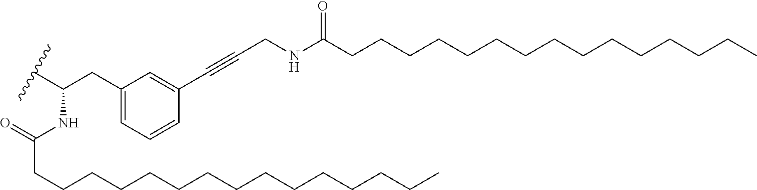 [SMILES: *[C@H](Cc1cccc(C#CCNC(=O)CCCCCCCCCCCCCCC)c1)NC(=O)CCCCCCCCCCCCCCC] |
| DTx-01-60 | 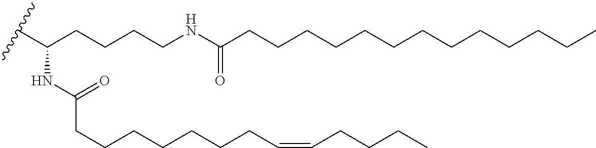 [SMILES: *[C@H](CCCCNC(=O)CCCCCCCCCCCCC)NC(=O)CCCCCCC/C=C\CCCC] |
| DTx-01-61 | 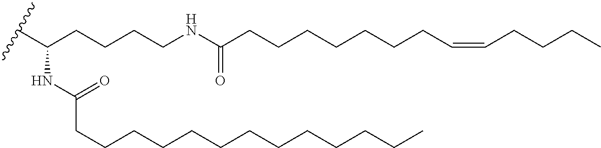 [SMILES: *[C@H](CCCCNC(=O)CCCCCCC/C=C\CCCC)NC(=O)CCCCCCCCCCCCC] |
| DTx-01-62 | 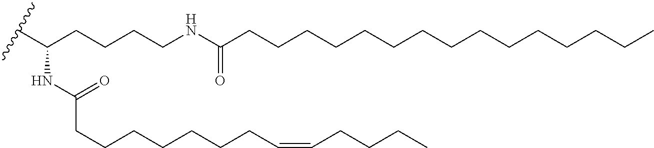 [SMILES: *[C@H](CCCCNC(=O)CCCCCCCCCCCCCCC)NC(=O)CCCCCCC/C=C\CCCC] |
| DTx-01-63 | 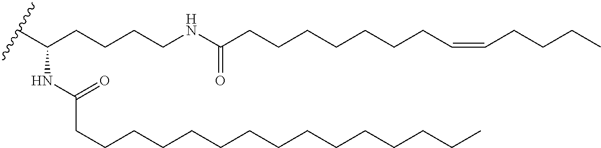 [SMILES: *[C@H](CCCCNC(=O)CCCCCCC/C=C\CCCC)NC(=O)CCCCCCCCCCCCCCC] |
| DTx-01-64 | 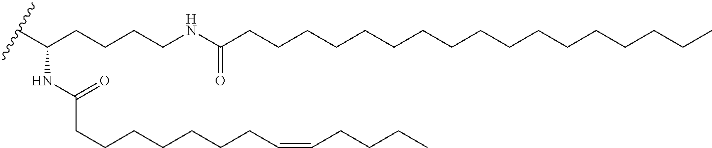 [SMILES: *[C@H](CCCCNC(=O)CCCCCCCCCCCCCCCCC)NC(=O)CCCCCCC/C=C\CCCC] |
| DTx-01-65 | 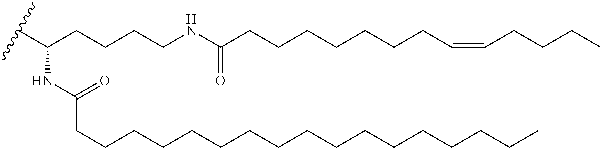 [SMILES: *[C@H](CCCCNC(=O)CCCCCCC/C=C\CCCC)NC(=O)CCCCCCCCCCCCCCCCC] |
| DTx-01-66 | 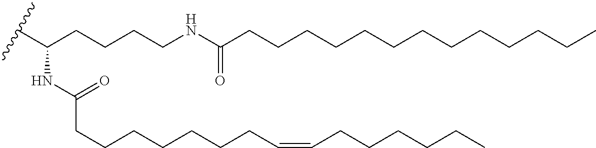 [SMILES: *[C@H](CCCCNC(=O)CCCCCCCCCCCCC)NC(=O)CCCCCCC/C=C\CCCCCC] |
| DTx-01-67 | 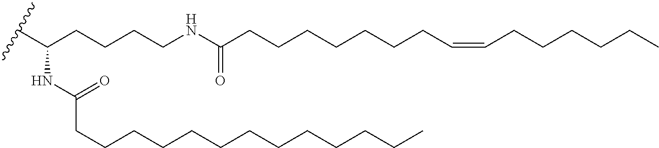 [SMILES: *[C@H](CCCCNC(=O)CCCCCCC/C=C\CCCCCC)NC(=O)CCCCCCCCCCCCC] |
| DTx-01-68 | 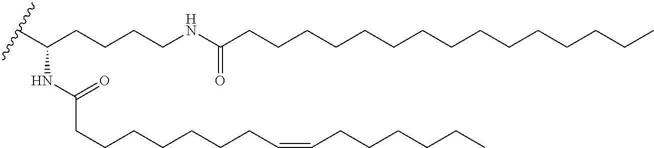 [SMILES: *[C@H](CCCCNC(=O)CCCCCCCCCCCCCCC)NC(=O)CCCCCCC/C=C\CCCCCC] |
| DTx-01-69 | 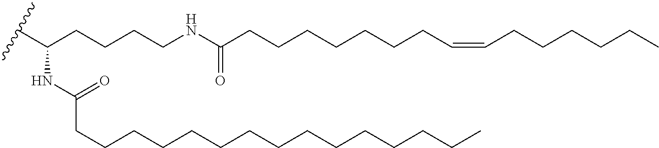 [SMILES: *[C@H](CCCCNC(=O)CCCCCCC/C=C\CCCCCC)NC(=O)CCCCCCCCCCCCCCC] |
| DTx-01-70 | 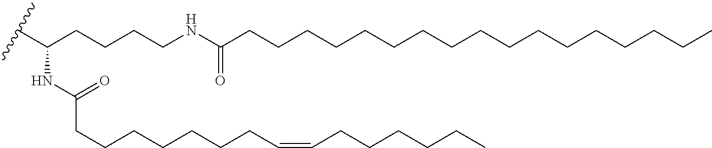 [SMILES: *[C@H](CCCCNC(=O)CCCCCCCCCCCCCCCCC)NC(=O)CCCCCCC/C=C\CCCCCC] |
| DTx-01-71 | 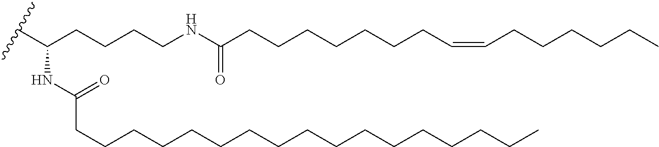 [SMILES: *[C@H](CCCCNC(=O)CCCCCCC/C=C\CCCCCC)NC(=O)CCCCCCCCCCCCCCCCC] |
| DTx-01-72 | 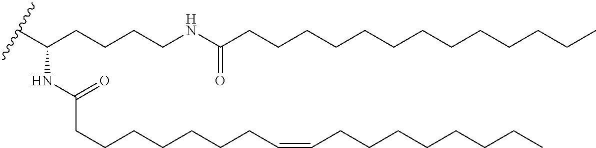 [SMILES: *[C@H](CCCCNC(=O)CCCCCCCCCCCCC)NC(=O)CCCCCCC/C=C\CCCCCCCC] |
| DTx-01-73 | 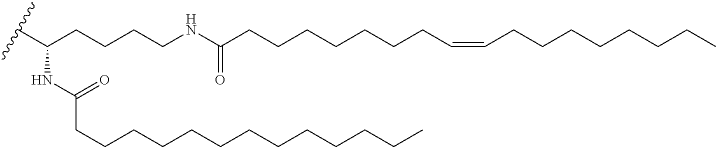 [SMILES: *[C@H](CCCCNC(=O)CCCCCCC/C=C\CCCCCCCC)NC(=O)CCCCCCCCCCCCC] |
| DTx-01-74 | 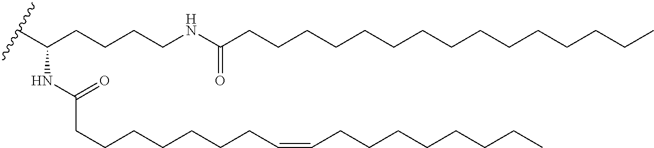 [SMILES: *[C@H](CCCCNC(=O)CCCCCCCCCCCCCCC)NC(=O)CCCCCCC/C=C\CCCCCCCC] |
| DTx-01-75 | 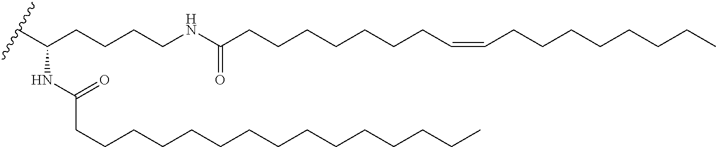 [SMILES: *[C@H](CCCCNC(=O)CCCCCCC/C=C\CCCCCCCC)NC(=O)CCCCCCCCCCCCCCC] |
| DTx-01-76 | 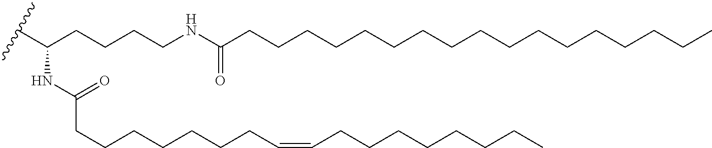 [SMILES: *[C@H](CCCCNC(=O)CCCCCCCCCCCCCCCCC)NC(=O)CCCCCCC/C=C\CCCCCCCC] |
| DTx-01-77 | 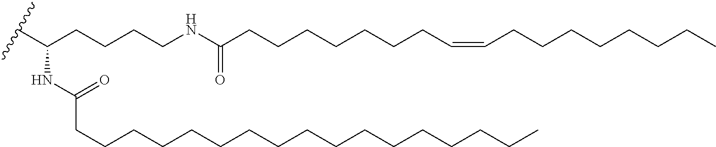 [SMILES: *[C@H](CCCCNC(=O)CCCCCCC/C=C\CCCCCCCC)NC(=O)CCCCCCCCCCCCCCCCC] |
| DTx-01-78 | 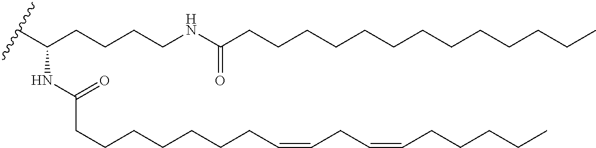 [SMILES: *[C@H](CCCCNC(=O)CCCCCCCCCCCCC)NC(=O)CCCCCCC/C=C\C/C=C\CCCCC] |
| DTx-01-79 | 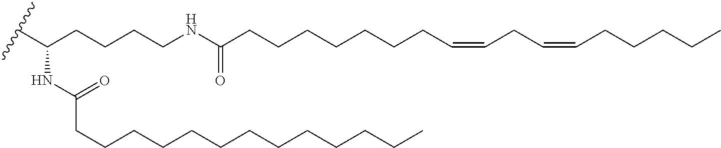 [SMILES: *[C@H](CCCCNC(=O)CCCCCCC/C=C\C/C=C\CCCCC)NC(=O)CCCCCCCCCCCCC] |
| DTx-01-80 | 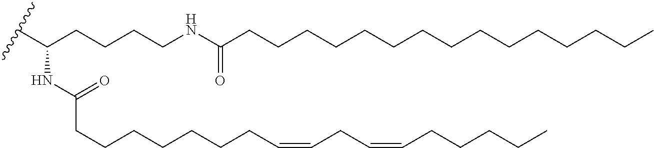 [SMILES: *[C@H](CCCCNC(=O)CCCCCCCCCCCCCCC)NC(=O)CCCCCCC/C=C\C/C=C\CCCCC] |
| DTx-01-81 | 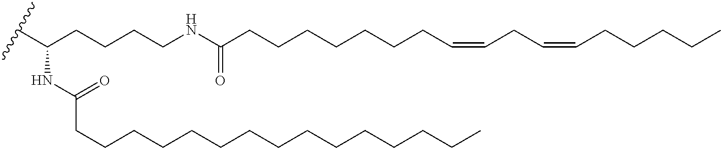 [SMILES: *[C@H](CCCCNC(=O)CCCCCCC/C=C\C/C=C\CCCCC)NC(=O)CCCCCCCCCCCCCCC] |
| DTx-01-82 | 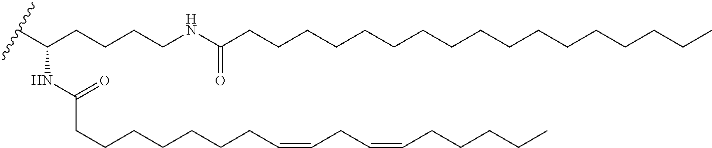 [SMILES: *[C@H](CCCCNC(=O)CCCCCCCCCCCCCCCCC)NC(=O)CCCCCCC/C=C\C/C=C\CCCCC] |
| DTx-01-83 | 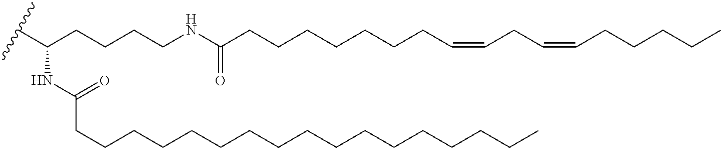 [SMILES: *[C@H](CCCCNC(=O)CCCCCCC/C=C\C/C=C\CCCCC)NC(=O)CCCCCCCCCCCCCCCCC] |
| DTx-01-84 | 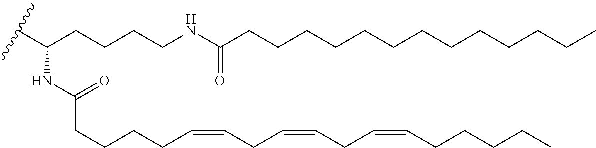 [SMILES: *[C@H](CCCCNC(=O)CCCCCCCCCCCCC)NC(=O)CCCC/C=C\C/C=C\C/C=C\CCCCC] |
| DTx-01-85 | 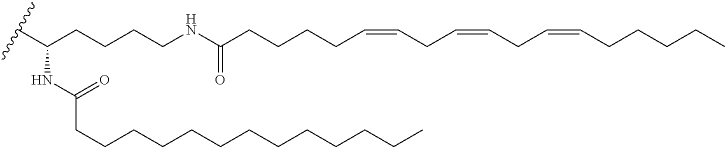 [SMILES: *[C@H](CCCCNC(=O)CCCC/C=C\C/C=C\C/C=C\CCCCC)NC(=O)CCCCCCCCCCCCC] |
| DTx-01-86 | 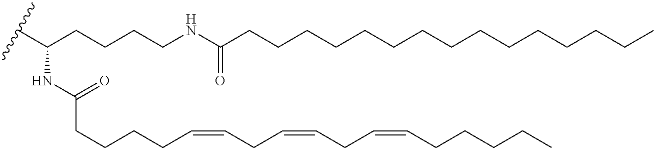 [SMILES: *[C@H](CCCCNC(=O)CCCCCCCCCCCCCCC)NC(=O)CCCC/C=C\C/C=C\C/C=C\CCCCC] |
| DTx-01-87 | 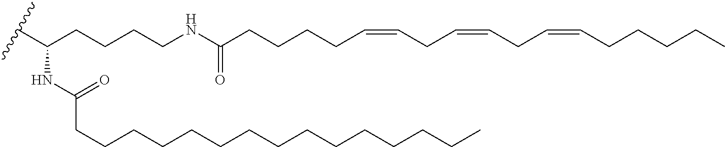 [SMILES: *[C@H](CCCCNC(=O)CCCC/C=C\C/C=C\C/C=C\CCCCC)NC(=O)CCCCCCCCCCCCCCC] |
| DTx-01-88 | 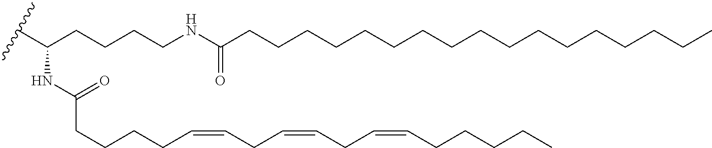 [SMILES: *[C@H](CCCCNC(=O)CCCCCCCCCCCCCCCCC)NC(=O)CCCC/C=C\C/C=C\C/C=C\CCCCC] |
| DTx-01-89 | 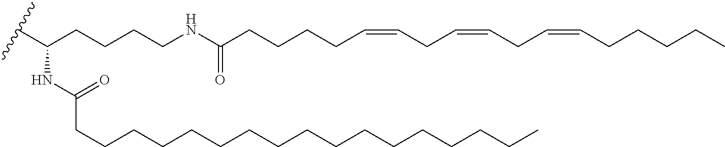 [SMILES: *[C@H](CCCCNC(=O)CCCC/C=C\C/C=C\C/C=C\CCCCC)NC(=O)CCCCCCCCCCCCCCCCC] |
| DTx-01-90 | 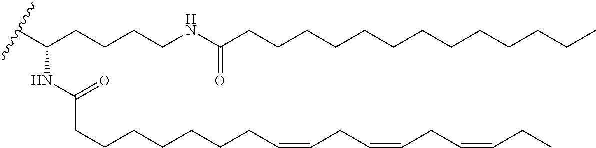 [SMILES: *[C@H](CCCCNC(=O)CCCCCCCCCCCCC)NC(=O)CCCCCCC/C=C\C/C=C\C/C=C\CC] |
| DTx-01-91 | 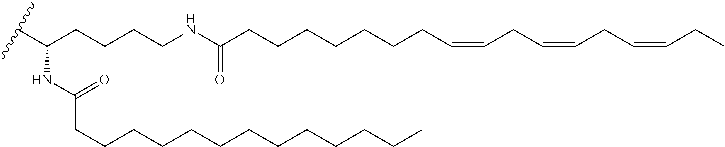 [SMILES: *[C@H](CCCCNC(=O)CCCCCCC/C=C\C/C=C\C/C=C\CC)NC(=O)CCCCCCCCCCCCC] |
| DTx-01-92 | 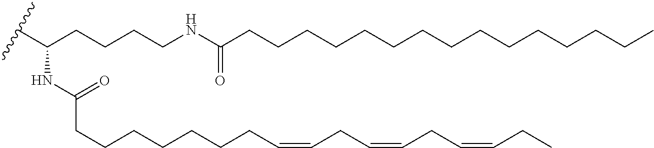 [SMILES: *[C@H](CCCCNC(=O)CCCCCCCCCCCCCCC)NC(=O)CCCCCCC/C=C\C/C=C\C/C=C\CC] |
| DTx-01-93 | 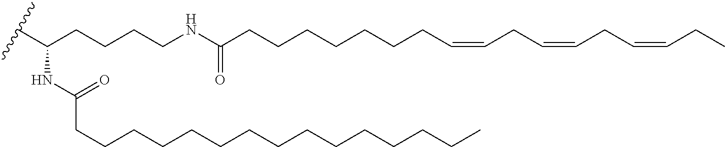 [SMILES: *[C@H](CCCCNC(=O)CCCCCCC/C=C\C/C=C\C/C=C\CC)NC(=O)CCCCCCCCCCCCCCC] |
| DTx-01-94 | 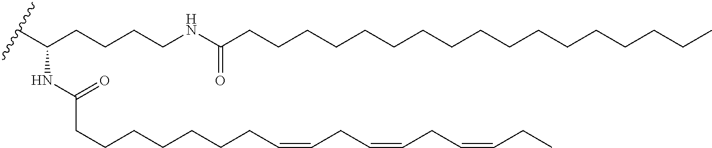 [SMILES: *[C@H](CCCCNC(=O)CCCCCCCCCCCCCCCCC)NC(=O)CCCCCCC/C=C\C/C=C\C/C=C\CC] |
| DTx-01-95 | 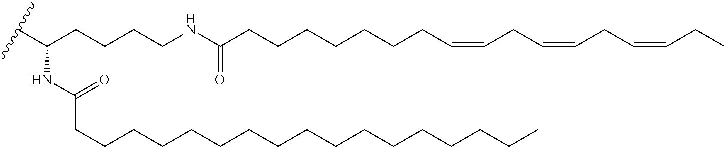 [SMILES: *[C@H](CCCCNC(=O)CCCCCCC/C=C\C/C=C\C/C=C\CC)NC(=O)CCCCCCCCCCCCCCCCC] |
| DTx-01-96 | 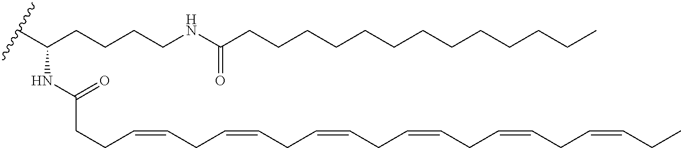 [SMILES: *[C@H](CCCCNC(=O)CCCCCCCCCCCCC)NC(=O)CC/C=C\C/C=C\C/C=C\C/C=C\C/C=C\C/C=C\CC] |
| DTx-01-97 | 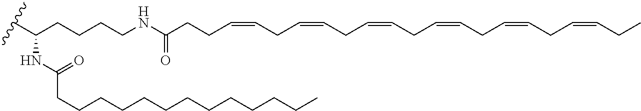 [SMILES: *[C@H](CCCCNC(=O)CC/C=C\C/C=C\C/C=C\C/C=C\C/C=C\C/C=C\CC)NC(=O)CCCCCCCCCCCCC] |
| DTx-01-98 | 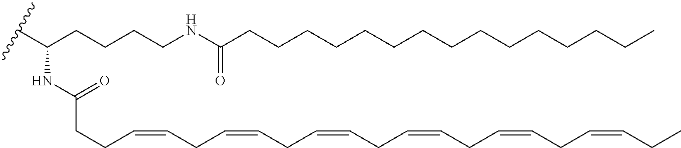 [SMILES: *[C@H](CCCCNC(=O)CCCCCCCCCCCCCCC)NC(=O)CC/C=C\C/C=C\C/C=C\C/C=C\C/C=C\C/C=C\CC] |
| DTx-01-99 | 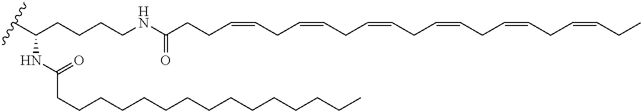 [SMILES: *[C@H](CCCCNC(=O)CC/C=C\C/C=C\C/C=C\C/C=C\C/C=C\C/C=C\CC)NC(=O)CCCCCCCCCCCCCCC] |
| DTx-01-100 | 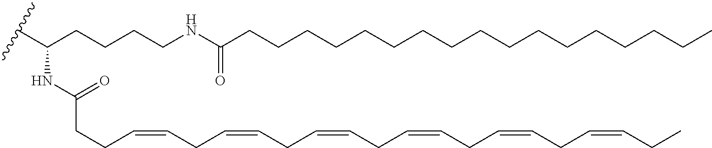 [SMILES: *[C@H](CCCCNC(=O)CCCCCCCCCCCCCCCCC)NC(=O)CC/C=C\C/C=C\C/C=C\C/C=C\C/C=C\C/C=C\CC] |
| DTx-01-101v2 | 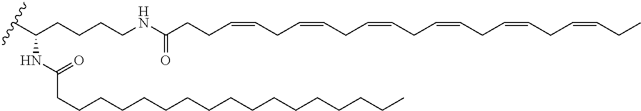 [SMILES: *[C@H](CCCCNC(=O)CC/C=C\C/C=C\C/C=C\C/C=C\C/C=C\C/C=C\CC)NC(=O)CCCCCCCCCCCCCCCCC] |

In embodiments, the single-stranded oligonucleotide includes one or more modified nucleotides. In embodiments, the oligonucleotide includes one or more modified nucleotides. In embodiments, a modified nucleotide includes a modified sugar moiety. In embodiments, a modified nucleotide includes a modified internucleotide linkage. In embodiments, a modified nucleotide includes a modified nucleobase. In embodiments, a modified nucleotide includes a modified 5′-terminal phosphate group. In embodiments, a modified nucleotide includes a modification at the 5′ carbon of the pentafuranosyl sugar. In embodiments, a modified nucleotide includes a modification at the 3′ carbon of the pentafuranosyl sugar. In embodiments, a modified nucleotide includes a modification at the 2′ carbon of the pentafuranosyl sugar.

In embodiments, the single-stranded oligonucleotide includes one or more modified sugar moieties. In embodiments, the oligonucleotide includes one or more modified sugar moieties. In embodiments, a modified sugar moiety includes a 2′-modification, i.e. the sugar moiety is modified at the 2′ carbon of the pentafuranosyl sugar, relative to the naturally occurring 2′-OH of RNA or the 2′-H of DNA. In embodiments, a 2′-modification is selected from 2′-fluoro, 2′-OCF3, 2′-O—CH₃ (also referred to as “2′-OMe” or “2′-O-methyl”), 2′-OCH₂CH₂OCH₃ (also referred to as “2′-O-methoxyethyl” or “2-MOE”), 2′-O(CH₂)₂SCH₃, O—(CH₂)₂—O—N(CH₃)₂, —O(CH₂)₂O(CH₂)₂N(CH₃)₂, and —O—CH₂—C(═O)—N(H)CH₃. In embodiments, the 2′ modification is a 2′-fluoro modification. In embodiments, the 2′ modification is a 2′-O-methyl modification. In embodiments, the 2′ modification is a 2′-O-methoxyethyl modification.

In embodiments, the 2′ modification is a bicyclic sugar modification, where the ribose has a covalent linkage between the 2′ and 4′ carbons. Nucleotides including such modified sugar moieties may be referred to as “bicyclic nucleic acids” or “BNA.” In embodiments, the covalent linkage of a bicyclic sugar modification is a 4′-CH₂—O-2′ linkage (methyleneoxy), also known as “LNA.” In embodiments, the covalent linkage of a bicyclic sugar modification is a 4′-(CH₂)₂—O-2′ linkage (ethyleneoxy), also known as “ENA.” In embodiments, the covalent linkage of a bicyclic sugar modification is a 4′-CH(CH₃)—O-2′ linkage (methyl(methyleneoxy)), also known as “constrained ethyl” or “cEt.” In embodiments, the covalent linkage of a bicyclic sugar modification is a 4′-CH(CH₂—OMe)-O-2′ linkage, also known as “c-MOE.” In embodiments, the covalent linkage of a bicyclic sugar modification is a 4′-CH₂—N(CH₃)—O-2′ linkage. In embodiments, the covalent linkage of a bicyclic sugar modification is a 4′-CH₂—N(H)—O-2′ linkage. In embodiments, the bicyclic sugar modification is a D sugar in the alpha configuration. In certain such embodiments, the bicyclic sugar modification is a D sugar in the beta configuration. In certain such embodiments, the bicyclic sugar modification is an L sugar in the alpha configuration. In certain such embodiments, the bicyclic sugar modification is an L sugar in the beta configuration.

In embodiments, a modified sugar moiety is an acyclic nucleoside derivative lacking the bond between the 2′ carbon and 3′ carbon of the sugar ring, also known as an “unlocked sugar modification.”

In embodiments, a modified sugar moiety is a morpholino moiety, where the pentafuranosyl sugar is replaced with a six-membered methylenemorpholine ring.

In embodiments, a modified nucleotide includes a modification at the 6′ carbon of the morpholino moiety. In embodiments, a modified nucleotide includes a modification at the 3′ nitrogen of the morpholino moiety. In embodiments, a modified nucleotide includes a modification at the 2′ carbon of the morpholino moiety.

In embodiments, the oxygen of the pentafuranosyl sugar is replace with a sulfur, to form a thio-sugar. In embodiments, a thio-sugar is modified at the 2′ carbon.

In embodiments, a modified sugar moiety is a hexitol nucleic acid moiety, where the pentafuranosyl sugar is replaced with a six-membered anhydrohexitol ring. In embodiments, a hexitol nucleotide is modified at the 3′ position.

In embodiments, the single-stranded oligonucleotide includes one or more modified internucleotide linkages. In embodiments, the oligonucleotide includes one or more modified internucleotide linkages. In embodiments, a modified internucleotide linkage is a phosphorothioate linkage. In embodiments, a modified internucleotide linkage is a phosphorodiamidite linkage. In embodiments, a modified internucleotide linkage is a methylphosphonate internucleotide linkage. In embodiments, a modified internucleotide linkage is a boranophosphonate linkage. In embodiments, the modified internucleotide linkage is an O-methylphosphoroamidite linkage. In embodiments, the modified internucleotide linkage is a phosphoroamidate linkage. In embodiments, the single-stranded oligonucleotide contains a positive backbone. In embodiments, the single-stranded oligonucleotide contains a non-ionic backbone.

In embodiments, the single-stranded oligonucleotide includes one or more modified nucleobases. In embodiments, the oligonucleotide includes one or more modified nucleobase. In embodiments, a modified nucleobase is selected from 5-hydroxymethyl cytosine, 7-deazaguanine and 7-deazaadenine. In embodiments, a modified nucleobase is selected from 7-deaza-adenine, 7-deazaguanosine, 2-aminopyridine and 2-pyridone. In embodiments, a modified nucleobase is selected from 5-substituted pyrimidines, 6-azapyrimidines and N-2, N-6 and 0-6 substituted purines, including 2 aminopropyladenine, 5-propynyluracil and 5-propynylcytosine.

In embodiments, the single-stranded oligonucleotide induces skipping of an exon of the dystrophin pre-mRNA. In embodiments, the single-stranded oligonucleotide induces skipping of exon 45 of the dystrophin pre-mRNA. In embodiments, the single-stranded oligonucleotide induces skipping of exon 51 of the dystrophin pre-mRNA. In embodiments, the single-stranded oligonucleotide induces skipping of exon 53 of the dystrophin pre-mRNA.

In embodiments, nucleobase sequence of the single-stranded oligonucleotide is complementary to a splice donor site, a splice acceptor site, an exonic splicing enhancer (ESE), a splicing branch point, an exon recognition sequence, or a splice enhancer of the dystrophin pre-mRNA.

In embodiments the nucleobase sequence of the single-stranded oligonucleotide is complementary to an annealing site of the dystrophin pre-mRNA. Certain annealing sites and nucleobase sequences are provided in Table 2. In embodiments, an annealing site is selected from any one of the annealing sites in Table 2. In embodiments, the nucleobase sequence of the single-stranded oligonucleotide is selected from any one of the nucleobase sequences in Table 2.

In embodiments, the nucleobase sequence of the single-stranded oligonucleotide comprises at least 8 contiguous nucleobases of a nucleobase sequence selected from Table 2. In embodiments, the nucleobase sequence of the single-stranded oligonucleotide comprises at least 9 contiguous nucleobases of a nucleobase sequence selected from Table 2. In embodiments, the nucleobase sequence of the single-stranded oligonucleotide comprises at least 10 contiguous nucleobases of a nucleobase sequence selected from Table 2. In embodiments, the nucleobase sequence of the single-stranded oligonucleotide comprises at least 11 contiguous nucleobases of a nucleobase sequence selected from Table 2. In embodiments, the nucleobase sequence of the single-stranded oligonucleotide comprises at least 12 contiguous nucleobases of a nucleobase sequence selected from Table 2. In embodiments, the nucleobase sequence of the single-stranded oligonucleotide comprises at least 13 contiguous nucleobases of a nucleobase sequence selected from Table 2. In embodiments, the nucleobase sequence of the single-stranded oligonucleotide comprises at least 14 contiguous nucleobases of a nucleobase sequence selected from Table 2. In embodiments, the nucleobase sequence of the single-stranded oligonucleotide comprises at least 15 contiguous nucleobases of a nucleobase sequence selected from Table 2. In embodiments, the nucleobase sequence of the single-stranded oligonucleotide comprises at least 16 contiguous nucleobases of a nucleobase sequence selected from Table 2. In embodiments, the nucleobase sequence of the single-stranded oligonucleotide comprises at least 17 contiguous nucleobases of a nucleobase sequence selected from Table 2. In embodiments, the nucleobase sequence of the single-stranded oligonucleotide comprises at least 18 contiguous nucleobases of a nucleobase sequence selected from Table 2. In embodiments, the nucleobase sequence of the single-stranded oligonucleotide comprises at least 19 contiguous nucleobases of a nucleobase sequence selected from Table 2. In embodiments, the nucleobase sequence of the single-stranded oligonucleotide comprises at least 20 contiguous nucleobases of a nucleobase sequence selected from Table 2. In embodiments, the nucleobase sequence of the single-stranded oligonucleotide comprises at least 21 contiguous nucleobases of a nucleobase sequence selected from Table 2. In embodiments, the nucleobase sequence of the single-stranded oligonucleotide comprises at least 22 contiguous nucleobases of a nucleobase sequence selected from Table 2. In embodiments, the nucleobase sequence of the single-stranded oligonucleotide comprises at least 23 contiguous nucleobases of a nucleobase sequence selected from Table 2. In embodiments, the nucleobase sequence of the single-stranded oligonucleotide comprises at least 24 contiguous nucleobases of a nucleobase sequence selected from Table 2. In embodiments, the nucleobase sequence of the single-stranded oligonucleotide comprises at least 25 contiguous nucleobases of a nucleobase sequence selected from Table 2. In embodiments, the nucleobase sequence of the single-stranded oligonucleotide comprises at least 26 contiguous nucleobases of a nucleobase sequence selected from Table 2. In embodiments, the nucleobase sequence of the single-stranded oligonucleotide comprises at least 27 contiguous nucleobases of a nucleobase sequence selected from Table 2. In embodiments, the nucleobase sequence of the single-stranded oligonucleotide comprises at least 28 contiguous nucleobases of a nucleobase sequence selected from Table 2. In embodiments, the nucleobase sequence of the single-stranded oligonucleotide comprises at least 29 contiguous nucleobases of a nucleobase sequence selected from Table 2. In embodiments, the nucleobase sequence of the single-stranded oligonucleotide comprises at least 30 contiguous nucleobases of a nucleobase sequence selected from Table 2.

TABLE 2
Annealing Sites and Nucleobase Sequences Targeting
Dystrophin

| | | SEQ |
|---|---|---|
| | | ID |
| Annealing Site | Nucleobase Sequence (5′ to 3′) | NO: |
| H51A(+66+95) | CUCCAACAUCAAGGAAGAUGGCAUUUCUAG | 1 |
| H53A(+36+60) | GUUGCCUCCGGUUCUGAAGGUGUUC | 2 |
| H45A(−03+19) | CAATGCCATCCTGGAGTTCCTG | 3 |
| H51A(+68+87) | UCAAGGAAGAUGGCAUUUCU | 4 |
| H53A(+36+56) | CGCTGCCCAATGCCAUCC | 5 |
| H45A(−10+8) | CCTCCGGTTCTGAAGGTGTTC | 6 |
| H10A(+130+149) | UUAGAAAUCUCUCCUUGUGC | 7 |
| H10A(+98+119) | UCCUCAGCAGAAAGAAGCCACG | 8 |
| H10A(−33−14) | UAAAUUGGGUGUUACACAAU | 9 |
| H11A(+118+140) | CUUGAAUUUAGGAGAUUCAUCUG | 10 |
| H11A(+75+97) | CAUCUUCUGAUAAUUUUCCUGUU | 11 |
| H11D(+11−09) | AGGACUUACUUGCUUUGUUU | 12 |
| H11D(+26+49) | CCCUGAGGCAUUCCCAUCUUGAAU | 13 |
| H12A(+11+30) | UUCUGGAGAUCCAUUAAAAC | 14 |
| H12A(+52+75) | UCUUCUGUUUUUGUUAGCCAGUCA | 15 |
| H12A(−10+10) | UCUAUGUAAACUGAAAAUUU | 16 |
| H13A(+55+75) | UUCAUCAACUACCACCACCAU | 17 |
| H13A(+77+100) | CAGCAGUUGCGUGAUCUCCACUAG | 18 |
| H13D(+06−19) | CUAAGCAAAAUAAUCUGACCUUAAG | 19 |
| H14A(+14+35) | CAUCUACAGAUGUUUGCCCAUC | 20 |
| H14A(+37+64) | CUUGUAAAAGAACCCAGCGGUCUUCUGU | 21 |
| H14A(+51+73) | GAAGGAUGUCUUGUAAAAGAACC | 22 |
| H14A(+61+80) | CAUUUGAGAAGGAUGUCUUG | 23 |
| H14A(−12+12) | AUCUCCCAAUACCUGGAGAAGAGA | 24 |
| H14D(+14−10) | CAUGACACACCUGUUCUUCAGUAA | 25 |
| H14D(−02+18) | ACCUGUUCUUCAGUAAGACG | 26 |
| H15A(+08+28) | UUUCUGAAAGCCAUGCACUAA | 27 |
| H15A(+48+71) | UCUUUAAAGCCAGUUGUGUGAAUC | 28 |
| H15A(−12+19) | GCCAUGCACUAAAAAGGCACUGCAAGACAUU | 29 |
| H15D(+17−08) | GUACAUACGGCCAGUUUUUGAAGAC | 30 |
| H16A(+105+126) | GUUAUCCAGCCAUGCUUCCGUC | 31 |
| H16A(+12+37) | UGGAUUGCUUUUUCUUUUCUAGAUCC | 32 |
| H16A(+45+67) | GAUCUUGUUUGAGUGAAUACAGU | 33 |
| H16A(+87+109) | CCGUCUUCUGGGUCACUGACUUA | 34 |
| H16A(+92+116) | CAUGCUUCCGUCUUCUGGGUCACUG | 35 |
| H16A(−06+19) | CUAGAUCCGCUUUUAAAACCUGUUA | 36 |
| H16A(−06+25) | UCUUUUCUAGAUCCGCUUUUAAAACCUGUUA | 37 |
| H16A(−07+13) | CCGCUUUUAAAACCUGUUAA | 38 |
| H16A(−07+19) | CUAGAUCCGCUUUUAAAACCUGUUAA | 39 |
| H16A(−12+19) | CUAGAUCCGCUUUUAAAACCUGUUAAAACAA | 40 |
| H16D(+05−20) | UGAUAAUUGGUAUCACUAACCUGUG | 41 |
| H16D(+12−11) | GUAUCACUAACCUGUGCUGUAC | 42 |
| H19A(+35+53) | CAGCAGUAGUUGUCAUCUGC | 43 |
| H19A(+35+65) | GCCUGAGCUGAUCUGCUGGCAUCUUGCAGUU | 44 |
| H20A(+149+168) | CUGCUGGCAUCUUGCAGUu | 45 |
| H20A(+149+170) | CAGCAGUAGUUGUCAUCUGCUC | 46 |
| H20A(+185+203) | AUCUGCAUUAACACCCUCUAGAAAG | 47 |
| H20A(+30+53) | CCGGCUGUUCAGUUGUUCUGAGGC | 48 |
| H20A(+44+63) | AUUCGAUCCACCGGCUGUUC | 49 |
| H20A(+44+71) | CUGGCAGAAUUCGAUCCACCGGCUGUUC | 50 |
| H20A(−08+17) | CCGGCUGUUCAGUUGUUCUGAGGC | 51 |
| H20A(−11+17) | AUCUGCAUUAACACCCUCUAGAAAGAAA | 52 |
| H20D(+08−20) | GAAGGAGAAGAGAUUCUUACCUUACAAA | 53 |
| H21A(+08+31) | GUUGAAGAUCUGAUAGCCGGUUGA | 54 |
| H21A(+85+106) | CUGCAUCCAGGAACAUGGGUCC | 55 |
| H21A(+85+108) | GUCUGCAUCCAGGAACAUGGGUC | 56 |
| H21A(−06+16) | GCCGGUUGACUUCAUCCUGUGC | 57 |
| H21D(+18−07) | UACUUACUGUCUGUAGCUcuuucu | 58 |
| H22A(+125+106) | CUGCAAUUCCCCGAGUCUCUGC | 59 |
| H22A(+22+45) | CACUCAUGGUCUCCUGAUAGCGCA | 60 |
| H22A(+47+69) | ACUGCUGGACCCAUGUCCUGAUG | 61 |
| H22A(+80+101) | CUAAGUUGAGGUAUGGAGAGU | 62 |
| H22D(+13−11) | UAUUCACAGACCUGCAAUucccc | 63 |
| H23A(+18+39) | UAGGCCACUUUGUUGCUCUUGC | 64 |
| H23A(+34+59) | ACAGUGGUGCUGAGAUAGUAUAGGCC | 65 |
| H23A(+72+90) | UUCAGAGGGCGCUUUCUUC | 66 |
| H24A(+48+70) | GGGCAGGCCAUUCCUCCUUCAGA | 67 |
| H24A(+9+36) | CTGGGCUGAAUUGUCUGAAUAUCACUG | 68 |
| H24A(−02+22) | UCUUCAGGGUUUGUAUGUGAUUCU | 69 |
| H25A(+131+156) | CUGUUGGCACAUGUGAUCCCACUGAG | 70 |
| H25D(+16−08) | GUCUAUACCUGUUGGCACAUGUGA | 71 |
| H26A(+132+156) | UGCUUUCUGUAAUUCAUCUGGAGUU | 72 |
| H26A(+68+92) | UGUGUCAUCCAUUCGUGCAUCUCUG | 73 |
| H26A(−07+19) | CCUCCUUUCUGGCAUAGACCUUCCAC | 74 |
| H27A(+82+106) | UUAAGGCCUCUUGUGCUACAGGUGG | 75 |
| H27A(−4+19) | GGGCCUCUUCUUUAGCUCUCUGA | 76 |
| H27D(+19−03) | GACUUCCAAAGUCUUGCAUUUC | 77 |
| H28A(+99+124) | CAGAGAUUUCCUCAGCUCCGCCAGGA | 78 |
| H28A(−05+19) | GCCAACAUGCCCAAACUUCCUAAG | 79 |
| H28D(+16−05) | CUUACAUCUAGCACCUCAGAG | 80 |
| H29A(+18+42) | AUUUGGGUUAUCCUCUGAAUGUCGC | 81 |
| H29A(+57+81) | UCCGCCAUCUGUUAGGGUCUGUGCC | 82 |
| H29D(+17−05) | CAUACCUCUUCAUGUAGUUCUC | 83 |
| H30A(+122+147) | CAUUUGAGCUGCGUCCACCUUGUCUG | 84 |
| H30A(+25+50) | UCCUGGGCAGACUGGAUGCUCUGUUC | 85 |
| H30D(+19−04) | UUGCCUGGGCUUCCUGAGGCAUU | 86 |
| H31A(+05+25) | GACUUGUCAAAUCAGAUUGGA | 87 |
| H31D(+03−22) | UAGUUUCUGAAAUAACAUAUACCUG | 88 |
| H31D(+04−20) | GUUUCUGAAAUAACAUAUACCUGU | 89 |
| H31D(+06−18) | UUCUGAAAUAACAUAUACCUGUGC | 90 |
| H32A(+10+32) | CGAAACUUCAUGGAGACAUCUUG | 91 |
| H32A(+151+170) | CAAUGAUUUAGCUGUGACUG | 92 |
| H32A(+49+73) | CUUGUAGACGCUGCUCAAAAUUGGC | 93 |
| H32D(+04−16) | CACCAGAAAUACAUACCACA | 94 |
| H33A(+30+56) | GUCUUUAUCACCAUUUCCACUUCAGAC | 95 |
| H33A(+53+76) | UCUGUACAAUCUGACGUCCAGUCU | 96 |
| H33A(+64+88) | CCGUCUGCUUUUUCUGUACAAUCUG | 97 |
| H33D(+09−11) | CAUGCACACACCUUUGCUcc | 98 |
| H34A(+143+165) | CCAGGCAACUUCAGAAUCCAAAU | 99 |
| H34A(+46+70) | CAUUCAUUUCCUUUCGCAUCUUACG | 100 |
| H34A(+72+96) | CUGUAGCUGCCAGCCAUUCUGUCAAG | 101 |
| H34A(+83+104) | UCCAUAUCUGUAGCUGCCAGCC | 102 |
| H34A(+95+120) | UGAUCUCUUUGUCAAUUCCAUAUCUG | 103 |
| H34A(−20+10) | UUUCUGUUACCUGAAAAGAAUUAUAAUGAA | 104 |
| H34D(+10−20) | UUCAGUGAUAUAGGUUUUACCUUUCCCCAG | 105 |
| H35A(+116+135) | CCAGUUACUAUUCAGAAGAC | 106 |
| H35A(+141+161) | UCUUCUGCUCGGGAGGUGACA | 107 |
| H35A(+24+43) | UCUUCAGGUGCACCUUCUGU | 108 |
| H36A(+26+50) | UGUGAUGUGGUCCACAUUCUGGUCA | 109 |
| H36A(−02+18) | CCAUGUGUUUCUGGUAUUcc | 110 |
| H37A(+134+157) | UUCUGUGUGAAAUGGCUGCAAAUC | 111 |
| H37A(+26+50) | CGUGUAGAGUCCACCUUUGGGCGUA | 112 |
| H37A(+82+105) | UACUAAUUUCCUGCAGUGGUCACC | 113 |
| H38A(+59+83) | UGCUGAAUUUCAGCCUCCAGUGGUU | 114 |
| H38A(+88+112) | UGAAGUCUUCCUCUUUCAGAUUCAC | 115 |
| H38A(−01+19) | CCUUCAAAGGAAUGGAGGcc | 116 |
| H39A(+102+121) | UUGUCUGUAACAGCUGCUGU | 117 |
| H39A(+39+58) | GUUGUAAGUUGUCUCCUCuu | 118 |
| H39A(+62+85) | CUGGCUUUCUCUCAUCUGUGAUUC | 119 |
| H39D(+10−10) | GCUCUAAUACCUUGAGAGCA | 120 |
| H3A(+30+50) | CUCCCAUCCUGUAGGUCACUG | 121 |
| H3A(+30+54) | GCGCCUCCCAUCCUGUAGGUCACUG | 122 |
| H3A(+30+60) | UAGGAGGCGCCUCCCAUCCUGUAGGUCACUG | 123 |
| H3A(+35+65) | AGGUCUAGGAGGCGCCUCCCAUCCUGUAGGU | 124 |
| H3A(+37+61) | CUAGGAGGCGCCUCCCAUCCUGUAG | 125 |
| H3A(−06+20) | UCAAUAUGCUGCUUCCCAAACUGAAA | 126 |
| H3D(+19−03) | UACCAGUUUUUGCCCUGUCAGG | 127 |
| H3D(+46−21) | CUUCGAGGAGGUCUAGGAGGCGCCUC | 128 |
| H40A(+129+153) | CUUUAUUUUCCUUUCAUCUCUGGGC | 129 |
| H40A(−05+17) | CUUUGAGACCUCAAAUCCUU | 130 |
| H42A(+86+109) | GGGCUUGUGAGACAUGAGUGAUUU | 131 |
| H42A(−04+23) | AUCGUUUCUUCACGGACAGUGUGCUGG | 132 |
| H42D(+19−02) | ACCUUCAGAGGACUCCUCUUGC | 133 |
| H43A(+101+120) | GGAGAGAGCUUCCUGUAGcu | 134 |
| H43A(+78+100) | UCACCCUUUCCACAGGCGUUGCA | 135 |
| H43D(+10−15) | UAUGUGUUACCUACCCUUGUCGGUC | 136 |
| H44A(+85+104) | UUUGUGUCUUUCUGAGAAAC | 137 |
| H44A(−06+14) | AUCUGUCAAAUCGCCUGCAG | 138 |
| H44D(+10−10) | AAAGACUUACCUUAAGAUAC | 139 |
| H45A(+125+151) | UGCAGACCUCCUGCCACCGCAGAUUCA | 140 |
| H45A(+71+90) | UGUUUUUGAGGAUUGCUGAA | 141 |
| H45A(+91+110) | UCCUGUAGAAUACUGGCAUC | 142 |
| H45A(−06+20) | CCAAUGCCAUCCUGGAGUUCCUGUAA | 143 |
| H45D(+16−04) | CUACCUCUUUUUUCUGUCUG | 144 |
| H46A(+107+137) | CAAGCUUUUCUUUUAGUUGCUGCUCUUUUCC | 145 |
| H46A(+50+77) | CUGCUUCCUCCAACCAUAAAACAAAUUC | 146 |
| H46A(+86+115) | CUCUUUUCCAGGUUCAAGUGGGAUACUAGC | 147 |
| H46A(+90+109) | UCCAGGUUCAAGUGGGAUAC | 148 |
| H46A(−10+20) | UAUUCUUUUGUUCUUCUAGCCUGGAGAAAG | 149 |
| H46D(+16−04) | UUACCUUGACUUGCUCAAGC | 150 |
| H47A(+76+100) | GCUCUUCUGGGCUUAUGGGAGCACU | 151 |
| H47A(−9+12) | UUCCACCAGUAACUGAAACAG | 152 |
| H47D(+25−02) | CUUCCACUCAGAGCUCAGAUCUUCUAA | 153 |
| H4A(+11+40) | UGUUCAGGGCAUGAACUCUUGUGGAUCCUU | 154 |
| H4A(+13+32) | GCAUGAACUCUUGUGGAUcc | 155 |
| H4D(+04−16) | CCAGGGUACUACUUACAUUA | 156 |
| H4D(−24−44) | AUCGUGUGUCACAGCAUCCAG | 157 |
| H50A(+02+30) | CCACUCAGAGCUCAGAUCUUCUAACUUCC | 158 |
| H50A(+07+33) | GGGAUCCAGUAUACUUACAGGCUCC | 159 |
| H50D(+07−18) | ACCUUUAUCCACUGGAGAUUUGUCUGC | 160 |
| H51A(+111+134) | UUCUGUCCAAGCCCGGUUGAAAUC | 161 |
| H51A(+175+195) | CACCCACCAUCACCCUCUGUG | 162 |
| H51A(+199+220) | AUCAUCUCGUUGAUAUCCUCAA | 163 |
| H51A(+61+90) | ACAUCAAGGAAGAUGGCAUUUCUAGUUUGG | 164 |
| H51A(+66+90) | ACAUCAAGGAAGAUGGCAUUUCUAG | 165 |
| H51A(+66+95) | CUCCAACAUCAAGGAAGAUGGCAUUUCUAG | 166 |
| H51A(−01+25) | ACCAGAGUAACAGUCUGAGUAGGAGC | 167 |
| H51A/D(+08−17) | AUCAUUUUUUCUCAUACCUUCUGCUAGGAGCUA | 168 |
| &(−15+) | AAA | |
| H51D(+08−17) | AUCAUUUUUUCUCAUACCUUCUGCU | 169 |
| H51D(+16−07) | CUCAUACCUUCUGCUUGAUGAUC | 170 |
| H52A(+12+41) | UCCAACUGGGGACGCCUCUGUUCCAAAUCC | 171 |
| H52A(+17+37) | ACUGGGGACGCCUCUGUUCCA | 172 |
| H52A(+93+112) | CCGUAAUGAUUGUUCUAGcc | 173 |
| H52A(−07+14) | UCCUGCAUUGUUGCCUGUAAG | 174 |
| H52D(+05−15) | UGUUAAAAAACUUACUUCGA | 175 |
| H53A(+07+26) | AUCCCACUGAUUCUGAAUUC | 176 |
| H53A(+124+145) | UUGGCUCUGGCCUGUCCUAAGA | 177 |
| H53A(+150+176) | UGUAUAGGGACCCUCCUUCCAUGACUC | 178 |
| H53A(+23+47) | CUGAAGGUGUUCUUGUACUUCAUCC | 179 |
| H53A(+39+62) | CUGUUGCCUCCGGUUCUGAAGGUG | 180 |
| H53A(+39+69) | CAUUCAACUGUUGCCUCCGGUUCUGAAGGUG | 181 |
| H53A(+45+69) | CAUUCAACUGUUGCCUCCGGUUCUG | 182 |
| H53A(−07+18) | GAUUCUGAAUUCUUUCAACUAGAAU | 183 |
| H53A(−12+10) | AUUCUUUCAACUAGAAUAAAAG | 184 |
| H53D(+09−18) | GGUAUCUUUGAUACUAACCUUGGUUUC | 185 |
| H53D(+14−07) | UACUAACCUUGGUUUCUGUGA | 186 |
| H53D(+20−05) | CUAACCUUGGUUUCUGUGAUUUUCU | 187 |
| H5A(+05+35) | ACGAUGUCAGUACUUCCAAUAUUCACUAAAU | 188 |
| H5A(+10+34) | CGAUGUCAGUACUUCCAAUAUUCAC | 189 |
| H5A(+15+45) | AUUUCCAUCUACGAUGUCAGUACUUCCAAUA | 190 |
| H5A(+20+50) | UUAUGAUUUCCAUCUACGAUGUCAGUACUUC | 191 |
| H5A(−07+20) | CCAAUAUUCACUAAAUCAACCUGUUAA | 192 |
| H5D(+10−15) | CAUCAGGAUUCUUACCUGCCAGUGG | 193 |
| H5D(+16−02) | ACCUGCCAGUGGAGGAUU | 194 |
| H5D(+18−12) | CAGGAUUCUUACCUGCCAGUGGAGGAUUAU | 195 |
| H5D(+25−05) | CUUACCUGCCAGUGGAGGAUUAUAUUCCAAA | 196 |
| H5D(−04−21) | ACCAUUCAUCAGGAUUCU | 197 |
| H6D(+04−21) | UGUCUCAGUAAUCUUCUUACCUAU | 198 |
| H6D(+18−04) | UCUUACCUAUGACUAUGGAUGAGA | 199 |
| H7A(+02+26) | CACUAUUCCAGUCAAAUAGGUCUGG | 200 |
| H7A(+45+67) | UGCAUGUUCCAGUCGUUGUGUGG | 201 |
| H7A(−18+03) | GGCCUAAAACACAUACACAUA | 202 |
| H7D(+15−10) | AUUUACCAACCUUCAGGAUCGAGUA | 203 |
| H8A(−03+18) | GAUAGGUGGUAUCAACAUCUG | 204 |
| H8A(−06+14) | GGUGGUAUCAACAUCUGUAA | 205 |
| H8A(−06+18) | GAUAGGUGGUAUCAACAUCUGUAA | 206 |
| H8A(−07+18) | GAUAGGUGGUAUCAACAUCUGUAAG | 207 |
| H8A(−10+10) | GUAUCAACAUCUGUAAGCAC | 208 |
| H10A(−05+16) | CAGGAGCUUCCAAAUGCUGCA | 209 |
| H10A(−05+24) | CUUGUCUUCAGGAGCUUCCAAAUGCUGCA | 210 |

In embodiments, the annealing site is H51A(+66+95), which is the site from the 66^{th} to the 95^{th} nucleotide from the start of exon 51 of human dystrophin pre-mRNA. In embodiments, the annealing site is H53A(+36+60), which is the site from the 36^{th} to 60^{th} nucleotide from the start of exon 53 of human dystrophin pre-mRNA. In embodiments, the annealing site is H45A(−03+19), which is the site from last 3 nucleotides of the intron preceding exon 45 to the 19^{th} nucleotide of exon 45 of human dystrophin pre-mRNA. In embodiments, the annealing site is H51A(+68+87), which is the site from the 68^{th} to the 87^{th} nucleotide from the start of exon 51 of human dystrophin pre-mRNA. In embodiments, the annealing site is H53A(+36+56), which is the site from the 36^{th} to 56^{th} nucleotide from the start of exon 53 of human dystrophin pre-mRNA. In embodiments, the annealing site is H45A(−10+8), which is the site from the last 10 nucleotides of the intron preceding exon 45 to the 8^{th} nucleotide from the start of exon 45 of human dystrophin pre-mRNA.

In embodiments, the nucleobase sequence of the single-stranded oligonucleotide is 5′-CUCCAACAUCAAGGAAGAUGGCAUUUCUAG-3′ (SEQ ID NO: 1). In embodiments, the nucleobase sequence of the single-stranded oligonucleotide is 5′-GUUGCCUCCGGUUCUGAAGGUGUUC-3′ (SEQ ID NO: 2). In embodiments, the nucleobase sequence of the single-stranded oligonucleotide is 5′-CAATGCCATCCTGGAGTTCCTG-3′ (SEQ ID NO: 3). In embodiments, the nucleobase sequence of the single-stranded oligonucleotide is 5′-UCAAGGAAGAUGGCAUUUCU-3′ (SEQ ID NO: 4). In embodiments, the nucleobase sequence of the single-stranded oligonucleotide is 5′-CGCTGCCCAATGCCAUCC-3′ (SEQ ID NO: 5). In embodiments, the nucleobase sequence of the single-stranded oligonucleotide is 5′-CCTCCGGTTCTGAAGGTGTTC-3′ (SEQ ID NO: 6).

In embodiments, the single-stranded oligonucleotide is 8 to 30 nucleotides in length. In embodiments, the single-stranded oligonucleotide is 10 to 30 nucleotides in length. In embodiments, the single-stranded oligonucleotide is 15 to 30 nucleotides in length. In embodiments, the single-stranded oligonucleotide is 20 to 30 nucleotides in length. In embodiments, the single-stranded oligonucleotide is 25 to 30 nucleotides in length. In embodiments, the single-stranded oligonucleotide is 8 nucleotides in length. In embodiments, the single-stranded oligonucleotide is 8 nucleotides in length. In embodiments, the single-stranded oligonucleotide is 9 nucleotides in length. In embodiments, the single-stranded oligonucleotide is 10 nucleotides in length. In embodiments, the single-stranded oligonucleotide is 11 nucleotides in length. In embodiments, the single-stranded oligonucleotide is 12 nucleotides in length. In embodiments, the single-stranded oligonucleotide is 13 nucleotides in length. In embodiments, the single-stranded oligonucleotide is 14 nucleotides in length. In embodiments, the single-stranded oligonucleotide is 15 nucleotides in length. In embodiments, the single-stranded oligonucleotide is 16 nucleotides in length. In embodiments, the single-stranded oligonucleotide is 17 nucleotides in length. In embodiments, the single-stranded oligonucleotide is 18 nucleotides in length. In embodiments, the single-stranded oligonucleotide is 19 nucleotides in length. In embodiments, the single-stranded oligonucleotide is 20 nucleotides in length. In embodiments, the single-stranded oligonucleotide is 21 nucleotides in length. In embodiments, the single-stranded oligonucleotide is 22 nucleotides in length. In embodiments, the single-stranded oligonucleotide is 23 nucleotides in length. In embodiments, the single-stranded oligonucleotide is 24 nucleotides in length. In embodiments, the single-stranded oligonucleotide is 25 nucleotides in length. In embodiments, the single-stranded oligonucleotide is 26 nucleotides in length. In embodiments, the single-stranded oligonucleotide is 27 nucleotides in length. In embodiments, the single-stranded oligonucleotide is 28 nucleotides in length. In embodiments, the single-stranded oligonucleotide is 29 nucleotides in length. In embodiments, the single-stranded oligonucleotide is 30 nucleotides in length.

In embodiments, a single-stranded oligonucleotide comprises one or more sugar moieties independently selected from a 2′-O-methyl sugar modification, a 2′-O-methoxyethyl sugar modification, and a 2′-fluoro sugar modification. In embodiments, a single-stranded oligonucleotide comprises one or more sugar moieties independently selected from a 2′-O-methyl sugar modification and a 2′-O-methoxyethyl sugar modification. In embodiments, each nucleotide of the single-stranded oligonucleotide comprises a modified sugar moiety independently selected from a 2′-O-methyl sugar modification, a 2′-O-methoxyethyl sugar modification, and a 2′-fluoro sugar modification. In embodiments, each nucleotide of the single-stranded oligonucleotide comprises a modified sugar moiety independently selected from a 2′-O-methyl sugar modification, and a 2′-O-methoxyethyl sugar modification.

In embodiments, the nucleobase sequence of the single-stranded oligonucleotide is 5′-CUCCAACAUCAAGGAAGAUGGCAUUUCUAG-3′ (SEQ ID NO: 1), each nucleotide comprises a morpholino moiety, and each morpholino moiety is linked by a phosphorodiamidite linkage. In embodiments, the nucleobase sequence of the single-stranded oligonucleotide is 5′-CUCCAACAUCAAGGAAGAUGGCAUUUCUAG-3′ (SEQ ID NO: 1), each nucleotide of the single-stranded nucleotide comprises a 2′-O-methoxyethyl sugar modification, and wherein each internucleotide linkage is a phosphorothioate linkage. In embodiments, the nucleobase sequence of the single-stranded oligonucleotide is 5′-CUCCAACAUCAAGGAAGAUGGCAUUUCUAG-3′ (SEQ ID NO: 1), each nucleotide of the single-stranded oligonucleotide comprises a 2′-O-methyl sugar modification, and wherein each internucleotide linkage is a phosphorothioate linkage.

In embodiments, the nucleobase sequence of the single-stranded oligonucleotide is 5′-GUUGCCUCCGGUUCUGAAGGUGUUC-3′ (SEQ ID NO: 2), each nucleotide comprises a morpholino moiety, and each morpholino moiety is linked by a phosphorodiamidite linkage. In embodiments, the nucleobase sequence of the single-stranded oligonucleotide is 5′-GUUGCCUCCGGUUCUGAAGGUGUUC-3′ (SEQ ID NO: 2), each nucleotide of the single-stranded nucleotide comprises a 2′-O-methoxyethyl sugar modification, and wherein each internucleotide linkage is a phosphorothioate linkage. In embodiments, the nucleobase sequence of the single-stranded oligonucleotide is 5′-GUUGCCUCCGGUUCUGAAGGUGUUC-3′(SEQ ID NO: 2), each nucleotide of the single-stranded oligonucleotide comprises a 2′-O-methyl sugar modification, and wherein each internucleotide linkage is a phosphorothioate linkage.

In embodiments, the nucleobase sequence of the single-stranded oligonucleotide is 5′-CAATGCCATCCTGGAGTTCCTG-3′ (SEQ ID NO: 3), each nucleotide comprises a morpholino moiety, and each morpholino moiety is linked by a phosphorodiamidite linkage. In embodiments, the nucleobase sequence of the single-stranded oligonucleotide is 5′-CAATGCCATCCTGGAGTTCCTG-3′ (SEQ ID NO: 3), each nucleotide of the single-stranded nucleotide comprises a 2′-O-methoxyethyl sugar modification, and wherein each internucleotide linkage is a phosphorothioate linkage. In embodiments, the nucleobase sequence of the single-stranded oligonucleotide is 5′-CAATGCCATCCTGGAGTTCCTG-3′ (SEQ ID NO: 3), each nucleotide of the single-stranded oligonucleotide comprises a 2′-O-methyl sugar modification, and wherein each internucleotide linkage is a phosphorothioate linkage.

In embodiments, the nucleobase sequence of the single-stranded oligonucleotide is 5′-UCAAGGAAGAUGGCAUUUCU-3′ (SEQ ID NO: 4), each nucleotide comprises a morpholino moiety, and each morpholino moiety is linked by a phosphorodiamidite linkage. In embodiments, the nucleobase sequence of the single-stranded oligonucleotide is 5′-UCAAGGAAGAUGGCAUUUCU-3′ (SEQ ID NO: 4), each nucleotide of the single-stranded nucleotide comprises a 2′-O-methoxyethyl sugar modification, and wherein each internucleotide linkage is a phosphorothioate linkage. In embodiments, the nucleobase sequence of the single-stranded oligonucleotide is 5′-UCAAGGAAGAUGGCAUUUCU-3′ (SEQ ID NO: 4), each nucleotide of the single-stranded oligonucleotide comprises a 2′-O-methyl sugar modification, and wherein each internucleotide linkage is a phosphorothioate linkage.

In embodiments, the nucleobase sequence of the single-stranded oligonucleotide is 5′-CGCTGCCCAATGCCAUCC-3′ (SEQ ID NO: 5), each nucleotide of the single-stranded oligonucleotide comprises a 2′ modification, wherein the 2′ modification is a 2′-O-methyl modification or a bicyclic sugar modification with a 4′-(CH₂)₂—O-2′ linkage, and wherein each linkage is a phosphodiester linkage.

In embodiments, the nucleobase sequence of the single-stranded oligonucleotide is 5′-UCAAGGAAGAUGGCAUUUCU-3′ (SEQ ID NO: 4), each nucleotide of the single-stranded oligonucleotide comprises a 2′-modification, wherein the 2′ modification is a 2′-O-methyl modification or a 2′-fluoro modification, and wherein each internucleotide linkage is selected from a phosphorothoiate linkage of the Sp conformation and a phosphodiester linkage.

In embodiments, the nucleobase sequence of the single-stranded oligonucleotide is 5′-CCTCCGGTTCTGAAGGTGTTC-3′ (SEQ ID NO: 6), each nucleotide comprises a morpholino moiety, and each morpholino moiety is linked by a phosphorodiamidite linkage.

Certain single-stranded oligonucleotide structures are provided in Table 4, where each nucleotide is described by the “Nsl” notation where N=nucleobase, s=sugar, and 1=internucleotide linker. Also shown are the 5′ end 3′ terminal ends ofeach oligonucleotide. A description for each sugar, linker, 5′ end and 3′ end abbreviation is shown in Table 3. Nucleobases are represented by A, G, T, C, U, and mC (5-methyl cytosine). Single-stranded oligonucleotides conjugated to an uptake motifare shown in Table 5, where the uptake domain and linker are indicated.

In embodiments, the single-stranded oligonucleotide (A) of formula (IV) is a structure provided in Table 4. In embodiments, a compound including a single-stranded oligonucleotide is a structure provided in Table 5.

TABLE 3
Nucleotide Sugar and Linkage Abbreviations

| Abbreviation | Definition |
|---|---|
| e | 2′-O-methoxyethyl sugar modification |
| f | 2′-fluoro sugar modification |
| m | 2′-O-methyl sugar modification |
| b | aminooxy bicyclic sugar modification |
| k | cEt sugar modification |
| y | ENA sugar modification |
| o | morpholino moiety |
| d | 2′-deoxy (DNA) |
| r | 2′-hydroxy (RNA) |
| o | phosphodiester linkage |
| s | phosphorothioate linkage |
| *s | phosphorothioate linkage in Sp configuration |
| ps | phosphorodiamidate linkage |
| a | amide linkage |
| 5′-SARC | Sarcosine linked to the 6′ carbon of a 5′ terminal |
| | nucleotide having a morpholino moiety |
| 5′-HO | Hydroxyl linked to the 5′ carbon of a 5′ terminal |
| | nucleotide having a pentafuranosyl sugar |
| 3′-H | Hydrogen linked to the 3′ nitrogen of a 3′ |
| | terminal nucleotide having a morpholino moiety |
| 3′-OH | Hydroxyl linked to the 3′ carbon of a 3′ terminal |
| | nucleotide having a pentafuranosyl sugar |
| EN5C6 | C6 linker at the 5′ end of an oligonucleotide |
| | linked via a phosphodiester linkage |
| EN3C6 | C6 linker at the 3′ end of an oligonucleotide |
| | including phosphodiester linkages |
| EP5C6 | C6 linker at the 5′ end of an oligonucleotide |
| | including morpholino moieties |
| EP3C6 | C6 linker used at the 3′ end of an oligonucleotide |
| | including morpholino moieties; linked to the 3′- |
| | nitrogen via an amide (a) linkage |
| EN3C7 | C7 linker used at the 3′ end of an oligonucleotide |
| | including phosphodiester linkages |
| EP3AMC | A substituted piperidine-containing linker used at |
| | the 3′ end of an oligonucleotide including |
| | morpholino moieties; linked to the 3′-nitrogen via |
| | an amide (a) linkage. |
| EP3TEG | Triethyleneglycol-containing linker used at the 3′ |
| | end of an oligonucleotide including morpholino |
| | moieties; linked to the 3′-nitrogen via an amide |
| | (a) linkage. |

TABLE 4
Single-Stranded Oligonucleotides Targeted to Dystrophin

| | | SEQ |
|---|---|---|
| Compound | | ID |
| Number | Nsl Notation | NO: |
| DT-000089 | [5′- | 211 |
| | HO][mCes][Tes][mCes][mCes][Aes][Aes][mCes][Aes][Tes][mCes][Aes] | |
| | [Aes][Ges][Ges][Aes][Aes][Ges][Aes][Tes][Ges][Ges][mCes][Aes][Tes] | |
| | [Tes][Tes][mCes][Tes][Aes][Ge][OH-3′] | |
| DT-000090 | [5′- | 212 |
| | HO][Ums][Cms][Ams][Ams][Gms][Gms][Ams][Ams][Gms][Ams][Ums] | |
| | [Gms][Gms][Cms][Ams][Ums][Ums][Ums][Cms][Um][OH-3′] | |
| DT-000091 | [5′- | 213 |
| | HO][Tes][mCes][Aes][Aes][Ges][Ges][Aes][Aes][Ges][Aes][Tes][Ges] | |
| | [Ges][mCes][Aes][Tes][Tes][Tes][mCes][Te][OH-3′] | |
| DT-000094 | [5′-SARC][Gop][Top][Top][Gop][Cop][Cop][Top][Cop][Cop][Gop] | 214 |
| | [Gop][Top][Top][Cop][Top][Gop][Aop][Aop][Gop][Gop][Top][Gop][Top] | |
| | [Top][Co][H-3′] | |
| DT-000095 | [5′- | 215 |
| | HO][Gms][Ums][Ums][Gms][Cms][Cms][Ums][Cms][Cms][Gms][Gms] | |
| | [Ums][Ums][Cms][Ums][Gms][Ams][Ams][Gms][Gms][Ums][Gms][Ums] | |
| | [Ums][Cm][OH-3′] | |
| DT-000096 | [5′- | 214 |
| | HO][Ges][Tes][Tes][Ges][Ces][mCes][Tes][mCes][mCes][Ges][Ges][Tes] | |
| | [Tes][mCes][Tes][Ges][Aes][Aes][Ges][Ges][Tes][Ges][Tes][Tes][mCe] | |
| | [OH-3′] | |
| DT-000097 | [5′-SARC][Top][Cop][Aop][Aop][Gop][Gop][Aop][Aop][Gop][Aop] | 213 |
| | [Top] [Gop][Gop][Cop][Aop][Top][Top][Top][Cop][To][H-3′] | |
| DT-000098 | [5′-SARC][Cop][Top][Cop][Cop][Aop][Aop][Cop][Aop][Top][Cop] | 211 |
| | [Aop] [Aop][Gop][Gop][Aop][Aop][Gop][Aop][Top][Gop][Gop][Cop] | |
| | [Aop][Top][Top][Top][Cop][Top][Aop][Go][H-3′] | |
| DT-000340 | [5′-SARC][Cop][Cop][Top][Cop][Cop][Gop][Gop][Top][Top][Cop] | 216 |
| | [Top][Gop][Aop][Aop][Gop][Gop][Top][Gop][Top][Top][Co][-H-3′] | |
| DT-000341 | [5′-SARC][Cop][Aop][Aop]Top][Gop][Cop][Cop][Aop][Top][Cop] | 217 |
| | [Cop][Top][Gop][Gop][Aop][Gop][Top][Top][Cop][Cop][Top][Go][H-3′] | |
| DT-000348 | [5′-HO][Ufs][Cfs][Afs][Afs][Gfs][Gfs][Amo][Afs][Gmo][Ams][Ufs] | 212 |
| | [Gmo][Gmo][Cfs][Afs][Ufs][Ufs][Ufs][Cfs][Uf][OH-3′] | |
| DT- | [5- | 212 |
| 000348.1 | HO][Uf*s][Cf*s][Af*s][Af*s][Gf*s][Gf*s][Amo][Af*s][Gmo][Ams][Uf*s] | |
| | [Gmo][Gmo][Cf*s][Af*s][Uf*s][Uf*s][Uf*s][Cf*s][Uf][OH-3′] | |
| DT-000349 | [5′-HO][Cys][Gms][Cys][Tys][Gms][Cms][Cys][Cys][Ams][Ams] | 218 |
| | [Tys][Gms][Cys][Cys][Ams][Ums][Cys][Cy][OH-3′] | |

TABLE 5
Conjugated Dystrophin-Targeting Compounds

| | | SEQ |
|---|---|---|
| Compound | | ID |
| Number | Nsl Notation | NO: |
| DT-000188 | [5′-SARC][Top][Cop][Aop][Aop][Gop][Gop][Aop][Aop][Gop][Aop] | 213 |
| | [Top][Gop][Gop][Cop][Aop][Top][Top][Top][Cop][Toa] | |
| | [EP3C6][DTx-01-08] | |
| DT-000189 | [5′-SARC][Gop][Top][Top][Gop][Cop][Cop][Top][Cop][Cop][Gop] | 214 |
| | [Gop][Top][Top][Cop][Top][Gop][Aop][Aop][Gop][Gop][Top][Gop] | |
| | [Top][Top][Coa][EP3C6][DTx-01-08] | |
| DT-000192 | [5′- | 214 |
| | HO][Ges][Tes][Tes][Ges][Ces][mCes][Tes][mCes][mCes][Ges][Ges][Tes] | |
| | [Tes][mCes][Tes][Ges][Aes][Aes][Ges][Ges][Tes][Ges][Tes][Tes][mCeo] | |
| | [EN3C7][DTx-01-08] | |
| DT-000193 | [5′- | 212 |
| | HO][Ums][Cms][Ams][Ams][Gms][Gms][Ams][Ams][Gms][Ams][Ums] | |
| | [Gms] [Gms][Cms][Ams][Ums][Ums][Ums][Cms][Umo] | |
| | [EN3C7][DTx-01-08] | |
| DT-000194 | [5′- | 213 |
| | HO][Tes][mCes][Aes][Aes][Ges][Ges][Aes][Aes][Ges][Aes][Tes][Ges] | |
| | [Ges][mCes][Aes][Tes][Tes][Tes][mCes][Teo] | |
| | [EN3C7][DTx-01-08] | |
| DT-000342 | [5′-SARC][Cop][Top][Cop][Cop][Aop][Aop][Cop][Aop][Top][Cop] | 211 |
| | [Aop] [Aop][Gop][Gop][Aop][Aop][Gop][Aop][Top][Gop][Gop][Cop] | |
| | [Aop][Top][Top][Top][Cop][Top][Aop][Goa] | |
| | [EP3C6][DTx-01-08] | |
| DT-000343 | [DTx-01-08][EP5GTPAp] | 211 |
| | [Cop][Top][Cop][Cop][Aop][Aop][Cop][Aop][Top][Cop][Aop][Aop][Gop] | |
| | [Gop][Aop][Aop][Gop][Aop][Top][Gop][Gop][Cop][Aop][Top][Top] | |
| | [Top][Cop][Top][Aop][Go][H-3′] | |
| DT-000344 | [5′-SARC][Cop][Cop][Top][Cop][Cop][Gop][Gop][Top][Top] | 216 |
| | [Cop][Top][Gop][Aop][Aop][Gop][Gop][Top][Gop][Top][Top][Coa] | |
| | [EP3C6][DTx-01-08] | |
| DT-000345 | [5′-SARC][Cop][Aop][Aop][Top][Gop][Cop][Cop][Aop][Top][Cop] | 217 |
| | [Cop][Top][Gop][Gop][Aop][Gop][Top][Top][Cop][Cop][Top][Goa] | |
| | [EP3C6][DTx-01-08] | |
| DT-000346 | [DTx-01-08][EP5GTPAp] | 217 |
| | [Cop][Aop][Aop][Top][Gop][Cop][Cop][Aop][Top][Cop][Cop][Top] | |
| | [Gop][Gop][Aop][Gop][Top][Top][Cop][Cop][Top][Go][H-3′] | |

In embodiments, the compound further includes a ligand. In embodiments, the ligand may include one or more selected from a synthetic compound, a peptide, an antibody, a carbohydrate, or an additional nucleic acid. In embodiments, the ligand may include one or more selected from a peptide, an antibody, a carbohydrate, or an additional nucleic acid. In embodiments, the uptake motif independently includes one or more selected from a peptide, an antibody, a carbohydrate, or an additional nucleic acid. In embodiments, one or more update motifs include one or more selected from a peptide, an antibody, a carbohydrate, or an additional nucleic acid. In embodiments, the ligand may replace the update motif. In embodiments, one or more ligand may replace one or more update motifs.

In embodiments, the compound further includes a ligand. In embodiments, the ligand may include one or more selected from a synthetic compound, a peptide, an antibody, a carbohydrate, or an additional nucleic acid. In embodiments, the ligand may include one or more selected from a peptide, an antibody, a carbohydrate, or an additional nucleic acid. In embodiments, the uptake motif independently includes one or more selected from a peptide, an antibody, a carbohydrate, or an additional nucleic acid. In embodiments, one or more uptake motifs include one or more selected from a peptide, an antibody, a carbohydrate, or an additional nucleic acid. In embodiments, the ligand may replace the uptake motif. In embodiments, one or more ligand may replace one or more uptake motifs.

### Pharmaceutical Compositions

Also provided herein are pharmaceutical formulations or pharmaceutical composition. I n embodiments, the pharmaceutical formulation (e.g., composition) includes a compound (e.g. formula (IV) or (IV-a)) described above including in any aspects, embodiments, claims, figures tables (e.g., Tables 1-5 and A-O), examples, or schemes (e.g., Schemes I, II, III, and IV), and a pharmaceutically acceptable excipient.

The pharmaceutical composition may be prepared and administered in a wide variety of dosage formulations. Compounds described may be administered orally, rectally, or by injection (e.g. intravenously, intramuscularly, intracutaneously, subcutaneously, intraduodenally, or intraperitoneally).

For preparing pharmaceutical compositions from compounds described herein, pharmaceutically acceptable carriers can be either solid or liquid. Solid form preparations include powders, tablets, pills, capsules, cachets, suppositories, and dispersible granules. A solid carrier may be one or more substance that may also act as diluents, flavoring agents, binders, preservatives, tablet disintegrating agents, or an encapsulating material.

In powders, the carrier may be a finely divided solid in a mixture with the finely divided active component. In tablets, the active component may be mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight. Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The pharmaceutical compositions may additionally include components to provide sustained release and/or comfort. Such components include high molecular weight, anionic mucomimetic polymers, gelling polysaccharides, and finely-divided drug carrier substrates.

The pharmaceutical composition may be intended for intravenous use. The pharmaceutically acceptable excipient can include buffers to adjust the pH to a desirable range for intravenous use. Many buffers including salts of inorganic acids such as phosphate, borate, and sulfate are known.

In an aspect, provided is a cell containing a compound including a single-stranded oligonucleotide (A) (e.g. (IV) or (IV-a)) described above including in any aspects, embodiments, claims, figures, tables (e.g., Tables 1-5 and A-O), examples, or schemes (e.g., Schemes I, II, III, and IV).

In embodiments, the cell containing the compound including a singles-stranded oligonucleotide (A) may include, but be not limited to, a fibroblast cell, a kidney cell, an endothelial cell, an adipose cell, a neuronal cell, a muscle cell, a hepatocyte cell, a T lymphocyte, and a B lymphocyte. In embodiments, the cell containing the compound including a single-stranded oligonucleotide (A) may include, but be not limited to, a human umbilical vein endothelial cell, NIH3T3 cell, RAW264.7 cell, a HEK293 cell or SH-SY5Y cell.

### Methods and Use

In an aspect, provided is a method including contacting a cell with a compound as described herein. In embodiments, the method includes contacting a cell with one or more compounds as described herein, including in any aspects, embodiments, claims, figures, tables (e.g., Tables 1-5 and A-O), examples, or schemes (e.g., Schemes I, II, III, and IV). In embodiments, the contacting occurs in vitro. In embodiments, the contacting occurs ex vivo. In embodiments, the contacting occurs in vivo.

In an aspect, provided is a method including inducing skipping of an exon of the dystrophin pre-mRNA in a cell. In embodiments, the method includes contacting a cell with one or more compounds as described herein, including in any aspects, embodiments, claims, figures, tables (e.g., Tables 1-5 and A-O), examples, or schemes (e.g., Schemes I, II, III, and IV). In embodiments, the contacting occurs in vitro. In embodiments, the contacting occurs ex vivo. In embodiments, the contacting occurs in vivo.

In an aspect, provided is a method of administering to a subject a compound as described herein. In embodiments, the method includes administering to a subject one or more compounds as described herein, including in any aspects, embodiments, claims, figures, tables (e.g., Tables 1-5 and A-O), examples, or schemes (e.g., Schemes I, II, III, and IV). In embodiments, the subject has Duchenne muscular dystrophy. In embodiments, the subject has Duchene muscular dystrophy and is determined to have a mutation in the dystrophin gene that is amenable to exon skipping.

In an aspect, provided is a method of treating Duchenne muscular dystrophy. In embodiments, the method includes administering to a subject one or more compounds as described herein, including in any aspects, embodiments, claims, figures, tables (e.g., Tables 1-5 and A-O), examples, or schemes (e.g., Schemes I, II, III, and IV).

In embodiments related to contacting a cell, one or more exons of the pre-mRNA of dystrophin is skipped at an increased level relative to the absence of the compound. In embodiments related to administration to a subject, one or more exons of the pre-mRNA of dystrophin is skipped at an increased level relative to the absence of the compound.

In embodiments related to administration to a subject, the administration is systemic administration, which may include, without limitation, subcutaneous administration, intravenous administration, intramuscular administration, and oral administration. In embodiments related to administration to a subject, the administration is local administration, which may include, without limitation, intravitreal administration, intrathecal administration, and intraventricular administration.

In an aspect, provided is a use of a compound as described herein in a therapy. In an aspect, provided is a use of a compound as described herein in the preparation of a medicament.

In an aspect, provided is a method of introducing a single-stranded oligonucleotide into a cell within a subject. In embodiments, the method includes administering to said subject a compound as described herein.

### EMBODIMENTS

Embodiment 1. A compound having the structure:

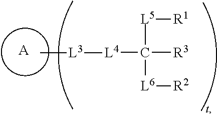

wherein

A is a single-stranded oligonucleotide having a nucleobase sequence complementary to a portion of the dystrophin pre-mRNA; t is an integer from 1 to 5;

L³ and L⁴ are independently a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;

L⁵ is -L^{5A}-L^{5B}-L^{5C}-L^{5D}-L^{5E}-.

L⁶ is -L^{6A}-L^{6B}-L^{6C}-L^{6D}-L^{6E}-;

R¹ and R² are independently unsubstituted C₁-C₂₅ alkyl, wherein at least one of R¹ and R² is unsubstituted C₉-C₁₉ alkyl;

R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L^{5A}, L^{5B}, L^{5C}, L^{5D}, L^{5E}, L^{6A}, L^{6B}, L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; and each R²³, R²⁴ and R²⁵ is independently hydrogen or unsubstituted C₁-C₁₀ alkyl.

Embodiment 2. The compound of Embodiment 1, wherein t is 1.

Embodiment 3. The compound of Embodiment 1, wherein t is 2.

Embodiment 4. The compound of Embodiment 1, wherein t is 3.

Embodiment 5. The compound of one of Embodiments 1 to 4, wherein each of R²³, R²⁴ and R²⁵ is independently hydrogen or unsubstituted C₁-C₃ alkyl.

Embodiment 6. The compound of one of Embodiments 1 to 4, wherein one L³ is attached to a 3′ carbon of the oligonucleotide.

Embodiment 7. The compound of one of Embodiments 1 to 4, wherein one L³ is attached to a 3′ nitrogen of the oligonucleotide.

Embodiment 8. The compound of one of Embodiments 1 to 4, wherein one L³ is attached to a 5′ carbon of the oligonucleotide.

Embodiment 9. The compound of one of Embodiments 1 to 4, wherein one L³ is attached to a 6′ carbon of the oligonucleotide.

Embodiment 10. The compound of one of Embodiments 1 to 4, wherein one L³ is attached to a nucleobase of the oligonucleotide.

Embodiment 11. The compound of one of Embodiments 1 to 10, wherein L³ and L⁴ are independently a bond, —NH—, —O—, —C(O)—, —C(O)O—, —OC(O)—, —OPO₂—O—, —O—P(O)(S)—O—, —O—P(O)(CH₃)—O—, —O—P(S)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, — P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene or substituted or unsubstituted heteroalkylene.

Embodiment 12. The compound of one of Embodiments 1 to 11, wherein L³ is independently

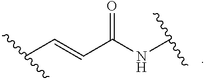 [SMILES: */C=C/C(=O)N*]

Embodiment 13. The compound of one of Embodiments 1 to 11, wherein L³ is independently —OPO₂—O— or —OP(O)(S)—O—.

Embodiment 14. The compound of one of Embodiments 1 to 11, wherein L³ is independently —O—.

Embodiment 15. The compound of any one of Embodiments 1 to 11, wherein L³ is independently —C(O)—.

Embodiment 16. The compound of any one of Embodiments 1 to 11, wherein L³ is independently —O—P(O)(N(CH₃)₂)—N—.

Embodiment 17. The compound of one of Embodiments 1 to 14, wherein L⁴ is independently substituted or unsubstituted alkylene or substituted or unsubstituted heteroalkylene.

Embodiment 18. The compound of one of Embodiments 1 to 17, wherein L⁴ is independently -L⁷-NH—C(O)— or -L⁷-C(O)—NH—, wherein L⁷ is substituted or unsubstituted alkylene.

Embodiment 19. The compound of one of Embodiments 1 to 18, wherein L⁴ is independently

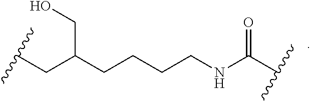 [SMILES: *CC(CO)CCCCNC(*)=O]

Embodiment 20. The compound of one of Embodiments 1 to 18, wherein L⁴ is independently

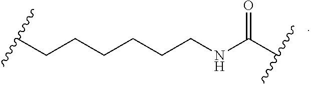 [SMILES: *CCCCCCNC(*)=O]

Embodiment 20. The compound of one of Embodiments 1 to 20, wherein -L³-L⁴- is independently —O-L⁷-NH—C(O)— or —O-L⁷-C(O)—NH—, wherein L⁷ is independently substituted or unsubstituted alkylene, substituted or unsubstituted heteroalkylene, or substituted or unsubstituted heteroalkenylene.

Embodiment 22. The compound of Embodiment 21, wherein -L³-L⁴- is independently —O-L⁷-NH—C(O)—, wherein L⁷ is independently substituted or unsubstituted C₅-C₈alkylene.

Embodiment 23. The compound of Embodiment 22, wherein -L³-L⁴- is independently

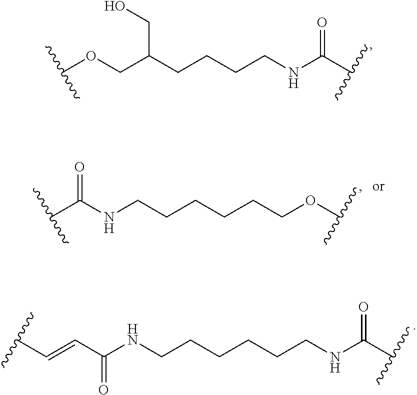 [SMILES: *OCC(CO)CCCCNC(*)=O ; *OCCCCCCNC(*)=O ; */C=C/C(=O)NCCCCCCNC(*)=O]

Embodiment 24. The compound of one of Embodiments 1 to 11, wherein -L³-L⁴- is independently —OPO₂—O-L⁷-NH—C(O)—, —OP(O)(S)—O-L⁷-NH—C(O)—, —OPO₂—O-L⁷-C(O)—NH— or —OP(O)(S)—O-L⁷-C(O)—NH—, wherein L⁷ is independently substituted or unsubstituted alkylene.

Embodiment 25. The compound of Embodiment 24, wherein -L³-L⁴- is independently —OPO₂—O-L⁷-NH—C(O)— or —OP(O)(S)—O-L⁷-NH—C(O)—, wherein L⁷ is independently substituted or unsubstituted C₅-C₈alkylene.

Embodiment 26. The compound of Embodiment 25, wherein -L³-L⁴- is independently

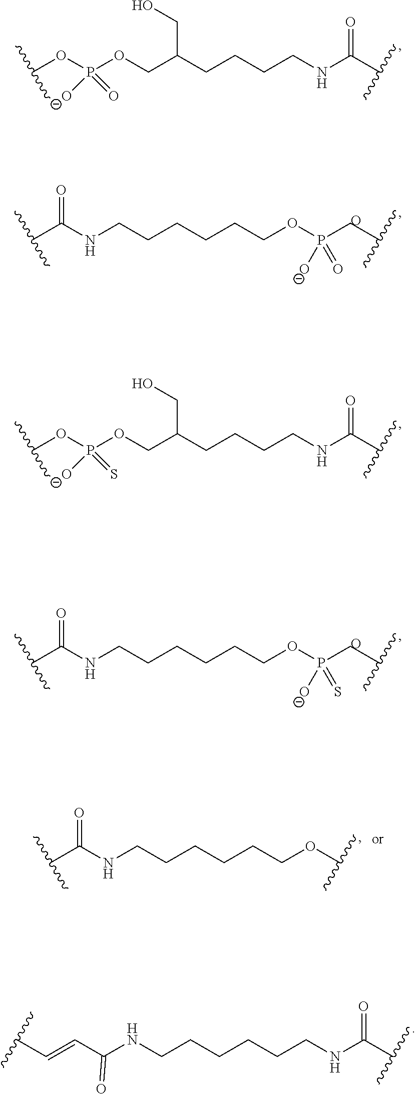

Embodiment 27. The compound of Embodiment 27, wherein an -L³-L⁴- is independently

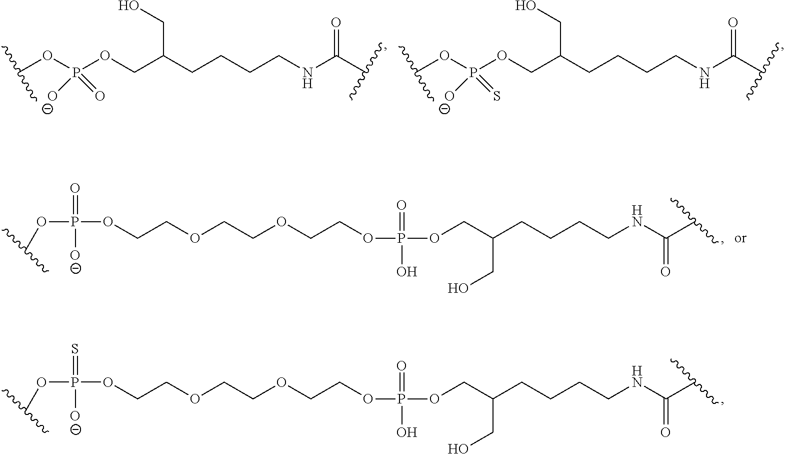 [SMILES: *OP(=O)([O-])OCC(CO)CCCCNC(*)=O ; *OP([O-])(=S)OCC(CO)CCCCNC(*)=O ; *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]

and is attached to a 3′ carbon of oligonucleotide.

Embodiment 28. The compound of Embodiment 26, wherein an -L³-L⁴- is independently

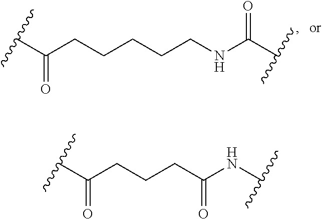 [SMILES: *C(=O)CCCCCNC(*)=O ; *NC(=O)CCCC(*)=O]

and is attached to a 3′ nitrogen of the oligonucleotide.

Embodiment 29. The compound of Embodiment 26, wherein an -L³-L⁴- is independently

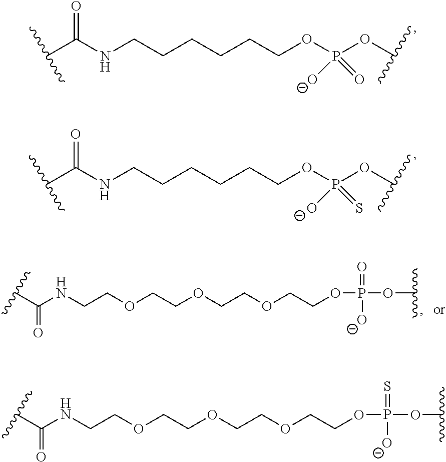 [SMILES: *OP(=O)([O-])OCCCCCCNC(*)=O ; *OP([O-])(=S)OCCCCCCNC(*)=O ; *OP(=O)([O-])OCCOCCOCCOCCNC(*)=O ; *OP([O-])(=S)OCCOCCOCCOCCNC(*)=O]

and is attached to a 5′ carbon of the oligonucleotide.

Embodiment 30. The compound of Embodiment 26, wherein an -L³-L⁴- is independently

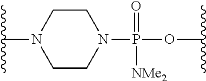 [SMILES: *OP(=O)(N(C)C)N1CCN(*)CC1]

and is attached to a 6′ carbon of the oligonucleotide.

Embodiment 31. The compound of Embodiment 26, wherein an -L³-L⁴- is independently

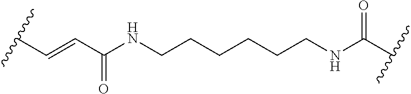 [SMILES: */C=C/C(=O)NCCCCCCNC(*)=O]

and is attached to a nucleobase of the oligonucleotide.

Embodiment 32. The compound of one of Embodiments 1 to 31, wherein R³ is independently hydrogen.

Embodiment 33. The compound of one of Embodiments 1 to 32, wherein L⁶ is independently —NHC(O)—, —C(O)NH—, substituted or unsubstituted alkylene, or substituted or unsubstituted heteroalkylene.

Embodiment 34. The compound of Embodiment 33, wherein L⁶ is independently —NHC(O)—.

Embodiment 35. The compound of Embodiment 33, wherein

L^{6A} is independently a bond or unsubstituted alkylene;

L^{6B} is independently a bond, —NHC(O)—, or unsubstituted arylene;

L^{6C} is independently a bond, unsubstituted alkylene, or unsubstituted arylene;

L^{6D} is independently a bond or unsubstituted alkylene; and

L^{6E} is independently a bond or —NHC(O)—.

Embodiment 36. The compound of Embodiment 33, wherein

L^{6A} is independently a bond or unsubstituted C₁-C₈ alkylene;

L^{6B} is independently a bond, —NHC(O)—, or unsubstituted phenylene;

L^{6C} is independently a bond, unsubstituted C₂-C₈ alkynylene, or unsubstituted phenylene;

L^{6D} is independently a bond or unsubstituted C₁-C₈ alkylene; and

L^{6E} is independently a bond or —NHC(O)—.

Embodiment 37. The compound of one of Embodiments 1 to 32, wherein L⁶ is independently a bond,

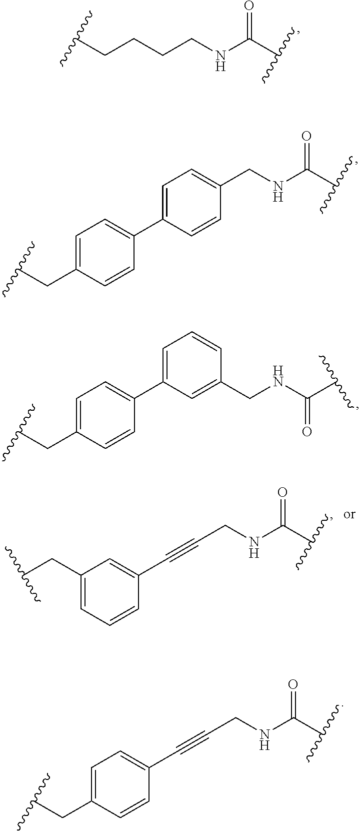 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

Embodiment 38. The compound of one of Embodiments 1 to 37, wherein L⁵ is independently —NHC(O)—, —C(O)NH—, substituted or unsubstituted alkylene, or substituted or unsubstituted heteroalkylene

Embodiment 39. The compound of one of Embodiments 1 to 37, wherein L⁵ is independently —NHC(O)—.

Embodiment 40. The compound of one of Embodiments 1 to 37, wherein

L^{5A} is independently a bond or unsubstituted alkylene;

L^{5B} is independently a bond, —NHC(O)—, or unsubstituted arylene;

L^{5C} is independently a bond, unsubstituted alkylene, or unsubstituted arylene;

L^{5D} is independently a bond or unsubstituted alkylene; and

L^{5E} is independently a bond or —NHC(O)—.

Embodiment 41. The compound of one of Embodiments 1 to 37, wherein

L^{5A} is independently a bond or unsubstituted C₁-C₈ alkylene;

L^{5B} is independently a bond, —NHC(O)—, or unsubstituted phenylene;

L^{5C} is independently a bond, unsubstituted C₂-C₈ alkynylene, or unsubstituted phenylene;

L^{5D} is independently a bond or unsubstituted C₁-C₈ alkylene; and

L^{5E} is independently a bond or —NHC(O)—.

Embodiment 42. The compound of one of Embodiments 1 to 37, wherein L⁵ is independently a bond,

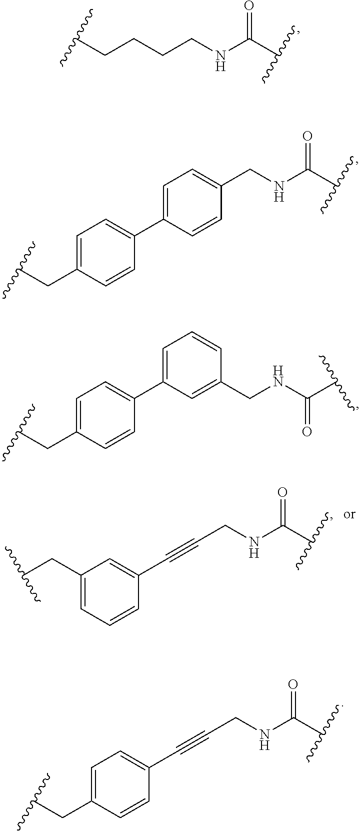 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

Embodiment 43. The compound of one of Embodiments 1 to 37, wherein R¹ is unsubstituted C₁-C₁₇ alkyl.

Embodiment 44. The compound of one of Embodiments 1 to 37, wherein R¹ is unsubstituted C₁₁-C₁₇ alkyl.

Embodiment 45. The compound of one of Embodiments 1 to 37, wherein R¹ is unsubstituted C₁₃-C₁₇ alkyl.

Embodiment 46. The compound of one of Embodiments 1 to 37, wherein R¹ is unsubstituted C₁₄-C₁₅ alkyl.

Embodiment 47. The compound of one of Embodiments 1 to 37, wherein R¹ is unsubstituted unbranched C₁-C₁₇ alkyl.

Embodiment 48. The compound of one of Embodiments 1 to 37, wherein R¹ is unsubstituted unbranched C₁₁-C₁₇ alkyl.

Embodiment 49. The compound of one of Embodiments 1 to 37, wherein R¹ is unsubstituted unbranched C₁₃-C₁₇ alkyl.

Embodiment 50. The compound of one of Embodiments 1 to 37, wherein R¹ is unsubstituted unbranched C₁₄-C₁₅ alkyl.

Embodiment 51. The compound of one of Embodiments 1 to 37, wherein R¹ is unsubstituted unbranched saturated C₁-C₁₇ alkyl.

Embodiment 52. The compound of one of Embodiments 1 to 37, wherein R¹ is unsubstituted unbranched saturated C₁₁-C₁₇ alkyl.

Embodiment 53. The compound of one of Embodiments 1 to 37, wherein R¹ is unsubstituted unbranched saturated C₁₃-C₁₇ alkyl.

Embodiment 54. The compound of one of Embodiments 1 to 37, wherein R¹ is unsubstituted unbranched saturated C₁₄-C₁₅ alkyl.

Embodiment 55. The compound of one of Embodiments 1 to 54, wherein R² is unsubstituted C₁-C₁₇ alkyl.

Embodiment 56. The compound of one of Embodiments 1 to 54, wherein R² is unsubstituted C₁₁-C₁₇ alkyl.

Embodiment 57. The compound of one of Embodiments 1 to 54, wherein R² is unsubstituted C₁₃-C₁₇ alkyl.

Embodiment 58. The compound of one of Embodiments 1 to 54, wherein R² is unsubstituted C₁₄-C₁₅ alkyl.

Embodiment 59. The compound of one of Embodiments 1 to 54, wherein R² is unsubstituted unbranched C₁-C₁₇ alkyl.

Embodiment 60. The compound of one of Embodiments 1 to 54, wherein R² is unsubstituted unbranched C₁₁-C₁₇ alkyl.

Embodiment 61. The compound of one of Embodiments 1 to 54, wherein R² is unsubstituted unbranched C₁₃-C₁₇ alkyl.

Embodiment 62. The compound of one of Embodiments 1 to 54, wherein R² is unsubstituted unbranched C₁₄-C₁₅ alkyl.

Embodiment 63. The compound of one of Embodiments 1 to 54, wherein R² is unsubstituted unbranched saturated C₁-C₁₇ alkyl.

Embodiment 64. The compound of one of Embodiments 1 to 54, wherein R² is unsubstituted unbranched saturated C₁₁-C₁₇ alkyl.

Embodiment 65. The compound of one of Embodiments 1 to 54, wherein R² is unsubstituted unbranched saturated C₁₃-C₁₇ alkyl.

Embodiment 66. The compound of one of Embodiments 1 to 54, wherein R² is unsubstituted unbranched saturated C₁₄-Cis alkyl.

Embodiment 67. The compound of one of Embodiments 1 to 54, wherein the single-stranded oligonucleotide induces skipping of an exon of the dystrophin pre-mRNA.

Embodiment 68. The compound of Embodiment 67, wherein the nucleobase sequence of the single-stranded oligonucleotide is complementary to a splice donor site, a splice acceptor site, an exonic splicing enhancer (ESE), a splicing branch point, an exon recognition sequence, or a splice enhancer of the dystrophin pre-mRNA.

Embodiment 69. The compound of Embodiment 67, wherein the nucleobase sequence of the single-stranded oligonucleotide is complementary to an annealing site selected from Table 2.

Embodiment 70. The compound of one of Embodiments 1 to 69, wherein the single-stranded oligonucleotide is 25 to 30 nucleotides in length.

Embodiment 71. The compound of one of Embodiments 1 to 69, wherein the nucleobase sequence of the single-stranded oligonucleotide is selected from a nucleobase sequence in Table 2.

Embodiment 72. The compound of Embodiment 71, wherein the nucleobase sequence of the single-stranded oligonucleotide is 5′-CUCCAACAUCAAGGAAGAUGGCAUUUCUAG-3′ (SEQ ID NO: 1).

Embodiment 73. The compound of Embodiment 71, wherein the nucleobase sequence of the single-stranded oligonucleotide is 5′-GUUGCCUCCGGUUCUGAAGGUGUUC-3′ (SEQ ID NO: 2).

Embodiment 74. The compound of Embodiment 71, wherein the nucleobase sequence of the single-stranded oligonucleotide is 5′-CAATGCCATCCTGGAGTTCCTG-3′ (SEQ ID NO: 3).

Embodiment 75. The compound of Embodiment 71, wherein the nucleobase sequence of the single-stranded oligonucleotide is 5′-UCAAGGAAGAUGGCAUUUCU-3′ (SEQ ID NO: 4).

Embodiment 76. The compound of Embodiment 71, wherein the nucleobase sequence of the single-stranded oligonucleotide is 5′-CGCTGCCCAATGCCAUCC-3′ (SEQ ID NO: 5).

Embodiment 77. The compound of Embodiment 71, wherein the nucleobase sequence of the single-stranded oligonucleotide is 5′-CCTCCGGTTCTGAAGGTGTTC-3′ (SEQ ID NO: 6).

Embodiment 78. The compound of any one of Embodiments 1 to 77, wherein the single-stranded oligonucleotide comprises one or more modified sugar moieties.

Embodiment 79. The compound of one of Embodiments 1 to 77, wherein each nucleotide of the single-stranded oligonucleotide comprises a modified sugar moiety.

Embodiment 80. The compound of Embodiment 79, wherein the modified sugar moiety comprises a 2′ modification.

Embodiment 81. The compound of Embodiment 79 or 80, wherein the 2′-modification is selected from a 2′-fluoro modification, a 2′-O-methyl modification, a 2′-O-methoxyethyl modification, and a bicyclic sugar modification.

Embodiment 82. The compound of Embodiment 81, wherein the bicyclic sugar modification is selected from a 4′-CH(CH₃)—O-2′ linkage, a 4′-(CH₂)₂—O-2′ linkage, a 4′-CH(CH₃)—O-2′ linkage, a 4′-CH(CH₂—OMe)-O-2′ linkage, a 4′-CH(CH₂)—N(H)—O-2′ linkage, or a 5′-CH(CH₂)—N(CH₃)—O-2′.

Embodiment 83. The compound of Embodiment 78, wherein the modified sugar moiety is an unlocked sugar modification.

Embodiment 84. The compound of Embodiment 78, wherein the modified sugar moiety is a morpholino moiety.

Embodiment 85. The compound of one of Embodiments 1 to 84, wherein the single-stranded oligonucleotide comprises one or more modified internucleotide linkages.

Embodiment 86. The compound of Embodiment 85, wherein the modified internucleotide linkage selected from a phosphorothioate linkage and a phosphorodiamidite linkage.

Embodiment 87. The compound of any one of Embodiments 1 to 86, wherein each internucleotide linkage of the single-stranded oligonucleotide is a chirally controlled internucleotide linkage.

Embodiment 88. The compound of Embodiment 87, wherein the single-stranded oligonucleotide comprises a plurality of internucleotide linkages of the Sp conformation.

Embodiment 89. The compound of Embodiment 87 or 88, wherein the single-stranded oligonucleotide comprises a plurality of internucleotide linkages of the Rp conformation.

Embodiment 90. The compound of one of Embodiments 72 to 74, wherein each nucleotide of the single-stranded nucleotide comprises a morpholino moiety, and wherein each morpholino moiety is linked by a phosphorodiamidite linkage.

Embodiment 91. The compound of Embodiment 72, wherein each nucleotide of the single-stranded nucleotide comprises a 2′-O-methoxyethyl sugar modification, and wherein each internucleotide linkage is a phosphorothioate linkage.

Embodiment 92. The compound of Embodiment 72, wherein each nucleotide of the single-stranded oligonucleotide comprises a 2′-O-methyl sugar modification, and wherein each internucleotide linkage is a phosphorothioate linkage.

Embodiment 93 The compound of Embodiment 73, wherein each nucleotide of the single-stranded nucleotide comprises a 2′-O-methoxyethyl sugar modification, and wherein each internucleotide linkage is a phosphorothioate linkage.

Embodiment 94. The compound of Embodiment 73, wherein each nucleotide of the single-stranded oligonucleotide comprises a 2′-O-methyl sugar modification, and wherein each internucleotide linkage is a phosphorothioate linkage.

Embodiment 95. The compound of Embodiment 74, wherein each nucleotide of the single-stranded nucleotide comprises a 2′-O-methoxyethyl sugar modification, and wherein each internucleotide linkage is a phosphorothioate linkage.

Embodiment 96. The compound of Embodiment 74, wherein each nucleotide of the single-stranded oligonucleotide comprises a 2′-O-methyl sugar modification, and wherein each internucleotide linkage is a phosphorothioate linkage.

Embodiment 97. The compound of Embodiment 75, wherein each nucleotide of the single-stranded oligonucleotide comprises a 2′-O-methyl sugar modification, and wherein each internucleotide linkage is a phosphorothioate linkage.

Embodiment 98. The compound of Embodiment 75, wherein each nucleotide of the single-stranded nucleotide comprises a 2′-O-methoxyethyl sugar modification, and wherein each internucleotide linkage is a phosphorothioate linkage.

Embodiment 99. The compound of Embodiment 75, wherein each nucleotide of the single-stranded oligonucleotide comprises a morpholino moiety, and wherein each morpholino moiety is linked by a phosphorodiatmite linkage.

Embodiment 100. The compound of Embodiment 75, wherein each nucleotide of the single-stranded oligonucleotide comprises a 2′-modification, wherein the 2′ modification is a 2′-O-methyl modification or a 2′-fluoro modification, and wherein each internucleotide linkage is selected from a phosphorothoiate linkage of the Sp conformation and a phosphodiester linkage.

Embodiment 101. The compound of Embodiment 76, wherein each nucleotide of the single-stranded oligonucleotide comprises a 2′ modification, wherein the 2′ modification is a 2′-O-methyl modification or a bicyclic sugar modification with a 4′-(CH₂)₂—O-2′ linkage, and wherein each linkage is a phosphodiester linkage.

Embodiment 102. The compound of Embodiment 77, wherein each nucleotide comprises a morpholino moiety, and wherein each morpholino moiety is linked by a phosphorodiamidite linkage.

Embodiment 103. The compound of one of Embodiments 1 to 67, wherein the single-stranded oligonucleotide is a structure selected from a structure in Table 4.

Embodiment 104. A method comprising contacting a cell with a compound of one of Embodiments 1 to 103.

Embodiment 105. The method of Embodiment 104, wherein the contacting occurs in vitro.

Embodiment 106. The method of Embodiment 104, wherein the contacting occurs in vivo.

Embodiment 107. A method of inducing skipping of an exon of the dystrophin pre-mRNA in a cell, comprising contacting the cell with a compound of one of Embodiments 1 to 103.

Embodiment 108. The method of Embodiment 107, wherein the cell is in vitro.

Embodiment 109. The method of Embodiment 107, wherein the cell is in vivo.

Embodiment 110. A method comprising administering to a subject in need thereof the compound of one of Embodiments 1 to 103.

Embodiment 111. A method of treating Duchenne muscular dystrophy, comprising administering to a subject in need thereof the compound of one of Embodiments 1 to 103.

Embodiment 112. The method of Embodiment 110 or 111, wherein the subject is determined to have a mutation in the dystrophin gene that is amenable to exon skipping.

Embodiment 113. The method of one of Embodiments 107 to 112, wherein one or more exons of the pre-mRNA of dystrophin is skipped at an increased level relative to the absence of the compound.

Embodiment 114. A compound of any of Embodiments 1 to 103, for use in therapy.

Embodiment 115. A compound of any of Embodiments 1 to 103, for use in the preparation of a medicament.

Embodiment 116. A pharmaceutical composition comprising a pharmaceutically acceptable excipient and the compound of any of Embodiments 1 to 103.

### EXAMPLES

The following examples will further describe the present disclosure, and are used for the purposes of illustration only, and should not be considered as limiting.

The compounds disclosed herein may be synthesized by methods described below, or by modification of these methods. Ways of modifying the methodology include, among others, temperature, solvent, reagents, etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include, e.g., those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

### Syntheses of Uptake Motifs

### Synthesis of DTx-01-01

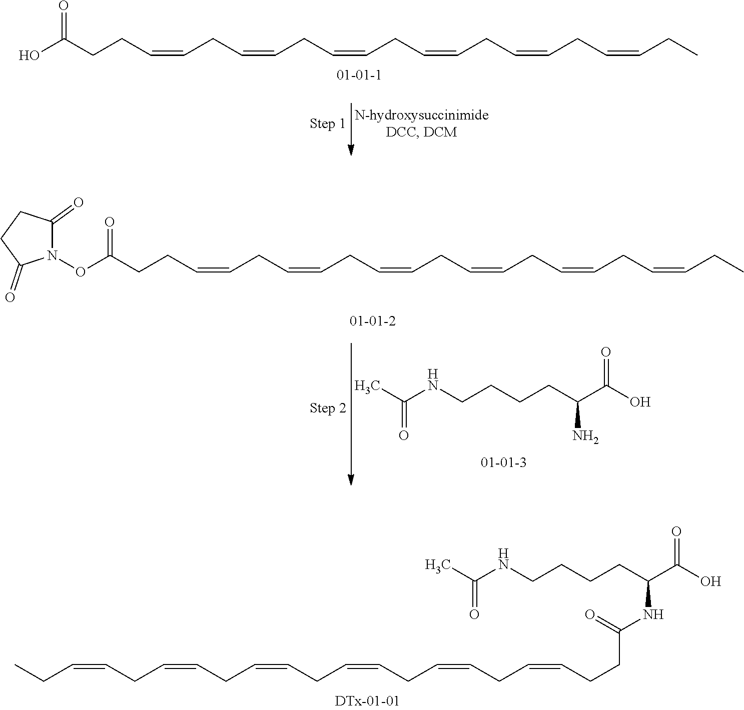

### Step 1: Synthesis of Intermediate 01-01-2

To a stirred solution of 01-01-1 (5.0 g, 0.015 mol) in DCM (500 mL) at RT was added DMAP (0.17 g, 0.0015 mol), DCC (4.86 g, 0.016 mol), followed by N-hydroxysuccinimide (1.92 g, 0.016 mol). The resulting mixture was stirred at RT. After 16 h, the reaction mixture was filtered through a sintered funnel. The filtrate was evaporated to yield crude 01-01-2 as a pale-yellow liquid (6.0 g, 92.50%), which was used in the next step without further purification.

### Step 2: Synthesis of Lipid Motif DTx-01-01

To a stirred solution of 01-01-3 (1.3 g, 0.006 mol) in DMF (20 m) at RT was added slowly Et₃N (3 mL, 0.020 mol) and then 01-01-2 (2.93 g, 0.007 mol). The resulting mixture was stirred at RT. After 16 h, the reaction mixture was quenched with ice water dropwise and then extracted with EtOAc. The combined organic extract was washed with ice water, brine, dried over Na₂SO₄, and then evaporated to yield crude DTx-01-01, which was purified by column chromatography (3% MeOH in DCM) to afford lipid motif DTx-01-01 as a viscous, brown liquid (1.3 g, 51%). LCMS m/z (M+H)⁺: 499.4; ¹H-NMR (400 MHz, DMSO-d6): δ 0.92 (t, J=7.6 Hz, 3H), 1.24-1.66 (m, 10H), 1.82 (s, 3H), 2.02-2.33 (m, 7H), 2.73-2.98 (m, 9H), 3.94 (br s, 1H), 5.27-5.34 (m, 10H), 7.70 (br s, 1H), 7.78 (br s, 1H).

### Synthesis of DTx-01-03

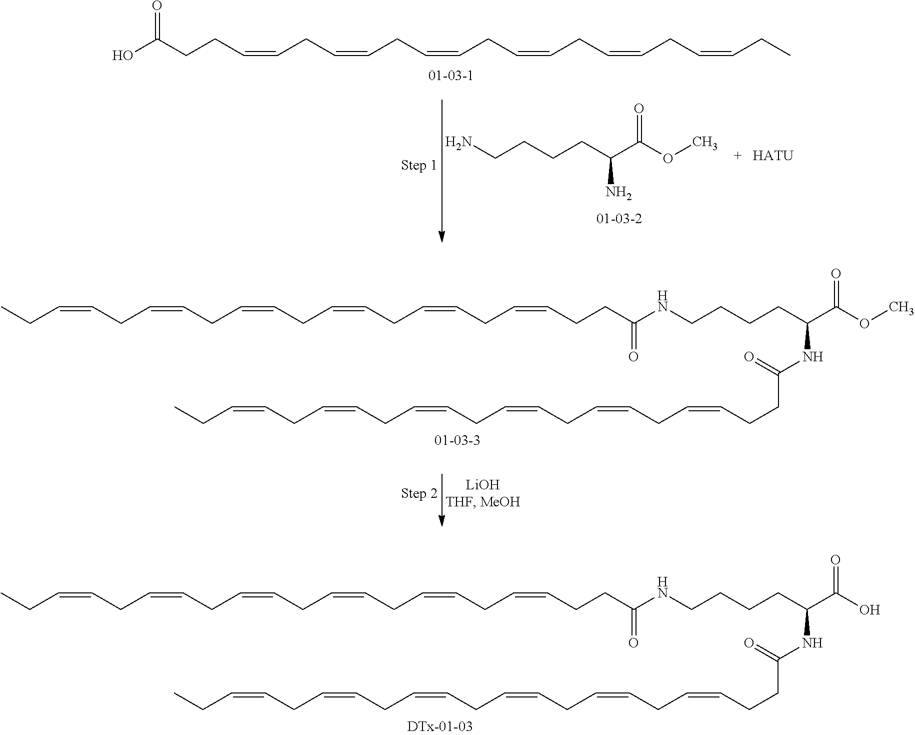

### Step 1: Synthesis of Intermediate 01-03-3

To a stirred solution of 01-03-1 (15 g, 0.045 mol) in DMF (300 mL) at RT was added slowly DIPEA (39.86 mL, 0.11 mol), HATU (17.1 g, 0.045 mol), and 01-03-2 (3.6 g, 0.022 mol). The resulting mixture was stirred at RT. After 16 h, the reaction mixture was quenched with ice water dropwise and extracted with DCM. The combined organic extract was washed with ice water, brine, dried over Na₂SO₄, and then evaporated to yield crude 01-03-3, which was purified by column chromatography (20% EtOAc in petroleum ether) to afford 01-03-3 as a viscous, pale brown liquid (11.2 g, 63.7%).

### Step 2: Synthesis of Lipid Motif DTx-01-03

To a stirred solution of 01-03-3 (10 g, 0.012 mol) in MeOH (100 mL) at 0° C. was added slowly LiOH (1.07 g, 0.025 mol) in water (50 mL). The resulting mixture was stirred at RT. After 4h, ice water was added dropwise to the reaction mixture. The mixture was acidified with 1.5 M HCl and then extracted with DCM. The combined organic extract was washed with ice water, brine, dried over Na₂SO₄, and then evaporated to yield crude DTx-01-03, which was purified by column chromatography (3% MeOH in DCM) to afford lipid motif DTx-01-03 as a viscous, pale brown liquid (7.5 g, 77%). LCMS m/z (M+H)⁺: 767.5; ¹H-NMR (400 MHz, DMSO-d6): δ 0.954 (t, J=3.6 Hz, 6H), 1.23-1.66 (m, 8H), 1.99-2.33 (m, 12H), 2.69-2.82 (m, 22H), 4.13 (t, J=3.6 Hz, 1H), 5.25-5.36 (m, 22H), 7.76 (t, J=5.2 Hz, 1H), 8.03 (d, J=7.6 Hz, 1H), 12.5 (br s, 1H).

### Synthesis of Lipid Motif DTx-01-06

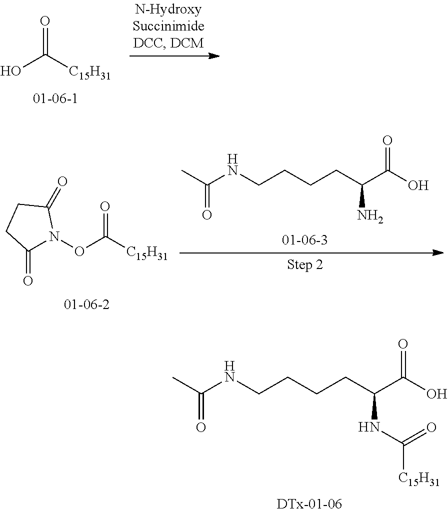

### Step 1: Synthesis of Intermediate 01-06-2

To a stirred solution of linear fatty acid 01-06-1 (5.0 g, 0.018 mol) in DCM (100 mL) at RT was added DMAP (0.208 g, 0.0018 mol), DCC (5.22 g, 0.018 mol), and then N-hydroxysuccinimide (2.07 g, 0.018 mol). The resulting mixture was stirred at RT. After 16 h, the reaction mixture was filtered through a sintered funnel. The filtrate was evaporated to yield crude 01-06-2 as an off-white solid (6.0 g, 88%), which was used in the next step without further purification.

### Step 2: Synthesis of Lipid Motif DTx-01-06

To a stirred solution of 01-06-3 (1.02 g, 0.054 mol) in DMF (40 mL) at RT was added slowly Et₃N (2.3 mL, 0.016 mol) and 01-06-2 (2 g, 0.047 mol). The resulting mixture was stirred at RT. After 16 h, the reaction mixture was quenched with ice water dropwise and then extracted with EtOAc. The combined organic extract was washed with chilled water, brine, dried over Na₂SO₄, and then evaporated to yield crude DTx-01-06, which was purified by column chromatography (3% MeOH in DCM) to afford lipid motif DTx-01-06 as an off-white solid (2.0 g, 88%). MS (ESI) m/z (M+H)⁺: 427.4; ¹H-NMR (400 MHz, DMSO-d6): δ 0.97 (t, J=7.2 Hz, 3H), 1.36-1.77 (m, 31H), 1.83 (s, 3H), 2.09 (t, J=6.4 Hz, 2H), 2.98 (d, J=6.0 Hz, 2H), 5.57 (d, J=8.0 Hz, 2H), 7.79 (br s, 1H), 7.97 (d, J=7.6 Hz, 1H).

### Synthesis of Lipid Motif DTx-01-08

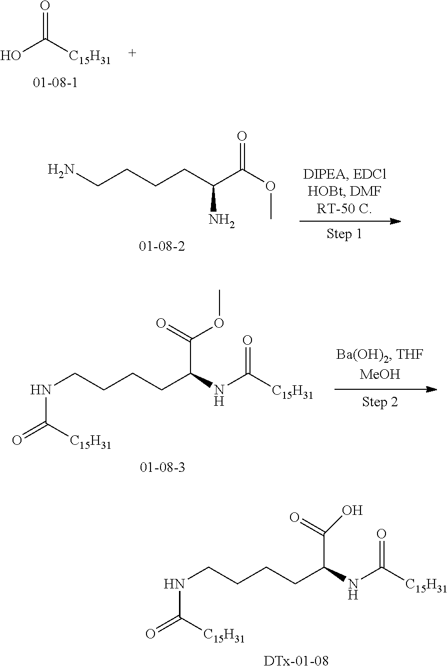

### Step 1: Synthesis of Compound 01-08-3

To a stirred solution of linear fatty acid 01-08-1 (25.58 g, 0.099 mol) in DMF (500 mL) at RT was added DIPEA (42.66 mL, 0.245 mol) and compound 01-08-2 (8.0 g, 0.049 mol), followed by EDCl (18.97 g, 0.099 mol) and HOBt (13.37 g, 0.099 mol). The resulting mixture was stirred at 50° C. After 16 h, the reaction mixture was quenched with ice water and extracted with DCM. The combined organic extract was washed with water, brine, dried over Na₂SO₄, and then evaporated to give crude 01-08-3, which was recrystallized (20% MTBE in petroleum ether) to afford 01-08-3 as an off-white solid (18 g, 56%).

### Step 2: Synthesis of Lipid Motif DTx-01-08

To a stirred solution of 01-08-3 (10 g, 0.0156 mol) in MeOH and THF (1:1; 200 mL) at RT was added slowly Ba(OH)₂ (9.92 g, 0.031 mol, dissolved in MeOH). The resulting mixture was stirred at RT. After 6 h, the reaction mixture was quenched with ice water dropwise, and then acidified with 1.5 M HCl. The mixture was filtered, and the precipitate was recrystallized (MTBE in petroleum ether) to afford lipid motif DTx-01-08 as an off-white solid (7.2 g, 74.2%). MS (ESI) m/z (M+H)⁺: 623.6; ¹H-NMR (400 MHz, CDCl₃): δ 0.868 (m, 6H), 1.25-1.69 (m, 58H), 2.03 (t, J=7.2 Hz, 2H), 2.11 (t, J=7.6 Hz, 2H), 2.99 (q, J=8.4 Hz, 2H), 4.15-4.20 (m, 1H), 7.42 (br s, 1H), 7.65 (d, J=7.6 Hz, 1H), 12.09 (br s, 1H).

### Synthesis of Lipid Motif DTx-01-11

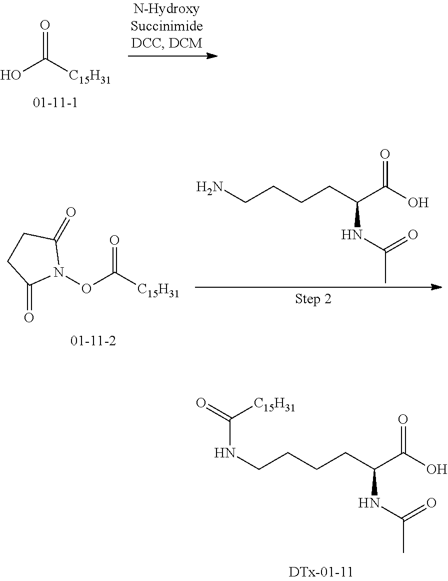

### Step 1: Synthesis of Intermediate 01-11-2

To a stirred solution of linear fatty acid 01-11-1 (5.0 g, 0.018 mol) in DCM (100 mL) at RT was added DMAP (0.208 g, 0.0018 mol) and DCC (5.22 g, 0.018 mol), followed by N-hydroxysuccinimide (2.07 g, 0.018 mol). The resulting mixture was stirred at RT. After 16 h, the reaction mixture was filtered through a sintered funnel. Evaporation of the filtrate yielded crude 01-11-2 as an off-white solid (6.0 g, 88%), which was used directly in the next step without further purification.

### Step 2: Synthesis of Lipid Motif DTx-01-11

To a stirred solution of 01-11-3 (2.05 g, 0.01 mol) in DMF (80 mL) at RT was added slowly Et₃N (4.6 mL, 0.032 mol) and 01-11-2 (4.0 g, 0.01 mol). The resulting mixture was stirred at RT. After 16 h, the reaction mixture was quenched with ice water dropwise and then extracted with EtOAc. The combined organic extract was washed with ice water, brine, dried over Na₂SO₄, and then evaporated to yield crude DTx-01-11, which was purified by column chromatography (3% MeOH in DCM) to afford lipid motif DTx-01-11 as an off-white solid (3.1 g, 66.5%). MS (ESI) m/z (M+H)⁺: 427.4; ¹H-NMR (400 MHz, DMSO-d6): δ 0.85 (t, J=6.8 Hz, 3H), 1.23-1.73 (m, 31H), 1.83 (s, 3H), 2.02 (t, J=7.2 Hz, 2H), 3.00 (q, J=6.0 Hz, 2H), 4.10 (dd, J=8.4, 4.4 Hz, 2H), 7.74 (d, J=5.2 Hz, 1H), 8.07 (br s, 1H), 12.45 (br s, 1H).

### Synthesis of Lipid Motif DTx-01-13

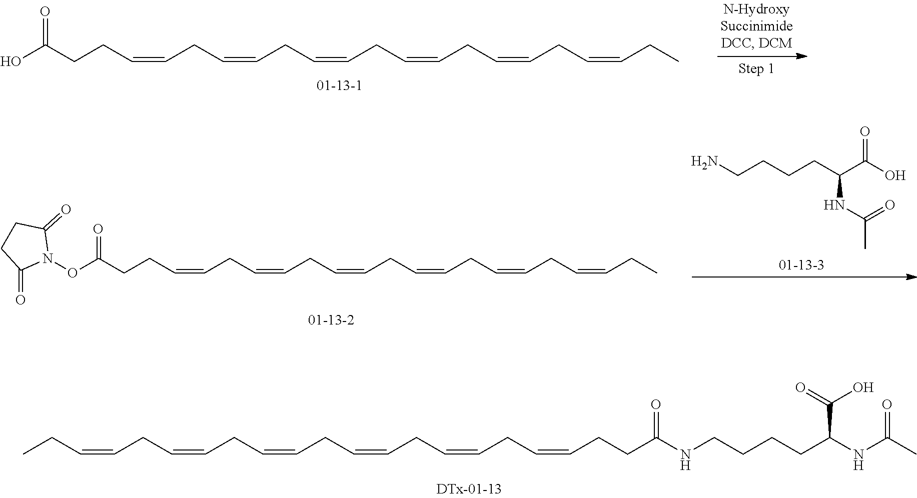

### Step 1: Synthesis of Intermediate 01-13-2

To a stirred solution of 01-13-1 (5.0 g, 0.015 mol) in DCM (500 mL) at RT was added DMAP (0.17 g, 0.0015 mol) and DCC (4.86 g, 0.016 mol), followed by N-hydroxysuccinimide (1.92 g, 0.016 mol). The resulting mixture was stirred at RT. After 16 h, the reaction mixture was filtered through a sintered funnel, and the filtrate was evaporated to yield crude 01-13-2 as a pale yellow liquid (6.0 g, 92.5%). The crude intermediate was used directly in the next step without further purification.

### Step 2: Synthesis of Lipid Motif DTx-01-13

To a stirred solution of 01-13-3 (1.3 g, 0.006 mol) in DMF (20 mL) at RT was added slowly Et₃N (3 mL, 0.020 mol) and 01-13-2 (2.93 g, 0.007 mol). The resulting mixture was stirred at RT. After 16 h, the reaction mixture was quenched with ice water dropwise and extracted with EtOAc. The combined organic extract was washed with ice water, brine, dried over Na₂SO₄, and then evaporated to yield crude DTx-01-13, which was purified by column chromatography (3% MeOH in DCM) to afford lipid motif DTx-01-13 as a viscous, brown liquid (2.1 g, 61%). LCMS m/z (M+H)⁺: 499.4; ¹H-NMR (400 MHz, DMSO-d6): δ 0.90 (t, J=7.2 Hz, 3H), 1.22-1.67 (m, 7H), 1.75 (s, 3H), 1.98-2.27 (m, 7H), 2.73-2.95 (m, 9H), 2.96 (dd, J=12.4, 6.4 Hz, 2H), 4.06-4.09 (m, 1H), 5.23-5.37 (m, 10H), 7.79 (br s, 1H), 7.91 (t, J=7.6 Hz, 1H).

### Synthesis of Lipid Motif DTx-01-30

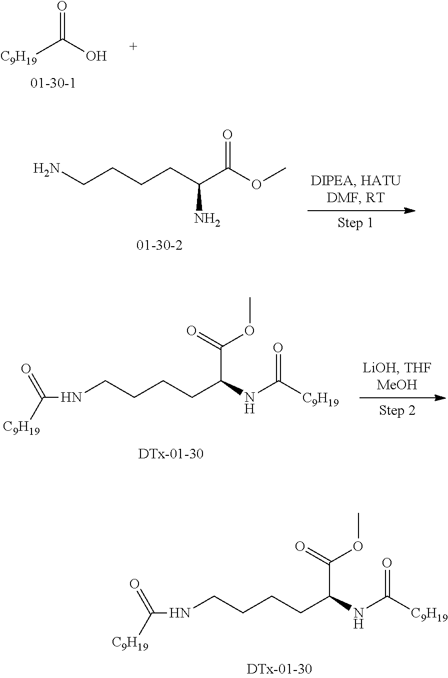

### Step 1: Synthesis of Intermediate 01-30-3

To a stirred solution of 01-30-2 (3 g, 0.01 mol) in DMF (50 mL) at RT was added slowly DIPEA (13.8 mL, 0.077 mol), linear fatty acid 01-30-1 (4.4 g, 0.0154 mol), and HATU (5.87 g, 0.0154 mol). The resulting mixture was stirred at RT. After 16 h, the reaction mixture was quenched with ice water. The precipitate was isolated by filtration, and then dried in vacuo to afford 01-30-3 as an off-white solid (3.2 g, 53.15%).

### Step 2: Synthesis of Lipid Motif DTx-01-30

To a stirred solution of 01-30-3 (3.2 g, 0.0068 mol) in MeOH (30 mL), THF (30 mL), and water (3 mL), was added LiOH.H₂O (0.86 g, 0.0251 mol). The resulting reaction mixture was stirred 16 h. Subsequently, the reaction mixture was concentrated under vacuum and then neutralized with 1.5 N HCl. The precipitate was isolated via filtration, washed with water, and dried under vacuum to yield crude DTx-01-30. Recrystallization (80% DCM in hexane) afforded lipid motif DTx-01-30 as an off-white solid (2.2 g, 73.3%). LCMS m/z (M+H)⁺: 455.5; ¹H-NMR (400 MHz, DMSO-d6): δ 0.88-0.92 (t, J=7.2 Hz, 6H), 1.17-1.55 (m, 33H), 1.64 (t, J=7.0 Hz, 1H), 2.00 (t, J=7.2 Hz, 2H), 2.06-2.10 (m, 2H), 2.97-2.99 (m, 2H), 4.11 (t, J=8.4 Hz, 1H), 7.71 (s, 1H), 7.96 (d, J=7.6 Hz, 1H), 12.47 (br s, 1H).

### Synthesis of Lipid Motif DTx-01-31

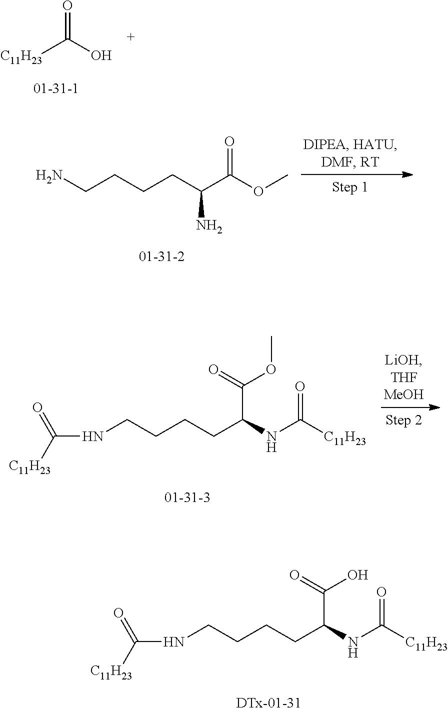

### Step 1: Synthesis of Intermediate 01-31-3

To a stirred solution of 01-31-2 (3 g, 0.0128 mol) in DMF (50 mL) at RT was added slowly DIPEA (13.8 mL, 0.077 mol), linear fatty acid 01-31-1 (3.1 g, 0.0154 mol), and HATU (5.87 g, 0.0154 mol). The resulting mixture was stirred at RT. After 16 h, the reaction mixture was quenched with ice water. Solids were isolated by filtration and dried in vacuo to afford 01-01-3 as an off-white solid (3.4 g 50.7%).

### Step 2: Synthesis of Lipid Motif DTx-01-31

To a stirred solution of 01-01-3 (3 g, 0.0057 mol) in MeOH (10 mL), THF (10 mL), and water (3 mL), was added LiOH.H₂O (0.8 g, 0.0019 mol). The reaction mixture was stirred 16 h. Subsequently, the reaction mixture was concentrated under vacuum and then neutralized with 1.5 N HCl. The precipitate was solid was isolated via filtration, washed with water, and dried under vacuum, yielding crude DTx-01-31. Recrystallization (80% DCM in hexane) afforded lipid motif DTx-01-31 as an off-white solid (2.3 g, 79.3%). LCMS m/z (M+H)⁺: 511.5; ¹H-NMR (400 MHz, DMSO-d6): δ 0.86-0.90 (t, J=7.2 Hz, 6H), 1.33-1.54 (m, 42H), 1.64 (t, J=7.9 Hz, 1H), 1.98-2.08 (m, 4H), 2.96 (t, J=6.3 Hz, 2H), 4.02-4.18 (m, 1H), 7.71-7.79 (m, 2H).

### Synthesis of Lipid Motif DTx-01-32

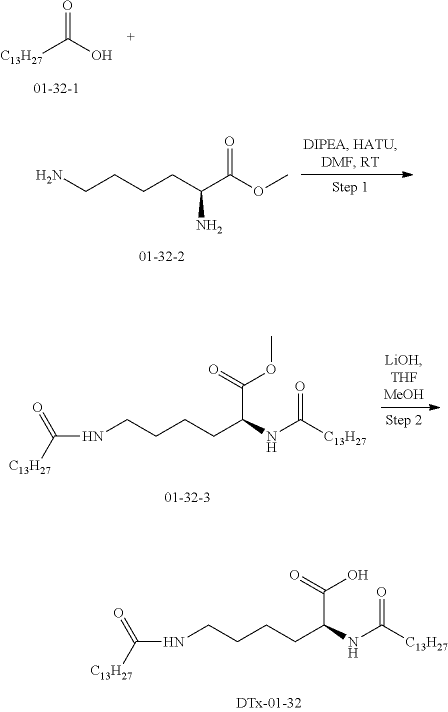

### Step 1: Synthesis of Intermediate 01-32-3

To a stirred solution of 01-32-2 (3 g, 0.01 mol) in DMF (50 mL) at RT was added slowly DIPEA (13.8 mL, 0.077 mol), linear fatty acid 01-32-1 (4.4 g, 0.0154 mol), and HATU (5.87 g, 0.0154 mol). The resulting mixture was stirred at 60° C. After 16 h, the reaction mixture was quenched with ice water, the solids isolated by filtration, and the solids dried under vacuum to afford 01-32-3 as an off-white solid (3.5 g, 53.2%).

### Step 2: Synthesis of Lipid Motif DTx-01-32

To a stirred solution of 01-32-3 (3.5 g, 0.0051 mol) in MeOH (10 mL), THF (10 mL), and water (3 mL), was added LiOH.H₂O (0.8 g, 0.0154). The reaction mixture was stirred 16 h. Subsequently, the reaction mixture was concentrated under vacuum and neutralized with 1.5 N HCl. The solids were isolated by filtration, washed with water, and dried under vacuum, affording crude DTx-01-32. Recrystallization (80% DCM in hexane) yielded lipid motif DTx-01-32 as an off-white solid (2.3 g, 79.3%). LCMS m/z (M+H)⁺: 567.2; ¹H-NMR (400 MHz, TFA-d): δ 0.87-0.98 (m, 6H), 1.20-1.58 (m, 41H), 1.74-1.92 (m, 8H), 2.18-2.21 (m, 2H), 2.73 (t, J=7.6 Hz, 2H), 3.05 (t, J=7.6 Hz, 2H), 3.60 (t, J=7.8 Hz, 2H).

### Synthesis of Lipid Motif DTx-01-33

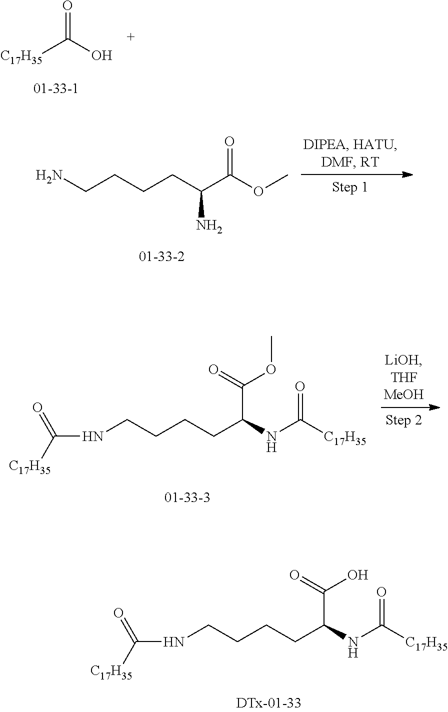

### Step 1: Synthesis of Intermediate 01-33-3

To a stirred solution of 01-33-2 (5 g, 0.0312 mol) in DMF (100 mL) at RT was added slowly DIPEA (32 mL, 0.1872 mol), linear fatty acid 01-33-1 (26.6 g, 0.0936 mol), and HATU (41.5 g, 0.1092 mol) slowly at RT. After 16 h, the reaction mixture was quenched with ice water. Crude 01-33-3 was isolated by filtration from the reaction mixture and dried in vacuo. Purification by trituration with THF afforded 01-33-3 as an off-white solid (8.5 g, 39.5%).

### Step 2: Synthesis of Lipid Motif DTx-01-33

To a stirred solution of 01-33-3 (5 g, 0.0072 mol) in MeOH (75 mL), THF (75 mL), and water (3 mL), was added LiOH.H₂O (0.60 g, 0.0144 mol). The reaction mixture was stirred 16 h. Subsequently, the reaction mixture was concentrated under vacuum and neutralized with 1.5 N HCl. The solids were filtered, washed with water, and dried under vacuum, affording crude DTx-01-33. Recrystallization (IPA) yielded lipid motif DTx-01-33 as an off-white solid (2.3 g, 47%). LCMS m/z (M+H)⁺: 680; ¹H-NMR (400 MHz, TFA-d): δ 1.10-1.18 (m, 6H), 1.62-1.80 (m, 57H), 2.06-2.20 (m, 8H), 2.49-2.50 (m, 2H), 2.96-3.01 (m, 2H), 3.32-3.35 (m, 2H), 3.87-3.98 (m, 2H).

### Synthesis of Lipid Motif DTx-01-34

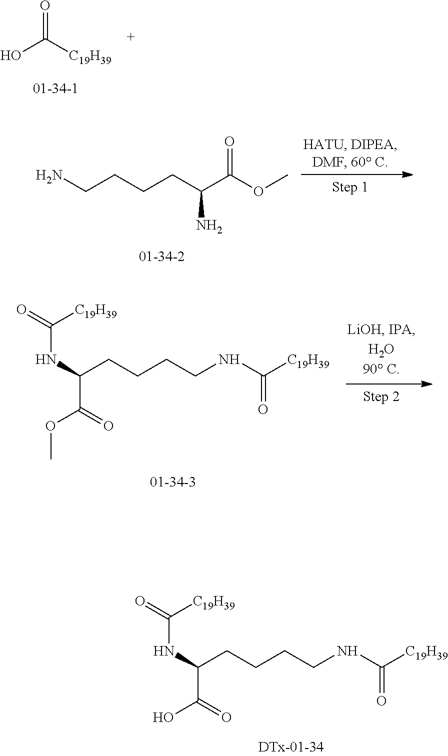

### Step 1: Synthesis of Intermediate 01-34-3

To a stirred solution of 01-34-2 (5 g, 0.0312 mol) in DMF (100 mL) at RT was added slowly DIPEA (32 mL, 0.1872 mol), linear fatty acid 01-34-1 (29.2 g, 0.0936 mol), and HATU (41.5 g, 0.1092 mol). The resulting mixture was stirred at 50° C. After 16 h, the reaction mixture was quenched with ice water, the solids isolated by filtration, and then the solids dried under vacuum. Purification of the solids by trituration with THF afforded 01-34-3 as an off-white solid (10 g, 43%).

### Step 2: Synthesis of Lipid Motif DTx-01-34

To a stirred solution of 01-34-3 (5 g, 0.0066 mol) in 9:1 IPA:water (150 mL) was added LiOH.H₂O (0.56 g, 0.0133 mol). The reaction mixture was stirred at 90° C. After 1 h, the reaction mixture was concentrated under vacuum and then neutralized with 1.5 N HCl. The precipitate was isolated via filtration, washed with water, and dried under vacuum. Recrystallization (IPA) of the precipitate afforded lipid motif DTx-01-34 as an off-white solid (3.2 g, 65%). LCMS m/z (M+H)+: 736.2; ¹H-NMR (400 MHz, TFA-d): δ 1.13-1.17 (m, 6H), 1.48-1.79 (m, 65H), 2.05-2.19 (m, 8H), 2.48-2.49 (m, 2H), 2.95-2.96 (m, 2H), 3.28-3.34 (m, 2H), 3.85-3.96 (m, 2H).

### Synthesis of Lipid Motif DTx-01-35

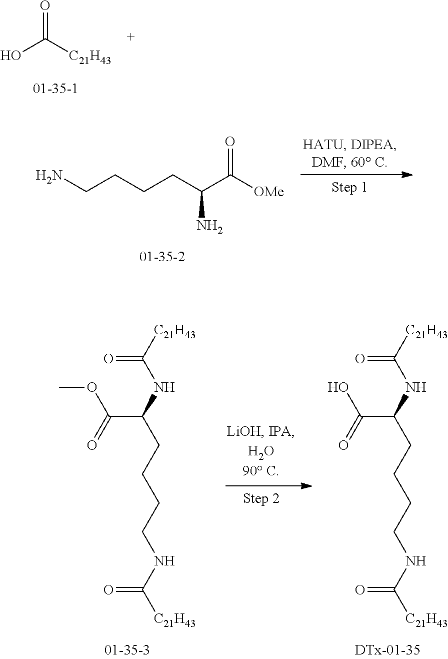

### Step 1: Synthesis of Intermediate 01-35-3

To a stirred solution of 01-35-2 (5 g, 0.0312 mol) in DMF (100 mL) at RT was added slowly DIPEA (32 mL, 0.1872 mol), linear fatty acid 01-35-1 (31.8 g, 0.0936 mol), and HATU (41.5 g, 0.1092 mol). The resulting mixture was stirred at 60° C. After 16 h, the reaction mixture was quenched with ice water, the solids isolated by filtration, and then the solids dried under vacuum. Purification of the solids by trituration with THF yielded 01-35-3 as an off-white solid (7 g, 28%).

### Step 2: Synthesis of Lipid Motif DTx-01-35

To a stirred solution of 01-35-3 (5 g, 0.0062 mol) in 9:1 IPA:water (150 mL) was added LiOH.H₂O (0.52 g, 0.0124 mol). The reaction mixture was stirred at 90° C. After 1 h, the reaction mixture was concentrated under vacuum and then neutralized with 1.5 N HCl. The solids were isolated by filtration, washed with water, and dried under vacuum, yielding crude DTx-01-35. Recrystallization in IPA afforded lipid motif DTx-01-35 as an off-white solid (3.1 g, 63%). LCMS m/z (M+H)⁺: 792.2; ¹H-NMR (400 MHz, TFA-d): δ 1.06-1.22 (m, 6H), 1.49-1.88 (m, 73H), 1.99-2.29 (m, 8H), 2.49-2.51 (m, 2H), 2.95-3.10 (m, 2H), 3.32-3.34 (m, 2H), 3.86-3.90 (m, 2H).

### Synthesis of Lipid Motif DTx-03-06

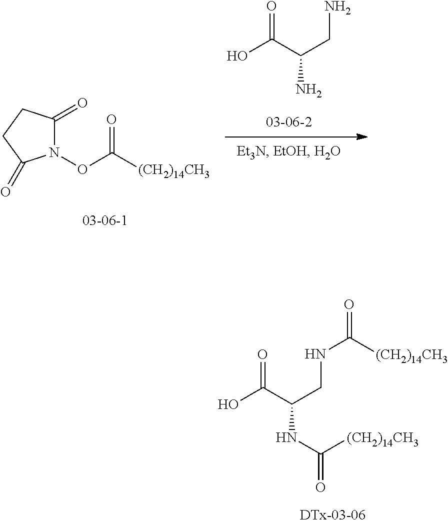

To a stirred solution of 03-06-2 (1.2 g, 0.0068 mol) in 65% aq. EtOH (40 mL) at RT was added slowly Et₃N (4.75 mL, 0.034 mol) and NHS-linear fatty acid 03-06-1 (6.0 g, 0.017 mol). The resulting mixture was stirred at 75° C. After 16 h, the reaction mixture was neutralized with 1.5 N HCl. The precipitate was isolated by filtration, washed with water, and dried. Purification of the precipitate by trituration with DCM afforded lipid motif DTx-03-06 as an off-white solid (2.3 g, 57%). LCMS m/z (M+H)⁺: 581.5; ¹H-NMR (400 MHz, TFA-d): δ 0.78-0.82 (m, 6H), 1.21-1.40 (m, 49H), 1.62-1.79 (m, 4H), 2.35-2.46 (m, 2H), 2.96-2.30 (m, 2H), 3.89-4.03 (m, 2H).

### Synthesis of Lipid Motif DTx-03-08

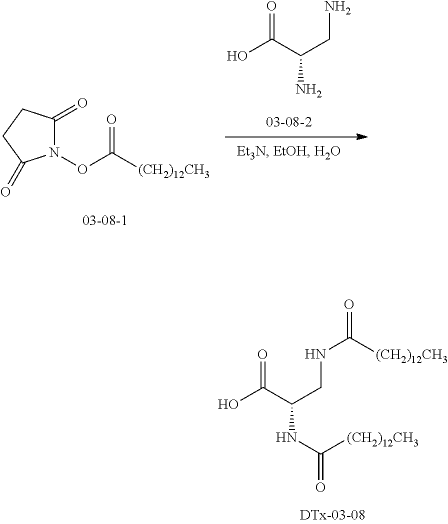

To a stirred solution of 03-08-2 (0.64 g, 6.2 mmol) in 65% aq. EtOH (40 mL) at RT was added slowly Et₃N (4.3 mL, 31 mmol) and NHS-linear fatty acid 03-08-1 (5.0 g, 15.4 mmol). The resulting mixture was stirred at 75° C. After 16 h, the reaction mixture was neutralized with 1.5 N HCl. The precipitate was isolated by filtration, washed with water, and dried. Purification of the precipitate by trituration with DCM afforded lipid motif DTx-03-08 as an off-white solid (2.1 g, 65%). LCMS m/z (M+H)⁺: 525.4; ¹H-NMR (400 MHz, TFA-d): δ 0.81-0.97 (m, 6H), 1.22-1.61 (m, 42H), 1.63-1.83 (m, 2H), 1.84-2.11 (m, 3H), 2.41-2.61 (m, 2H), 3.01-3.21 (m, 1H), 3.95-4.05 (m, 1H), 4.06-4.18 (m, 1H).

### Synthesis of Lipid Motif DTx-03-09

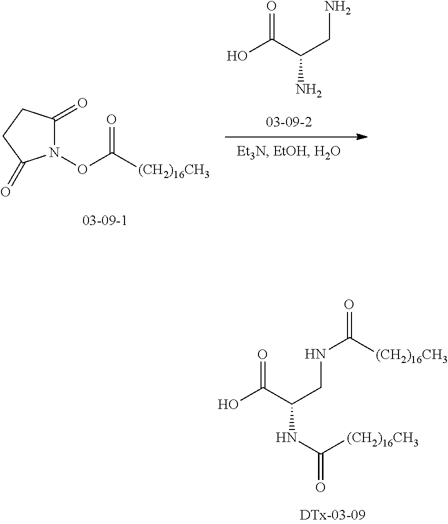

To a stirred solution of 03-09-2 (0.340 g, 3.8 mmol) in 65% aq. EtOH (40 mL) at RT was added slowly Et₃N (2.6 mL, 19 mmol) and NHS-linear fatty acid 03-09-1 (5.2 g, 8.16 mmol). The resulting mixture was stirred at 75° C. After 16 h, the reaction mixture was neutralized with 1.5 N HCl. The precipitate was isolated by filtration, washed with water, and dried. Purification of the precipitate by trituration with DCM afforded lipid motif DTx-03-09 as an off-white solid (2.3 g, 95%). ¹H-NMR (400 MHz, TFA-d): δ 0.85-0.99 (m, 6H), 1.21-1.52 (m, 56H), 1.69-1.91 (m, 4H), 2.49-2.71 (m, 4H), 4.05-4.31 (m, 2H), 4.76-5.06 (m, 1H).

### Synthesis of Lipid Motif DTx-06-06

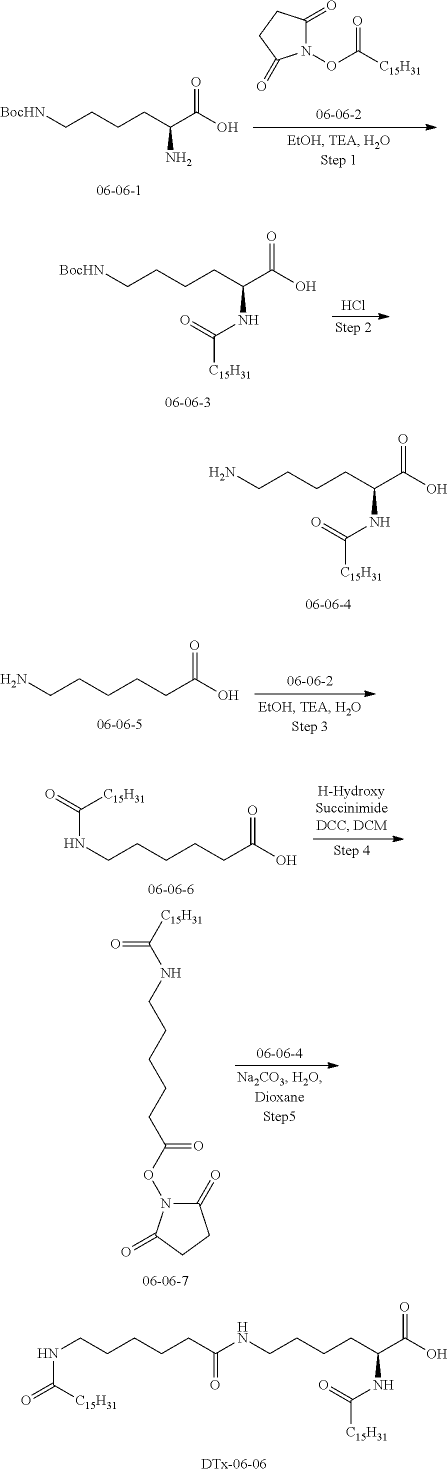

### Step 1: Synthesis of Intermediate 06-06-3

To a stirred solution of 06-06-1 (4.6 g, 0.0169 mol) in 65% aq. EtOH (60 mL) at RT was added slowly Et₃N (5.9 mL, 0.042 mol) and NHS-linear fatty acid 06-06-2 (6 g, 0.00186 mol). The resulting mixture was stirred at 75° C. After 16 h, the reaction mixture was neutralized with 1.5 N HCl. The precipitate was isolated by filtration, washed with water, and dried. Purification of the precipitate by column chromatography (3% MeOH in DCM) afforded 06-06-3 as an off-white solid (5.0 g, 62%).

### Step 2: Synthesis of Intermediate 06-06-4

To a stirred solution of 06-06-3 (7 g, 0.014 mol) in 1,4-dioxane (50 mL) at RT was added slowly 4 M HCl in 1,4-dioxane (50 mL). The resulting mixture was stirred at RT. After 16 h, the reaction mixture was concentrated under reduced pressure to yield crude 06-06-4, which was triturated with diethyl ether to afford 06-06-4 as an off-white solid (4.5 g, 81%).

### Step 3: Synthesis of Intermediate 06-06-6

To a stirred solution of 06-06-5 (5 g, 0.038 mol) in 65% aq. EtOH (40 mL) at RT was added slowly Et₃N (13.3 mL, 0.095 mol) and NHS-linear fatty acid 06-06-2 (13 g, 0.038 mol). The resulting mixture was stirred at 75° C. After 16 h, the reaction mixture was neutralized with 1.5 N HCl. The precipitate was isolated via filtration, washed with water, and dried, affording 06-06-6 as an off-white solid (4.2 g, 30%).

### Step 4: Synthesis of Intermediate 06-06-7

To a stirred solution of 06-06-6 (3.8 g, 0.010 mol) in DCM (80 mL) at RT was added DMAP (0.12 g, 0.001 mol) and DCC (2.1 g, 0.010 mol), followed by N-hydroxysuccinimide (1.17 g, 0.010 mol). The resulting mixture was stirred at RT 16 h. Subsequently, the reaction mixture was filtered through a sintered funnel, and then the filtrate evaporated, yielding crude 06-06-7 as an off-white solid (4.7 g, 100%), which was used in the next step without further purification.

### Step 5: Synthesis of Lipid Motif DTx-06-06

To a stirred solution of 06-06-4 (4 g, 0.009 mol) in 1 M Na₂CO₃ (50 mL) and 1,4-dioxane (100 mL) at RT was added slowly 06-06-7 (4.5 g, 0.096 mol). The resulting mixture was stirred at RT. After 16 h, the reaction mixture was neutralized with 1.5 N HCl. The precipitate was isolated by filtration, washed with water, and dried. Purification of the precipitate by trituration with MeOH afforded lipid motif DTx-06-06 as an off-white solid (2.3 g, 32%). LCMS m/z (M+H)⁺: 737.6; ¹H-NMR (400 MHz, TFA-d): δ 0.77-0.79 (m, 6H), 1.22-1.52 (m, 51H), 1.68-1.81 (m, 11H), 2.10-2.18 (m, 2H), 2.50-2.67 (m, 5H), 2.94-2.98 (m, 2H), 3.49-3.60 (m, 4H).

### Synthesis of Lipid Motif DTx-06-08

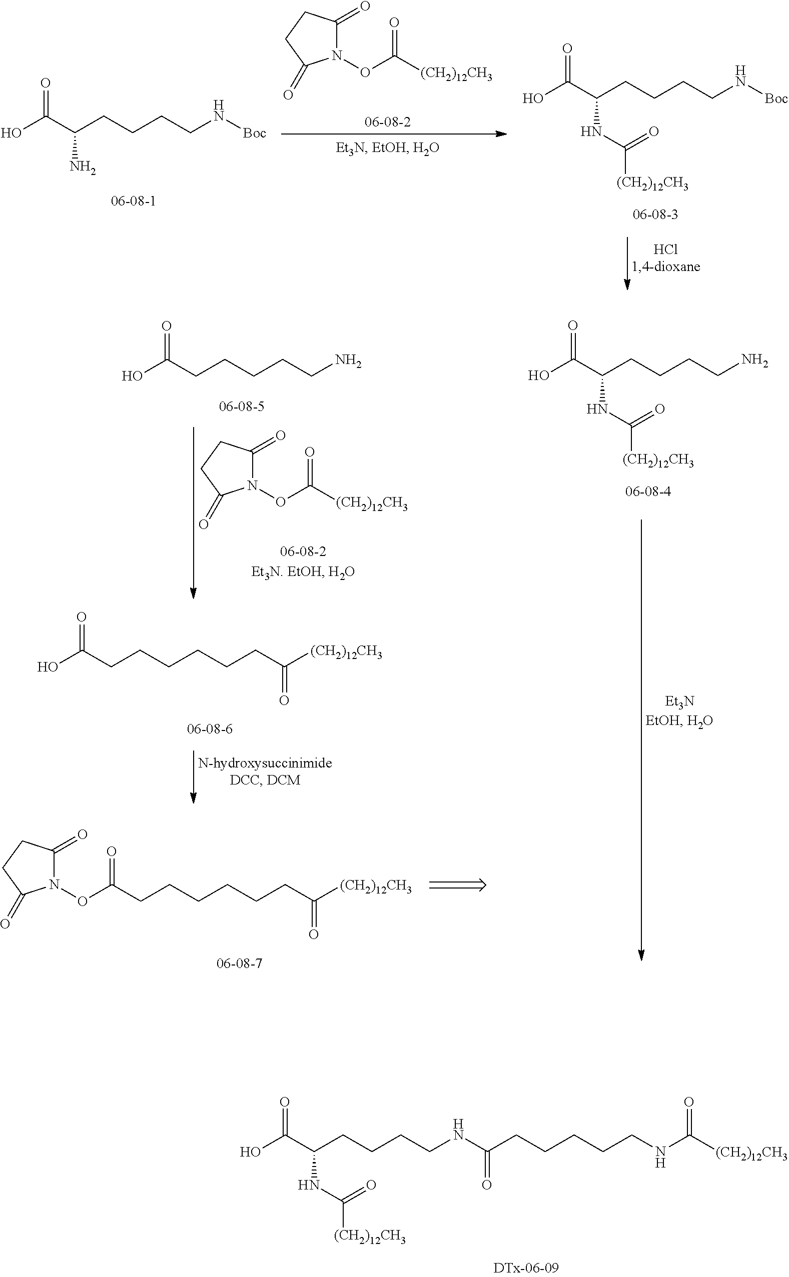

### Step 1: Synthesis of Intermediate 06-08-3

To a stirred solution of 06-08-1 (2.6 g, 10.6 mmol) in 65% aq. EtOH (50 mL) at RT was added slowly Et₃N (7.4 mL, 53 mmol) and NHS-linear fatty acid 06-08-2 (5 g, 15.4 mmol). The resulting mixture was stirred at 75° C. After 16 h, the reaction mixture was neutralized with 1.5 N HCl. The precipitate was isolated by filtration, washed with water, and dried. Purification of the precipitate by column chromatography (3% MeOH in DCM) afforded 06-08-3 as an off-white solid (3.3 g, 69%).

### Step 2: Synthesis of Intermediate 06-08-4

To a stirred solution of 06-08-3 (3.3 g, 7.23 mmol) in 1,4-dioxane (30 mL) at RT was added slowly 4 M HCl in 1,4-dioxane (30 mL). The resulting mixture was stirred at RT. After 16 h, the reaction mixture was concentrated under reduced pressure to yield crude 06-08-4, which was triturated with diethyl ether to afford 06-08-4 as an off-white solid (2.2 g, 85%).

### Step 3: Synthesis of Intermediate 06-08-6

To a stirred solution of 06-08-5 (5 g, 38 mmol) in 65% aq. EtOH (40 mL) at RT was added slowly Et₃N (13.3 mL, 95 mmol) and NHS-linear fatty acid 06-08-2 (12.5 g, 38 mmol). The resulting mixture was stirred at 75° C. After 16 h, the reaction mixture was neutralized with 1.5 N HCl. The precipitate was isolated via filtration, washed with water, and dried, affording 06-08-6 as an off-white solid (3.6 g, 28%).

### Step 4: Synthesis of Intermediate 06-08-7

To a stirred solution of 06-08-6 (3.6 g, 10.5 mmol) in DCM (80 mL) at RT was added DMAP (0.13 g, 1.1 mmol) and DCC (2.2 g, 10.5 mmol), followed by N-hydroxysuccinimide (1.2 g, 10.5 mmol). The resulting mixture was stirred at RT 16 h. Subsequently, the reaction mixture was filtered through a sintered funnel, and then the filtrate evaporated, yielding crude 06-08-7 as an off-white solid (4.2 g, 91%), which was used in the next step without further purification.

### Step 5: Synthesis of Lipid Motif DTx-06-08

To a stirred solution of 06-08-4 (2.2 g, 6.2 mmol) in 65% aq. EtOH (100 mL) at RT was added Et₃N (4.3 mL, 31 mmol) followed by 06-08-7 (4.2 g, 9.6 mmol). The resulting mixture was stirred at RT. After 16 h, the reaction mixture was neutralized with 1.5 N HCl. The precipitate was isolated by filtration, washed with water, and dried. Purification of the precipitate by trituration with MeOH afforded lipid motif DTx-06-08 as an off-white solid (2.1 g, 50%). LCMS m/z (M+H)⁺: 680.6; ¹H-NMR (400 MHz, TFA-d): δ 0.85-0.97 (m, 6H), 1.22-1.55 (m, 42H), 1.56-1.76 (m, 4H), 1.77-1.95 (m, 10H), 1.97-2.11 (m, 1H), 2.13-2.30 (m, 1H), 2.60-2.72 (m, 2H), 2.75 (m, 4H), 3.55-3.72 (m, 4H), 4.85-4.95 (m, 1H).

### Synthesis of Lipid Motif DTx-06-09

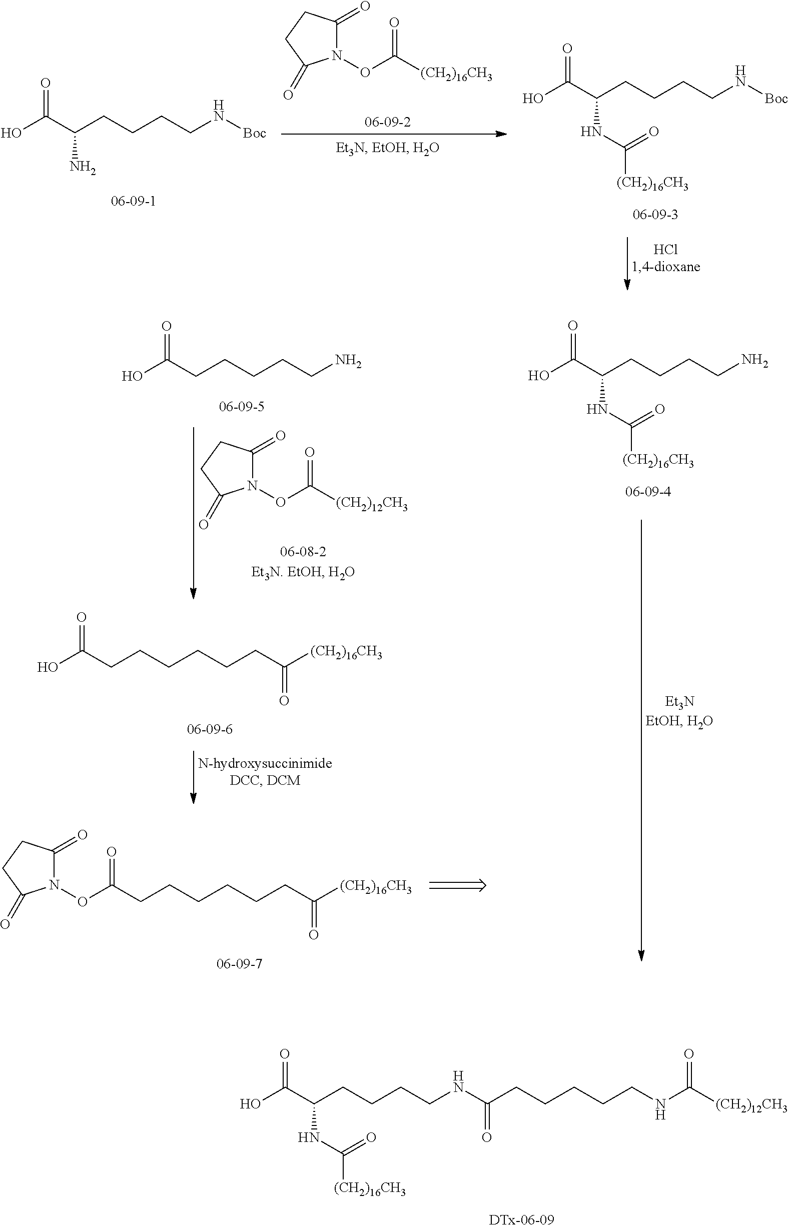

### Step 1: Synthesis of Intermediate 06-09-3

To a stirred solution of 06-09-1 (2.1 g, 8.7 mmol) in 65% aq. EtOH (50 mL) at RT was added slowly Et₃N (6.1 mL, 44 mmol) and NHS-linear fatty acid 06-08-2 (5 g, 13.1 mmol). The resulting mixture was stirred at 75° C. After 16 h, the reaction mixture was neutralized with 1.5 N HCl. The precipitate was isolated by filtration, washed with water, and dried. Purification of the precipitate by column chromatography (3% MeOH in DCM) afforded 06-09-3 as an off-white solid (2.8 g, 64%).

### Step 2: Synthesis of Intermediate 06-09-4

To a stirred solution of 06-09-3 (2.8 g, 7.23 mmol) in 1,4-dioxane (25 mL) at RT was added slowly 4 M HCl in 1,4-dioxane (25 mL). The resulting mixture was stirred at RT. After 16 h, the reaction mixture was concentrated under reduced pressure to yield crude 06-09-4, which was triturated with diethyl ether to afford 06-09-4 as an off-white solid (2.1 g, 95%).

### Step 3: Synthesis of Intermediate 06-09-6

To a stirred solution of 06-09-5 (5 g, 38 mmol) in 65% aq. EtOH (40 mL) at RT was added slowly Et₃N (13.3 mL, 95 mmol) and NHS-linear fatty acid 06-09-2 (14.5 g, 38 mmol). The resulting mixture was stirred at 75° C. After 16 h, the reaction mixture was neutralized with 1.5 N HCl. The precipitate was isolated via filtration, washed with water, and dried, affording 06-09-6 as an off-white solid (3.5 g, 23%).

### Step 4: Synthesis of Intermediate 06-09-7

To a stirred solution of 06-09-6 (3.5 g, 8.8 mmol) in DCM (60 mL) at RT was added DMAP (0.11 g, 0.9 mmol) and DCC (1.8 g, 8.8 mmol), followed by N-hydroxysuccinimide (1.0 g, 8.8 mmol). The resulting mixture was stirred at RT 16 h. Subsequently, the reaction mixture was filtered through a sintered funnel, and then the filtrate evaporated, yielding crude 06-09-7 as an off-white solid (4.0 g, 92%), which was used in the next step without further purification.

### Step 5: Synthesis of Lipid Motif DTx-06-09

To a stirred solution of 06-09-4 (2.1 g, 5.1 mmol) in 65% aq. EtOH (100 mL) at RT was added Et₃N (3.6 mL, 26 mmol) followed by 06-09-7 (4.0 g, 8.1 mmol). The resulting mixture was stirred at RT. After 16 h, the reaction mixture was neutralized with 1.5 N HCl. The precipitate was isolated by filtration, washed with water, and dried. Purification of the precipitate by trituration with MeOH afforded lipid motif DTx-06-09 as an off-white solid (2.1 g, 52%). ¹H-NMR (400 MHz, TFA-d): δ 0.81-0.97 (m, 6H), 1.22-1.69 (m, 60H), 1.72-1.91 (m, 10H), 1.95-2.05 (m, 1H), 2.10-2.25 (m, 1H), 2.59-2.71 (m, 2H), 2.78-2.85 (m, 4H), 3.52-3.80 (m, 4H), 4.80-4.92 (m, 1H).

### Synthesis of Lipid Motif DTx-01-36

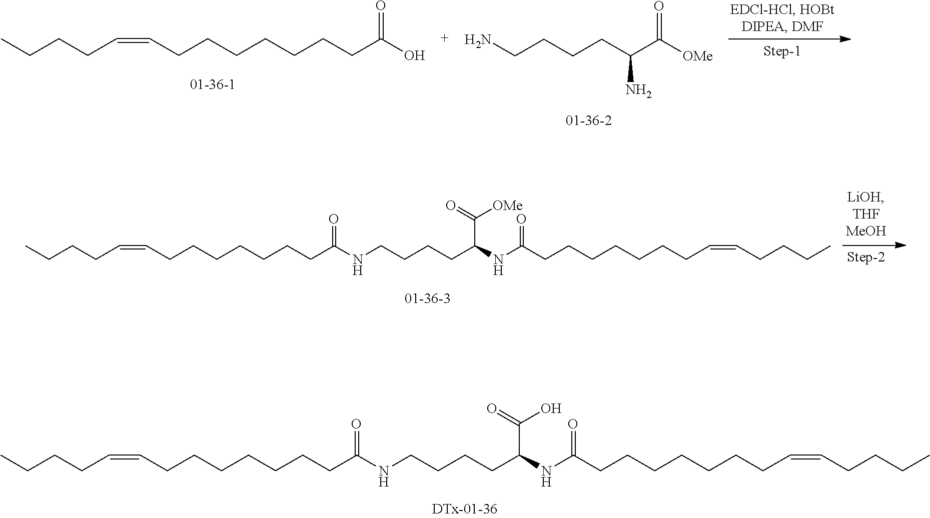

Step 1: To a stirred solution of 01-36-1 (0.73 g, 0.0032 mol) in DMF (6 mL) was added DIPEA (1.16 mL, 0.0064 mol), 01-36-2 (0.3 g, 0.0013 mol) followed by EDCl (0.543 g, 0.0028 mol), HOBt (0.382 g, 0.0028 mol) at RT. The resulting mixture was stirred at RT for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-36-3 as an off white solid. (0.54 g, 61%)

Step 2: To a stirred solution of compound 01-36-3 (0.5 g, 0.0009 mol) in MeOH, THF (10 mL; 1:1) and H₂O (0.25 mL) was added LiOH.H₂O (0.071 g, 0.0018 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-36 as an off white solid. (0.35 g, 73%)

### Analytics of DTx-01-36

¹H-NMR- (400 MHz, DMSO-d6): δ 0.84 (t, J=6.8 Hz, 6H), 1.27-1.66 (m, 35H), 1.98-2.10 (m, 12H), 2.93-2.99 (m, 2H), 4.08-4.14 (m, 1H), 5.27-5.35 (m, 4H), 7.71 (t, J=5.2 Hz, 1H), 7.96 (d, J=7.6 Hz, 1H), 12.49 (bs, 1H). LCMS: 563.5 (M+1).

### Synthesis of Lipid Motif DTx-01-39

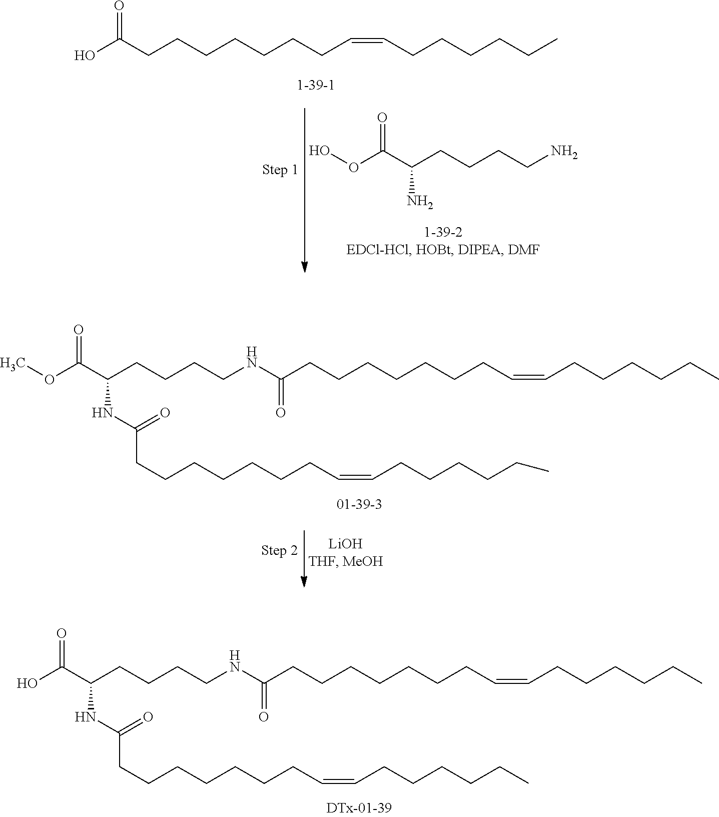

Step 1: To a stirred solution of compound 01-39-1 (2.04 g, 0.0080 mol) in DMF (20 mL) was added DIPEA (2.96 mL, 0.016 mol), compound 01-39-2 (0.75 g, 0.0032) followed by EDCl (1.35 g, 0.0070 mol), HOBt (0.95 g, 0.0070 mol) at RT. The resulting mixture was stirred at 50° C. for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-39-3 as an off white solid. (1.9 g, 79%)

Step 2: To a stirred solution of compound 01-39-3 (1.5 g, 0.0023 mol) in MeOH, THF (30 mL; 1:1) and H₂O (3 mL) was added LiOH.H₂O (0.194 g, 0.0046 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-39 as yellow solid. (1.2 g, 82%)

### Analytics of DTx-01-39

¹H-NMR- (400 MHz, DMSO-d6): δ 0.83 (t, J=6.8 Hz, 6H), 1.23-1.78 (m, 42H), 1.96-2.08 (m, 12H), 2.98 (d, J=5.6 Hz, 2H), 4.08-4.10 (m, 1H), 5.28-5.31 (m, 4H), 7.71 (t, J=5.2 Hz, 1H), 7.95 (d, J=8.4 Hz, 1H), 12.43 (bs, 1H). LCMS: 619.5 (M+1).

### Synthesis of Lipid Motif DTx-01-43

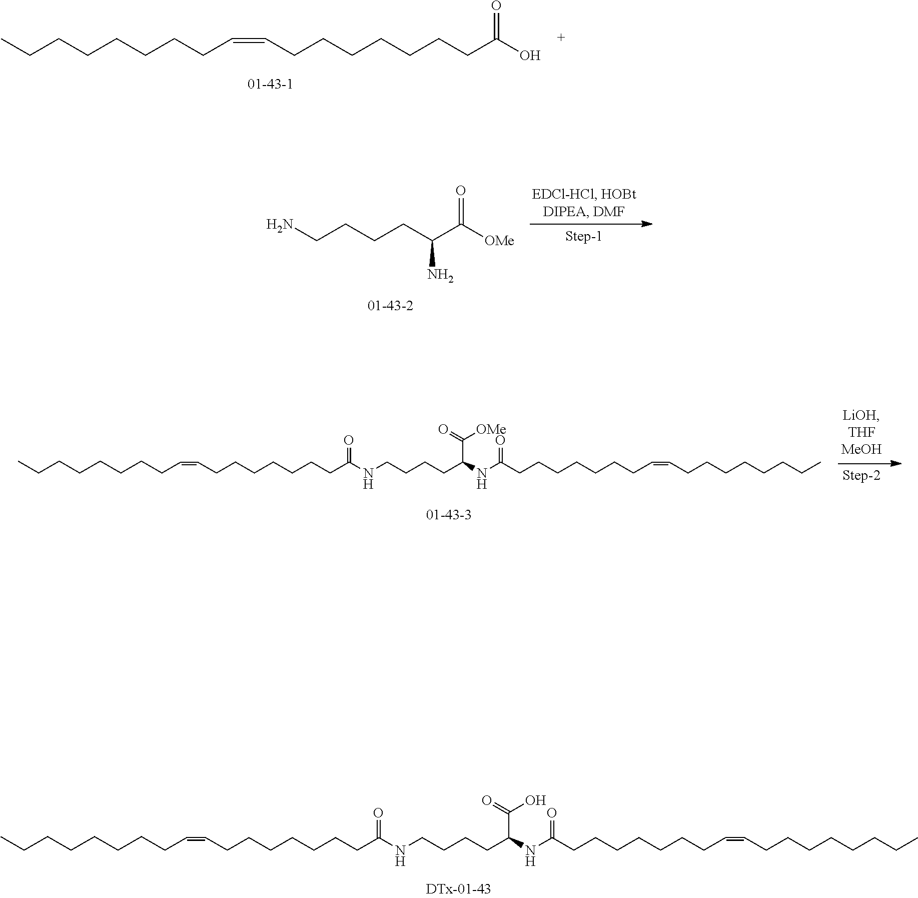

Step 1: To a stirred solution of compound 01-43-1 (3.5 g, 0.0107 mol) in DMF (50 mL) was added DIPEA (3.9 mL, 0.021 mol), compound 01-43-2 dihydrochloride (1 g, 0.0043 mol) followed by EDCl (1.8 g, 0.0094 mol), HOBt (1.2 g, 0.0094 mol) at RT. The resulting mixture was stirred at RT for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-43-3 as an off white solid. (2.6 g, 88.7%)

Step 2: To a stirred solution of compound 01-43-3 (2.5 g, 0.0036 mol) in MeOH, THF (40 mL; 1:1) and H₂O (2 mL) was added LiOH.H₂O (0.297 g, 0.0072 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-43 as an off white solid. (2.1 g, 90.6%)

### Analytics of DTx-01-43

¹H-NMR- (400 MHz, DMSO-d6): δ 0.83 (t, J=6.8 Hz, 6H), 1.05-1.65 (m, 48H), 1.96-2.16 (m, 14H), 2.98-2.99 (m, 2H), 4.11-4.16 (m, 1H), 5.29-5.37 (m, 4H), 7.71 (bs, 1H), 7.92 (d, J=6.4 Hz, 1H). LCMS: 676.5 (M+1).

### Synthesis of Lipid Motif DTx-01-44

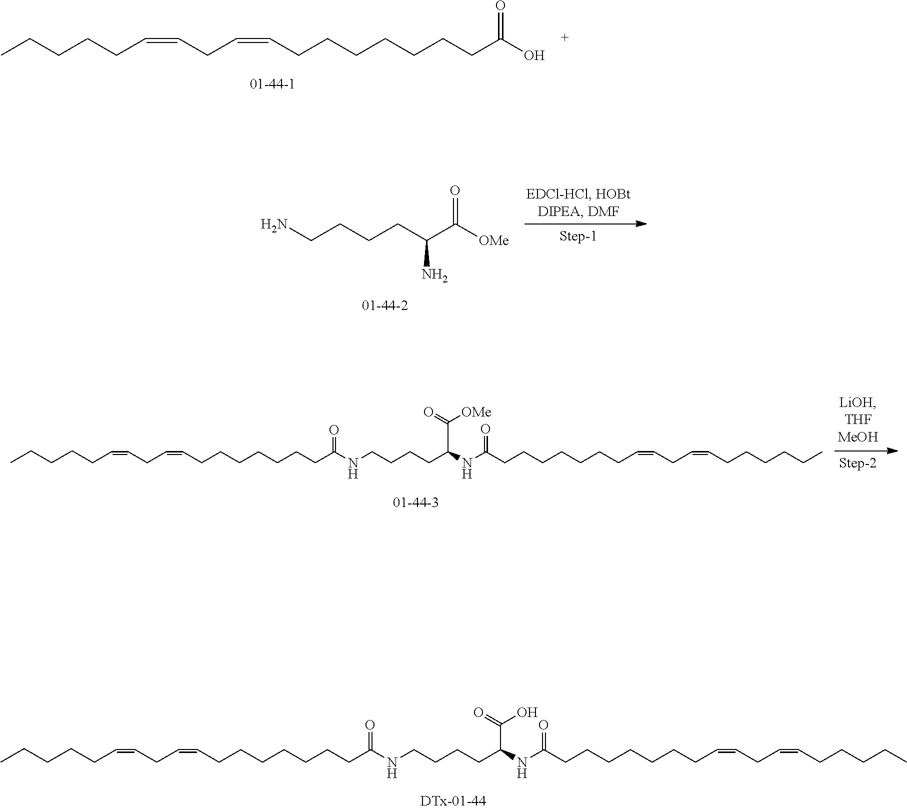

Step 1: To a stirred solution of compound 01-44-1 (5.1 g, 0.0018 mol) in DMF (50 mL) was added DIPEA (6.7 mL, 0.036 mol), compound 01-44-2 (1.7 g, 0.0072 mol) followed by EDCl (3.06 g, 0.016 mol), HOBt (2.16 g, 0.016 mol) at RT. The resulting mixture was stirred at RT for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-44-3 as an off white solid. (5 g, 85%)

Step 2: To a stirred solution of compound 01-44-3 (5 g, 0.0072 mol) in MeOH, THF (150 mL; 1:1) and H₂O (3 mL) was added LiOH.H₂O (0.60 g, 0.0144 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-44 as pale yellow viscous liquid. (2.2 g, 45%)

### Analytics of DTx-01-44

¹H-NMR- (400 MHz, DMSO-d6): δ 0.86 (t, J=5.2 Hz, 6H), 1.25-1.70 (m, 38H), 2.01-2.18 (m, 12H), 2.73 (t, J=6.4 Hz, 4H), 2.98-3.00 (m, 2H), 4.12-4.24 (m, 1H), 5.29-5.36 (m, 8H), 7.72 (t, J=5.2 Hz, 1H), 7.95 (d, J=8.0 Hz, 1H), 12.45 (bs, 1H). LCMS: 672.6 (M+1).

### Synthesis of Lipid Motif DTx-01-45

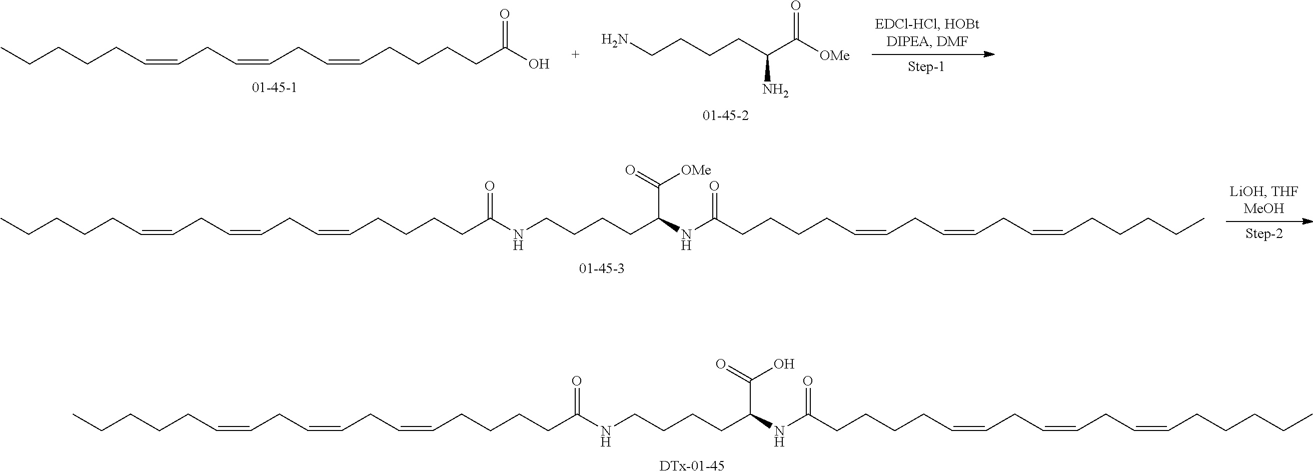

Step 1: To a stirred solution of compound 01-45-1 (0.656 g, 0.0023 mol) in DMF (5 mL) was added DIPEA (1.00 mL, 0.0053 mol), compound 04-45-2 dihydrochloride (0.25 g, 0.0011 mol) followed by EDCl (0.45 g, 0.0023 mol), HOBt (0.318 g, 0.0023 mol) at RT. The resulting mixture was stirred at RT for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-45-3 as an off white solid. (0.61 g, 83.56%)

Step 2: To a stirred solution of compound 04-45-3 (0.6 g, 0.0008 mol) in MeOH, THF (12 mL; 1:1) and H₂O (0.6 mL) was added LiOH.H₂O (0.074 g, 0.0018 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-45 as an off white solid. (0.55 g, 94.8%)

### Analytics of DTx-01-45

¹H-NMR- (400 MHz, DMSO-d6): δ 0.86 (t, J=6.0 Hz, 6H), 1.27-1.50 (m, 26H), 2.01-2.10 (m, 12H), 2.77-2.80 (m, 8H), 2.96-2.98 (m, 2H), 3.98-4.01 (m, 1H), 5.32-5.37 (m, 12H), 7.61 (bs, 1H), 7.75 (bs, 1H). LCMS: 668.4 (M+1).

### Synthesis of DTx-01-46

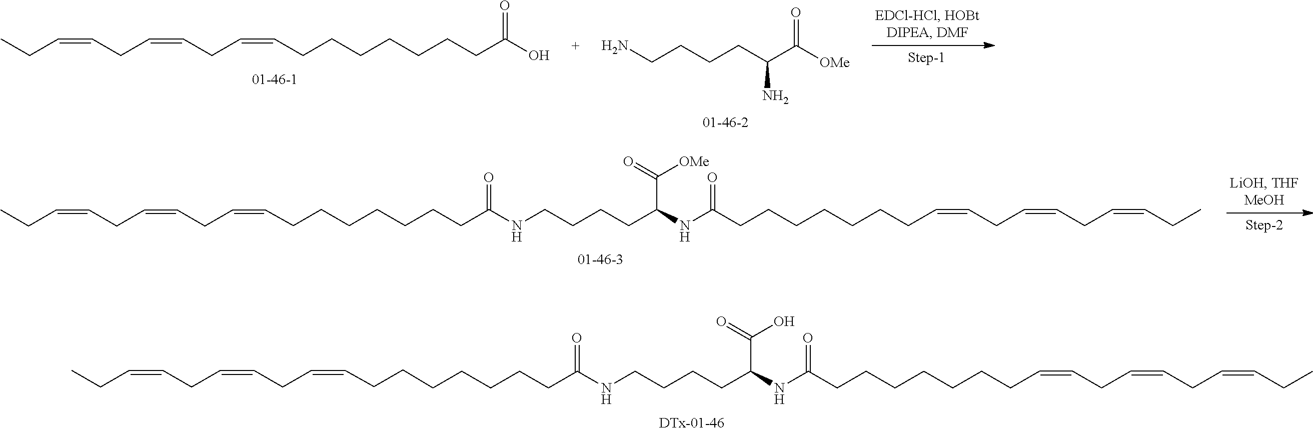

Step 1: To a stirred solution of compound 01-46-1 (2.00 g, 0.0071 mol) in DMF (20 mL) was added DIPEA (2.6 mL, 0.0143 mol), compound 01-46-2 (0.67 g, 0.0029 mol) followed by EDCl (1.20 g, 0.0063 mol), HOBt (0.085 g, 0.0063 mol) at RT. The resulting mixture was stirred at RT for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-46-3 as an off white solid. (1.8 g, 78%)

Step 2: To a stirred solution of compound 01-46-3 (2.4 g, 0.0035 mol) in MeOH, THF (75 mL; 1:1) and H₂O (2.5 mL) was added LiOH.H₂O (0.0288 g, 0.0070 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-46 as pale yellow viscous liquid. (1.5 g, 64%)

### Analytics of DTx-01-46

¹H-NMR- (400 MHz, DMSO-d6): δ 0.91 (t, J=7.6 Hz, 6H), 1.24-1.68 (m, 31H), 2.01-2.10 (m, 10H), 2.78 (t, J=6.0 Hz, 4H), 2.88-2.99 (m, 3H), 5.27-5.36 (m, 1H), 5.29-5.36 (m, 12H), 7.71 (t, J=5.2 Hz, 1H), 7.96 (d, J=8.0 Hz, 1H). LCMS: 668.6 (M+1).

### Synthesis of DTx-08-01

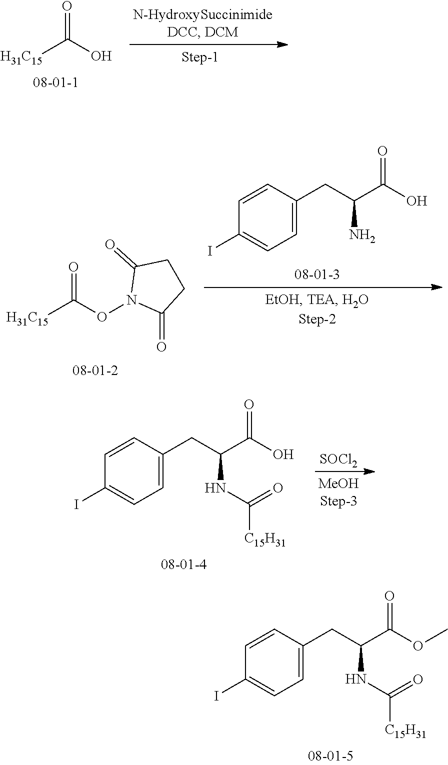

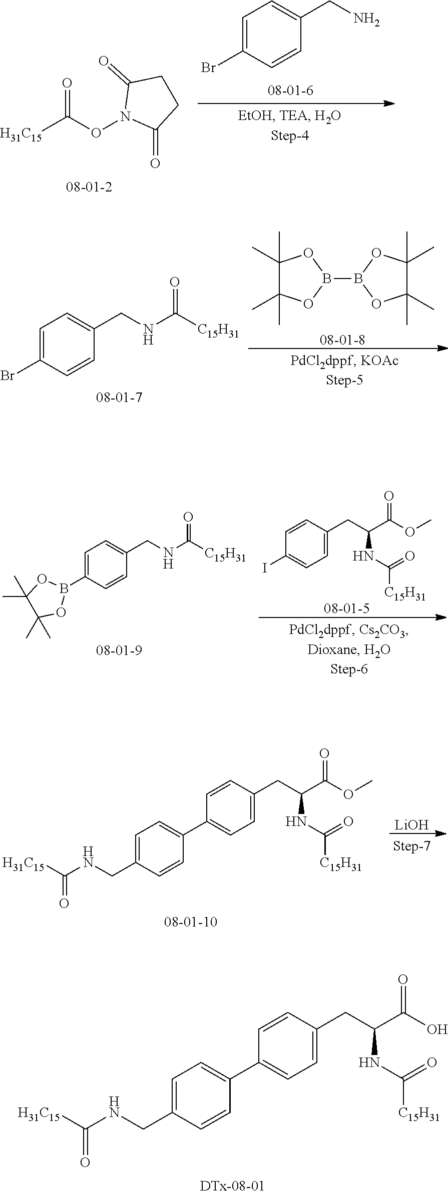

Step 1: To a stirred solution of compound 08-01-1 (10 g, 0.0389 mol) in DCM (200 mL) was added DMAP (0.47 g, 0.0038 mol), DCC (8.04 g, 0.0389 mol) followed by N-hydroxysuccinimide (4.48 g, 0.0389 mol) at RT. The resulting mixture was stirred at RT for 16 h. The reaction was monitored by LCMS. The reaction mixture was filtered through sintered funnel, the filtrate was evaporated to give crude product 08-01-02 as an off white solid which was directly proceeded for next step (10 g, 72%).

Step 2: To a stirred solution of compound 08-01-2 (10 g, 0.0283 mol) in 65% aq. ethanol (100 mL) was added Et₃N (11.8 mL, 0.0849 mol), compound 08-01-3 (10.6 g, 0.0368 mol) slowly at RT. The resulting mixture was stirred at 75° C. for 16 h. The reaction was monitored by LCMS. The reaction mixture was neutralized with 1.5 N HCl, precipitated solid was filtered, washed with water and dried to get the product 08-01-4 as an off white solid. (11 g, 73%)

Step 3: To a stirred solution of compound 08-01-4 (11 g, 0.0207 mol) in methanol (110 mL) was added thionyl chloride (44 mL) slowly at RT. The resulting mixture was stirred at RT for 16 h. The reaction was monitored by LCMS. The reaction mixture was concentrated under reduced pressure to get crude product which was triturated with diethyl ether to get pure compound of 08-01-5 as an off white solid (9 g, 80%).

Step 4: To a stirred solution of compound 08-01-2 (5 g, 0.0141 mol) in 65% aq. ethanol (50 mL) was added Et₃N (6 mL, 0.0424 mol), compound 08-01-6 (3.3 g, 0.0184 mol) slowly at RT. The resulting mixture was stirred at 75° C. for 16 h. The reaction was monitored by LCMS. The reaction mixture was neutralized with 1.5 N HCl, precipitated solid was filtered, washed with water and dried to get the product 08-01-7 as an off white solid. (5.1 g, 85%)

Step 5: To a stirred solution of compound 08-01-7 (5 g, 0.0117 mol) in dioxane (100 mL) was added 08-01-8 ((4,4,4′,4′,5,5,5′,5′-octamethyl-2,2′-bi(1,3,2-dioxaborolane) (4.4 g, 0.0176 mol)) and AcOK (3.4 g, 0.0353 mol). After degassing with nitrogen, Pd(dppf)Cl₂ (0.48 g, 0.0005 mol) was added to the reaction mixture. The resulting mixture was stirred at 90° C. for 12 h. The reaction mixture was monitored by LCMS, the reaction mixture was filtered through celite bed and concentrated under vacuum to give crude product which was further purified by column chromatography using 3% MeOH in DCM as eluent to get the product 01-08-9 as brown solid. (4.8 g, 86%)

Step 6: To a stirred solution of compound 01-08-5 (4.5 g, 0.0082 mol) in dioxane (90 mL) and water (9 mL) was added compound 01-08-9 (4.68 g, 0.0099 mol) and Cs₂CO₃ (8.1 g, 0.0248 mol). After degassing with nitrogen, Pd(dppf)Cl₂ (0.67 g, 0.0008 mol) was added to the reaction mixture. The resulting mixture was stirred at 90° C. for 3 h. The reaction mixture was monitored by LCMS, the reaction mixture was filtered through celite bed and concentrated under vacuum to give crude product which was further purified by column chromatography using 3% MeOH in DCM as eluent to get the product 01-08-10 as brown solid. (1 g, 14.2%)

Step 7: To a stirred solution of compound 01-08-10 (1 g, 0.0013 mol) in MeOH, THF (6.5 mL; 13 mL) and H₂O (6.5 mL) was added LiOH.H₂O (0.16 g, 0.0039 mol) and the reaction mixture was stirred at 50° C. for 3 h. The reaction mixture was monitored by LCMS, the reaction mixture was concentrated under vacuum. The resultant product was neutralized with 1.5 N HCl, the solid which was precipitated was filtered, washed with water and dried under vacuum to get the crude product. The crude product was triturated with MeOH to obtained pure DTx-08-01 as off white solid (0.5 g, 51%).

### Analytics of DTx-08-01

¹H-NMR- (400 MHz, TFA-dl): δ 0.78-0.79 (m, 6H), 1.08-1.49 (m, 48H), 1.49-1.50 (m, 2H), 1.72-1.83 (m, 2H), 2.69-2.71 (m, 2H), 5.77-2.82 (m, 2H), 3.41 (d, J=14.8 Hz, 1H), 3.53 (d, J=14.4 Hz, 1H), 4.66 (s, 2H), 5.16-5.18 (m, 1H), 7.23 (d, J=8.0 Hz, 2H), 7.33 (d, J=8.0 Hz, 2H), 7.58 (t, J=2.4 Hz, 4H). LCMS: 748.6 (M+1).

### Synthesis of DTx-09-01

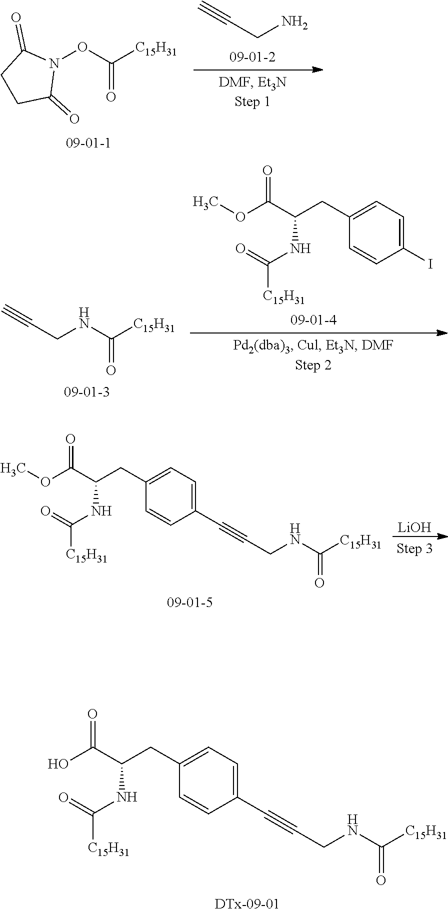

Step 1: To a stirred solution of compund 09-01 (10 g, 0.0283 mol) in DMF (100 mL) was added Et₃N (11.7 mL, 0.0849 mol), compound 09-01-2 (2.02 g, 0.0368 mol) slowly at RT. The resulting mixture was stirred at 50° C. for 16 h. The reaction was monitored by LCMS. The reaction mixture was neutralized with 1.5 NHCl, precipitated solid was filtered, washed with water and dried to get the product 09-01-3 as an off white solid. (4.5 g, 55%)

Step 2: To a stirred solution of compound 09-01-4 (5 g, 0.092 mol) in DMF (50 mL) was added compound 09-01-3 (3.5 g, 0.0119 mol), TEA (15 mL) and CuI (0.20 g, 0.0011 mol). After degassing with nitrogen, Pd₂(dba)₃ (0.67 g, 0.0007 mol) was added to the reaction mixture. The resulting mixture was stirred at 50° C. for 3 h. The reaction mixture was monitored by LCMS, the reaction mixture was filtered through celite bed and concentrated under vacuum to give crude product which was further purified by column chromatography using 2500 EtOAc in Hexane as eluent to get the product 09-01-5 as off white solid. (1 g, 15.6%)

Step 3: To a stirred solution of compound 09-01-5 (1 g, 0.0014 mol) in MeOH, THF (6.5 mL; 13 mL) and H₂O (6.5 mL) was added LiOH.H₂O (0.17 g, 0.0042 mol) and the reaction mixture was stirred at 50° C. for 2 h. The reaction mixture was monitored by LCMS, the reaction mixture was concentrated under vacuum to give crude which was neutralized with 1.5 N HCl, precipitated solid was filtered, washed with water and dried under vacuum to get the crude product. The crude product was further purified by column chromatography using 3%5 MeOH in DCM as eluent to get the product DTx-09-01 as off pale brown solid (0.5 g, 51%)

### Analytics of DTx-09-01

¹H-NMR- (400 MHz, TFA-dl): δ 0.89-0.92 (m, 6H), 1.20-1.40 (m, 49H), 1.67-1.70 (m, 2H), 1.82-1.86 (m, 2H), 2.71-2.75 (m, 2H), 5.91-2.95 (m, 2H), 3.47 (d, J=14.8 Hz, 1H), 3.61 (d, J=14.8 Hz, 1H), 4.52 (s, 2H), 7.25 (d, J=8.0 Hz, 2H), 7.50 (d, J=8.0 Hz, 2H). LCMS: 696.5 (M+1).

### Synthesis of DTx-10-01

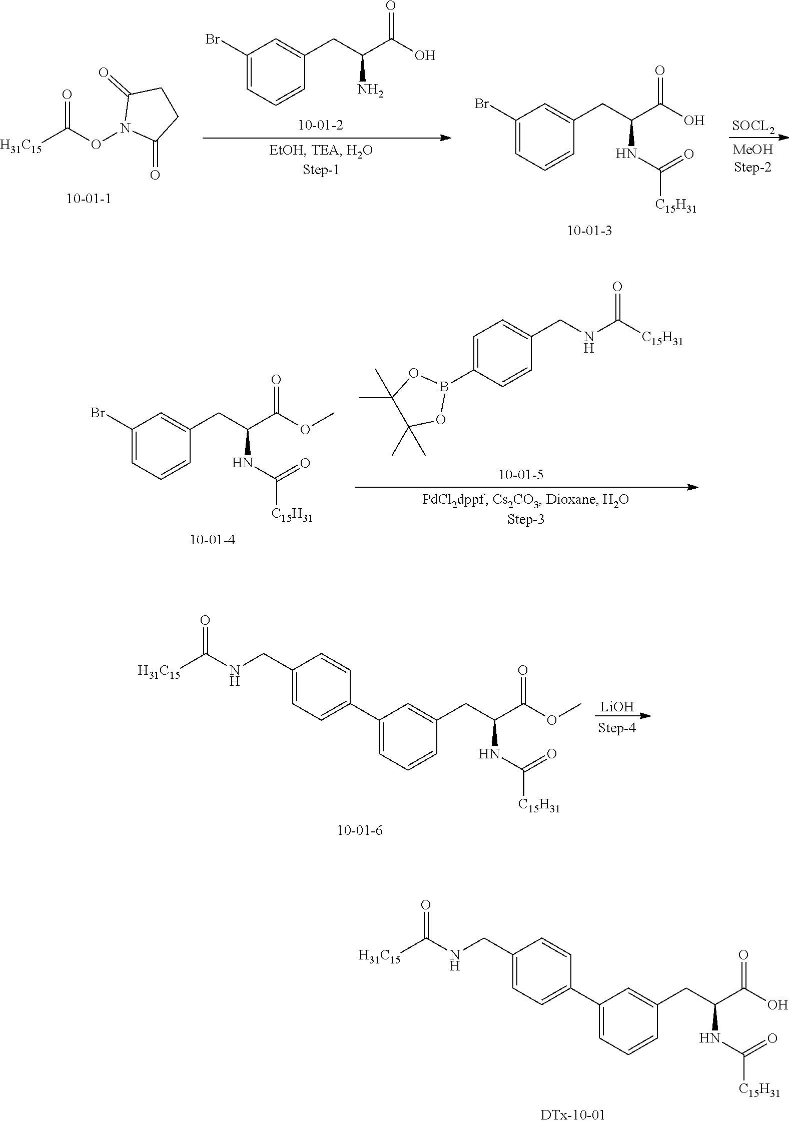

Step 1: To a stirred solution of compound 10-01-1 (5 g, 0.0141 mol) in 65% aq. ethanol (50 mL) was added Et₃N (10 mL, 0.0707 mol), compound 10-01-2 (3.45 g, 0.0141 mol) slowly at RT. The resulting mixture was stirred at 75° C. for 16 h. The reaction was monitored by LCMS. The reaction mixture was neutralized with 1.5 N HCl, precipitated solid was filtered, washed with water and dried to get the product 10-01-3 as an off white solid. (5.5 g, 80.6%)

Step 2: To a stirred solution of compound 10-01-3 (5.5 g, 0.0113 mol) in methanol (550 mL) was added thionyl chloride (22 mL) slowly at RT. The resulting mixture was stirred at RT for 16 h. The reaction was monitored by LCMS. The reaction mixture was concentrated under reduced pressure to get crude product which was triturated with diethyl ether to get pure compound of 10-01-4 as an off white solid (4.3 g, 76%).

Step 3: To a stirred solution of compound 10-01-4 (4.3 g, 0.0086 mol) in dioxane (90 mL) and water (9 mL) was added compound 10-01-5 (4.5 g, 0.00952 mol) and Cs₂CO₃ (8.4.6 g, 0.0259 mol). After degassing with nitrogen, Pd(dppf)Cl₂ (0.7 g, 0.0008 mol) was added to the reaction mixture. The resulting mixture was stirred at 90° C. for 3 h. The reaction mixture was monitored by LCMS, the reaction mixture was filtered through celite bed and concentrated under vacuum to give crude product which was further purified by column chromatography using 3% MeOH in DCM as eluent to get the product 10-01-6 as brown solid. (1.1 g, 16.68%)

Step 4: To a stirred solution of compound 10-01-6 (1.1 g, 0.0014 mol) in MeOH, THF (6.5 mL; 13 mL) and H₂O (6.5 mL) was added LiOH.H₂O (0.18 g, 0.0042 mol) and the reaction mixture was stirred at 50° C. for 3 h. The reaction mixture was monitored by LCMS, the reaction mixture was concentrated under vacuum. The resultant product was neutralized with 1.5 N HCl, the solid which was precipitated was filtered, washed with water and dried under vacuum to get the crude product. The crude product was triturated with MeOH to obtained pure DTx-10-01 as off white solid (0.7 g, 64%).

### Analytics of DTx-10-01

¹H-NMR- (400 MHz, TFA-dl): δ 0.78-0.80 (m, 6H), 1.13-1.45 (m, 50H), 1.73-1.75 (m, 2H), 2.39-2.43 (m, 1H), 2.70-2.74 (m, 2H), 3.14-3.20 (m, 1H), 3.46-3.51 (m, 2H), 4.68 (s, 2H), 5.17-5.20 (m, 1H), 7.17 (d, J=7.2 Hz, 1H), 7.33-7.43 (m, 4H), 7.50 (d, J=7.6 Hz, 1H), 7.57-7.58 (m, 2H). LCMS: 748.5 (M+1)

### Synthesis of DTx-11-01

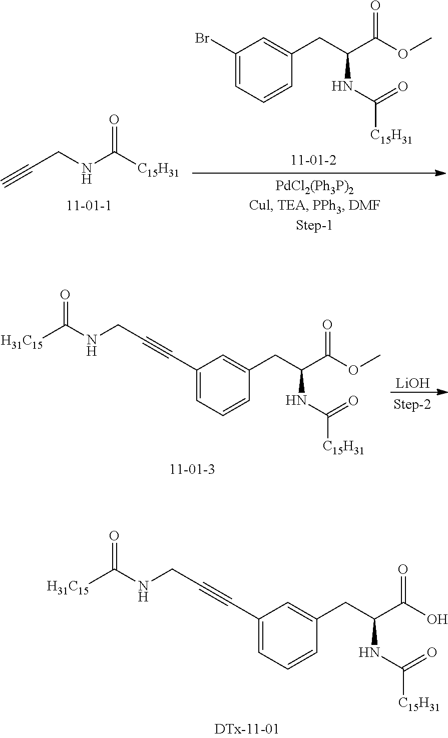

Step 1: To a stirred solution of compound 11-01-1 (2.68 g, 0.0091 mol) in DMF (35 mL) in a sealed tube was added compound 11-01-2 (3.5 g, 0.0070 mol), TEA (18 mL), PPh₃ (0.18 g, 0.0007 mol) and Cul (0.16 g, 0.0008 mol). After degassing with nitrogen, PdCl₂(Ph₃P)₂ (0.39 g, 0.0005 mol) was added to the reaction mixture. The resulting mixture was stirred at 110° C. for 3 h. The reaction mixture was monitored by LCMS, the reaction mixture was filtered through celite bed and concentrated under vacuum to give crude product which was further purified by column chromatography using 25% EtOAc in Hexane as eluent to get the product 11-01-3 as off white solid. (1 g, 20%)

Step 2: To a stirred solution of compound 11-01-3 (1 g, 0.0014 mol) in MeOH, THF (6.5 mL; 13 mL) and H₂O (6.5 mL) was added LiOH.H₂O (0.17 g, 0.0042 mol) and the reaction mixture was stirred at 50° C. for 2 h. The reaction mixture was monitored by LCMS, the reaction mixture was concentrated under vacuum to give crude which was neutralized with 1.5 N HCl, precipitated solid was filtered, washed with water and dried under vacuum to get the crude product. The crude product was further purified by column chromatography using 3% MeOH in DCM as eluent to get the product DTx-11-01 as off pale brown solid (0.7 g, 71%).

### Analytics of DTx-11-01

¹H-NMR- (400 MHz, TFA-dl): δ 0.87-0.90 (m, 6H), 1.31-1.47 (m, 48H), 1.65-1.68 (m, 2H), 1.81-1.85 (m, 2H), 2.71-2.74 (m, 2H), 2.89-2.95 (m, 2H), 3.42 (d, J=14.8 Hz, 1H), 3.57 (d, J=14.8 Hz, 1H), 4.50 (s, 2H), 5.20-5.24 (m, 1H), 7.25 (d, J=7.6 Hz, 1H), 7.34 (s, 1H), 7.39 (t, J=8.0 Hz, 1H), 7.47 (d, J=7.6 Hz, 1H). LCMS: 696.5 (M+1).

### Synthesis of DTx-04-01

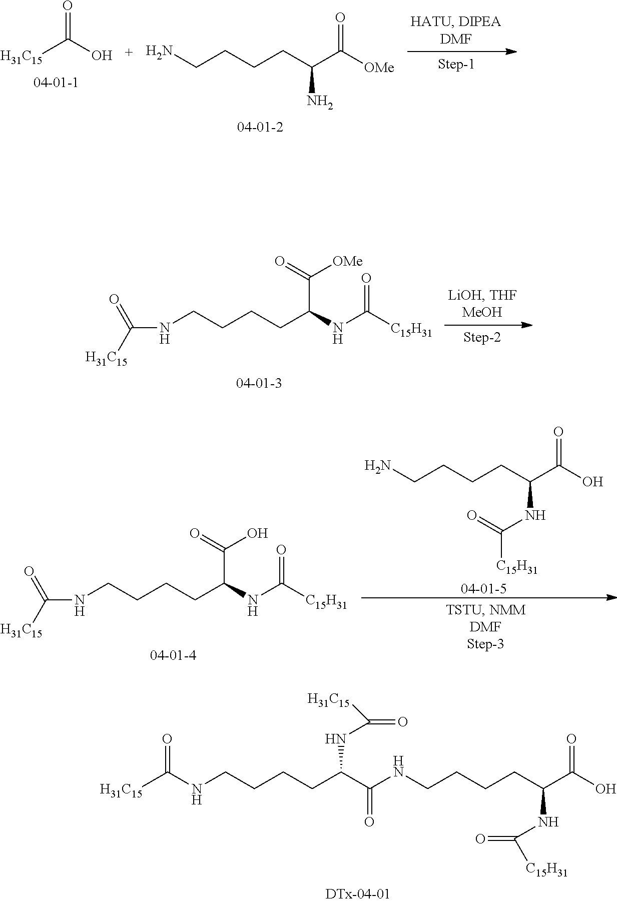

Step 1: To a stirred solution of compound 04-01-2 (5 g, 0.021 mol) in DMF (100 mL) was added DIPEA (19.7 mL, 0.107 mol), compound 04-01-1 (13.73 g, 0.053 mol) HATU (12.23 g, 0.032 mol) slowly at RT. The resulting mixture was stirred at RT for 16 h. The reaction was monitored by LCMS. The reaction mixture was quenched with ice cold water and filtered the solid, dried the solid under the vacuum to get the product 04-01-3 as off white solid (9.1 g, 67%).

Step 2: To a stirred solution of compound 04-01-3 (5 g, 0.0078 mol) in MeOH, THF (100 mL; 1:1) and H₂O (5 mL) was added LiOH.H₂O (0.660 g, 0.0157 mol) and the reaction mixture was stirred at RT for 16 h. The reaction mixture was monitored by LCMS, the reaction mixture was concentrated under vacuum to give crude which was neutralized with 1.5 N HCl, the solid which was precipitated was filtered, washed with water and dried under vacuum to get the product 04-01-4 as off white solid (3.9 g, 80%).

Step 3: To a stirred solution of compound 04-01-4 (3.0 g, 0.0048 mol) in DMF (60 mL) was added NMM (15 mL), followed by TSTU (2.18 g, 0.0096 mol) at RT. The resulting mixture was stirred at RT for 2 h. Compound 5 (3.69 g, 0.0096 mol) was added to the reaction mixture at 0° C. and then stirred at RT for 16 h. The reaction mixture was neutralized with 1.5 N HCl, precipitated solid was filtered, washed with water and dried. The crude product was triturated with MeOH to get the product DTx-04-01 as an off white solid. (2.8 g, 58%).

### Analytics of DTx-04-01

¹H-NMR (400 MHz, TFA-d): δ 1.09-1.13 (m, 9H), 1.57-2.16 (m, 84H), 2.38-2.44 (m, 3H), 2.77-2.94 (m, 4H), 3.18-3.31 (m, 5H), 3.69-3.81 (m, 5H), 4.87-4.92 (m, 1H). LCMS: 990.8 (M+1).

### Synthesis of DTx-05-01

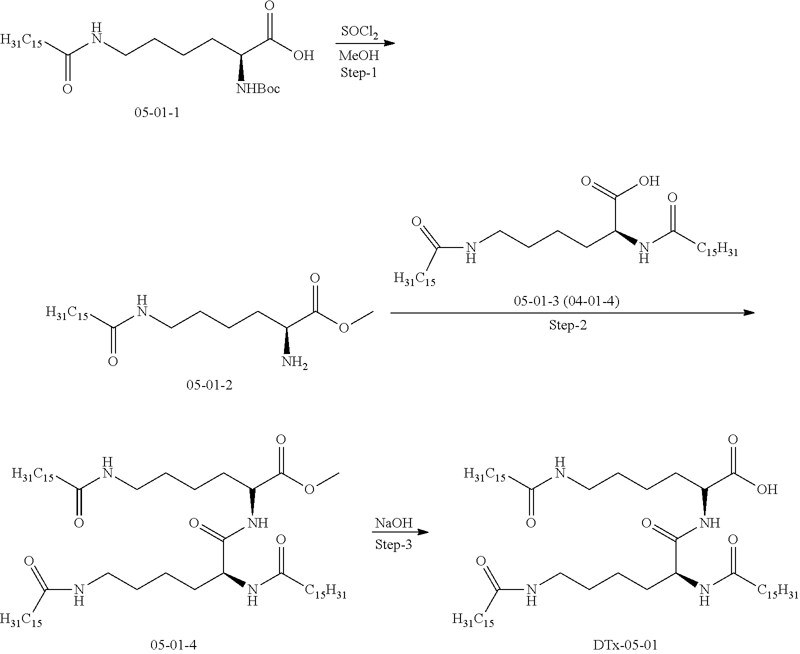

Step 1: To a stirred solution of compound 05-01-1 (5 g, 0.0103 mol) in methanol (50 mL) was added thionyl chloride (3.8 mL, 0.0516 mol) slowly at 0° C. The resulting mixture was stirred at RT for 16 h. The resulting mixture was evaporated and triturated with diethyl ether to give compound 05-01-2 as an off white solid which was directly proceeded for next step (3.5 g, 85%).

Step 2: To a stirred solution of compound 05-01-2 (2.89 g, 0.0067 mol) in DMF (35 mL) was added DIPEA (1.55 mL, 0.0084 mol), compound 05-01-3 (3.5 g, 0.0056 mol) and HBTU (2.12 g, 0.0056 mol) slowly at 0° C. The resulting mixture was stirred at 50° C. for 16 h. The reaction was monitored by LCMS. The reaction mixture was neutralized with 1.5 N HCl, precipitated solid was filtered, washed with water and dried to give compound 05-01-4 as pale brown solid. (3.2 g, 69%).

Step 3: To a stirred solution of compound 05-01-4 (3.2 g, 0.0031 mol) in MeOH, THF (60 mL; 1:1) and H₂O (3 mL) was added NaOH (0.25 g, 0.0062 mol) and the reaction mixture was stirred at 50° C. for 16 h. The reaction mixture was monitored by LCMS, the reaction mixture was concentrated and neutralized with 1.5 N HCl. The precipitated solid was filtered, washed with water and dried. The crude product was triturated with MeOH to give DTx-05-01 as pale brown solid. (2.3 g, 73%).

### Analytics of DTx-05-01

¹H-NMR- (400 MHz, TFA-d): δ 0.87-0.89 (m, 9H), 1.60-1.80 (m, 76H), 1.94-2.14 (m, 15H), 2.55-2.59 (m, 2H), 2.70-2.75 (m, 4H), 3.59-3.60 (m, 4H), 4.73-4.76 (m, 1H). LCMS: 990.8 (M+1).

### Synthesis of DTx-01-50 & DTx-01-52

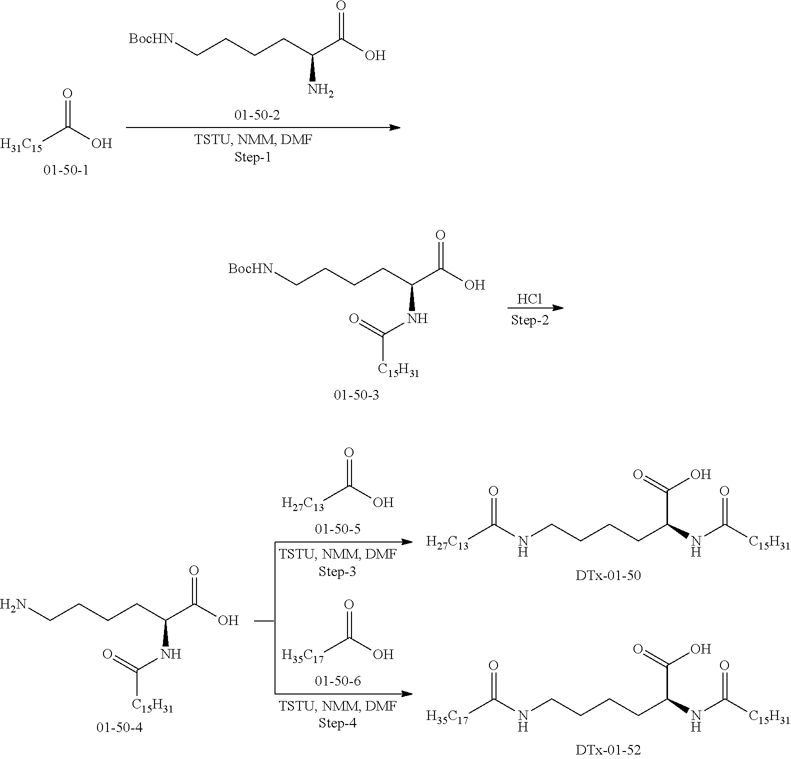

Step 1: To a stirred solution of 01-50-1 (5.0 g, 0.019 mol) in DMF (50 mL) was added NMM (25 mL), followed by TSTU (6.46 g, 0.021 mol) at RT. The resulting mixture was stirred at RT for 2 h. 01-50-2 (7.2 g, 0.029 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product 01-50-3 as brown solid. (9.1 g, 96%).

Step 2: To a stirred solution of compound 01-50-3 (9.1 g, 0.018 mol) in 1,4 dioxane (45 mL) was added 4 M HCl in dioxane (45 mL) slowly at RT. The resulting mixture was stirred at RT for 16 h. The reaction mixture was concentrated under reduced pressure to get crude product which was triturated with diethyl ether to get pure compound of 01-50-4 as an off white solid (6.5 g, 82%).

Step 3: To a stirred solution of compound 01-50-5 (1.5 g, 0.0065 mol) in DMF (45 mL) was added NMM (23 mL), followed by TSTU (2.17 g, 0.0072 mol) at RT. The resulting mixture was stirred at RT for 2 h. 01-50-4 (3.32 g, 0.0078 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product DTx-01-50 as pale brown solid. (2.1 g, 53%). LCMS: 595.5 (M+1). 1H-NMR- (400 MHz, TFA-d): δ 0.93-0.95 (m, 6H), 1.38-1.65 (m, 44H), 1.65-1.69 (m, 2H), 1.84-2.06 (m, 7H), 2.20-2.24 (m, 1H), 2.67 (t, J=7.6 Hz, 2H), 2.82 (t, J=7.9 Hz, 2H), 3.68 (t, J=6.8 Hz, 2H), 4.93 (t, J=8.0 Hz, 1H).

Step 4: To a stirred solution of compound 6 (1.5 g, 0.0052 mol) in DMF (45 mL) was added NMM (23 mL), followed by TSTU (1.74 g, 0.0058 mol) at RT. The resulting mixture was stirred at RT for 2 h. Compound 4 (2.66 g, 0.0063 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product DTx-01-52 as pale brown solid. (2.2 g, 64%). LCMS: 652.5 (M+1).

¹H-NMR- (400 MHz, TFA-d): δ 0.93-0.94 (m, 6H), 1.37-1.59 (m, 52H), 1.66-1.68 (m, 2H), 1.84-2.05 (m, 7H), 2.20-2.23 (m, 1H), 2.67 (t, J=7.3 Hz, 2H), 2.81 (t, J=7.5 Hz, 2H), 3.69 (t, J=6.2 Hz, 2H), 4.92 (t, J=4.9 Hz, 1H).

### Synthesis of DTx-01-51 & DTx-01-54

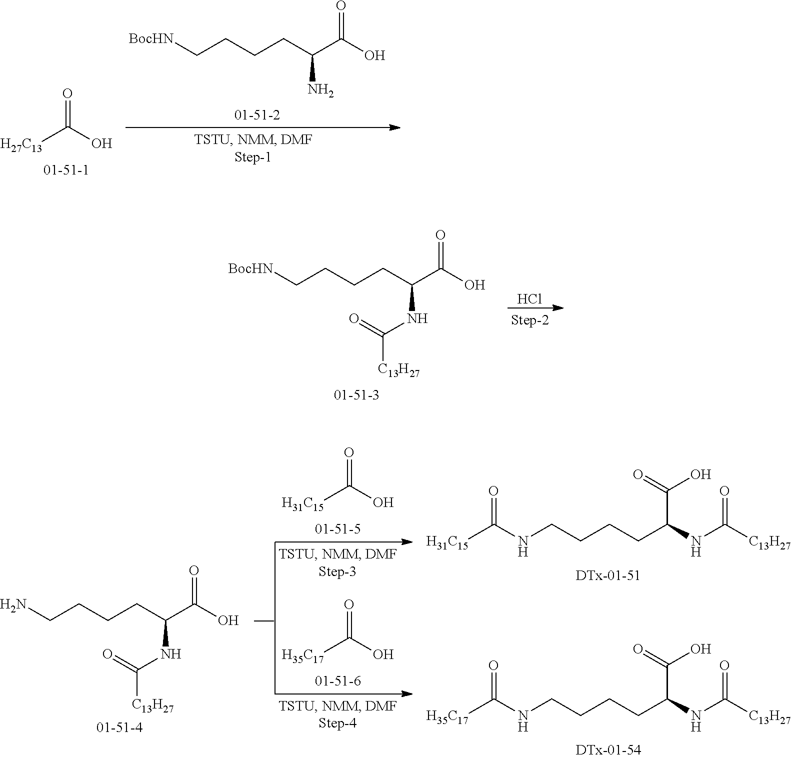

Step 1: To a stirred solution of 01-51-1 (5.0 g, 0.021 mol) in DMF (50 mL) was added NMM (25 mL), followed by TSTU (7.25 g, 0.024 mol) at RT. The resulting mixture was stirred at RT for 2 h. Compound 01-51-2 (8.09 g, 0.032 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product 01-51-3 as brown solid. (9 g, 90%).

Step 2: To a stirred solution of compound 01-51-3 (9 g, 0.014 mol) in 1,4 dioxane (45 mL) was added 4 M HCl in dioxane (45 mL) slowly at RT. The resulting mixture was stirred at RT for 16 h. The reaction mixture was concentrated under reduced pressure to get crude product which was triturated with diethyl ether to get pure compound of 01-51-4 as an off white solid (6.6 g, 81%).

Step 3: To a stirred solution of compound 01-51-5 (1.5 g, 0.0058 mol) in DMF (45 mL) was added NMM (23 mL), followed by TSTU (1.93 g, 0.0064 mol) at RT. The resulting mixture was stirred at RT for 2 h. Compound 01-51-4 (2.76 g, 0.0070 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product DTx-01-51 as pale brown solid. (2.4 g, 68%). LCMS: 595.5 (M+1). 1H-NMR- (400 MHz, TFA-d): δ 0.89-0.92 (m, 6H), 1.34-1.50 (m, 44H), 1.63-1.65 (m, 2H), 1.81-2.08 (m, 7H), 2.20-2.21 (m, 1H), 2.63 (t, J=7.3 Hz, 2H), 2.78 (t, J=7.4 Hz, 2H), 3.65 (t, J=6.4 Hz, 2H), 4.89 (t, J=7.1 Hz, 1H).

Step 4: To a stirred solution of compound 01-51-6 (1.5 g, 0.0052 mol) in DMF (45 mL) was added NMM (23 mL), followed by TSTU (1.74 g, 0.0058 mol) at RT. The resulting mixture was stirred at RT for 2 h. Compound 01-51-4 (2.49 g, 0.0063 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product DTx-01-54 as pale brown solid. (2.2 g, 66%). LCMS: 624.6 (M+1).

¹H-NMR- (400 MHz, TFA-d): δ 0.89-0.90 (m, 6H), 1.32-1.57 (m, 49H), 1.62-1.64 (m, 2H), 1.74-1.99 (m, 6H), 2.14-2.18 (m, 1H), 2.61 (t, J=7.6 Hz, 2H), 2.76 (t, J=7.6 Hz, 2H), 3.62 (t, J=7.0 Hz, 2H), 4.85-4.88 (m, 1H).

### Synthesis of DTx-01-53 & DTx-01-55

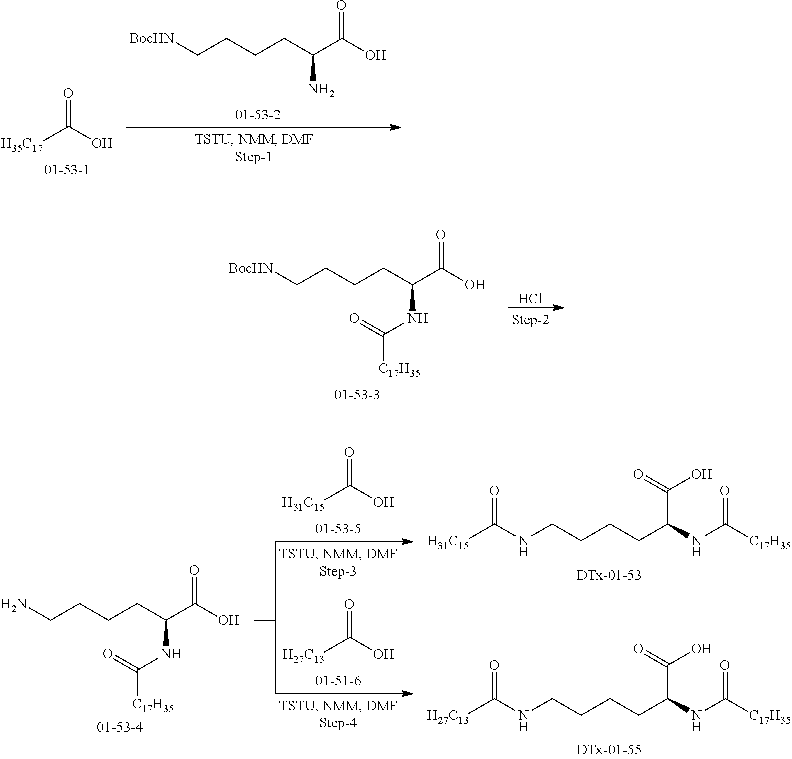

Step 1: To a stirred solution of compound 1 (5.0 g, 0.017 mol) in DMF (50 mL) was added NMM (25 mL), followed by TSTU (5.82 g, 0.019 mol) at RT. The resulting mixture was stirred at RT for 2 h. Compound 2 (5.18 g, 0.021 mol) was added to the reaction mixture at 0° C. and then stirred at 70° C. for 5 h and then concentrated. The reaction mixture was neutralized with 1.5 N HCl, precipitated solid was filtered, washed with water and dried. The crude product was triturated with MeOH to get the product 3 as brown solid. (8.6 g, 95%).

Step 2: To a stirred solution of compound 3 (8.6 g, 0.016 mol) in 1,4 dioxane (43 mL) was added 4 M HCl in dioxane (43 mL) slowly at RT. The resulting mixture was stirred at RT for 16 h. The reaction mixture was concentrated under reduced pressure to get crude product which was triturated with diethyl ether to get pure compound of 4 as an off white solid (7 g, 93%).

Step 3: To a stirred solution of compound 5 (1.5 g, 0.0058 mol) in DMF (45 mL) was added NMM (23 mL), followed by TSTU (1.94 g, 0.0064 mol) at RT. The resulting mixture was stirred at RT for 2 h. Compound 4 (3.15 g, 0.0070 mol) was added to the reaction mixture at 0° C. and then stirred at 70° C. for 5 h and then concentrated. The reaction mixture was neutralized with 1.5 N HCl, precipitated solid was filtered, washed with water and dried. The crude product was triturated with MeOH to get the product DTx-01-53 as pale brown solid. (2.2 g, 57%). LCMS: 652.6 (M+1).

¹H-NMR- (400 MHz, TFA-d): δ 0.82-0.85 (m, 6H), 1.27-1.50 (m, 52H), 1.54-1.58 (m, 2H), 1.73-1.94 (m, 7H), 2.07-2.14 (m, 1H), 2.56 (t, J=8.0 Hz, 2H), 2.71 (t, J=8.0 Hz, 2H), 3.58 (t, J=6.8 Hz, 2H), 4.81-4.84 (m, 1H).

Step 4: To a stirred solution of compound 6 (1.5 g, 0.0065 mol) in DMF (45 mL) was added NMM (23 mL), followed by TSTU (2.17 g, 0.0072 mol) at RT. The resulting mixture was stirred at RT for 2 h. Compound 4 (3.53 g, 0.0078 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product DTx-01-55 as pale brown solid. (2.3 g, 56%). LCMS: 624.6 (M+1).

¹H-NMR- (400 MHz, TFA-d): δ 0.90-0.93 (m, 6H), 1.35-1.49 (m, 48H), 1.60-1.63 (m, 2H), 1.77-2.02 (m, 7H), 2.17-2.21 (m, 1H), 2.64 (t, J=7.6 Hz, 2H), 2.78 (t, J=7.7 Hz, 2H), 3.65 (t, J=7.0 Hz, 2H), 4.88-4.91 (m, 1H).

The motifs presented in the above synthesis schemes, as well as additional motifs, are listed in the tables provided herein.

### Conjugating the Uptake Motifs to Single-Stranded Oligonucleotides

As described in Schemes I, II, III, and IV below, uptake motifs were conjugated to single-stranded oligonucleotides. The herein schemes shown are representative for conjugation to the 5′ end, to the 3′ end, and to the 5′ and 3′ ends of a single-stranded oligonucleotide. The synthesis schemes may be modified by one skilled in the art, to conjugate a specific motif provided herein to a single-stranded oligonucleotide provided.

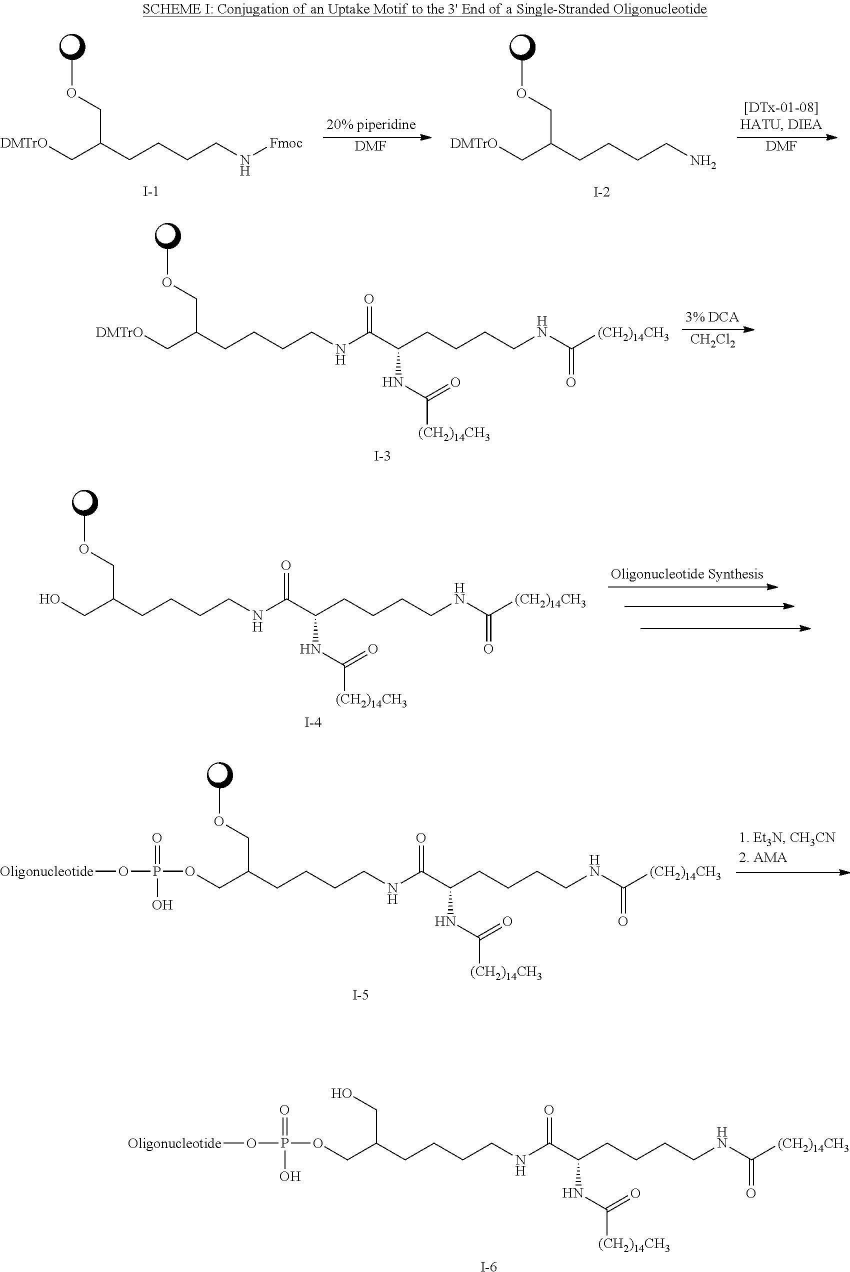

Scheme I above illustrates the preparation of a single-stranded oligonucleotide with an uptake motif at the 3′ end. In summary, 3′-amino CPG beads I-1 (Glen Research, Catalog No. 20-2958) modified with the DMT and Fmoc-protected C7 linker illustrated above were treated with 20% piperidine/DMF to afford Fmoc-deprotected amino C7 CPG beads I-2. Lipid motif DTx-01-08 was then coupled to I-2 using HATU and DIEA in DMF to produce lipid-loaded CPG beads I-3, which were treated by 3% dichloroacetic acid (DCA) in DCM to remove the DMT protecting group and afford I-4. Oligonucleotide synthesis on I-4 was accomplished via standard phosphoramidite chemistry and yielded modified oligonucleotide-bounded CPG beads I-5. Removal of phosphate protecting groups was achieved by treatment with triethylamine in acetonitrile. Subsequent treatment of I-5 with AMA [ammonium hydroxide (28%)/methylamine (40%) (1:1, v/v)] cleaved the DTx-01-08-conjugated modified oligonucleotide from the beads. The oligonucleotide was then purified by RP-HPLC and characterized by MALDI-TOF MS using the [M+H] peak.

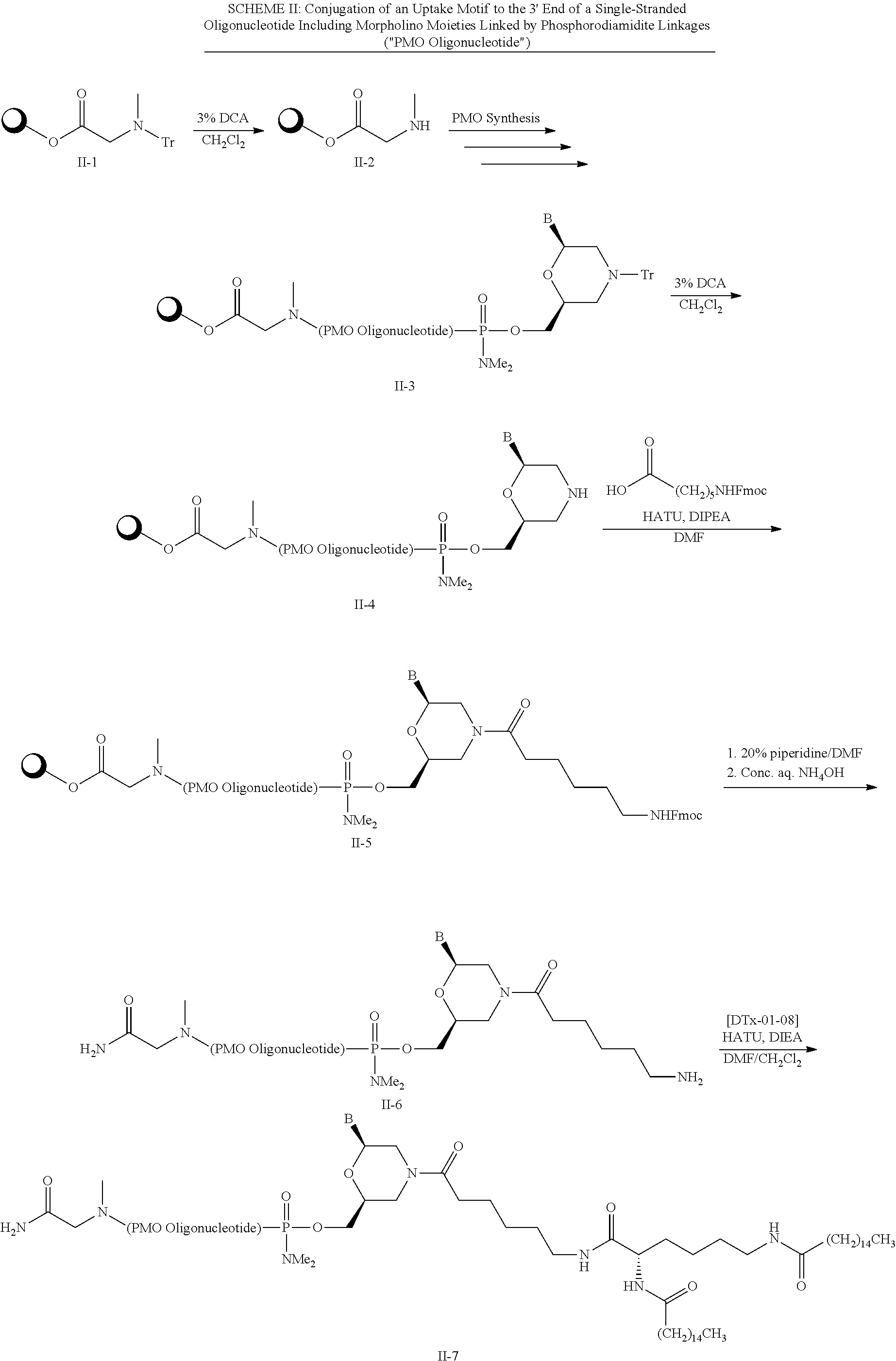

Scheme II above illustrates the preparation of a single-stranded oligonucleotide with morpholino moieties linked by phosphorodiamidite linkages (PMO oligonucleotide) with an uptake motif at the 3′ end. Synthesis resin loaded with trityl-protected sarcosine (II-1) is treated with 3% dichloroacetic acid (DCA) in dichloromethane to liberate the secondary amine in II-2. This resin is used to initiate oligonucleotide synthesis using commercially available monomers. The residual trityl protecting group on the resin-bound PMO oligonucleotide (II-3) is removed by treatment with 3% dichloromethane (DCA) in dichloromethane to generate II-4. Fmoc-protected 6-amino-hexanoic acid was coupled to II-4 using HATU and DIPEA in DMF to produce the C₆-modified II-5. Treatment with 20% piperidine in DMF resulted in removal of Fmoc to generate II-6. The uptake motif DTx-01-08 was subsequently coupled to II-6 using HATU and DIEA in DMF to produce the resin-bound lipid-conjugated oligonucleotide. Final treatment with concentrated aqueous ammonia liberates the fully deprotected PMO II-7. The oligonucleotide was then purified by RP-HPLC and characterized by MALDI-TOF MS using the [M+H] peak.

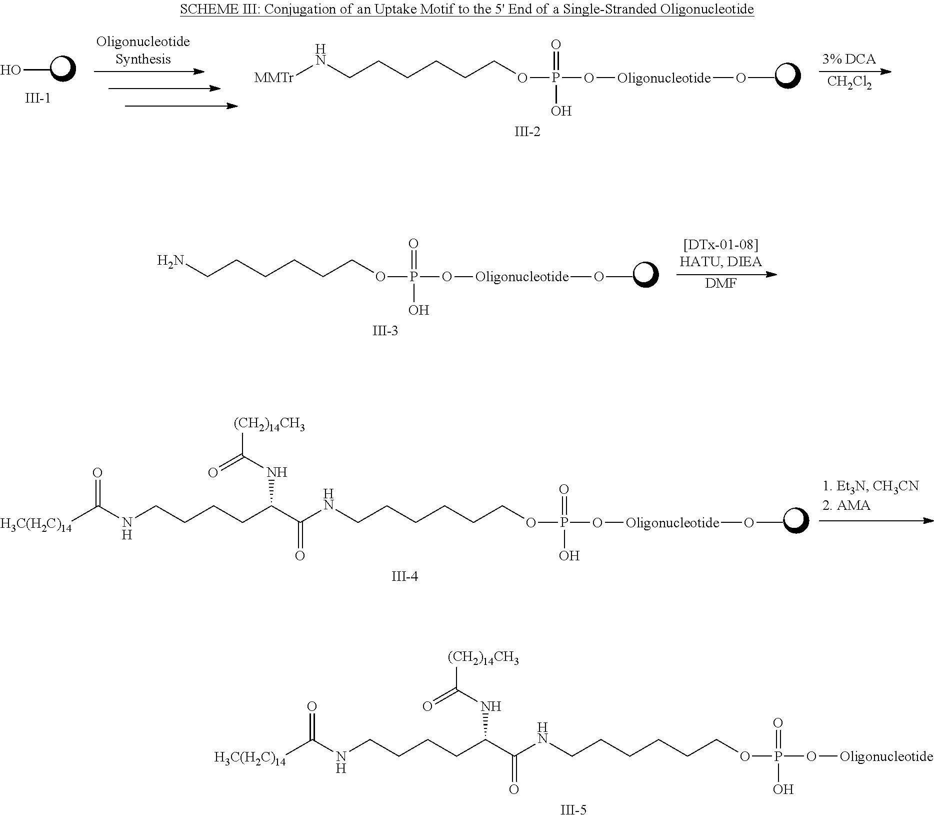

Scheme III above illustrates the preparation of a sense strand of a double-stranded oligonucleotide conjugated with an uptake motif at the 5′ end. In summary, oligonucleotide synthesis was performed on CPG beads III-1 (Glen Research, Catalog No. 20-5041-xx) via standard phosphoramidite chemistry. The final coupling was with a phosphoramidite (Glen Research, Catalog No. 10-1906) that incorporated a monomethoxytrityl (MMTr) protected 6-carbon alkyl amine as shown in structure III-2. The MMTr was removed with 3% dichloroacetic acid (DCA) in DCM to yield III-3. The free alkyl amine was coupled to the lipid motif (DTx-01-08) using HATU and DIEA in DMF to yield III-4. Stepwise deprotection with triethylamine in acetonitrile (to remove phosphate protecting groups) and AMA [ammonium hydroxide (28%)/methylamine (40%) (1:1, v/v)] (to remove base protecting groups and cleave the oligonucleotide from the synthesis resin) yielded crude III-5. Purification using RP-HPLC yielded material that was ready for testing. Purity and identity of III-5 were confirmed by analytical RP-HPLC and MALDI-TOF MS using the [M+H] peak, respectively.

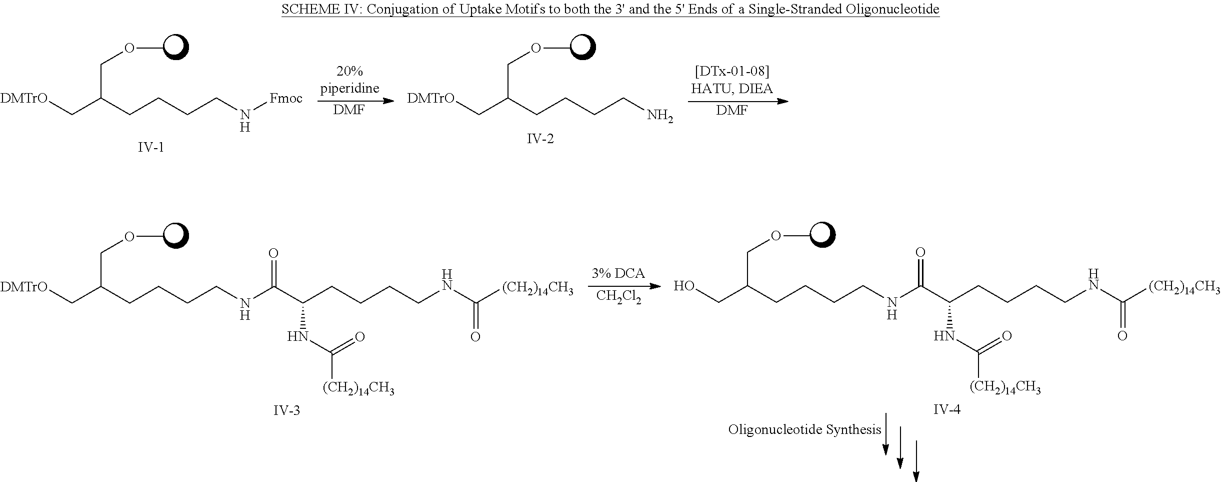

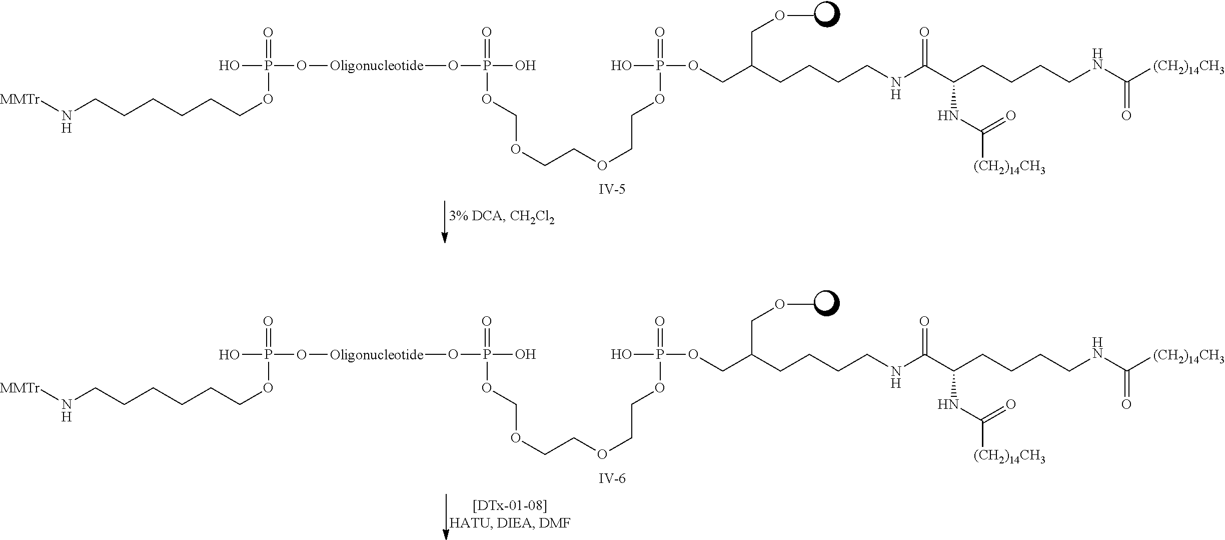

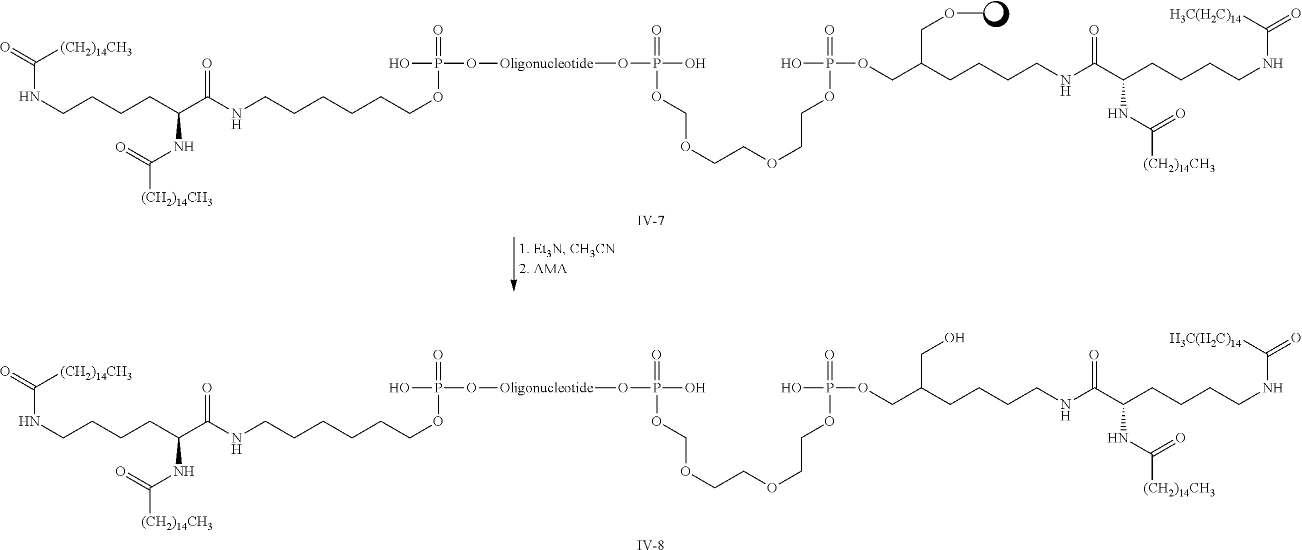

Scheme IV above illustrates the preparation of an oligonucleotide conjugated with an uptake motifs at both the 5′ and 3′ ends. In summary, 3′-amino CPG beads IV-1 (Glen Research, Catalog No. 20-2958) modified with the DMT and Fmoc-protected C7 linker illustrated above were treated with 20% piperidine/DMF to afford Fmoc-deprotected amino C₇ CPG beads IV-2. In this example, the motif DTx-01-08 was then coupled to IV-2 using HATU and DIEA in DMF to produce the loaded CPG beads IV-3, which were subsequently treated with 3% dichloroacetic acid (DCA) in DCM to remove the DMT protecting group and afford IV-4. Oligonucleotide synthesis was completed using IV-4. The final coupling was with a phosphoramidite (Glen Research, Catalog No. 10-1906) that incorporated a monomethoxytrityl (MMTr) protected 6-carbon alkyl amine as shown in structure IV-5. After removal of MMT with 3% dichloroacetic acid (DCA) in DCM, III-6 was coupled to the motif DTx-01-08 using HATU and DIEA in DMF to yield IV-7. Stepwise deprotection with triethylamine in acetonitrile (to remove phosphate protecting groups) and AMA [ammonium hydroxide (28%)/methylamine (40%) (1:1, v/v)] (to remove base protecting groups and cleave the oligonucleotide from the synthesis resin) yielded crude IV-8. Purification using RP-HPLC yielded a material ready for testing. Purity and identity of IV-8 were confirmed by analytical RP-HPLC and MALDI-TOF MS using the [M+H] peak, respectively.

### Biological Data

siRNA Sequences

| | | SEQ |
|---|---|---|
| siRNA | | ID |
| Identifier | Sequence and Chemistry | NO: |
| DT-00003 | Passeneer Sequence (5′ to 3′) | 238 |
| | fG mA fU mG fA mU fG mU fU fU fG mA fA mA fC mU fA mU | |
| | fU*T*T | |
| | Guide Sequence (5′ to 3′) | 239 |
| | PO4-mA fA mU fA mG fU mU fU mC mA mA fA mC fA mU fC mA fU | |
| | mC*T*T | |
| DTxO-0038 | Passenger Sequence (5′ to 3′) | 240 |
| | fA*mC*fC mU fG mA fU mC fA mU fU mA fU mA fG mA fU*mA*fA | |
| | Guide Sequence (5′ to 3′) | 241 |
| | PO4- eT*fU*mA fU mC fU mA fU mA fA mU fG mA fU mC fA mG fG | |
| | mU *T *T | |

### Cell Culture

HEK293 cells were purchased from ATCC and cultured in DMEM containing 10% Fetal Bovine Serum (FBS), 2 mM L-glutamine, 1X non-essential amino acids, 100 U/mL penicillin and 100 mg/mL streptomycin in a humidified 37 C incubator with 5% CO2.

HUVEC cells were purchased from Cell Applications (San Diego, Calif.) and cultured in their proprietary HUVEC cell media containing 2% serum, 100 U/mL penicillin and 100 mg/mL streptomycin

C2C12 cells were obtained from ATCC (Manassas, Va.) and maintained in DMEM containing 10% Fetal Bovine Serum (FBS), 2 mM L-glutamine, 100 U/mL penicillin and 100 mg/mL streptomycin in a humidified 37 C incubator with 5% CO2.

Human skeletal muscle cells (HSkMCs) were obtained from Cell Applications (San Diego, Calif.) and maintained in proprietary growth medium (Cell Applications, cat #151-500) in a humidified 37 C incubator with 5% CO2.

### Transfection of HEK293 Cells

24 hours prior to transfection, HEK293 cells were plated into 96 well plates at 10,000 cells/well in 90 uL of antibiotic free media. The oligonucleotide or oligonucleotide conjugates were diluted in PBS to 100× of the desired final concentration. Separately, Lipofectamine RNAiMax (Life Technologies) was diluted 1:66.7 in media lacking supplements (e.g. FBS, antibiotic etc.). The 100× oligonucleotide in PBS was then complexed with RNAiMAX by adding 1 part oligonucleotide in PBS to 9 parts lipofectamine/media. Following incubation for 20 minutes, 10 uL of the oligonucleotide:RNAiMAX complexes were added to the cells plated 24 hours prior containing 90 uL of antibiotic free media. The complexes were removed 24 hours following and replaced with media containing antibiotics. RNA was isolated 48 hours following transfection.

### Transfection of C2C12 and HSkM Cells

24 hours prior to transfection, C2C12 cells were plated at 300,000 cells per well in a 6-well collagen I-coated plate in growth medium (DMEM+10% fetal bovine serum+pen/strep) and HSKMCs were plated at 300,000 cells per well in a 6-well collagen I-coated plate with HSkMC growth medium (Cell Applications, cat #151-500). Immediately prior to transfection, the oligonucleotide or oligonucleotide conjugates were diluted in PBS to 100× of the desired final concentration. Separately, Lipofectamine RNAiMax (Life Technologies) was diluted 1:66.7 in media lacking supplements (C2C12: DMEM only; HSkMCs: Cell Applications, cat #151DF-250). The 100× oligonucleotide in PBS was then complexed with RNAiMAX by adding 1 part oligonucleotide in PBS to 9 parts lipofectamine/media. Following incubation for 20 minutes, 200 uL of the oligonucleotide:RNAiMAX complexes were added to the cells plated 24 hours prior containing 1800 uL of antibiotic free media (C2C12: DMEM+10% fetal bovine serum; HSkMCs: Cell Applications Cat #151DA-250). The complexes were removed 24 hours following and replaced with differentiation media containing antibiotics (C2C12: DMEM+2% horse serum+pen/strep+1 uM Insulin; HSkMCs: Cell Applications, cat #151D-250). Differentiation media was replaced 48 hours later. RNA was then isolated as described below 96 hours following the initial incubation with differentiation media.

### Free Uptake Experiment in HUVEC Cells

HUVEC cells were plated at 10,000 cells/well on 96 well collagen-coated plates. The day after plating, the HUVEC media was removed and the cells were washed twice with PBS containing calcium and magnesium. Following the last wash, cells were incubated with compounds at various concentrations in serum free HUVEC media for 24 hours. After 24 hours, compound containing media was removed and replaced with normal HUVEC media for 24 additional hours. Cells were then washed twice with PBS containing calcium and magnesium and then prepared for RNA isolation according to the manufacturer's protocol (see above). In an alternative paradigm, cells were incubated with compounds at various concentrations in normal HUVEC media containing 2% serum for 48 hours. After 48 hours, cells were washed twice with PBS containing calcium and magnesium and then prepared for RNA isolation according to the manufacturer's protocol (see below).

### Free Uptake in C2C12 and HSkM Cells

C2C12 and HSkMCs were plated at 300,000 per well in a 6-well collagen I-coated plate in their respective growth media (C2C12: DMEM+10% fetal bovine serum+pen/strep; HSkMC: Cell Applications Cat #cat #151-500). 24 hours following plating, oligonucleotide or oligonucleotide conjugates were added to cells at the desired concentration in 2 mL of differentiation media (C2C12 DMEM+2% horse serum+pen/strep+1 uM insulin, sterile filtered; HSkMC: Cell Applications cat #151D-250). Differentiation media containing compounds were replaced 48 hours later. RNA was then isolated as described below 96 hours following the initial incubation with differentiation media.

### RNA Isolation

RNA was isolated from C2C12 and HSkMCs utilizing the RNeasy 96 kit (Qiagen) according to the manufacturer's protocol. For mouse studies where RNA was isolated from tissue, a modified version of the manufacturer's protocol was utilized. Mice were euthanized and harvested mouse tissues were placed in 2 ml screw-cap tubes containing 0.5 mL RNAlater (Sigma-Aldrich). Tubes were placed at 4° C. for 24 hours then transferred to −80° C. for long term storage. For RNA extraction, work was performed in a fume hood. Tubes were thawed and tissue was transferred to new 2 ml screw-cap tubes containing 300-400 μL 1.4 mm ceramic beads. 0.5 mL Trizol was added to each tube followed by homogenization on a bead beater at max speed, 2× for 30 seconds. 100 μL chloroform was added and tubes were inverted 5-6 times then spun in a tabletop centrifuge, max speed for 10 minutes at 4° C. 100 μL of the aqueous layer was removed and transferred to a 2 mL deep well plate, then mixed with 150 μL 100% ethanol. 250 μL of each sample was transferred to an RNeasy 96 plate and RNA was isolated according to manufacturer instructions (Qiagen) with the exception that RW1 buffer was replaced with RWT buffer (Qiagen).

### Quantitative PCR

Nested PCR was performed on serially-diluted RNA from C2C12, HSkMCs or mouse tissues in a one-step RT-PCR reaction with primers designed to preamplify regions of interest in the mouse or human dystrophin gene using the thermal cycler conditions on a SimpliAmp thermal cycler (ThermoFisher Scientific): RT (30 minutes at 45° C., 15 seconds at 94° C.) and preamplification (15 cycles of 1 minute at 95° C., 1 minute at 55° C. and 3 minutes at 72° C.). Following pre-amplification, quantitative PCR was performed using utilizing primers (Thermofisher Scientific; IDTDNA), TaqMan probes (Thermofisher Scientific; IDTDNA) and TaqMan fast universal PCR master mix (Thermofisher scientific) on a StepOnePlus real-time PCR system (Thermofisher scientific) according to manufacturer's instructions. The primer-probe pairs were designed to specifically amplify the exon-sipped product of interest or alternatively, to quantify told dystrophin transcript. All primers and probes are listed in the tables below.

Droplet Digital PCR (ddPCR)

Isolated RNA was reverse transcribed using the High Capacity cDNA Reverse Transcription Kit (Thermo Fisher, cat #4368813) according to the manufacturer's protocol. cDNA from the reverse transcription reaction was subsequently prepared for PCR by combining primers ofinterest, a TaqMan probe and ddPCR supermix for probe (Bio-Rad, Cat #186-3024).

The PCR reaction mix was then loaded into the middle wells of a DG8TM Cartridge for a QX100 Droplet Generator (Bio-Rad) and 70 ul Droplet Generation Oil (Bio-Rad, cat #186-3005) was loaded into the bottom wells before the cartridge was placed into the droplet generator to produce about 20,000 droplets per sample. After generating droplets, each sample was transferred to a 96-well PCR plate and run on a thermal cycler with the following conditions: 95° C. for 10 minutes, 40 cycles of 95° C. for 15 seconds and 60° C. for 1 minute, then 98° C. for 10 miutes to inactivate the enzyme. Following PCR amplification, the PCR plate was read in a QX100 Droplet Reader and data was analyzed using QuantaSoft Software to count PCR-positive and PCR-negative droplets, providing absolute quantification of target DNA.

TABLE P1
Mouse Exon 23 Primers and Probes

| | | | SEQ |
|---|---|---|---|
| | | | ID |
| Name | Sequence/Assay | Catalog | NO: |
| DMD | 5′-CAGAATTCTGCCAATTGCTGAG-3′ | Custom | 219 |
| Preamplification | | | |
| Forward | | | |
| DMD | 5′-TTCTTCAGCTTGTGTCATCC-3′ | Custom | 220 |
| Preamplification | | | |
| Reverse | | | |
| Dystrophin | 5′-CTTCCGTCTCCATCAATGAACT-3′ | Mm.PT.58.9 | 221 |
| control probe | | 899092 | |
| Primer 1 | | (IDT) | |
| Dystrophin | 5′-GCTTATGTTGCCACCTCTGA-3′ | Mm.PT.58.9 | 222 |
| control probe | | 899092 | |
| Primer 2 | | (IDT) | |
| Dystrophin | 5′- | Mm.PT.58.9 | 223 |
| control probe | /HEX/CAGAGCCCC/ZEN/TATCCTTCACAGCAT/ | 899092 | |
| | 3IABkFQ/-3′ | (IDT) | |
| Probe on 22-24 | 5′-AAAGCAAACTCTCTGTACCTTATCT-3′ | Custom | 224 |
| junction Primer | | | |
| 1 | | | |
| Probe on 22-24 | 5′-CATCAACTTCAGCCATCCATTTC-3′ | Custom | 225 |
| junction Primer | | | |
| 2 | | | |
| Probe on 22-24 | 5′-/56- | Custom | 226 |
| junction Probe | FAM/TGTGATTCT/ZEN/GTAATTTCCCGAGTCTCT | | |
| | CC/3IABkFQ/-3′ | | |

TABLE P2
Human Exon 51 and 53 Primers and Probes

| | | | SEQ |
|---|---|---|---|
| | | | ID |
| Name | Sequence/Assay | Assay name | NO: |
| Preamp 51 and | 5′-GCTGCTGTGGTTATCTCCTATTA-3′ | Custom | 227 |
| 53 Forward | | | |
| Preamp 51 and | 5′-CCTATGTACATCGTTCTGCTTCT-3′ | Custom | 228 |
| 53 Reverse | | | |
| Dystrophin | 5′-GCAGTAACATTGAGCCAAGTG-3′ | Hs.PT.58.21 | 229 |
| control | | 17196.g | |
| Forward | | (IDT) | |
| Dystrophin | 5′-TCCAGTCTCATCCAGTCTAGG-3′ | Hs.PT.58.21 | 230 |
| control | | 17196.g | |
| Reverse | | (IDT) | |
| Dystrophin | 5′- | Hs.PT.58.21 | 231 |
| control probe | /HEX/AATAAGCCA/ZEN/GAGATCGAAGCGGCC/ | 17196.g | |
| | 3IABkFQ/-3′ | (IDT) | |
| Probe on 50-52 | 5′-TGAGTGGAAGGCGGTAAAC-3′ | Custom | 232 |
| junction | | | |
| Forward | | | |
| Probe on 50-52 | 5′-CGCCTCTGTTCCAAATCCT-3′ | Custom | 233 |
| junction | | | |
| Reverse | | | |
| Probe on 50-52 | 5′-/56- | Custom | 234 |
| junction Probe | FAM/CCACTATTG/ZEN/GAGCCTGCAACAATGC/ | | |
| | 3IABkFQ/-3′ | | |
| Probe on 52-54 | 5′-CCAGCAATCAAGAGGCTAGAA-3′ | Custom | 235 |
| junction | | | |
| Forward | | | |
| Probe on 52-54 | 5′-AGAAGTTTCAGGGCCAAGTC-3′ | Custom | 236 |
| junction | | | |
| Reverse | | | |
| Probe on 52-54 | 5′-/56- | Custom | 237 |
| junction Probe | FAM/TCATTACGG/ZEN/ATCGAACAGTTGGCC/ | | |
| | 3IABkFQ/-3′ | | |

### In Vivo Characterization

Wildtype C57Bl6/J mice and mdx mice were purchased from Jackson Laboratories (Bar Harbor, Me.). Following acclimatization for a minimum of 7 days, the mice were injected with a single dose of vehicle or the compound of interest via intravenous injection. 7 or 14 days following injection, the mice were euthanized by CO₂ asphyxiation followed by secondary confirmation of euthanasia via cervical dislocation. The tissues of interest were then removed and 30-300 mg placed in RNALater immediately following dissection. 24 hours later, the tissue was removed from the RNALater, blotted dry and placed into trizol in tubes containing lysing matrix D beads from MPBiomedical. The tissue was homogenized using the MPBio FastPrep-24 system and RNA isolated as described above.

### Results

### Conjugates Increase Exon Skipping in Skeletal Muscle Cells

The uptake by cells of single-stranded oligonucleotides without any delivery vehicle is inefficient, particularly in muscle cells. Provided herein are numerous long chain fatty acid (LCFA) motifs (“uptake motifs”) that improve uptake of oligonucleotides into cells. By way of example, the uptake motif DTx-01-08, improves the uptake of siRNA into cells in the absence of transfection reagent, relative to unconjugated siRNA (Table A). Briefly, human skeletal muscle cells were exposed to either PBS, an unconjugated PTEN siRNA (DT-00003) or a DTx-01-08 conjugated PTEN siRNA (DT-000146) in a free uptake experiment. 96 hours following exposure, RNA was isolated and PTEN mRNA quantified via qPCR. Compound DT-000146, the DTx-01-08-conjugated PTEN siRNA, dose-dependently repressed PTEN mRNA expression whereas neither PBS nor DT-00003, unconjugated PTEN siRNA, had any affect to repress PTEN gene expression (Table A). Other motifs provided herein similarly improve uptake of siRNA into cells.

TABLE A
Effect of DTx Conjugated siRNA in Human Skeletal
Muscle Cells in Free Uptake Assays

| | % PTEN Remaining Relative to PBS | | | | | |
|---|---|---|---|---|---|---|
| | DT-000146 | | DT-00003 | | PBS | |
| | Mean | S.E.M. | Mean | S.E.M. | Mean | S.E.M. |
| PBS | — | — | — | — | 101.2 | 9.103 |
| 10 nM | 97.35 | 3.906 | 95.94 | 5.007 | — | — |
| 30 nM | 95.03 | 5.253 | N.T. | N.T. | — | — |
| 100 nM | 84.25 | 3.513 | 110.4 | 13.47 | — | — |
| 300 nM | 59.15 | 2.724 | N.T. | N.T. | — | — |
| 1000 nM | 34.84 | 4.163 | 91.61 | 2.284 | — | — |

The ability of uptake motifs to promote siRNA activity in vivo in tissues impacted during DMD pathogenesis were evalulated by injecting the compounds listed in Table B into C57Bl6/J mice via a single intravenous injection. The siRNA utilized targeting PTEN but was distinct from the siRNA evaluated in Table A. Compounds DT-000155, DT-000175, DT-000176, DT-000177 and DT-000178 were tested in a first study. Compounds DT-000155, DT-000179, DT-000180, and DT-000183 were tested in a second study. C57Bl6/J mice were injected intravenously with a single dose of either PBS or 30 mpk of PTEN siRNA containing uptake motifs. Seven days following injection, mice were euthanized and tissues extracted and RNA isolated. Mean repression of PTEN mRNA expression was calculated from 5 replicates per treatment. Many of the compounds dose dependently inhibited PTEN mRNA expression in muscle, heart and diaphragm to a greater degree than mice treated with vehicle (PBS) (Table C₁, D1 and El for the first study; Tables C₂, D2, and E2 for the second study). These data demonstrate that uptake motifs improve the activity of siRNA in vivo.

TABLE B
Examples of Uptake Motif siRNA Conjugates

| | Uptake Domain | Linker | Conjugation Site | siRNA |
|---|---|---|---|---|
| DT-000155 | DTx-01-08 | C7 | 3′ | DTx-0038 |
| DT-000175 | DTx-01-32 | C7 | 3′ | DTx-0038 |
| DT-000176 | DTx-01-33 | C7 | 3′ | DTx-0038 |
| DT-000177 | DTx-01-50 | C7 | 3′ | DTx-0038 |
| DT-000178 | DTx-01-51 | C7 | 3′ | DTx-0038 |
| DT-000179 | DTx-01-52 | C7 | 3′ | DTx-0038 |
| DT-000180 | DTx-01-54 | C7 | 3′ | DTx-0038 |
| DT-000181 | DTx-01-55 | C7 | 3′ | DTx-0038 |
| DTx-0038 | N/A | N/A | N/A | DTx-0038 |

TABLE C-1
Knockdown of PTEN mRNA in muscle following a single intravenous
injection of DTx conjugated siRNA into C57Bl6/J mice

| | |
|---|---|
| Vehicle | 30 mg/kg |

| | Mean | SEM | Mean | SEM |
|---|---|---|---|---|
| PBS | 100.6 | 3.57 | — | — |
| DT-000155 | — | — | 48.02 | 3.50 |
| DT-000175 | — | — | 43.61 | 2.55 |
| DT-000176 | — | — | 82.93 | 3.74 |
| DT-000177 | — | — | 46.48 | 2.52 |
| DT-000178 | — | — | 47.88 | 4.84 |

TABLE C-2
Knockdown of PTEN mRNA in muscle following a single intravenous
injection of DTx conjugated siRNA into C57Bl6/J mice

| | |
|---|---|
| Vehicle | 30 mg/kg |

| | Mean | SEM | Mean | SEM |
|---|---|---|---|---|
| PBS | 100.7 | 3.813 | — | — |
| DT-000155 | — | — | 52.26 | 4.17 |
| DT-000179 | — | — | 32.45 | 5.87 |
| DT-000180 | — | — | 22.57 | 5.30 |
| DT-000181 | — | — | 51.55 | 4.32 |

TABLE D-1
Knockdown of PTEN mRNA in heart following a single intravenous
injection of DTx conjugated siRNA into C57Bl6/J mice

| | |
|---|---|
| Vehicle | 30 mg/kg |

| | Mean | S.E.M | Mean | S.E.M |
|---|---|---|---|---|
| PBS | 100.7 | 4.1 | — | — |
| DT-000155 | — | — | 65.9 | 3.273 |
| DT-000175 | — | — | 61.66 | 4.989 |
| DT-000176 | — | — | 77.18 | 3.368 |
| DT-000177 | — | — | 73.7 | 3.896 |
| DT-000178 | — | — | 75.37 | 5.772 |

TABLE D-2
Knockdown of PTEN mRNA in heart following a single intravenous
injection of DTx conjugated siRNA into C57Bl6/J mice

| | |
|---|---|
| Vehicle | 30 mg/kg |

| | Mean | SEM | Mean | SEM |
|---|---|---|---|---|
| PBS | 100.8 | 4.371 | — | — |
| DT-000155 | — | — | 65.21 | 3.964 |
| DT-000179 | — | — | 74.63 | 3.708 |
| DT-000180 | — | — | 68.57 | 9.983 |
| DT-000181 | — | — | 65.79 | 5.032 |

TABLE E-1
Knockdown of PTEN mRNA in diaphragm following
a single intravenous injection of DTx
conjugated siRNA into C57Bl6/J mice

| | |
|---|---|
| Vehicle | 30 mg/kg |

| | Mean | SEM | Mean | SEM |
|---|---|---|---|---|
| PBS | 100.5 | 3.379 | — | — |
| DT-000155 | — | — | 47.52 | 4.673 |
| DT-000175 | — | — | 45.78 | 3.941 |
| DT-000176 | — | — | 59.79 | 2.177 |
| DT-000177 | — | — | 56.8 | 1.808 |
| DT-000178 | — | — | 77.69 | 7.978 |

TABLE E-2
Knockdown of PTEN mRNA in diaphragm following
a single intravenous injection of DTx
conjugated siRNA into C57Bl6/J mice

| | |
|---|---|
| Vehicle | 30 mg/kg |

| | Mean | SEM | Mean | SEM |
|---|---|---|---|---|
| PBS | 100.4 | 3.111 | — | — |
| DT-000155 | — | — | 49.01 | 4.934 |
| DT-000179 | — | — | 54.93 | 1.821 |
| DT-000180 | — | — | 66.1 | 7.977 |
| DT-000181 | — | — | 49.25 | 1.823 |

Single-stranded oligonucleotides complementary to mouse dystrophin, designed to induce skipping of exon 23, were tested in differentiated skeletal muscle cells both as unconjugated molecules and as molecules conjugated to the DTx-01-08 uptake motif utilized above for siRNA above (Table A-E). The nucleobase sequence of the oligonucleotides tested is the same nucleobase sequence of an oligonucleotide that is complementary to annealing site M23D(+07-18) and has previously been shown to induce skipping of exon 23 of the mouse dystrophin gene (Alter et al., Nat Med. 2006, 12(2):175-177). Additionally, this previously reported oligonucleotide restores dystrophin expression and improves functional outcomes in the mdx mouse model of DMD, which carries a nonsense mutation in exon 23 that prevents expression of a functional dystrophin protein (Alter et al., Nat Med. 2006, 12(2):175-177). The oligonucleotides have the same nucleobase sequence, with variations in sugar moieties and internucleotide linkages (Table F).

TABLE F
Single-Stranded Oligonucleotides Targeting Mouse Dystrophin

| | | SEQ |
|---|---|---|
| | | ID |
| Compound | Nucleobase Sequence and Chemical Notation | NO: |
| DT-000088 | [5′-HO][Ges][Ges][mCes][mCes][Aes][Aes][Aes][mCes][mCes] | 242 |
| | [mCes][Ges][Ges][mCes][Tes][Tes][Aes][mCes][mCes][Tes][Ges] | |
| | [Aes][Aes][Aes][Te][OH-3′] | |
| DT-000092 | [5′-HO][Gms][Gms][Cms][Cms][Ams][Ams][Ams][Cms][Cms][Ums] | 243 |
| | [Cms][Gms][Gms][Cms][Ums][Ums][Ams][Cms][Cms] | |
| | [Ums][Gms][Ams][Ams][Ams][Um][OH-3′] | |
| DT-000099 | [5′-SARC][Gop][Gop][Cop][Cop][Aop] | 242 |
| | [Aop][Aop][Cop][Cop][Top][Cop][Gop][Gop][Cop][Top][Top] | |
| | [Aop][Cop][Cop][Top][Gop][Aop][Aop][Aop][To][PMO-H-3′] | |

An assay was developed in C2C12 cells to detect mouse dystrophin lacking exon 23. DT-000088 was transfected into C2C12 cells at a dose of 1000 nM. RNA was isolated, reverse transcribed and PCR performed utilizing primers to pre-amplify a dystrophin gene product containing exons 20-26 or a dystrophin gene product outside of this region (for quantification of total dystrophin). Quantitative PCR was then performed utilizing primers and a Tagman probe designed to exclusively amplify exon 23-skipped dystrophin or to amplify total dystrophyin. A dose response curve was generated by including different amounts of input RNA into the RT reaction to confirm that the assay was in the linear range. PBS and an siRNA targeting PTEN were utilized as negative controls. Linearity was established for both total dystrophin and dsytrophin lacking exon 23 (Table G). A follow up experiment was performed where increasing doses of DT-000088 were transfected into C2C12 cells. DT-000088 dose dependently increased the expression of exon-skipped 23 dystrophin whereas no product was detected following transfection with PBS or a PTEN siRNA (DT-000003) (Table H).

TABLE G
Transfection of DT-000088 into C2C12 Cells to Establish Assay Linearity

| | Dystrophin | | | Exon 23-Skipped | | |
|---|---|---|---|---|---|---|
| Input RNA [Log 2] | C_{T} [Log 2] | | | C_{T} [Log 2] | | |
| −1 | 21.17141 | | | 26.46741 | | |
| 0 | 20.31644 | Slope | −1.07 | 26.12942 | Slope | −1.07 |
| 1 | 19.24441 | r² | 0.99 | 24.77108 | r² | 0.98 |
| 2 | 18.15725 | | | 23.74967 | | |
| 3 | 17.11741 | | | 22.68581 | | |
| 4 | 15.79044 | | | 21.27438 | | |

TABLE H
Dose response transfection of DT-000088 into C2C12 Cells

% Exon-Skipped Dystrophin

| | Mean | S.E.M. |
|---|---|---|
| 0 nM (PBS) | N.D. | N.D. |
| 100 nM DT-000088 | 2.810 | 0.5551 |
| 300 nM DT-000088 | 5.035 | 1.979 |
| 1000 nM DT-000088 | 9.283 | 2.015 |
| PTEN (DT-000003) | N.D. | N.D. |

As noted above, the uptake by cells of single-stranded oligonucleotides without any delivery vehicle is inefficient, particularly in muscle cells where dystrophin is expressed. To evaluate oligonucleotides conjugated to an uptake motif, the DTx-01-08 motif was conjugated to the 3′ ends of DT-000088, DT-000092, and DT-000099, via a C7 linker (see, for example, Scheme I). As noted above, each of the oligonucleotides have the same nucleobase sequence and are designed to induce skipping of exon 23. The oligonucleotides tested are shown in Table I.

TABLE I
Conjugated Oligonucleotides Targeted to Mouse Dystrophin

| | | SEQ |
|---|---|---|
| | | ID |
| Compound | Nucleobase Sequence and Chemical Notation | NO: |
| DT-000190 | [5'-HO][Ges][Ges][mCes][mCes][Aes][Aes][Aes][mCes][mCes][Tes] | 242 |
| | [mCes][Ges][Ges][mCes][Tes][Tes][Aes][mCes][mCes][Tes][Ges] | |
| | [Aes][Aes][Aes][Teo][EN3C7][DTx-01-08] | |
| DT-000191 | [5'-HO][Gms][Gms][Cms][Cms][Ams][Ams][Ams][Cms][Cms][Ums] | 243 |
| | [Cms][Gms][Gms][Cms][Ums][Ums][Ams][Cms][Cms][Ums][Gms] | |
| | [Ams][Ams][Ams][Umo][EN3C7][DTx-01-08] | |
| DT-000187 | [5'-SARC][Gop][Gop][Cop][Cop][Aop][Aop][Aop][Cop][Cop][Top] | 242 |
| | [Cop][Gop][Gop][Cop][Top][Top][Aop][Cop][Cop][Top][Gop][Aop] | |
| | [Aop][Aop][Toa][EP3C6][DTx-01-08] | |

DT-00003, the siRNA targeted to PTEN, was used as a control. Under free uptake conditions, C2C12 cells were exposed to DT-000099, DT-000187, and DT-000088. DT-000187 increased the levels of exon-23 skipped dystrophin in a dose-dependent manner whereas DT-000099 and DT-000088 only had a very minor effect at the highest dose (Table J; “N.D.” indicates “not detected”). In this experiment, the DTx-01-08-conjugated oligonucleotide was far more potent and efficacious at generating exon 23-skipped mRNA than either unconjugated oligonucleotide. A similar free uptake experiment was performed comparing the unconjugated antisense molecules DT-000088, DTx-000099, DT-000092 and their respective DTx-01-08 conjugates DT-000190, DT-000191 and DT-0000187 (Table K; “N.D.” indicates “not detected”). In all cases, the DTx-01-08 conjugated oligonucleotides were more effective to induce the expression of exon 23-skipped dystrophin relative to the unconjugated oligonucleotides.

TABLE J
Free Uptake Experiment of DTx-Conjugates in C2C12 Cells

| | DT-000187 | | DT-000099 | | DT-000088 | | DT-000003 | |
|---|---|---|---|---|---|---|---|---|
| | Mean | S.E.M. | Mean | S.E.M. | Mean | S.E.M. | Mean | S.E.M. |
| PBS | N.D. | N.D. | N.D. | N.D. | N.D. | N.D. | N.D. | N.D. |
| 30 nM | 0.3861 | 0.2227 | N.D. | N.D. | N.D. | N.D. | N.D. | N.D. |
| 100 nM | 0.7942 | 0.3896 | N.D. | N.D. | N.D. | N.D. | N.D. | N.D. |
| 300 nM | 1.077 | 0.4474 | N.D. | N.D. | N.D. | N.D. | N.D. | N.D. |
| 1000 nM | 4.519 | 0.874 | N.D. | N.D. | N.D. | N.D. | N.D. | N.D. |
| 3000 nM | 15.58 | 5.678 | 1.374 | 0.5066 | 0.231 | 0.2302 | N.D. | N.D. |

TABLE K
Free Uptake Experiment of DTx-Conjugates in C2C12 Cells

| | Mean | S.E.M. |
|---|---|---|
| PBS | N.D. | N.D. |
| 3000 nM DT-000088 | 0.07164 | 0.005093 |
| 3000 nM DT-000191 | 0.9428 | 0.1913 |
| 3000 nM DT-000092 | N.D. | N.D. |
| 3000 nM DT-000190 | 5.709 | 0.1747 |
| 1000 nM DT-000099 | 0.01728 | 0.00694 |
| 1000 nM DT-000187 | 5.623 | 1.573 |

mdx mice, a mouse model with a frameshift mutation in exon 23, were dosed with a single intravenous injection of 30 mg/kg of either the unconjugated DT-000092 or the DTx-01-08-conjugated DT-000190. Various tissues were collected 14 days following a single dose, RNA was isolated and exon 23-skipped transcripts were quantified by droplet digital PCR. DT-000190 was more effective to induce the production of exon 23-skipped dystrophin transcripts than DT-00092 (Table L).

TABLE L
Mdx mice treated with DT-compounds

| | Exon-23 Skipped Dystrophin (Copies/75 ng) | | | | | |
|---|---|---|---|---|---|---|
| | Gastrocnemius | | Tricep | | Heart | |
| | Mean | S.E.M. | Mean | S.E.M. | Mean | S.E.M. |
| WT Mice | 0.32 | 0.196 | 0.24 | 0.147 | 0.84 | 0.2638 |
| Vehicle | 2.08 | 0.5783 | 2.2 | 0.4195 | 1.84 | 0.5154 |
| 30 mpk | 25.44 | 7.133 | 23.08 | 1.278 | 5.56 | 0.6242 |
| DT-000092 | | | | | | |
| 30 mpk | 78.52 | 14.72 | 46.48 | 7.906 | 16.64 | 2.874 |
| DT-000190 | | | | | | |

A similar set of in vitro experiments to that described above was performed to test oligonucleotides designed to induce human dystrophin exon skipping. Assays were developed to quantify transcripts lacking exon 51 or exon 53 using oligonucleotides having nucleobase sequences identical to those reported to induce exon 51 or exon 53 skipping (Echigoya et al., 2017, Mol Ther., 25(11): 2561-2572). For these assays, the following oligonucleotides were used (see also Table 4 above for full sequence and structures):
DT-000091: unconjugated oligonucleotide with 2′-O-methyl sugar modifications and phosphorothioate linkages
DT-000096: unconjugated oligonucleotide with 2′-O-methoxyethyl sugar modifications and phosphorothioate linkages

To confirm that exon 51- and exon 53-skipped dystrophin can be detected, DT-000091 and DT-000096 were transfected into primary human skeletal muscle cells at a concentration of 1000 nM. RNA was isolated, reverse transcribed and PCR performed utilizing primers to pre-amplify the regions flanking exon 51 and exon 53. Quantitative PCR was then performed utilizing primers and a Tagman probe designed to exclusively amplify either exon 51- or exon 53-skipped dystrophin. A dose response curve was generated by including different amounts of input RNA into the RT reaction to evaluate whether the assay was in the linear range. PBS and the antisense compound targeting the exon not being detected were utilized as negative controls. As expected, DT-000091 induced exon 51 skipping but did not promote exon 53 skipping (Table M) and DT-000096 induced exon 53 skipping but did not promote exon 51 skipping (Table N).

TABLE M
Quantification of Exon 51-Skipped Dystrophin
Following Transfection of DT Compounds

| | Exon-Skipped 51 Dystrophin | | | |
|---|---|---|---|---|
| | DT-000091 | | DT-000096 | |
| Input RNA [Log 2] | CT [Log 2] | S.E.M | CT [Log 2] | S.E.M |
| −1 | 19.104 | 0.1528 | 40 | 0 |
| 0 | 17.32171 | 0.0998 | 40 | 0 |
| 1 | 16.00801 | 0.1416 | 40 | 0 |
| 2 | 15.02207 | 0.1341 | 40 | 0 |
| 3 | 13.88943 | 0.0805 | 40 | 0 |
| 4 | 12.9744 | 0.0986 | 40 | 0 |

TABLE N
Quantification of Exon 53 Skipped Dystrophin
Following Transfection of DT Compounds

| | Exon-Skipped 53 Dystrophin | | | |
|---|---|---|---|---|
| | DT-000091 | | DT-000096 | |
| Input RNA [Log 2] | CT [Log 2] | S.E.M | CT [Log 2] | S.E.M |
| −1 | 40 | 0 | 29.29531 | 1.255656 |
| 0 | 40 | 0 | 27.39329 | 0.621982 |
| 1 | 40 | 0 | 25.87449 | 0.234311 |
| 2 | 40 | 0 | 24.8512 | 0.142665 |
| 3 | 40 | 0 | 23.17 | 0.428436 |
| 4 | 40 | 0 | 22.60526 | 0.215924 |

To evaluate oligonucleotides conjugated to an uptake motif, the DTx-01-08 motif was conjugated to the oligonucleotides designed to induce skipping of exon 51 or exon 53. The oligonucleotides tested were (for complete sequence and chemical notation, see Table 4 for unconjugated oligonucleotides and Table 5 for conjugated oligonucleotides):
DT-000090: exon 51; unconjugated oligonucleotide with 2′-O-methyl sugar modifications and phosphorothioate linkages
DT-000193: DTx-01-08-conjugated DT-000090
DT-000091: exon 51; unconjugated oligonucleotide with 2′-O-methoxyethyl sugar modifications and phosphorothioate linkages
DT-000194: DTx-01-08-conjugated DT-000091
DT-000094: exon 53; unconjugated oligonucleotide with morpholino moieties and phosphorodiamidite linkages
DT-000189: DTx-01-08-conjugated DT-000094
DT-000096: exon 53; unconjugated oligonucleotide with 2′-O-methoxyethyl sugar modifications and phosphorothioate linkages
DT-000192: DTx-01-08-conjugated DT-000096

The unconjugated DT-000090 and DT-00009 and their respective DTx-01-08 conjugates DT-000193 and DT-000194 were exposed to human skeletal muscle cells in free uptake assays at a concentration of 3000 nM. RNA was isolated 96 hours later and exon 51 skipped transcripts quantified. DT-000193 and DT-000194 were more effective to promote exon 51 skipping than DT-000090 and DT-000091 (Table O). Similarly, the unconjugated DT-000094 and DT-000096 and their respective DTx-01-08 conjugates DT-000189 and DT-000192 were exposed to human skeletal muscles in free uptake assays at a concentration of 3000 nM. RNA was isolated 96 hours later and exon-53 skipped transcripts quantified. DT-000189 and DT-000192 were more effective to promote exon 53 skipping than DT-000094 and DT-00096 (Table P). These data demonstrated marked improvement in exon-skipping activity upon conjugation with an uptake motif.

TABLE O
Relative Activity of DT Compounds to Promote Exon 51 Skipping
in Free Uptake experiments in Human Skeletal Muscle Cells

| | Delta C_{T} | | |
|---|---|---|---|
| | (Exon-Skipped 51 - Total Dystrophin) | | Relative |
| | Mean | S.E.M. | Difference |
| PBS | 30.7254 | 0.039 | 0.0065 |
| DT-000090 | 23.4665 | 0.874 | 1 |
| DT-000193 | 14.3406 | 0.218 | 558.7 |
| DT-000091 | 15.6949 | 0.209 | 218.5 |
| DT-000194 | 11.6010 | 0.474 | 3731.4 |

TABLE P
Relative Activity of DT Compounds to Promote Exon 53 Skipping
in Free Uptake experiments in Human Skeletal Muscle Cells

| | Delta C_{T} | | |
|---|---|---|---|
| | (Exon-Skipped 53 - Total Dystrophin) | | Relative |
| | Mean | S.E.M. | Difference |
| PBS | 29.25 | 0.113753142 | 0.09 |
| DT-000094 | 25.76 | 0.304165159 | 1.00 |
| DT-000189 | 20.11 | 0.772775905 | 50.39 |
| DT-000096 | 26.18 | 1.669980298 | 0.75 |
| DT-000192 | 16.49 | 0.16944014 | 615.95 |

Although the disclosure has been described with reference to embodiments and examples, it should be understood that numerous and various modifications can be made without departing from the spirit of the present disclosure.

What is claimed is:

1. A compound having the structure: 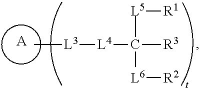
wherein
A is a single-stranded oligonucleotide having a nucleobase sequence complementary to a portion of the dystrophin pre-mRNA; t is an integer from 1 to 5;
L³ and L⁴ are independently a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
L⁵ is -L^{5A}-L^{5B}-L^{5C}-L^{5D}-L^{5E}-;
L⁶ is -L^{6A}-L^{6B}-L^{6C}-L^{6D}-L^{6E}-;
R¹ and R² are independently unsubstituted C₁-C₂₅ alkyl, wherein at least one of R¹ and R² is unsubstituted C₉-C₁₉ alkyl;
R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{5A}, L^{5B}, L^{5C}, L^{5D}, L^{5E}, L^{6A}, L^{6B}, L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; and
each R²³, R²⁴ and R²⁵ is independently hydrogen or unsubstituted C₁-C₁₀ alkyl.

2. The compound of claim 1, wherein t is 1.

3. The compound of claim 1, wherein t is 2.

4. The compound of claim 1, wherein t is 3.

5. The compound of claim 1, wherein each of R²³, R²⁴ and R²⁵ is independently hydrogen or unsubstituted C₁-C₃ alkyl.

6. The compound of claim 1, wherein one L³ is attached to a 3′ carbon of the oligonucleotide.

7. The compound of claim 1, wherein one L³ is attached to a 3′ nitrogen of the oligonucleotide.

8. The compound of claim 1, wherein one L³ is attached to a 5′ carbon of the oligonucleotide.

9. The compound of claim 1, wherein one L³ is attached to a 6′ carbon of the oligonucleotide.

10. The compound of claim 1, wherein one L³ is attached to a nucleobase of the oligonucleotide.

11. The compound of claim 1, wherein L³ and L⁴ are independently a bond, —NH—, —O—, —C(O)—, —C(O)O—, —OC(O)—, —OPO₂—O—, —O—P(O)(S)—O—, —O—P(O)(CH₃)—O—, —O—P(S)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, —P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene or substituted or unsubstituted heteroalkylene.

12. The compound of claim 1, wherein L³ is independently

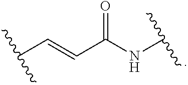 [SMILES: */C=C/C(=O)N*]

13. The compound of claim 1, wherein L³ is independently —OPO₂—O— or —OP(O)(S)—O—.

14. The compound of claim 1, wherein L³ is independently —O—.

15. The compound of claim 1, wherein L³ is independently —C(O)—.

16. The compound of claim 1, wherein L³ is independently —O—P(O)(N(CH₃)₂)—N—.

17. The compound of claim 1, wherein L⁴ is independently substituted or unsubstituted alkylene or substituted or unsubstituted heteroalkylene.

18. The compound of claim 1, wherein L⁴ is independently -L⁷-NH—C(O)— or -L⁷-C(O)—NH—, wherein L⁷ is substituted or unsubstituted alkylene.

19. The compound of claim 1, wherein L⁴ is independently

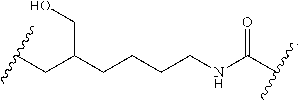 [SMILES: *CC(CO)CCCCNC(*)=O]

20. The compound of claim 1, wherein L⁴ is independently H

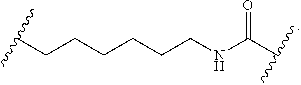 [SMILES: *CCCCCCNC(*)=O]

21. The compound of claim 1, wherein -L³-L⁴- is independently —O-L⁷-NH—C(O)— or —O-L⁷-C(O)—NH—, wherein L⁷ is independently substituted or unsubstituted alkylene, substituted or unsubstituted heteroalkylene, or substituted or unsubstituted heteroalkenylene.

22. The compound of claim 21, wherein -L³-L⁴- is independently —O-L⁷-NH—C(O)—, wherein L⁷ is independently substituted or unsubstituted C₅-C₅ alkylene.

23. The compound of claim 22, wherein -L³-L⁴- is independently

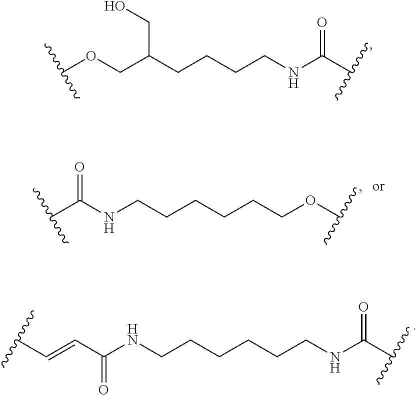 [SMILES: *OCC(CO)CCCCNC(*)=O ; *OCCCCCCNC(*)=O ; */C=C/C(=O)NCCCCCCNC(*)=O]

24. The compound of claim 1, wherein -L³-L⁴- is independently —OPO₂—O-L⁷-NH—C(O)—, —OP(O)(S)—O-L⁷-NH—C(O)—, —OPO₂—O-L⁷-C(O)—NH— or —OP(O)(S)—O-L⁷-C(O)—NH—, wherein L⁷ is independently substituted or unsubstituted alkylene.

25. The compound of claim 24, wherein -L³-L⁴- is independently —OPO₂—O-L⁷-NH—C(O)— or —OP(O)(S)—O-L⁷-NH—C(O)—, wherein L⁷ is independently substituted or unsubstituted C₅-C₅ alkylene.

26. The compound of claim 25, wherein -L³-L⁴- is independently

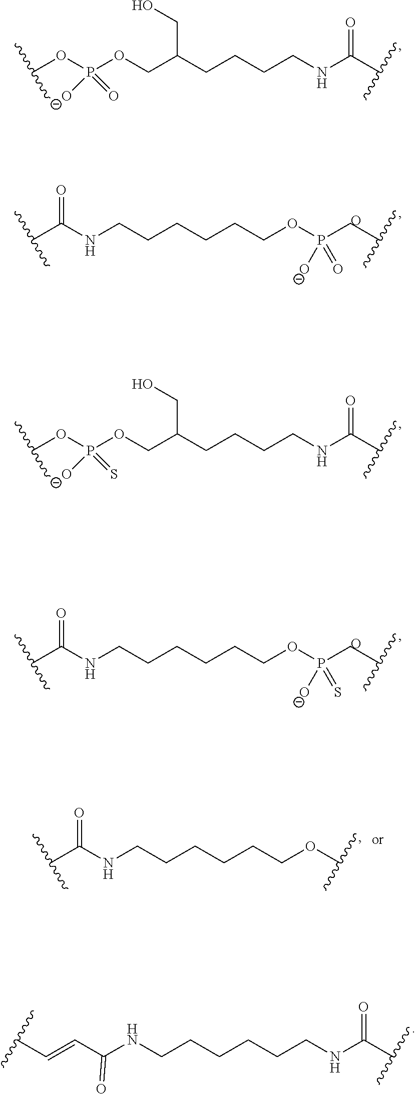

27. The compound of claim 26, wherein an -L³-L⁴- is independently 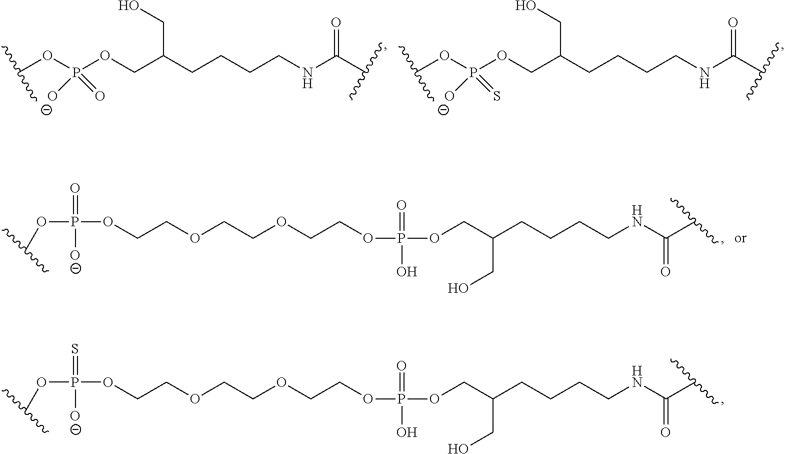 [SMILES: *OP(=O)([O-])OCC(CO)CCCCNC(*)=O ; *OP([O-])(=S)OCC(CO)CCCCNC(*)=O ; *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]
and is attached to a 3′ carbon of oligonucleotide.

28. The compound of claim 26, wherein an -L³-L⁴- is independently 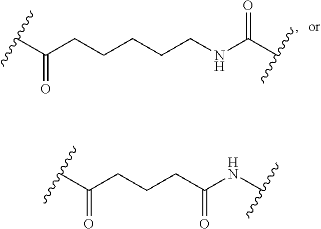 [SMILES: *C(=O)CCCCCNC(*)=O ; *NC(=O)CCCC(*)=O]
and is attached to a 3′ nitrogen of the oligonucleotide.

29. The compound of claim 26, wherein an -L³-L⁴- is independently 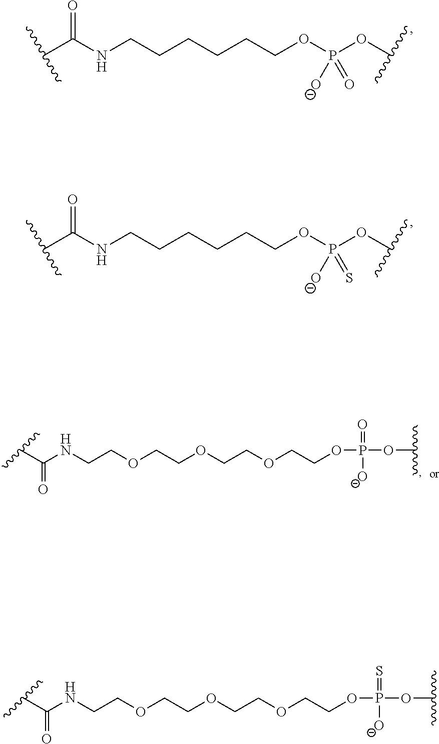 [SMILES: *OP(=O)([O-])OCCCCCCNC(*)=O ; *OP([O-])(=S)OCCCCCCNC(*)=O ; *OP(=O)([O-])OCCOCCOCCOCCNC(*)=O ; *OP([O-])(=S)OCCOCCOCCOCCNC(*)=O]
and is attached to a 5′ carbon of the oligonucleotide.

30. The compound of claim 26, wherein an -L³-L⁴- is independently 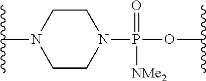 [SMILES: *OP(=O)(N(C)C)N1CCN(*)CC1]
and is attached to a 6′ carbon of the oligonucleotide.

31. The compound of claim 26, wherein an -L³-L⁴- is independently 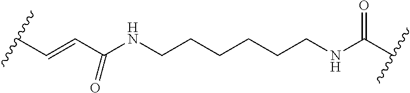 [SMILES: */C=C/C(=O)NCCCCCCNC(*)=O]
and is attached to a nucleobase of the oligonucleotide.

32. The compound of claim 1, wherein R³ is independently hydrogen.

33. The compound of claim 1, wherein L⁶ is independently —NHC(O)—, —C(O)NH—, substituted or unsubstituted alkylene, or substituted or unsubstituted heteroalkylene.

34. The compound of claim 33, wherein L⁶ is independently —NHC(O)—.

35. The compound of claim 33, wherein
L^{6A} is independently a bond or unsubstituted alkylene;
L^{6B} is independently a bond, —NHC(O)—, or unsubstituted arylene;
L^{6C} is independently a bond, unsubstituted alkylene, or unsubstituted arylene;
L^{6D} is independently a bond or unsubstituted alkylene; and
L^{6E} is independently a bond or —NHC(O)—.

36. The compound of claim 33, wherein
L⁶A is independently a bond or unsubstituted C₁-C₈ alkylene;
L⁶B is independently a bond, —NHC(O)—, or unsubstituted phenylene;
L⁶C is independently a bond, unsubstituted C₂-C₈ alkynylene, or unsubstituted phenylene;
L^{6D} is independently a bond or unsubstituted C₁-C₈ alkylene; and
L^{6E} is independently a bond or —NHC(O)—.

37. The compound of claim 1, wherein L⁶ is independently a bond,

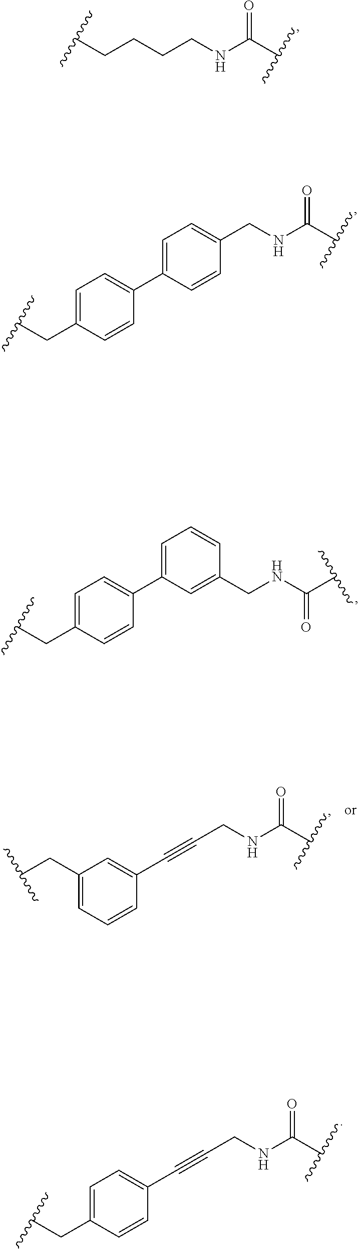 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

38. The compound of claim 1, wherein L⁵ is independently —NHC(O)—, —C(O)NH—, substituted or unsubstituted alkylene, or substituted or unsubstituted heteroalkylene.

39. The compound of claim 1, wherein L⁵ is independently —NHC(O)—.

40. The compound of claim 1, wherein
L^{5A} is independently a bond or unsubstituted alkylene;
L^{5B} is independently a bond, —NHC(O)—, or unsubstituted arylene;
L^{5C} is independently a bond, unsubstituted alkylene, or unsubstituted arylene;
L^{5D} is independently a bond or unsubstituted alkylene; and
L^{5E} is independently a bond or —NHC(O)—.

41. The compound of claim 1, wherein
L^{5A} is independently a bond or unsubstituted C₁-C₈ alkylene;
L^{5B} is independently a bond, —NHC(O)—, or unsubstituted phenylene;
L^{5C} is independently a bond, unsubstituted C₂-C₈ alkynylene, or unsubstituted phenylene;
L^{5D} is independently a bond or unsubstituted C₁-C₈ alkylene; and
L^{5E} is independently a bond or —NHC(O)—.

42. The compound of claim 1, wherein L⁵ is independently a bond,

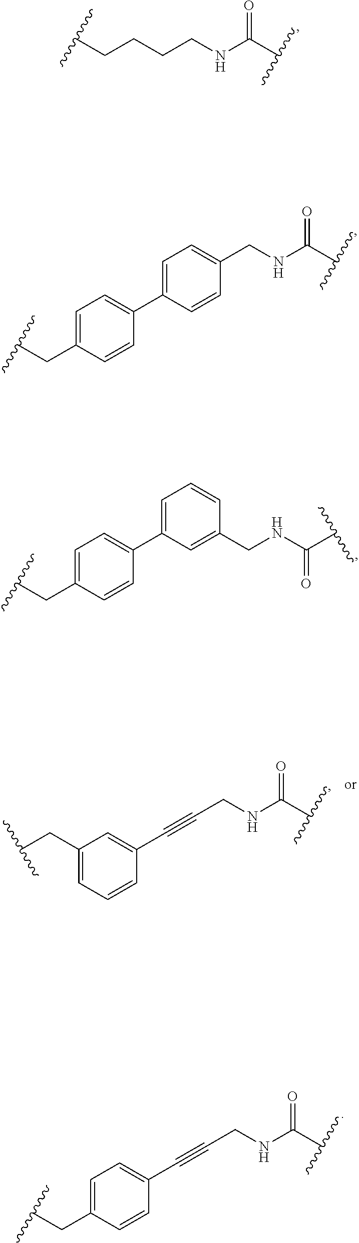 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

43. The compound of claim 1, wherein R¹ is unsubstituted C₁-C₁₇ alkyl.

44. The compound of claim 1, wherein R¹ is unsubstituted C₁₁-C₁₇ alkyl.

45. The compound of claim 1, wherein R¹ is unsubstituted C₁₃-C₁₇ alkyl.

46. The compound of claim 1, wherein R¹ is unsubstituted C₁₄-C₁₅ alkyl.

47. The compound of claim 1, wherein R¹ is unsubstituted unbranched C₁-C₁₇ alkyl.

48. The compound of claim 1, wherein R¹ is unsubstituted unbranched C₁₁-C₁₇ alkyl.

49. The compound of claim 1, wherein R¹ is unsubstituted unbranched C₁₃-C₁₇ alkyl.

50. The compound of claim 1, wherein R¹ is unsubstituted unbranched C₁₄-C₁₅ alkyl.

51. The compound of claim 1, wherein R¹ is unsubstituted unbranched saturated C₁-C₁₇ alkyl.

52. The compound of claim 1, wherein R¹ is unsubstituted unbranched saturated C₁₁-C₁₇ alkyl.

53. The compound of claim 1, wherein R¹ is unsubstituted unbranched saturated C₁₃-C₁₇ alkyl.

54. The compound of claim 1, wherein R¹ is unsubstituted unbranched saturated C₁₄-C₁₅ alkyl.

55. The compound of claim 1, wherein R² is unsubstituted C₁-C₁₇ alkyl.

56. The compound of claim 1, wherein R² is unsubstituted C₁₁-C₁₇ alkyl.

57. The compound of claim 1, wherein R² is unsubstituted C₁₃-C₁₇ alkyl.

58. The compound of claim 1, wherein R² is unsubstituted C₁₄-C₁₅ alkyl.

59. The compound of claim 1, wherein R² is unsubstituted unbranched C₁-C₁₇ alkyl.

60. The compound of claim 1, wherein R² is unsubstituted unbranched C₁₁-C₁₇ alkyl.

61. The compound of claim 1, wherein R² is unsubstituted unbranched C₁₃-C₁₇ alkyl.

62. The compound of claim 1, wherein R² is unsubstituted unbranched C₁₄-C₁₅ alkyl.

63. The compound of claim 1, wherein R² is unsubstituted unbranched saturated C₁-C₁₇ alkyl.

64. The compound of claim 1, wherein R² is unsubstituted unbranched saturated C₁₁-C₁₇ alkyl.

65. The compound of claim 1, wherein R² is unsubstituted unbranched saturated C₁₃-C₁₇ alkyl.

66. The compound of claim 1, wherein R² is unsubstituted unbranched saturated C₁₄-C₁₅ alkyl.

67. The compound of claim 1, wherein the single-stranded oligonucleotide induces skipping of an exon of the dystrophin pre-mRNA.

68. The compound of claim 67, wherein the nucleobase sequence of the single-stranded oligonucleotide is complementary to a splice donor site, a splice acceptor site, an exonic splicing enhancer (ESE), a splicing branch point, an exon recognition sequence, or a splice enhancer of the dystrophin pre-mRNA.

69. The compound of claim 67, wherein the nucleobase sequence of the single-stranded oligonucleotide is complementary to an annealing site selected from Table 2.

70. The compound of claim 1, wherein the single-stranded oligonucleotide is 25 to 30 nucleotides in length.

71. The compound of claim 1, wherein the nucleobase sequence of the single-stranded oligonucleotide is selected from a nucleobase sequence in Table 2.

72. The compound of claim 71, wherein the nucleobase sequence of the single-stranded oligonucleotide is 5′-CUCCAACAUCAAGGAAGAUGGCAUUUCUAG-3′ (SEQ ID NO: 1).

73. The compound of claim 71, wherein the nucleobase sequence of the single-stranded oligonucleotide is 5′-GUUGCCUCCGGUUCUGAAGGUGUUC-3′ (SEQ ID NO: 2).

74. The compound of claim 71, wherein the nucleobase sequence of the single-stranded oligonucleotide is 5′-CAATGCCATCCTGGAGTTCCTG-3′ (SEQ ID NO: 3).

75. The compound of claim 71, wherein the nucleobase sequence of the single-stranded oligonucleotide is 5′-UCAAGGAAGAUGGCAUUUCU-3′ (SEQ ID NO: 4).

76. The compound of claim 71, wherein the nucleobase sequence of the single-stranded oligonucleotide is 5′-CGCTGCCCAATGCCAUCC-3′ (SEQ ID NO: 5).

77. The compound of claim 71, wherein the nucleobase sequence of the single-stranded oligonucleotide is 5′-CCTCCGGTTCTGAAGGTGTTC-3′ (SEQ ID NO: 6).

78. The compound of any one of claims 1 to 77, wherein the single-stranded oligonucleotide comprises one or more modified sugar moieties.

79. The compound of claim 1, wherein each nucleotide of the single-stranded oligonucleotide comprises a modified sugar moiety.

80. The compound of claim 79, wherein the modified sugar moiety comprises a 2′ modification.

81. The compound of claim 79, wherein the 2′-modification is selected from a 2′-fluoro modification, a 2′-O-methyl modification, a 2′-O-methoxyethyl modification, and a bicyclic sugar modification.

82. The compound of claim 81, wherein the bicyclic sugar modification is selected from a 4′-CH(CH₃)—O-2′ linkage, a 4′-(CH₂)₂—O-2′ linkage, a 4′-CH(CH₃)—O-2′ linkage, a 4′-CH(CH₂—OMe)-O-2′ linkage, a 4′-CH(CH₂)—N(H)—O-2′ linkage, or a 5′-CH(CH₂)—N(CH₃)—O-2′.

83. The compound of claim 78, wherein the modified sugar moiety is an unlocked sugar modification.

84. The compound of claim 78, wherein the modified sugar moiety is a morpholino moiety.

85. The compound of claim 1, wherein the single-stranded oligonucleotide comprises one or more modified internucleotide linkages.

86. The compound of claim 85, wherein the modified internucleotide linkage selected from a phosphorothioate linkage and a phosphorodiamidite linkage.

87. The compound of any one of claims 1 to 86, wherein each internucleotide linkage of the single-stranded oligonucleotide is a chirally controlled internucleotide linkage.

88. The compound of claim 87, wherein the single-stranded oligonucleotide comprises a plurality of internucleotide linkages of the Sp conformation.

89. The compound of claim 87, wherein the single-stranded oligonucleotide comprises a plurality of internucleotide linkages of the Rp conformation.

90. The compound of claim 72, wherein each nucleotide of the single-stranded nucleotide comprises a morpholino moiety, and wherein each morpholino moiety is linked by a phosphorodiamidite linkage.

91. The compound of claim 72, wherein each nucleotide of the single-stranded nucleotide comprises a 2′-O-methoxyethyl sugar modification, and wherein each internucleotide linkage is a phosphorothioate linkage.

92. The compound of claim 72, wherein each nucleotide of the single-stranded oligonucleotide comprises a 2′-O-methyl sugar modification, and wherein each internucleotide linkage is a phosphorothioate linkage.

93. The compound of claim 73, wherein each nucleotide of the single-stranded nucleotide comprises a 2′-O-methoxyethyl sugar modification, and wherein each internucleotide linkage is a phosphorothioate linkage.

94. The compound of claim 73, wherein each nucleotide of the single-stranded oligonucleotide comprises a 2′-O-methyl sugar modification, and wherein each internucleotide linkage is a phosphorothioate linkage.

95. The compound of claim 74, wherein each nucleotide of the single-stranded nucleotide comprises a 2′-O-methoxyethyl sugar modification, and wherein each internucleotide linkage is a phosphorothioate linkage.

96. The compound of claim 74, wherein each nucleotide of the single-stranded oligonucleotide comprises a 2′-O-methyl sugar modification, and wherein each internucleotide linkage is a phosphorothioate linkage.

97. The compound of claim 75, wherein each nucleotide of the single-stranded oligonucleotide comprises a 2′-O-methyl sugar modification, and wherein each internucleotide linkage is a phosphorothioate linkage.

98. The compound of claim 75, wherein each nucleotide of the single-stranded nucleotide comprises a 2′-O-methoxyethyl sugar modification, and wherein each internucleotide linkage is a phosphorothioate linkage.

99. The compound of claim 75, wherein each nucleotide of the single-stranded oligonucleotide comprises a morpholino moiety, and wherein each morpholino moiety is linked by a phosphorodiatmite linkage.

100. The compound of claim 75, wherein each nucleotide of the single-stranded oligonucleotide comprises a 2′-modification, wherein the 2′ modification is a 2′-O-methyl modification or a 2′-fluoro modification, and wherein each internucleotide linkage is selected from a phosphorothoiate linkage of the Sp conformation and a phosphodiester linkage.

101. The compound of claim 76, wherein each nucleotide of the single-stranded oligonucleotide comprises a 2′ modification, wherein the 2′ modification is a 2′-O-methyl modification or a bicyclic sugar modification with a 4′-(CH₂)₂—O-2′ linkage, and wherein each linkage is a phosphodiester linkage.

102. The compound of claim 77, wherein each nucleotide comprises a morpholino moiety, and wherein each morpholino moiety is linked by a phosphorodiamidite linkage.

103. The compound of claim 1, wherein the single-stranded oligonucleotide is a structure selected from a structure in Table 4.

104. A method comprising contacting a cell with a compound of one of claims 1 to 103.

105. The method of claim 104, wherein the contacting occurs in vitro.

106. The method of claim 104, wherein the contacting occurs in vivo.

107. A method of inducing skipping of an exon of the dystrophin pre-mRNA in a cell, comprising contacting the cell with a compound of one of claims 1 to 103.

108. The method of claim 107, wherein the cell is in vitro.

109. The method of claim 107, wherein the cell is in vivo.

110. A method comprising administering to a subject in need thereof the compound of claim 1.

111. A method of treating Duchenne muscular dystrophy, comprising administering to a subject in need thereof the compound of claim 1.

112. The method of claim 110 or 111, wherein the subject is determined to have a mutation in the dystrophin gene that is amenable to exon skipping.

113. The method of claim 107 to 112, wherein one or more exons of the pre-mRNA of dystrophin is skipped at an increased level relative to the absence of the compound.

114. A compound of any of claim 1, for use in therapy.

115. A compound of claim 1, for use in the preparation of a medicament.

116. A pharmaceutical composition comprising a pharmaceutically acceptable excipient and the compound of claim 1.


SEQUENCE LISTING (243 sequences)

SEQ ID NO: 1 - RNA, 30 nt, Artificial Sequence
  Oligonucleotide
cuccaacaucaaggaagauggcauuucuag

SEQ ID NO: 2 - RNA, 25 nt, Artificial Sequence
  Oligonucleotide
guugccuccgguucugaagguguuc

SEQ ID NO: 3 - RNA, 16 nt, Artificial Sequence
  Oligonucleotide
caagccaccggagccg

SEQ ID NO: 4 - RNA, 20 nt, Artificial Sequence
  Oligonucleotide
ucaaggaagauggcauuucu

SEQ ID NO: 5 - RNA, 16 nt, Artificial Sequence
  Oligonucleotide
cgcgcccaagccaucc

SEQ ID NO: 6 - RNA, 14 nt, Artificial Sequence
  Oligonucleotide
ccccggcgaagggc

SEQ ID NO: 7 - RNA, 20 nt, Artificial Sequence
  Oligonucleotide
uuagaaaucucuccuugugc

SEQ ID NO: 8 - RNA, 22 nt, Artificial Sequence
  Oligonucleotide
uccucagcagaaagaagccacg

SEQ ID NO: 9 - RNA, 20 nt, Artificial Sequence
  Oligonucleotide
uaaauuggguguuacacaau

SEQ ID NO: 10 - RNA, 23 nt, Artificial Sequence
  Oligonucleotide
cuugaauuuaggagauucaucug

SEQ ID NO: 11 - RNA, 23 nt, Artificial Sequence
  Oligonucleotide
caucuucugauaauuuuccuguu

SEQ ID NO: 12 - RNA, 20 nt, Artificial Sequence
  Oligonucleotide
aggacuuacuugcuuuguuu

SEQ ID NO: 13 - RNA, 24 nt, Artificial Sequence
  Oligonucleotide
cccugaggcauucccaucuugaau

SEQ ID NO: 14 - RNA, 20 nt, Artificial Sequence
  Oligonucleotide
uucuggagauccauuaaaac

SEQ ID NO: 15 - RNA, 24 nt, Artificial Sequence
  Oligonucleotide
ucuucuguuuuuguuagccaguca

SEQ ID NO: 16 - RNA, 20 nt, Artificial Sequence
  Oligonucleotide
ucuauguaaacugaaaauuu

SEQ ID NO: 17 - RNA, 21 nt, Artificial Sequence
  Oligonucleotide
uucaucaacuaccaccaccau

SEQ ID NO: 18 - RNA, 24 nt, Artificial Sequence
  Oligonucleotide
cagcaguugcgugaucuccacuag

SEQ ID NO: 19 - RNA, 25 nt, Artificial Sequence
  Oligonucleotide
cuaagcaaaauaaucugaccuuaag

SEQ ID NO: 20 - RNA, 22 nt, Artificial Sequence
  Oligonucleotide
caucuacagauguuugcccauc

SEQ ID NO: 21 - RNA, 28 nt, Artificial Sequence
  Oligonucleotide
cuuguaaaagaacccagcggucuucugu

SEQ ID NO: 22 - RNA, 23 nt, Artificial Sequence
  Oligonucleotide
gaaggaugucuuguaaaagaacc

SEQ ID NO: 23 - RNA, 20 nt, Artificial Sequence
  Oligonucleotide
cauuugagaaggaugucuug

SEQ ID NO: 24 - RNA, 24 nt, Artificial Sequence
  Oligonucleotide
aucucccaauaccuggagaagaga

SEQ ID NO: 25 - RNA, 24 nt, Artificial Sequence
  Oligonucleotide
caugacacaccuguucuucaguaa

SEQ ID NO: 26 - RNA, 20 nt, Artificial Sequence
  Oligonucleotide
accuguucuucaguaagacg

SEQ ID NO: 27 - RNA, 21 nt, Artificial Sequence
  Oligonucleotide
uuucugaaagccaugcacuaa

SEQ ID NO: 28 - RNA, 24 nt, Artificial Sequence
  Oligonucleotide
ucuuuaaagccaguugugugaauc

SEQ ID NO: 29 - RNA, 31 nt, Artificial Sequence
  Oligonucleotide
gccaugcacuaaaaaggcacugcaagacauu

SEQ ID NO: 30 - RNA, 25 nt, Artificial Sequence
  Oligonucleotide
guacauacggccaguuuuugaagac

SEQ ID NO: 31 - RNA, 22 nt, Artificial Sequence
  Oligonucleotide
guuauccagccaugcuuccguc

SEQ ID NO: 32 - RNA, 26 nt, Artificial Sequence
  Oligonucleotide
uggauugcuuuuucuuuucuagaucc

SEQ ID NO: 33 - RNA, 23 nt, Artificial Sequence
  Oligonucleotide
gaucuuguuugagugaauacagu

SEQ ID NO: 34 - RNA, 23 nt, Artificial Sequence
  Oligonucleotide
ccgucuucugggucacugacuua

SEQ ID NO: 35 - RNA, 25 nt, Artificial Sequence
  Oligonucleotide
caugcuuccgucuucugggucacug

SEQ ID NO: 36 - RNA, 25 nt, Artificial Sequence
  Oligonucleotide
cuagauccgcuuuuaaaaccuguua

SEQ ID NO: 37 - RNA, 31 nt, Artificial Sequence
  Oligonucleotide
ucuuuucuagauccgcuuuuaaaaccuguua

SEQ ID NO: 38 - RNA, 20 nt, Artificial Sequence
  Oligonucleotide
ccgcuuuuaaaaccuguuaa

SEQ ID NO: 39 - RNA, 26 nt, Artificial Sequence
  Oligonucleotide
cuagauccgcuuuuaaaaccuguuaa

SEQ ID NO: 40 - RNA, 31 nt, Artificial Sequence
  Oligonucleotide
cuagauccgcuuuuaaaaccuguuaaaacaa

SEQ ID NO: 41 - RNA, 25 nt, Artificial Sequence
  Oligonucleotide
ugauaauugguaucacuaaccugug

SEQ ID NO: 42 - RNA, 22 nt, Artificial Sequence
  Oligonucleotide
guaucacuaaccugugcuguac

SEQ ID NO: 43 - RNA, 20 nt, Artificial Sequence
  Oligonucleotide
cagcaguaguugucaucugc

SEQ ID NO: 44 - RNA, 31 nt, Artificial Sequence
  Oligonucleotide
gccugagcugaucugcuggcaucuugcaguu

SEQ ID NO: 45 - RNA, 19 nt, Artificial Sequence
  Oligonucleotide
cugcuggcaucuugcaguu

SEQ ID NO: 46 - RNA, 22 nt, Artificial Sequence
  Oligonucleotide
cagcaguaguugucaucugcuc

SEQ ID NO: 47 - RNA, 25 nt, Artificial Sequence
  Oligonucleotide
aucugcauuaacacccucuagaaag

SEQ ID NO: 48 - RNA, 24 nt, Artificial Sequence
  Oligonucleotide
ccggcuguucaguuguucugaggc

SEQ ID NO: 49 - RNA, 20 nt, Artificial Sequence
  Oligonucleotide
auucgauccaccggcuguuc

SEQ ID NO: 50 - RNA, 28 nt, Artificial Sequence
  Oligonucleotide
cuggcagaauucgauccaccggcuguuc

SEQ ID NO: 51 - RNA, 24 nt, Artificial Sequence
  Oligonucleotide
ccggcuguucaguuguucugaggc

SEQ ID NO: 52 - RNA, 28 nt, Artificial Sequence
  Oligonucleotide
aucugcauuaacacccucuagaaagaaa

SEQ ID NO: 53 - RNA, 28 nt, Artificial Sequence
  Oligonucleotide
gaaggagaagagauucuuaccuuacaaa

SEQ ID NO: 54 - RNA, 24 nt, Artificial Sequence
  Oligonucleotide
guugaagaucugauagccgguuga

SEQ ID NO: 55 - RNA, 22 nt, Artificial Sequence
  Oligonucleotide
cugcauccaggaacaugggucc

SEQ ID NO: 56 - RNA, 23 nt, Artificial Sequence
  Oligonucleotide
gucugcauccaggaacauggguc

SEQ ID NO: 57 - RNA, 22 nt, Artificial Sequence
  Oligonucleotide
gccgguugacuucauccugugc

SEQ ID NO: 58 - RNA, 24 nt, Artificial Sequence
  Oligonucleotide
uacuuacugucuguagcucuuucu

SEQ ID NO: 59 - RNA, 22 nt, Artificial Sequence
  Oligonucleotide
cugcaauuccccgagucucugc

SEQ ID NO: 60 - RNA, 24 nt, Artificial Sequence
  Oligonucleotide
cacucauggucuccugauagcgca

SEQ ID NO: 61 - RNA, 23 nt, Artificial Sequence
  Oligonucleotide
acugcuggacccauguccugaug

SEQ ID NO: 62 - RNA, 21 nt, Artificial Sequence
  Oligonucleotide
cuaaguugagguauggagagu

SEQ ID NO: 63 - RNA, 23 nt, Artificial Sequence
  Oligonucleotide
uauucacagaccugcaauucccc

SEQ ID NO: 64 - RNA, 22 nt, Artificial Sequence
  Oligonucleotide
uaggccacuuuguugcucuugc

SEQ ID NO: 65 - RNA, 26 nt, Artificial Sequence
  Oligonucleotide
acaguggugcugagauaguauaggcc

SEQ ID NO: 66 - RNA, 19 nt, Artificial Sequence
  Oligonucleotide
uucagagggcgcuuucuuc

SEQ ID NO: 67 - RNA, 23 nt, Artificial Sequence
  Oligonucleotide
gggcaggccauuccuccuucaga

SEQ ID NO: 68 - RNA, 26 nt, Artificial Sequence
  Oligonucleotide
cgggcugaauugucugaauaucacug

SEQ ID NO: 69 - RNA, 24 nt, Artificial Sequence
  Oligonucleotide
ucuucaggguuuguaugugauucu

SEQ ID NO: 70 - RNA, 26 nt, Artificial Sequence
  Oligonucleotide
cuguuggcacaugugaucccacugag

SEQ ID NO: 71 - RNA, 24 nt, Artificial Sequence
  Oligonucleotide
gucuauaccuguuggcacauguga

SEQ ID NO: 72 - RNA, 25 nt, Artificial Sequence
  Oligonucleotide
ugcuuucuguaauucaucuggaguu

SEQ ID NO: 73 - RNA, 25 nt, Artificial Sequence
  Oligonucleotide
ugugucauccauucgugcaucucug

SEQ ID NO: 74 - RNA, 26 nt, Artificial Sequence
  Oligonucleotide
ccuccuuucuggcauagaccuuccac

SEQ ID NO: 75 - RNA, 25 nt, Artificial Sequence
  Oligonucleotide
uuaaggccucuugugcuacaggugg

SEQ ID NO: 76 - RNA, 23 nt, Artificial Sequence
  Oligonucleotide
gggccucuucuuuagcucucuga

SEQ ID NO: 77 - RNA, 22 nt, Artificial Sequence
  Oligonucleotide
gacuuccaaagucuugcauuuc

SEQ ID NO: 78 - RNA, 26 nt, Artificial Sequence
  Oligonucleotide
cagagauuuccucagcuccgccagga

SEQ ID NO: 79 - RNA, 24 nt, Artificial Sequence
  Oligonucleotide
gccaacaugcccaaacuuccuaag

SEQ ID NO: 80 - RNA, 21 nt, Artificial Sequence
  Oligonucleotide
cuuacaucuagcaccucagag

SEQ ID NO: 81 - RNA, 25 nt, Artificial Sequence
  Oligonucleotide
auuuggguuauccucugaaugucgc

SEQ ID NO: 82 - RNA, 25 nt, Artificial Sequence
  Oligonucleotide
uccgccaucuguuagggucugugcc

SEQ ID NO: 83 - RNA, 22 nt, Artificial Sequence
  Oligonucleotide
cauaccucuucauguaguucuc

SEQ ID NO: 84 - RNA, 26 nt, Artificial Sequence
  Oligonucleotide
cauuugagcugcguccaccuugucug

SEQ ID NO: 85 - RNA, 26 nt, Artificial Sequence
  Oligonucleotide
uccugggcagacuggaugcucuguuc

SEQ ID NO: 86 - RNA, 23 nt, Artificial Sequence
  Oligonucleotide
uugccugggcuuccugaggcauu

SEQ ID NO: 87 - RNA, 21 nt, Artificial Sequence
  Oligonucleotide
gacuugucaaaucagauugga

SEQ ID NO: 88 - RNA, 25 nt, Artificial Sequence
  Oligonucleotide
uaguuucugaaauaacauauaccug

SEQ ID NO: 89 - RNA, 24 nt, Artificial Sequence
  Oligonucleotide
guuucugaaauaacauauaccugu

SEQ ID NO: 90 - RNA, 24 nt, Artificial Sequence
  Oligonucleotide
uucugaaauaacauauaccugugc

SEQ ID NO: 91 - RNA, 23 nt, Artificial Sequence
  Oligonucleotide
cgaaacuucauggagacaucuug

SEQ ID NO: 92 - RNA, 20 nt, Artificial Sequence
  Oligonucleotide
caaugauuuagcugugacug

SEQ ID NO: 93 - RNA, 25 nt, Artificial Sequence
  Oligonucleotide
cuuguagacgcugcucaaaauuggc

SEQ ID NO: 94 - RNA, 20 nt, Artificial Sequence
  Oligonucleotide
caccagaaauacauaccaca

SEQ ID NO: 95 - RNA, 27 nt, Artificial Sequence
  Oligonucleotide
gucuuuaucaccauuuccacuucagac

SEQ ID NO: 96 - RNA, 24 nt, Artificial Sequence
  Oligonucleotide
ucuguacaaucugacguccagucu

SEQ ID NO: 97 - RNA, 25 nt, Artificial Sequence
  Oligonucleotide
ccgucugcuuuuucuguacaaucug

SEQ ID NO: 98 - RNA, 20 nt, Artificial Sequence
  Oligonucleotide
caugcacacaccuuugcucc

SEQ ID NO: 99 - RNA, 23 nt, Artificial Sequence
  Oligonucleotide
ccaggcaacuucagaauccaaau

SEQ ID NO: 100 - RNA, 25 nt, Artificial Sequence
  Oligonucleotide
cauucauuuccuuucgcaucuuacg

SEQ ID NO: 101 - RNA, 26 nt, Artificial Sequence
  Oligonucleotide
cuguagcugccagccauucugucaag

SEQ ID NO: 102 - RNA, 22 nt, Artificial Sequence
  Oligonucleotide
uccauaucuguagcugccagcc

SEQ ID NO: 103 - RNA, 26 nt, Artificial Sequence
  Oligonucleotide
ugaucucuuugucaauuccauaucug

SEQ ID NO: 104 - RNA, 30 nt, Artificial Sequence
  Oligonucleotide
uuucuguuaccugaaaagaauuauaaugaa

SEQ ID NO: 105 - RNA, 30 nt, Artificial Sequence
  Oligonucleotide
uucagugauauagguuuuaccuuuccccag

SEQ ID NO: 106 - RNA, 20 nt, Artificial Sequence
  Oligonucleotide
ccaguuacuauucagaagac

SEQ ID NO: 107 - RNA, 21 nt, Artificial Sequence
  Oligonucleotide
ucuucugcucgggaggugaca

SEQ ID NO: 108 - RNA, 20 nt, Artificial Sequence
  Oligonucleotide
ucuucaggugcaccuucugu

SEQ ID NO: 109 - RNA, 25 nt, Artificial Sequence
  Oligonucleotide
ugugaugugguccacauucugguca

SEQ ID NO: 110 - RNA, 20 nt, Artificial Sequence
  Oligonucleotide
ccauguguuucugguauucc

SEQ ID NO: 111 - RNA, 24 nt, Artificial Sequence
  Oligonucleotide
uucugugugaaauggcugcaaauc

SEQ ID NO: 112 - RNA, 25 nt, Artificial Sequence
  Oligonucleotide
cguguagaguccaccuuugggcgua

SEQ ID NO: 113 - RNA, 24 nt, Artificial Sequence
  Oligonucleotide
uacuaauuuccugcaguggucacc

SEQ ID NO: 114 - RNA, 25 nt, Artificial Sequence
  Oligonucleotide
ugcugaauuucagccuccagugguu

SEQ ID NO: 115 - RNA, 25 nt, Artificial Sequence
  Oligonucleotide
ugaagucuuccucuuucagauucac

SEQ ID NO: 116 - RNA, 20 nt, Artificial Sequence
  Oligonucleotide
ccuucaaaggaauggaggcc

SEQ ID NO: 117 - RNA, 20 nt, Artificial Sequence
  Oligonucleotide
uugucuguaacagcugcugu

SEQ ID NO: 118 - RNA, 20 nt, Artificial Sequence
  Oligonucleotide
guuguaaguugucuccucuu

SEQ ID NO: 119 - RNA, 24 nt, Artificial Sequence
  Oligonucleotide
cuggcuuucucucaucugugauuc

SEQ ID NO: 120 - RNA, 20 nt, Artificial Sequence
  Oligonucleotide
gcucuaauaccuugagagca

SEQ ID NO: 121 - RNA, 21 nt, Artificial Sequence
  Oligonucleotide
cucccauccuguaggucacug

SEQ ID NO: 122 - RNA, 25 nt, Artificial Sequence
  Oligonucleotide
gcgccucccauccuguaggucacug

SEQ ID NO: 123 - RNA, 31 nt, Artificial Sequence
  Oligonucleotide
uaggaggcgccucccauccuguaggucacug

SEQ ID NO: 124 - RNA, 31 nt, Artificial Sequence
  Oligonucleotide
aggucuaggaggcgccucccauccuguaggu

SEQ ID NO: 125 - RNA, 25 nt, Artificial Sequence
  Oligonucleotide
cuaggaggcgccucccauccuguag

SEQ ID NO: 126 - RNA, 26 nt, Artificial Sequence
  Oligonucleotide
ucaauaugcugcuucccaaacugaaa

SEQ ID NO: 127 - RNA, 22 nt, Artificial Sequence
  Oligonucleotide
uaccaguuuuugcccugucagg

SEQ ID NO: 128 - RNA, 26 nt, Artificial Sequence
  Oligonucleotide
cuucgaggaggucuaggaggcgccuc

SEQ ID NO: 129 - RNA, 25 nt, Artificial Sequence
  Oligonucleotide
cuuuauuuuccuuucaucucugggc

SEQ ID NO: 130 - RNA, 20 nt, Artificial Sequence
  Oligonucleotide
cuuugagaccucaaauccuu

SEQ ID NO: 131 - RNA, 24 nt, Artificial Sequence
  Oligonucleotide
gggcuugugagacaugagugauuu

SEQ ID NO: 132 - RNA, 27 nt, Artificial Sequence
  Oligonucleotide
aucguuucuucacggacagugugcugg

SEQ ID NO: 133 - RNA, 22 nt, Artificial Sequence
  Oligonucleotide
accuucagaggacuccucuugc

SEQ ID NO: 134 - RNA, 20 nt, Artificial Sequence
  Oligonucleotide
ggagagagcuuccuguagcu

SEQ ID NO: 135 - RNA, 23 nt, Artificial Sequence
  Oligonucleotide
ucacccuuuccacaggcguugca

SEQ ID NO: 136 - RNA, 25 nt, Artificial Sequence
  Oligonucleotide
uauguguuaccuacccuugucgguc

SEQ ID NO: 137 - RNA, 20 nt, Artificial Sequence
  Oligonucleotide
uuugugucuuucugagaaac

SEQ ID NO: 138 - RNA, 20 nt, Artificial Sequence
  Oligonucleotide
aucugucaaaucgccugcag

SEQ ID NO: 139 - RNA, 20 nt, Artificial Sequence
  Oligonucleotide
aaagacuuaccuuaagauac

SEQ ID NO: 140 - RNA, 27 nt, Artificial Sequence
  Oligonucleotide
ugcagaccuccugccaccgcagauuca

SEQ ID NO: 141 - RNA, 20 nt, Artificial Sequence
  Oligonucleotide
uguuuuugaggauugcugaa

SEQ ID NO: 142 - RNA, 20 nt, Artificial Sequence
  Oligonucleotide
uccuguagaauacuggcauc

SEQ ID NO: 143 - RNA, 26 nt, Artificial Sequence
  Oligonucleotide
ccaaugccauccuggaguuccuguaa

SEQ ID NO: 144 - RNA, 20 nt, Artificial Sequence
  Oligonucleotide
cuaccucuuuuuucugucug

SEQ ID NO: 145 - RNA, 31 nt, Artificial Sequence
  Oligonucleotide
caagcuuuucuuuuaguugcugcucuuuucc

SEQ ID NO: 146 - RNA, 28 nt, Artificial Sequence
  Oligonucleotide
cugcuuccuccaaccauaaaacaaauuc

SEQ ID NO: 147 - RNA, 30 nt, Artificial Sequence
  Oligonucleotide
cucuuuuccagguucaagugggauacuagc

SEQ ID NO: 148 - RNA, 20 nt, Artificial Sequence
  Oligonucleotide
uccagguucaagugggauac

SEQ ID NO: 149 - RNA, 30 nt, Artificial Sequence
  Oligonucleotide
uauucuuuuguucuucuagccuggagaaag

SEQ ID NO: 150 - RNA, 20 nt, Artificial Sequence
  Oligonucleotide
uuaccuugacuugcucaagc

SEQ ID NO: 151 - RNA, 25 nt, Artificial Sequence
  Oligonucleotide
gcucuucugggcuuaugggagcacu

SEQ ID NO: 152 - RNA, 21 nt, Artificial Sequence
  Oligonucleotide
uuccaccaguaacugaaacag

SEQ ID NO: 153 - RNA, 27 nt, Artificial Sequence
  Oligonucleotide
cuuccacucagagcucagaucuucuaa

SEQ ID NO: 154 - RNA, 30 nt, Artificial Sequence
  Oligonucleotide
uguucagggcaugaacucuuguggauccuu

SEQ ID NO: 155 - RNA, 20 nt, Artificial Sequence
  Oligonucleotide
gcaugaacucuuguggaucc

SEQ ID NO: 156 - RNA, 20 nt, Artificial Sequence
  Oligonucleotide
ccaggguacuacuuacauua

SEQ ID NO: 157 - RNA, 21 nt, Artificial Sequence
  Oligonucleotide
aucgugugucacagcauccag

SEQ ID NO: 158 - RNA, 29 nt, Artificial Sequence
  Oligonucleotide
ccacucagagcucagaucuucuaacuucc

SEQ ID NO: 159 - RNA, 25 nt, Artificial Sequence
  Oligonucleotide
gggauccaguauacuuacaggcucc

SEQ ID NO: 160 - RNA, 27 nt, Artificial Sequence
  Oligonucleotide
accuuuauccacuggagauuugucugc

SEQ ID NO: 161 - RNA, 24 nt, Artificial Sequence
  Oligonucleotide
uucuguccaagcccgguugaaauc

SEQ ID NO: 162 - RNA, 21 nt, Artificial Sequence
  Oligonucleotide
cacccaccaucacccucugug

SEQ ID NO: 163 - RNA, 22 nt, Artificial Sequence
  Oligonucleotide
aucaucucguugauauccucaa

SEQ ID NO: 164 - RNA, 30 nt, Artificial Sequence
  Oligonucleotide
acaucaaggaagauggcauuucuaguuugg

SEQ ID NO: 165 - RNA, 25 nt, Artificial Sequence
  Oligonucleotide
acaucaaggaagauggcauuucuag

SEQ ID NO: 166 - RNA, 30 nt, Artificial Sequence; identical to SEQ ID NO: 1
  Oligonucleotide

SEQ ID NO: 167 - RNA, 26 nt, Artificial Sequence
  Oligonucleotide
accagaguaacagucugaguaggagc

SEQ ID NO: 168 - RNA, 36 nt, Artificial Sequence
  Oligonucleotide
aucauuuuuucucauaccuucugcuaggagcuaaaa

SEQ ID NO: 169 - RNA, 25 nt, Artificial Sequence
  Oligonucleotide
aucauuuuuucucauaccuucugcu

SEQ ID NO: 170 - RNA, 23 nt, Artificial Sequence
  Oligonucleotide
cucauaccuucugcuugaugauc

SEQ ID NO: 171 - RNA, 30 nt, Artificial Sequence
  Oligonucleotide
uccaacuggggacgccucuguuccaaaucc

SEQ ID NO: 172 - RNA, 21 nt, Artificial Sequence
  Oligonucleotide
acuggggacgccucuguucca

SEQ ID NO: 173 - RNA, 20 nt, Artificial Sequence
  Oligonucleotide
ccguaaugauuguucuagcc

SEQ ID NO: 174 - RNA, 21 nt, Artificial Sequence
  Oligonucleotide
uccugcauuguugccuguaag

SEQ ID NO: 175 - RNA, 20 nt, Artificial Sequence
  Oligonucleotide
uguuaaaaaacuuacuucga

SEQ ID NO: 176 - RNA, 20 nt, Artificial Sequence
  Oligonucleotide
aucccacugauucugaauuc

SEQ ID NO: 177 - RNA, 22 nt, Artificial Sequence
  Oligonucleotide
uuggcucuggccuguccuaaga

SEQ ID NO: 178 - RNA, 27 nt, Artificial Sequence
  Oligonucleotide
uguauagggacccuccuuccaugacuc

SEQ ID NO: 179 - RNA, 25 nt, Artificial Sequence
  Oligonucleotide
cugaagguguucuuguacuucaucc

SEQ ID NO: 180 - RNA, 24 nt, Artificial Sequence
  Oligonucleotide
cuguugccuccgguucugaaggug

SEQ ID NO: 181 - RNA, 31 nt, Artificial Sequence
  Oligonucleotide
cauucaacuguugccuccgguucugaaggug

SEQ ID NO: 182 - RNA, 25 nt, Artificial Sequence
  Oligonucleotide
cauucaacuguugccuccgguucug

SEQ ID NO: 183 - RNA, 25 nt, Artificial Sequence
  Oligonucleotide
gauucugaauucuuucaacuagaau

SEQ ID NO: 184 - RNA, 22 nt, Artificial Sequence
  Oligonucleotide
auucuuucaacuagaauaaaag

SEQ ID NO: 185 - RNA, 27 nt, Artificial Sequence
  Oligonucleotide
gguaucuuugauacuaaccuugguuuc

SEQ ID NO: 186 - RNA, 21 nt, Artificial Sequence
  Oligonucleotide
uacuaaccuugguuucuguga

SEQ ID NO: 187 - RNA, 25 nt, Artificial Sequence
  Oligonucleotide
cuaaccuugguuucugugauuuucu

SEQ ID NO: 188 - RNA, 31 nt, Artificial Sequence
  Oligonucleotide
acgaugucaguacuuccaauauucacuaaau

SEQ ID NO: 189 - RNA, 25 nt, Artificial Sequence
  Oligonucleotide
cgaugucaguacuuccaauauucac

SEQ ID NO: 190 - RNA, 31 nt, Artificial Sequence
  Oligonucleotide
auuuccaucuacgaugucaguacuuccaaua

SEQ ID NO: 191 - RNA, 31 nt, Artificial Sequence
  Oligonucleotide
uuaugauuuccaucuacgaugucaguacuuc

SEQ ID NO: 192 - RNA, 27 nt, Artificial Sequence
  Oligonucleotide
ccaauauucacuaaaucaaccuguuaa

SEQ ID NO: 193 - RNA, 25 nt, Artificial Sequence
  Oligonucleotide
caucaggauucuuaccugccagugg

SEQ ID NO: 194 - RNA, 18 nt, Artificial Sequence
  Oligonucleotide
accugccaguggaggauu

SEQ ID NO: 195 - RNA, 30 nt, Artificial Sequence
  Oligonucleotide
caggauucuuaccugccaguggaggauuau

SEQ ID NO: 196 - RNA, 31 nt, Artificial Sequence
  Oligonucleotide
cuuaccugccaguggaggauuauauuccaaa

SEQ ID NO: 197 - RNA, 18 nt, Artificial Sequence
  Oligonucleotide
accauucaucaggauucu

SEQ ID NO: 198 - RNA, 24 nt, Artificial Sequence
  Oligonucleotide
ugucucaguaaucuucuuaccuau

SEQ ID NO: 199 - RNA, 24 nt, Artificial Sequence
  Oligonucleotide
ucuuaccuaugacuauggaugaga

SEQ ID NO: 200 - RNA, 25 nt, Artificial Sequence
  Oligonucleotide
cacuauuccagucaaauaggucugg

SEQ ID NO: 201 - RNA, 23 nt, Artificial Sequence
  Oligonucleotide
ugcauguuccagucguugugugg

SEQ ID NO: 202 - RNA, 21 nt, Artificial Sequence
  Oligonucleotide
ggccuaaaacacauacacaua

SEQ ID NO: 203 - RNA, 25 nt, Artificial Sequence
  Oligonucleotide
auuuaccaaccuucaggaucgagua

SEQ ID NO: 204 - RNA, 21 nt, Artificial Sequence
  Oligonucleotide
gauaggugguaucaacaucug

SEQ ID NO: 205 - RNA, 20 nt, Artificial Sequence
  Oligonucleotide
ggugguaucaacaucuguaa

SEQ ID NO: 206 - RNA, 24 nt, Artificial Sequence
  Oligonucleotide
gauaggugguaucaacaucuguaa

SEQ ID NO: 207 - RNA, 25 nt, Artificial Sequence
  Oligonucleotide
gauaggugguaucaacaucuguaag

SEQ ID NO: 208 - RNA, 20 nt, Artificial Sequence
  Oligonucleotide
guaucaacaucuguaagcac

SEQ ID NO: 209 - RNA, 21 nt, Artificial Sequence
  Oligonucleotide
caggagcuuccaaaugcugca

SEQ ID NO: 210 - RNA, 29 nt, Artificial Sequence
  Oligonucleotide
cuugucuucaggagcuuccaaaugcugca

SEQ ID NO: 211 - DNA, 30 bp, Artificial Sequence
  Oligonucleotide
ctccaacatcaaggaagatggcatttctag

SEQ ID NO: 212 - RNA, 20 nt, Artificial Sequence
  Oligonucleotide
ucaaggaagauggcauuucu

SEQ ID NO: 213 - DNA, 20 bp, Artificial Sequence
  Oligonucleotide
tcaaggaagatggcatttct

SEQ ID NO: 214 - DNA, 25 bp, Artificial Sequence
  Oligonucleotide
gttgcctccggttctgaaggtgttc

SEQ ID NO: 215 - RNA, 25 nt, Artificial Sequence
  Oligonucleotide
guugccuccgguucugaagguguuc

SEQ ID NO: 216 - RNA, 20 nt, Artificial Sequence
  Oligonucleotide
ucaaggaagauggcauuucu

SEQ ID NO: 217 - DNA, 22 bp, Artificial Sequence
  Oligonucleotide
caatgccatcctggagttcctg

SEQ ID NO: 218 - DNA, 18 bp, Artificial Sequence
  Oligonucleotide
  misc_feature (16)..(16): RNA
cgctgcccaatgccaucc

SEQ ID NO: 219 - DNA, 22 bp, Artificial Sequence
  PCR Primer
cagaattctgccaattgctgag

SEQ ID NO: 220 - DNA, 20 bp, Artificial Sequence
  PCR Primer
ttcttcagcttgtgtcatcc

SEQ ID NO: 221 - DNA, 22 bp, Artificial Sequence
  PCR Probe
cttccgtctccatcaatgaact

SEQ ID NO: 222 - DNA, 20 bp, Artificial Sequence
  PCR Probe
gcttatgttgccacctctga

SEQ ID NO: 223 - DNA, 24 bp, Artificial Sequence
  PCR Probe
cagagcccctatccttcacagcat

SEQ ID NO: 224 - DNA, 25 bp, Artificial Sequence
  PCR Primer
aaagcaaactctctgtaccttatct

SEQ ID NO: 225 - DNA, 23 bp, Artificial Sequence
  PCR Primer
catcaacttcagccatccatttc

SEQ ID NO: 226 - DNA, 29 bp, Artificial Sequence
  PCR Probe
tgtgattctgtaatttcccgagtctctcc

SEQ ID NO: 227 - DNA, 23 bp, Artificial Sequence
  PCR Primer
gctgctgtggttatctcctatta

SEQ ID NO: 228 - DNA, 23 bp, Artificial Sequence
  PCR Primer
cctatgtacatcgttctgcttct

SEQ ID NO: 229 - DNA, 21 bp, Artificial Sequence
  PCR Primer
gcagtaacattgagccaagtg

SEQ ID NO: 230 - DNA, 21 bp, Artificial Sequence
  PCR Primer
tccagtctcatccagtctagg

SEQ ID NO: 231 - DNA, 24 bp, Artificial Sequence
  PCR Probe
aataagccagagatcgaagcggcc

SEQ ID NO: 232 - DNA, 19 bp, Artificial Sequence
  PCR Probe
tgagtggaaggcggtaaac

SEQ ID NO: 233 - DNA, 19 bp, Artificial Sequence
  PCR Primer
cgcctctgttccaaatcct

SEQ ID NO: 234 - DNA, 25 bp, Artificial Sequence
  PCR Probe
ccactattggagcctgcaacaatgc

SEQ ID NO: 235 - DNA, 21 bp, Artificial Sequence
  PCR Primer
ccagcaatcaagaggctagaa

SEQ ID NO: 236 - DNA, 20 bp, Artificial Sequence
  PCR Primer
agaagtttcagggccaagtc

SEQ ID NO: 237 - DNA, 24 bp, Artificial Sequence
  PCR Probe
tcattacggatcgaacagttggcc

SEQ ID NO: 238 - DNA, 20 bp, Artificial Sequence
  Oligonucleotide
  misc_feature (1)..(19): RNA
augauguuugaaacuauutt

SEQ ID NO: 239 - DNA, 21 bp, Artificial Sequence
  Oligonucleotide
  misc_feature (1)..(19): RNA
aauaguuucaaacaucauctt

SEQ ID NO: 240 - RNA, 19 nt, Artificial Sequence
  Oligonucleotide
accugaucauuauagauaa

SEQ ID NO: 241 - DNA, 21 bp, Artificial Sequence
  Oligonucleotide
  misc_feature (2)..(19): RNA
tuaucuauaaugaucaggutt

SEQ ID NO: 242 - DNA, 25 bp, Artificial Sequence
  Oligonucleotide
ggccaaacctcggcttacctgaaat

SEQ ID NO: 243 - RNA, 25 nt, Artificial Sequence
  Oligonucleotide
ggccaaaccucggcuuaccugaaau